SEQUENCE LISTING (29 sequences)

SEQ ID NO: 1 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 2 - protein, 132 aa, synthetic construct
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 3 - protein, 133 aa, synthetic construct
APTSSSTKKTQLQLEHLLLDLQMILNGINNYKNPCLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 4 - protein, 133 aa, synthetic construct
APTSSSTKKTQLQLEHLLLDLQMILNGINNYKNPKLTCMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 5 - protein, 133 aa, synthetic construct
APTSSSTKKTQLQLEHLLLDLQMILNGINNYKNPKLTRCLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 6 - protein, 133 aa, synthetic construct
APTSSSTKKTQLQLEHLLLDLQMILNGINNYKNPKLTRMLCFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 7 - protein, 133 aa, synthetic construct
APTSSSTKKTQLQLEHLLLDLQMILNGINNYKNPKLTRMLTCKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 8 - protein, 133 aa, synthetic construct
APTSSSTKKTQLQLEHLLLDLQMILNGINNYKNPKLTRMLTFCFYMPKKATELKHLQCLEEELKPLEEVLNLAQSKNFHLRPRDLISNINVIVLELKGSETTFMCEYADETATIVEFLNRWITFSQSIISTLT

SEQ ID NO: 9 - protein, 133 aa, synthetic construct
APTSSSTKKTQLQLEHLLLDLQMILNGINNYKNPKLTRMLTFKCYMPKKATELKHLQCLEEELKPLEEVLNLAQSKNFHLRPRDLISNINVIVLELKGSETTFMCEYADETATIVEFLNRWITFSQSIISTLT

SEQ ID NO: 10 - protein, 133 aa, synthetic construct
APTSSSTKKTQLQLEHLLLDLQMILNGINNYKNPKLTRMLTFKFCMPKKATELKHLQCLEEELKPLEEVLNLAQSKNFHLRPRDLISNINVIVLELKGSETTFMCEYADETATIVEFLNRWITFSQSIISTLT

SEQ ID NO: 11 - protein, 133 aa, synthetic construct
APTSSSTKKTQLQLEHLLLDLQMILNGINNYKNPKLTRMLTFKFYMPKKATELKHLQCLEECLKPLEEVLNLAQSKNFHLRPRDLISNINVIVLELKGSETTFMCEYADETATIVEFLNRWITFSQSIISTLT

SEQ ID NO: 12 - protein, 133 aa, synthetic construct
APTSSSTKKTQLQLEHLLLDLQMILNGINNYKNPKLTRMLTFKFYMPKKATELKHLQCLEEELKCLEEVLNLAQSKNFHLRPRDLISNINVIVLELKGSETTFMCEYADETATIVEFLNRWITFSQSIISTLT

SEQ ID NO: 13 - protein, 133 aa, synthetic construct
APTSSSTKKTQLQLEHLLLDLQMILNGINNYKNPKLTRMLTFKFYMPKKATELKHLQCLEEELKPLECVLNLAQSKNFHLRPRDLISNINVIVLELKGSETTFMCEYADETATIVEFLNRWITFSQSIISTLT

SEQ ID NO: 14 - protein, 133 aa, synthetic construct
APTSSSTKKTQLQLEHLLLDLQMILNGINNYKNPKLTRMLTFKFYMPKKATELKHLQCLEEELKPLEECLNLAQSKNFHLRPRDLISNINVIVLELKGSETTFMCEYADETATIVEFLNRWITFSQSIISTLT

SEQ ID NO: 15 - protein, 133 aa, synthetic construct
APTSSSTKKTQLQLEHLLLDLQMILNGINNYKNPKLTRMLTFKFYMPKKATELKHLQCLEEELKPLEEVLNCAQSKNFHLRPRDLISNINVIVLELKGSETTFMCEYADETATIVEFLNRWITFSQSIISTLT

SEQ ID NO: 16 - protein, 132 aa, synthetic construct
PTSSSTKKTQLQLEHLLLDLQMILNGINNYKNPCLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 17 - protein, 132 aa, synthetic construct
PTSSSTKKTQLQLEHLLLDLQMILNGINNYKNPKLTCMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 18 - protein, 132 aa, synthetic construct
PTSSSTKKTQLQLEHLLLDLQMILNGINNYKNPKLTRCLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 19 - protein, 132 aa, synthetic construct
PTSSSTKKTQLQLEHLLLDLQMILNGINNYKNPKLTRMLCFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 20 - protein, 132 aa, synthetic construct
PTSSSTKKTQLQLEHLLLDLQMILNGINNYKNPKLTRMLTCKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 21 - protein, 132 aa, synthetic construct
PTSSSTKKTQLQLEHLLLDLQMILNGINNYKNPKLTRMLTFCFYMPKKATELKHLQCLEEELKPLEEVLNLAQSKNFHLRPRDLISNINVIVLELKGSETTFMCEYADETATIVEFLNRWITFSQSIISTLT

SEQ ID NO: 22 - protein, 132 aa, synthetic construct
PTSSSTKKTQLQLEHLLLDLQMILNGINNYKNPKLTRMLTFKCYMPKKATELKHLQCLEEELKPLEEVLNLAQSKNFHLRPRDLISNINVIVLELKGSETTFMCEYADETATIVEFLNRWITFSQSIISTLT

SEQ ID NO: 23 - protein, 132 aa, synthetic construct
PTSSSTKKTQLQLEHLLLDLQMILNGINNYKNPKLTRMLTFKFCMPKKATELKHLQCLEEELKPLEEVLNLAQSKNFHLRPRDLISNINVIVLELKGSETTFMCEYADETATIVEFLNRWITFSQSIISTLT

SEQ ID NO: 24 - protein, 132 aa, synthetic construct
PTSSSTKKTQLQLEHLLLDLQMILNGINNYKNPKLTRMLTFKFYMPKKATELKHLQCLEECLKPLEEVLNLAQSKNFHLRPRDLISNINVIVLELKGSETTFMCEYADETATIVEFLNRWITFSQSIISTLT

SEQ ID NO: 25 - protein, 132 aa, synthetic construct
PTSSSTKKTQLQLEHLLLDLQMILNGINNYKNPKLTRMLTFKFYMPKKATELKHLQCLEEELKCLEEVLNLAQSKNFHLRPRDLISNINVIVLELKGSETTFMCEYADETATIVEFLNRWITFSQSIISTLT

SEQ ID NO: 26 - protein, 132 aa, synthetic construct
PTSSSTKKTQLQLEHLLLDLQMILNGINNYKNPKLTRMLTFKFYMPKKATELKHLQCLEEELKPLECVLNLAQSKNFHLRPRDLISNINVIVLELKGSETTFMCEYADETATIVEFLNRWITFSQSIISTLT

SEQ ID NO: 27 - protein, 132 aa, synthetic construct
PTSSSTKKTQLQLEHLLLDLQMILNGINNYKNPKLTRMLTFKFYMPKKATELKHLQCLEEELKPLEECLNLAQSKNFHLRPRDLISNINVIVLELKGSETTFMCEYADETATIVEFLNRWITFSQSIISTLT

SEQ ID NO: 28 - protein, 132 aa, synthetic construct
PTSSSTKKTQLQLEHLLLDLQMILNGINNYKNPKLTRMLTFKFYMPKKATELKHLQCLEEELKPLEEVLNCAQSKNFHLRPRDLISNINVIVLELKGSETTFMCEYADETATIVEFLNRWITFSQSIISTLT

SEQ ID NO: 29 - protein, 133 aa, Homo sapiens [sequence omitted]

(19) United States
(12) Patent Application Publication
(10) Pub. No.: US 2026/0315607 A1
(43) Pub. Date: Oct. 15, 2026

(54) Novel Cancer Treatments With IL-2 Conjugates

(71) Applicant: Ascendis Pharma Oncology Division A/S, HellerupHellerup (DK)

(72) Inventors: Vibeke MILLER BREINHOLT, Hellerup (DK); Stina SINGEL, Palo Alto, CA (US)

(21) Appl. No.: 19/481,511

(22) PCT Filed: May 8, 2024
(86) PCT No.: PCT/EP2024/062703

§ 371 Date: Nov. 4, 2025

Related U.S. Application Data
(60) Provisional application No. 63/501,115, filed on May 9, 2023.
(60) Provisional application No. 63/578,017, filed on Aug. 22, 2023.
(60) Provisional application No. 63/591,684, filed on Oct. 19, 2023.

(51) Int. Cl. A61K 47/60; A61K 9/00; A61K 45/06; A61P 35/00; C07K 14/55

(52) U.S. Cl. CPC A61K 47/60; A61K 9/0019; A61K 45/06; A61P 35/00; C07K 14/55

(57) ABSTRACT

The present invention relates to an IL-2 conjugate or a pharmaceutically acceptable salt thereof for use in the treatment of cancer, wherein the IL-2 conjugate is administered in a dose ranging from 80 μg IL-2/kg to 160 μg IL-2/kg and wherein the IL-2 conjugate is of formula (I); and to related aspects.

The present invention relates to an IL-2 conjugate or a pharmaceutically acceptable salt thereof for use in the treatment of cancer, wherein the IL-2 conjugate is administered in a dose ranging from 80 μg IL-2/kg to 160 μg IL-2/kg and wherein the IL-2 conjugate is of formula (I); and to related aspects.

In healthy humans, the immune system can often discriminate between healthy cells and cancerous cells. Upon identifying a given cell as cancerous, the immune system typically eliminates it. However, when the immune system is compromised from e.g., acute or chronic defects or is overwhelmed, cancers can develop resulting from a compromised immune system's inability to differentiate, and then eliminate, cancer cells. In a patient suffering from cancer, administration of an immunomodulatory protein to the patient may help activate that patient's immune system so that the immune system's ability to eliminate cancer cells is enhanced. In this way, the cancer may be slowed from further growth, suppressed from potential spread or even eliminated.

One such immunomodulatory protein used in the treatment of patients suffering from certain cancers is interleukin-2 (IL-2). IL-2 is a naturally occurring cytokine that has activity as both a stimulator of natural killer cells (NK cells) and T-cells leading to proliferation and functional activity. IL-2 plays a central role in the generation, differentiation, survival, and homeostasis of immune effector cells. IL-2 is synthesized by activated CD4+ helper T cells, and through differential receptor interaction IL-2 can modulate the immune response towards immunity or tolerance.

IL-2 acts by binding to IL-2 receptors (IL-2R). Association of the α- (CD25), β- (CD122) and common γ- (γc, CD132) subunits results in the trimeric high-affinity IL-2Rα/β/γ. The dimeric intermediate affinity IL-2Rβ/γ consists of the β- and γ-subunits and binds IL-2 with 50-fold lower affinity. CD25 is not required for IL-2 signaling but confers the high affinity binding of the trimeric receptor, whereas the β- and γ-subunits mediate signal transduction. IL-2Rβ/γ is expressed on NK cells, monocytes, macrophages and resting CD4+ and CD8+ T cells, while IL-2Rα/β/γ is transiently induced on activated T and NK cells and is constitutively expressed on T regulatory cells. The ability of IL-2 to expand and activate innate and adaptive effector cells is the basis of its antitumor activity.

In patients, IL-2 can stimulate antitumor efficacy, characterized by increases in cytotoxic lymphocytes, including effector T and NK cells, when given at high-doses (i.e., 600 000-720 000 IU/kg body weight three times daily for up to 14 doses per cycle in humans). Presumably during this therapy all T cells are stimulated by IL-2 after high-doses are administered and when the therapy cycle ends, and as IL-2 levels drop at some point, T regulatory (Treg) cells expressing IL-2Rα/β/γ will outcompete effector T cells expressing IL-2Rβ/γ for the remaining wild type IL-2.

Aldesleukin, recombinant human IL-2, was the first cancer immunotherapy approved by the FDA in 1992. With use of appropriate supportive care, aldesleukin has demonstrated complete cancer regression in about 10% of patients treated for metastatic melanoma and renal cancer.

Approximately 70% of patients with complete responses have been cured, maintaining complete regression for more than 25 years after initial treatment.

Based on its antitumor efficacy, high-dose aldesleukin has been approved for treatment of patients with metastatic renal cell carcinoma and malignant melanoma. Aldesleukin is currently administered at a dose of 37 μg/kg every 8 hours via a 15-minute intravenous (IV) infusion for a maximum of 14 doses and the schedule may be repeated for another 14 doses after 9 days of rest.

However, its antitumor immunity is dose limited by severe cardiovascular, pulmonary, hepatic, gastrointestinal, neurologic and hematological side effects, such that it is only given to patients at specialized centers. Also, once administered IL-2 levels fall below the levels required for IL-2β/γ activity, activation of Tregs expressing high affinity IL-2Rα/β/γ will be favored, which can limit anti-tumor immunity. Thus, it is essential that the dose of IL-2 is high enough to maintain IL-2 levels above the required threshold while ensuring tolerable side-effects.

Preclinical experiments showed that IL-2-induced pulmonary edema (as a model of vascular leak syndrome) can be caused by interaction of IL-2 with CD25 on lung endothelial cells and that it can be abrogated by a CD25 blocking antibody, genetic disruption or the use of IL-2-antibody complexes. Another proposed mechanism by which IL-2 induces vascular leak syndrome involves activation of Eosinophils as these cells can express IL-2Rα/β/γ and IL-2 therapy in patients is associated with elevated systemic Eosinophils and IL-5 levels.

Especially when long-acting IL-2 is administered, it is essential that the dose is not too high, because if it is too high, the potentially severe side effects last longer than for example with aldesleukin.

Thus, there is a need for a more efficacious and safer treatment with IL-2 conjugates and it is an object of the present invention to at least partially overcome the shortcomings of current cancer treatment with aldesleukin and the shortcomings observed for IL-2 conjugates that have been tested in the clinic for the treatment of cancer, such as NKTR-214 and THOR-707.

In a first aspect the present invention relates to an IL-2 conjugate or a pharmaceutically acceptable salt thereof for use in the treatment of cancer, wherein the IL-2 conjugate is administered in a dose ranging from 80 μg IL-2/kg to 160 μg IL-2/kg and wherein the IL-2 conjugate is of formula (I)

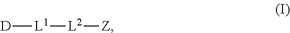

wherein
—D′— is an IL-2 moiety;
   M_{mod} is a first polymeric moiety that is stably conjugated to a side chain of an amino acid residue of —D′—;
—L¹— is a linker moiety covalently and reversibly attached to —D′—;
—L²— is absent or a spacer moiety; and
—Z is a second polymeric moiety.

A dose given as “X μg IL-2/kg of the IL-2 conjugate” means X μg of the IL-2 moiety —D′— present in the IL-2 conjugate, i.e., dosages for the IL-2 conjugate are given with respect to —D′— only and exclude other components, such as the first polymeric moiety —M_{mod}, the second polymeric moiety —Z and any moiety —L¹— and —L²— present in the IL-2 conjugate.

In a second aspect the present invention relates to a method of treating a patient having cancer, wherein the method comprises administering to such patient in a dose ranging from 80 μg IL-2/kg to 160 μg IL-2/kg an IL-2 conjugate or a pharmaceutically acceptable salt thereof of formula (I)

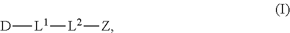

wherein
—D is —D′—M_{mod},
   wherein
   D′— is an IL-2 moiety;
   M_{mod} is a first polymeric moiety that is stably conjugated to a side chain of an amino acid residue of —D′—;
L¹— is a linker moiety covalently and reversibly attached to —D′—;
L²— is absent or a spacer moiety; and
Z is a second polymeric moiety.

In a third aspect the present invention relates to an IL-2 conjugate or a pharmaceutically acceptable salt thereof for use in the manufacture of a medicament for the treatment of cancer, wherein the IL-2 conjugate is administered in a dose ranging from 80 μg IL-2/kg to 160 μg IL-2/kg and wherein the IL-2 conjugate is of formula (I) D-L¹—L²—Z (I),

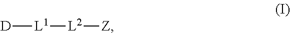

wherein
D is —D′—M_{mod},
   wherein
   D′— is an IL-2 moiety;
   M_{mod} is a first polymeric moiety that is stably conjugated to a side chain of an amino acid residue of —D′—;
L¹— is a linker moiety covalently and reversibly attached to —D′—;
L²— is absent or a spacer moiety; and
Z is a second polymeric moiety.

In a fourth aspect the present invention relates to a unit dosage form comprising a unit dose of an IL-2 conjugate or a pharmaceutically acceptable salt thereof, wherein the unit dosage form comprises 80 μg/kg to 160 μg/kg IL-2 and wherein the IL-2 conjugate is of formula (I)

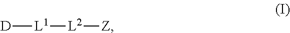

wherein
—D is —D′—M_{mod},
   wherein
   D′— is an IL-2 moiety;
   M_{mod} is a first polymeric moiety that is stably conjugated to a side chain of an amino acid residue of —D′—;
L¹— is a linker moiety covalently and reversibly attached to —D′—;
L²— is absent or a spacer moiety; and
Z is a second polymeric moiety.

It was surprisingly found that a dose ranging from 80 μg IL-2/kg to 160 μg IL-2/kg of the IL-2 conjugate of formula (I) is both safe and efficacious. This was unexpected, because other IL-2 drugs are administered at lower doses. For example, NKTR-214 is an IL-2 conjugate consisting of aldesleukin reversibly conjugated to six PEG chains. The highest NKTR-214 dose tested is 12 μg IL-2/kg, which caused dose-limiting toxicity and the recommended phase 2 dose (RP2D) of NKTR-214 is 6 μg IL-2/kg administered every three weeks (Bentebibel, et al. Cancer Discov. 2019; 9(6):711-721). Another example is THOR-707 (SAR444245), which is an IL-2 conjugate consisting of an IL-2 protein in which the proline at position 65 is replaced with the non-natural amino acid N6-(2-azidoethoxy)-carbonyl-1-lysine (AzK) and which is stably pegylated with a 30 kDa mPEG moiety. THOR-707 was tested in a dose escalation trial up to a dose of 40 μg IL-2/kg every two or three weeks and the RP2D of THOR-707 is 24 μg IL-2/kg every two or three weeks for monotherapy and in combination with pembrolizumab (Falchook, et al. Journal for ImmunoTherapy of Cancer, 2021;9:doi: 10.1 136/jitc-2021-SITC2021.481; Falchook, et al. Annals of Oncology, 2022, 33, S885-S886; Trial Registration NCT04009681).

Within the meaning of the present invention the terms are used as follows.

The term “patient” refers to a subject amenable to treatment or prophylaxis according to the invention, particularly a human subject.

As used herein, the term “dose” refers to the predetermined amount of the drug, such as IL-2, administered at one time to produce a certain degree of biological response in a patient. It is understood that the phrase “at one time” includes that the dose may be administered to the patient in one or more than one administration, which more than one administration may be administered consecutively or in parallel. In the case of consecutive administrations, the time between two administrations may range from 10 seconds to 8 hours, such as from one minute to 4 hours, from two minutes to 2 hours or from 5 minutes to 1 hour.

As used herein, the term “unit dose” means the amount of the drug administered to a patient in a single dose.

A “unit dosage form” refers to a dosage form configured for a single administration to a patient. For example, a unit dosage form can be a single vial or other container, such as a dual-chamber cartridge, an ampoule or a syringe, containing an amount of drug suitable for a single administration. Also an infusion bag may be a suitable container.

As used herein, the term “dosage regimen” is the combination of dose, and frequency with which a drug is administered. A dosage regimen can also include a route of administration (e.g., intravenous) and/or duration of administration.

As used herein, the term “therapeutically effective amount” means an amount sufficient to cure, alleviate or partially arrest the clinical manifestations of a given disease and its complications. Effective amounts for each purpose will depend on the severity of the disease or injury as well as the weight and general state of the subject. Within the scope of this invention, a “therapeutically effective amount” relates to dosages that achieve a therapeutic effect for an extended period of time, i.e., for at least one day, such as for two days, such as for three days, such as for one week, such as for two weeks, such as for three weeks, such as for four weeks, such as for eight weeks or longer.

As used herein, the term “drug” refers to a substance used in the treatment, cure, prevention or diagnosis of a disease or used to otherwise enhance physical or mental well-being of a patient. If a drug, such as IL-2, is conjugated to another moiety, the moiety of the resulting product that originated from the drug is referred to as “drug moiety”.

Any reference to a biologic drug herein, i.e., to a drug manufactured in, extracted from or semi-synthesized from biological sources, such as a protein drug, also covers biosimilar versions of said drug.

It is understood that the IL-2 conjugates of the present invention are prodrugs.

As used herein the term “prodrug” refers to a drug moiety reversibly and covalently connected to a specialized protective group through a reversible prodrug linker moiety which is a linker moiety comprising a reversible linkage with the drug moiety and wherein the specialized protective group alters or eliminates undesirable properties in the parent molecule. This also includes the enhancement of desirable properties in the drug and the suppression of undesirable properties. The specialized non-toxic protective group may also be referred to as “carrier”, such as for example —Z. A prodrug releases the reversibly and covalently bound drug moiety in the form of its corresponding drug. In other words, a prodrug is a conjugate comprising a drug moiety, which is covalently and reversibly conjugated to a carrier moiety via a reversible linker moiety, such as for example —L¹—, which covalent and reversible conjugation of the carrier to the reversible linker moiety is either directly or through a spacer, such as for example —L²—. The reversible linker may also be referred to as “reversible linker” or “reversible prodrug linker”. Such conjugate may release the formerly conjugated drug moiety in the form of a free drug, in which case the reversible linker or reversible prodrug linker is a traceless linker. It is understood that the free drug released from the conjugates of the present invention is an IL-2 protein, to which a moiety M_{mod} is covalently conjugated.

A “reversible linkage” is a linkage that is degradable, i.e., cleavable, in the absence of enzymes under physiological conditions (aqueous buffer at pH 7.4, 37° C.) with a half-life ranging from 1 hour to three months. A “stable linkage” is a linkage having a half-life under physiological conditions (aqueous buffer at pH 7.4, 37° C.) in the absence of enzymes of more than three months.

As used herein, the term “free form” of a drug means the drug in its unmodified, pharmacologically active form, e.g., after being released from the IL-2 conjugate or pharmaceutically acceptable salt thereof. It is understood that the free drug released from the conjugates of the present invention is an IL-2 protein, to which a moiety M_{mod} is covalently conjugated.

As used herein the term “spacer” refers to a moiety that connects at least two other moieties with each other.

In general, the term “interleukin-2” or “IL-2” refers to all IL-2 proteins, such as to IL-2 proteins from mammalian species, for example from primate species and in particular from human, as well as their variants, analogs, orthologs, homologs, and derivatives and fragments thereof, that are characterized by playing a central role in lymphocyte generation, survival and homeostasis, and also encompasses naturally occurring variants of IL-2, e.g. splice variants or allelic variants. Human wild-type IL-2 has the amino acid sequence of SEQ ID NO:29:

APTSSSTKKTQLQLEHLLLDLQMILNGINNYKNPKLTRMLTFKFY
MPKKATELKHLQCLEEELKPLEEVLNLAQSKNFHLRPRDLISNIN
VIVLELKGSETTFMCEYADETATIVEFLNRWITFCQSIISTLT

As used herein, the term “biased IL-2” refers to a modified IL-2, in which the ratio of the K_{D} of said biased IL-2 to IL-2Rα to the K_{D} of said biased IL-2 to IL-2Rβ is larger than the ratio of the K_{D} of aldesleukin of SEQ ID NO:2 to IL-2Rα to the K_{D} of aldesleukin to IL-2Rβ. This is described by the following formula:

$$\begin{matrix}\frac{{Ratio}_{biased IL - 2}}{{Ratio}_{aldesleukin}} > 1 \\ wherein \\ {Ratio}_{biased IL - 2} = \frac{{K}_{D} biased IL - 2 to IL - 2 R α}{{K}_{D} biased IL - 2 to IL - 2 β} \\ {Ratio}_{aldesleukin} = \frac{{K}_{D} aldesleukin to IL - 2 R α}{{K}_{D} aldesleukin to IL - 2 β}\end{matrix}$$

with
“K_{D} biased IL-2 to IL-2Rα” being the K_{D} of biased IL-2 to IL-2Rα,
“K_{D} biased IL-2 to IL-2Rβ” being the K_{D} of biased IL-2 to IL-2Rβ,
“K_{D} aldesleukin to IL-2Rα” being the K_{D} of aldesleukin to IL-2Rα, and
“K_{D} aldesleukin to IL-2Rβ” being the K_{D} of aldesleukin to IL-2Rβ.

Aldesleukin has the following sequence (SEQ ID NO:2):

PTSSSTKKTQLQLEHLLLDLQMILNGINNYKNPKLTRMLTFKFYM
PKKATELKHLQCLEEELKPLEEVLNLAQSKNFHLRPRDLISNINV
IVLELKGSETTFMCEYADETATIVEFLNRWITFSQSIISTLT

Binding affinity/kinetics needed to determine the K_{D} of biased IL-2 to IL-2Rα, the K_{D} of biased IL-2 to IL-2Rβ, the K_{D} of aldesleukin to IL-2Rα and the K_{D} of aldesleukin to IL-2Rβ may be assessed using surface plasmon resonance (SPR), measured on a Biacore instrument (GE Healthcare) as follows: A human Fc capture surface on a CM5 (or alternatively C1 or CM4) chip is prepared by covalent coating with anti-human Fc antibody or alternatively a protein A chip is used. Next, IL-2Rβ-Fc or IL-2Rα-Fc is immobilized on the chip. To measure the affinity/kinetic constants, serial dilutions of the analytes are made starting at for example between 1 nM and 2 μM or at 30 nM and 500 nM for IL-2 compounds. Analytes are each exposed to the receptor-modified chip for a suitable amount of time, such as for 1 to 30 minutes, which may for example be 2 minutes or may be 3 minutes and are then washed away for a suitable amount of time, such as 2 to 60 minutes, which may for example be 10 minutes. The resulting binding curves from the dilution series are fit to a 1:1 kinetic model to correlate observed response units (R) to the association and dissociation rate constants, kₐ and k_{d}:

$$R = \frac{{k}_{a} {CR}_{max}}{{k}_{a} C + {k}_{d}} \times ( 1 - {e}^{- ( {k}_{a} C + {k}_{d} ) t} )$$

wherein
t is time;
C is the concentration of the analyte; and
Rₘₐₓ is the maximum binding capacity of the surface.

If determined via a kinetic 1:1 model the ratio of the dissociation and association rates provides the equilibrium dissociation constant K_{D}.

Alternatively, the resulting binding curves from the dilution series are fit to a 1:1 steady state interaction model which calculates K_{D} for a 1:1 interaction from a plot of steady-state binding levels (R_{eq}) against analyte concentration (C):

$${R}_{eq} = \frac{C \times {R}_{max}}{{K}_{D} + C}$$

wherein
R_{eq} is the steady-state binding level;
C is the concentration of the analyte; and
Rₘₐₓ is the maximum binding capacity of the surface.

It is understood that not every calculation method may be possible for every biased IL-2 molecule. If, for example, the reactions are too fast, it may not be possible to use a 1:1 kinetic model and a 1:1 steady state interaction model may be used. If, for example, no equilibrium is obtained, it may not be possible to use a 1:1 interaction model and a 1:1 kinetic model may be used.

As used herein, the term “affinity” refers to the strength of the sum of non-covalent interactions between a single binding site of a molecule (such as a receptor) and its binding partner (such as a ligand). Unless indicated otherwise, as used herein, “affinity” refers to intrinsic binding affinity which reflects a 1:1 interaction between members of a binding pair (such as between a receptor and a ligand). The affinity of a molecule X for its partner Y can generally be represented by the equilibrium dissociation constant (K_{D}), which is the ratio of dissociation and association rate constants (k_{d} and kₐ, respectively) measured in a state of equilibrium. Thus, equivalent affinities may comprise different rate constants, as long as the ratio of the rate constants remains the same. Affinity can be measured by well-established methods known in the art, including those described herein.

As used herein, the terms “α-subunit of the IL-2 receptor” and “IL-2Rα” refer to human CD25.

As used herein, the terms “β-subunit of the IL-2 receptor” and “IL-2Rβ” refer to human CD122.

As used herein, the terms “γ-subunit of the IL-2 receptor” and “IL-2Rγ” refer to human CD132.

“Treating” refers to curing, reducing or inhibiting further deterioration of at least one sign or symptom of a disease or stabilizing at least one sign or symptom of disease. Treating can be determined by comparing sign(s) and symptom(s) in an individual patient before (baseline) and after receiving treatment or by comparing a population of treated patients to a control population as in a clinical trial or trial with an animal model.

As used herein, the term “PD-L1 positive” or “PD-L1 expression positive”, relating to cell surface programmed cell death ligand 1 (PD-L1) expression, refers to tumor tissue, for example a test tissue sample, in which greater than or equal to 1 percent of all tumor cells in the tumor tissue express PD-L1.

As used herein, the term “PD-L1 negative” or “PD-L1 expression negative” refers to tumor tissue, for example a test tissue sample, in which less than 1% of all tumor cells in the tumor tissue express PD-L1.

As used herein, the term “PD-L1 expression” means any detectable level of expression of PD-L1 protein on the cell surface or of PD-L1 messenger-RNA (mRNA) within a cell or tissue. PD-L1 protein expression may be detected with a diagnostic PD-L1 antibody in an immunohistochemistry (IHC) assay of a tumor tissue section or by flow cytometry.

Alternatively, PD-L1 protein expression by tumor cells may be detected by positron emission tomography (PET) imaging, using a binding agent, such as an antibody fragment, affibody and the like, that specifically binds to PD-L1. Techniques for detecting and measuring PD-L1 mRNA expression include real-time polymerase chain reaction (RT-PCR) and quantitative RT-PCR.

As used herein, the term “PD-1 positive” or “PD-1 expression positive”, relating to programmed cell death protein 1 (PD-1) expression, refers to tumor tissue, for example a test tissue sample, that is scored as expressing PD-1 based on the proportion, for example percentage, of cells, for example immune cells such as tumor-infiltrating lymphocytes such as CD8+ T cells, expressing PD-1.

As used herein, the term, “PD-1 negative” or “PD-1 expression negative” refers to tumor tissue, for example a test tissue sample, that is not scored as expressing PD-1.

As used herein, the term “PD-1 expression” means any detectable level of expression of PD-1 protein on the cell surface or of PD-1 mRNA within a cell or tissue. PD-1 protein expression may be detected with a diagnostic PD-1 antibody in an IHC assay of a tumor tissue section or by flow cytometry. Alternatively, PD-1 protein expression by immune cells may be detected by PET imaging, using a binding agent, such as an antibody fragment, affibody and the like, that specifically binds to PD-1. Techniques for detecting and measuring PD-1 mRNA expression include RT-PCR and real-time quantitative RT-PCR.

As used herein, the term “intratumoral administration” refers to a mode of administration, in which the drug is administered directly into tumor tissue. The term “intratumoral administration” may in certain embodiments also refer to administration pre- or post-resection into or onto the tumor bed. When tumor boundary is not well defined, it is also understood that intratumoral administration includes administration to tissue adjacent to the tumor cells (“peri-tumoral administration”). Exemplary tumors for intratumoral administration are solid tumors and lymphomas, which are disclosed in more detail elsewhere herein. Administration may occur via injection.

“Prophylaxis” refers to preventing, reducing risk of or delaying onset of at least one sign or symptom of disease in a population of patients (or animal models) at risk of the disease administered a drug according to the invention compared to a control population of patients (or animal models) at risk of the disease not treated with a drug according to the invention. The amount is also considered prophylactically effective if an individual treated patient achieves an outcome more favourable than the mean outcome in a control population of comparable patients not treated by methods of the invention.

As used herein, the term “reagent” means a chemical compound, which comprises at least one functional group for reaction with the functional group of another chemical compound or drug.

It is understood that a drug comprising a functional group (such as a primary or secondary amine or hydroxyl functional group) is also a reagent.

As used herein, the term “moiety” means a part of a molecule, which lacks one or more atom(s) compared to the corresponding reagent. If, for example, a reagent of the formula “H—X—H” reacts with another reagent and becomes part of the reaction product, the corresponding moiety of the reaction product has the structure “H—X—” or “—X—”, whereas each “-” indicates attachment to another moiety. Accordingly, a drug moiety is released from a reversible linkage as a drug.

It is understood that if the sequence or chemical structure of a group of atoms is provided which group of atoms is attached to two moieties or is interrupting a moiety, said sequence or chemical structure can be attached to the two moieties in either orientation, unless explicitly stated otherwise. For example, a moiety “—C(O)N(R¹)—” can be attached to two moieties or interrupting a moiety either as “—C(O)N(R¹)—” or as “—N(R¹)C(O)—”. Similarly, a moiety

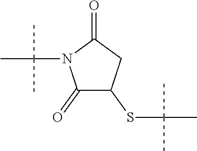

can be attached to two moieties or can interrupt a moiety either as

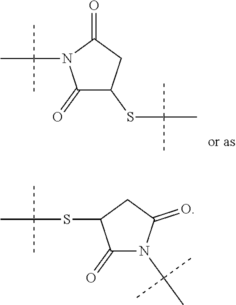

The term “substituted” as used herein means that one or more —H atom(s) of a molecule or moiety are replaced by a different atom or a group of atoms, which are referred to as “substituent”.

As used herein, the term “substituent” refers in certain embodiments to a moiety selected from the group consisting of halogen, —CN, —COOR^{x1}, —OR^{x1}, —C(O)R^{x1}, —C(O)N(R^{x1}R^{x1a}), —S(O)₂N(R^{x1}R^{x1a}) —S(O)N(R^{x1}R^{x1a}), —S(O)₂R^{x1}, —S(O)R^{x1}, —N(R^{x1})S(O)₂N(R^{x1a}R^{x1b}), —SR^{x1}, —N(R^{x1}R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a}R^{x1b}), —OC(O)N(R^{x1}R^{x1a}), —T⁰, C₁₋₅₀ alkyl, C₂₋₅₀ alkenyl, and C₂₋₅₀ alkynyl; wherein —T⁰, C₁₋₅₀ alkyl, C₂₋₅₀ alkenyl, and C₂₋₅₀ alkynyl are optionally substituted with one or more —R^{x2}, which are the same or different and wherein C₁₋₅₀ alkyl, C₂₋₅₀ alkenyl, and C₂₋₅₀ alkynyl are optionally interrupted by one or more groups selected from the group consisting of —T⁰—, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—; —R^{x1}, —R^{x1a}, —R^{x1b} are independently of each other selected from the group consisting of —H, —T⁰, C₁₋₅₀ alkyl, C₂₋₅₀ alkenyl, and C₂₋₅₀ alkynyl; wherein —T⁰—, C₁₋₅₀ alkyl, C₂₋₅₀ alkenyl, and C₂₋₅₀ alkynyl are optionally substituted with one or more —R^{x2}, which are the same or different and wherein C₁₋₅₀ alkyl, C₂₋₅₀ alkenyl, and C₂₋₅₀ alkynyl are optionally interrupted by one or more groups selected from the group consisting of —T⁰—, —C(O)O—, —O—, —C(O)—, —C(O)N(R^{x3})—, —S(O)₂N(R^{x3})—, —S(O)N(R^{x3})—; —S(O)₂—, —S(O)—, —N(R^{x3})S(O)₂N(R^{x3a})—, —S——N(R^{x3})—, —OC(OR^{x3})(R^{x3a})—, —N(R^{x3})C(O)N(R^{x3a})—, and —OC(O)N(R^{x3})—;

each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2} which are the same or different;

each —R^{x2} is independently selected from the group consisting of halogen, —CN, oxo (═O), —COOR^{x4}, —OR^{x4}, —C(O)R^{x4}, —C(O)N(R^{x4}R^{x4a}), —S(O)₂N(R^{x4}R^{x4a}), —S(O)N(R⁴R^{x4a}), —S(O)₂R^{x4}, —S(O)R^{x4}, —N(R^{x4})S(O)₂N(R^{x4a}R^{x4b}), —SR^{x4}, —N(R^{x4}R^{x4a}), —NO₂, —OC(O)R^{x4}, —N(R^{x4})C(O)R^{x4a}, —N(R^{x4})S(O)₂R^{x4a}, —N(R^{x4})S(O)R^{x4a}, —N(R^{x4})C(O)OR^{x4a}, —N(R^{x4})C(O)N(R^{x4a}R^{x4b}), —OC(O)N(R^{x4}R^{x4a}), and C₁₋₆ alkyl; wherein C₁₋₆ alkyl is optionally substituted with one or more halogen, which are the same or different;

each —R^{x3}, —R^{x3a}, —R^{x4}, —R^{x4a}, —R^{x4b} is independently selected from the group consisting of —H and C₁₋₆ alkyl; wherein C₁₋₆ alkyl is optionally substituted with one or more halogen, which are the same or different.

In certain embodiments a maximum of 6 —H atoms of an optionally substituted molecule are independently replaced by a substituent, e.g. 5 —H atoms are independently replaced by a substituent, 4 —H atoms are independently replaced by a substituent, 3 —H atoms are independently replaced by a substituent, 2 —H atoms are independently replaced by a substituent or 1 —H atom is replaced by a substituent.

The term “peptide” as used herein refers to a chain of at least 2 and up to and including 50 amino acid monomer moieties linked by peptide (amide) linkages. The term “peptide” also includes peptidomimetics, such as D-peptides, peptoids or beta-peptides, and covers such peptidomimetic chains with up to and including 50 monomer moieties.

As used herein, the term “protein” refers to a chain of more than 50 amino acid monomer moieties, which may also be referred to as “amino acid residues”, linked by peptide linkages, in which in certain embodiments no more than 12000 amino acid monomers are linked by peptide linkages, such as no more than 10000 amino acid monomer moieties, no more than 8000 amino acid monomer moieties, no more than 5000 amino acid monomer moieties or no more than 2000 amino acid monomer moieties.

It is understood that in the context of amino acid sequences single letters refer to the one-letter code of amino acids and not chemical atoms.

It is understood that the deletion or insertion of one or more amino acids in a sequence, such as SEQ ID NO: 1, may change the number, i.e. position, of a particular amino acid within such sequence and that in such case the corresponding, i.e. homologous, amino acid positions are included. This may be indicated by the phrase “or at the corresponding positions of homologs or variants thereof”, but such corresponding positions are included even in the absence of this phrase.

As used herein the terms “identical” and percent “identity”, in the context of two or more polynucleotide or polypeptide/protein sequences, refer to two or more sequences or subsequences that are the same or have a specified percentage of nucleotides or amino acid residues that are the same, when compared and aligned for maximum correspondence, as measured using a sequence comparison algorithm. An example of such an algorithm that is suitable for determining percent sequence identity is the BLAST algorithm (Altschul et al., J. Mol. Biol., 215: 403-410 (1990); Henikoff & Henikoff, Proc. Natl. Acad. Sci. USA, 89: 10915 (1989); Karlin & Altschul, Proc. Natl. Acad. Sci. USA, 90: 5873 5787 (1993)). Software for performing BLAST analyses is publicly available through the National Center for Biotechnology Information.

As used herein the term “about” or “approx.” in combination with a numerical value is used to indicate a range ranging from and including the numerical value plus and minus no more than 10% of said numerical value. For example, the phrase “about 200” is used to mean a range ranging from and including 200 +/−10%, i.e., ranging from and including 180 to 220. It is understood that a percentage given as “about 50%” does not mean “50% +/−10%”, i.e., ranging from and including 40 to 60%, but “about 50%” means ranging from and including 45 to 55%, i.e., plus and minus 10% of the numerical value which is 50.

As used herein, the term “polymer” means a molecule comprising repeating structural units, i.e., the monomers, connected by chemical bonds in a linear, circular, branched, crosslinked or dendrimeric way or a combination thereof, which may be of synthetic or biological origin or a combination of both. It is understood that a polymer may also comprise one or more other chemical group(s) and/or moiety/moieties, such as, for example, one or more functional group(s). Likewise, it is understood that also a peptide or protein is a polymer, even though the side chains of individual amino acid residues may be different. In certain embodiments a soluble polymer has a molecular weight of at least 0.5 kDa, e.g. a molecular weight of at least 1 kDa, a molecular weight of at least 2 kDa, a molecular weight of at least 3 kDa or a molecular weight of at least 5 kDa. If the polymer is soluble, it in certain embodiments has a molecular weight of at most 1000 kDa, such as at most 750 kDa, such as at most 500 kDa, such as at most 300 kDa, such as at most 200 kDa, such as at most 100 kDa. It is understood that for insoluble polymers, such as hydrogels, no meaningful molecular weight ranges can be provided.

As used herein, the term “polymeric” means a reagent or a moiety comprising one or more polymer(s) or polymer moiety/moieties. A polymeric reagent or moiety may optionally also comprise one or more other moiety/moieties, which are in certain embodiments selected from the group consisting of
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl; and
linkages selected from the group comprising

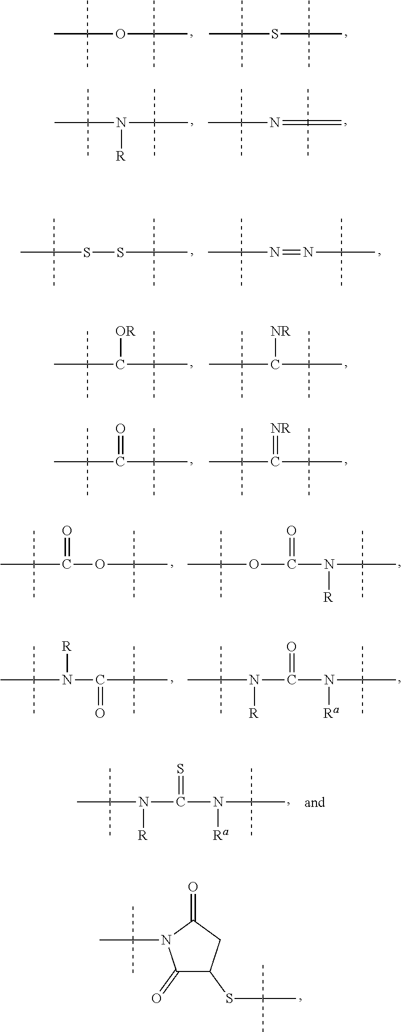

wherein
dashed lines indicate attachment to the remainder of the moiety or reagent, and —R and —R^{a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

The person skilled in the art understands that the polymerization products obtained from a polymerization reaction do not all have the same molecular weight, but rather exhibit a molecular weight distribution. Consequently, the molecular weight ranges, molecular weights, ranges of numbers of monomers in a polymer and numbers of monomers in a polymer as used herein, refer to the number average molecular weight and number average of monomers, i.e., to the arithmetic mean of the molecular weight of the polymer or polymeric moiety and the arithmetic mean of the number of monomers of the polymer or polymeric moiety.

Accordingly, in a polymeric moiety comprising “x” monomer units any integer given for “x” therefore corresponds to the arithmetic mean number of monomers. Any range of integers given for “x” provides the range of integers in which the arithmetic mean numbers of monomers lies. An integer for “x” given as “about x” means that the arithmetic mean numbers of monomers lies in a range of integers of x +/−10%.

As used herein, the term “number average molecular weight” means the ordinary arithmetic mean of the molecular weights of the individual polymers.

As used herein, the term “PEG-based” in relation to a moiety or reagent means that said moiety or reagent comprises PEG. In certain embodiments a PEG-based moiety or reagent comprises at least 10% (w/w) PEG, such as at least 20% (w/w) PEG, such as at least 30% (w/w) PEG, such as at least 40% (w/w) PEG, such as at least 50% (w/w), such as at least 60 (w/w) PEG, such as at least 70% (w/w) PEG, such as at least 80% (w/w) PEG, such as at least 90% (w/w) PEG, such as at least 95%. The remaining weight percentage of the PEG-based moiety or reagent are other moieties that in certain embodiments are selected from the following moieties and linkages:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl; and
linkages selected from the group comprising

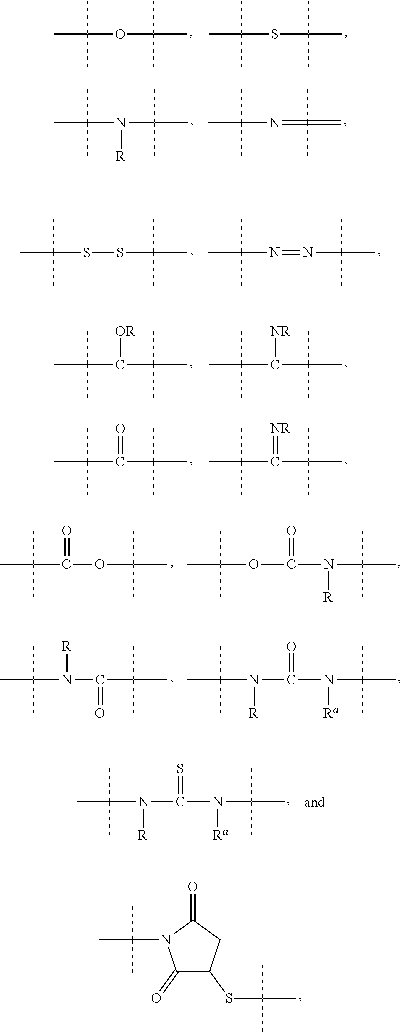

wherein
dashed lines indicate attachment to the remainder of the moiety or reagent, and —R and —R^{a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

The term “hyaluronic acid-based” is used accordingly.

As used herein, the term “PEG-based comprising at least X % PEG” in relation to a moiety or reagent means that said moiety or reagent comprises at least X % (w/w) ethylene glycol units (—CH₂CH₂O—), wherein the ethylene glycol units may be arranged blockwise, alternating or may be randomly distributed within the moiety or reagent and in certain embodiments all ethylene glycol units of said moiety or reagent are present in one block; the remaining weight percentage of the PEG-based moiety or reagent are other moieties that in certain embodiments are selected from the following moieties and linkages:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl; and
linkages selected from the group comprising

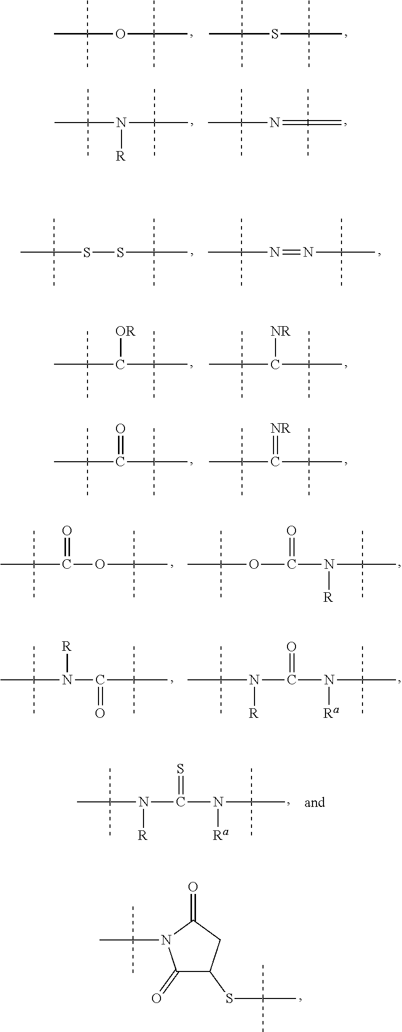

wherein
dashed lines indicate attachment to the remainder of the moiety or reagent, and —R and —R^{a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

The term “hyaluronic acid-based comprising at least X % hyaluronic acid” is used accordingly.

The term “interrupted” means that a moiety is inserted between two carbon atoms or—if the insertion is at one of the moiety's ends—between a carbon or heteroatom and a hydrogen atom.

As used herein, the term “C₁₋₄ alkyl” alone or in combination means a straight-chain or branched alkyl moiety having 1 to 4 carbon atoms. If present at the end of a molecule, examples of straight-chain or branched C₁₋₄ alkyl are methyl, ethyl, n-propyl, isopropyl, n-butyl, isobutyl, sec-butyl and tert-butyl. When two moieties of a molecule are linked by the C₁₋₄ alkyl, then examples for such C₁₋₄ alkyl groups are —CH₂—, —CH₂—CH₂—, —CH(CH₃)—, —CH₂—CH₂—CH₂—, —CH(C₂H₅)—, —C(CH₃)₂—. Each hydrogen of a C₁₋₄ alkyl carbon may optionally be replaced by a substituent as defined above. Optionally, a C₁₋₄ alkyl may be interrupted by one or more moieties as defined below.

As used herein, the term “C₁0.6 alkyl” alone or in combination means a straight-chain or branched alkyl moiety having 1 to 6 carbon atoms. If present at the end of a molecule, examples of straight-chain and branched C₁₋₆ alkyl groups are methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

When two moieties of a molecule are linked by the C₁₋₆ alkyl group, then examples for such C₁₋₆ alkyl groups are —CH₂—, —CH₂—CH₂—, —CH(CH₃)—, —CH₂—CH₂—CH₂—, —CH(C₂H₅)— and —C(CH₃)₂—. Each hydrogen atom of a C₁₋₆ carbon may optionally be replaced by a substituent as defined above. Optionally, a C₁₋₆ alkyl may be interrupted by one or more moieties as defined below.

Accordingly, “C₁₋₁₀ alkyl”, “C₁₋₂₀ alkyl” or “C₁₋₅₀ alkyl” means an alkyl chain having 1 to 10, 1 to 20 or 1 to 50 carbon atoms, respectively, wherein each hydrogen atom of the C₁₋₁₀, C₁₋₂₀ or C₁₋₅₀ carbon may optionally be replaced by a substituent as defined above. Optionally, a C₁₋₁₀ or C₁₋₅₀alkyl may be interrupted by one or more moieties as defined below.

As used herein, the term “C₂₋₆ alkenyl” alone or in combination means a straight-chain or branched hydrocarbon moiety comprising at least one carbon-carbon double bond having 2 to 6 carbon atoms. If present at the end of a molecule, examples are —CH═CH₂, —CH═CH—CH₃, —CH₂—CH═CH₂, —CH═CHCH₂—CH₃ and —CH═CH—CH═CH₂. When two moieties of a molecule are linked by the C₂₋₆ alkenyl group, then an example for such C₂₋₆ alkenyl is —CH═CH—. Each hydrogen atom of a C₂₋₆ alkenyl moiety may optionally be replaced by a substituent as defined above. Optionally, a C₂₋₆ alkenyl may be interrupted by one or more moieties as defined below.

Accordingly, the term “C₂₋₁₀ alkenyl”, “C₂₋₂₀ alkenyl” or “C₂₋₅₀ alkenyl” alone or in combination means a straight-chain or branched hydrocarbon moiety comprising at least one carbon-carbon double bond having 2 to 10, 2 to 20 or 2 to 50 carbon atoms. Each hydrogen atom of a C₂₋₁₀ alkenyl, C₂₋₂₀ alkenyl or C₂₋₅₀ alkenyl group may optionally be replaced by a substituent as defined above. Optionally, a C₂₋₁₀ alkenyl, C₂₋₂₀ alkenyl or C₂₋₅₀ alkenyl may be interrupted by one or more moieties as defined below.

As used herein, the term “C₂₋₆ alkynyl” alone or in combination means a straight-chain or branched hydrocarbon moiety comprising at least one carbon-carbon triple bond having 2 to 6 carbon atoms. If present at the end of a molecule, examples are —C—CH, —CH₂—C—CH, —CH₂—CH₂—C—CH and CH₂—C—C—CH₃. When two moieties of a molecule are linked by the alkynyl group, then an example is —C≡C—. Each hydrogen atom of a C₂₋₆ alkynyl group may optionally be replaced by a substituent as defined above. Optionally, one or more double bond(s) may occur. Optionally, a C₂₋₆ alkynyl may be interrupted by one or more moieties as defined below.

Accordingly, as used herein, the term “C₂₋₁₀ alkynyl”, “C₂₋₂₀ alkynyl” and “C₂₋₅₀ alkynyl” alone or in combination means a straight-chain or branched hydrocarbon moiety comprising at least one carbon-carbon triple bond having 2 to 10, 2 to 20 or 2 to 50 carbon atoms, respectively.

Each hydrogen atom of a C₂₋₁₀ alkynyl, C₂₋₂₀ alkynyl or C₂₋₅₀ alkynyl group may optionally be replaced by a substituent as defined above. Optionally, one or more double bond(s) may occur.

Optionally, a C₂₋₁₀ alkynyl, C₂₋₂₀ alkynyl or C₂₋₅₀ alkynyl may be interrupted by one or more moieties as defined below.

As mentioned above, a C₁₋₄ alkyl, C₁₋₆ alkyl, C₁₋₁₀ alkyl, C₁₋₂₀ alkyl, C₁₋₅₀ alkyl, C₂₋₆ alkenyl, C₂₋₁₀ alkenyl, C₂₋₂₀ alkenyl, C₂₋₅₀ alkenyl, C₂₋₆ alkynyl, C₂₋₁₀ alkynyl, C₂₋₂₀ alkenyl or C₂₋₅₀ alkynyl may optionally be interrupted by one or more moieties which are preferably selected from the group consisting of

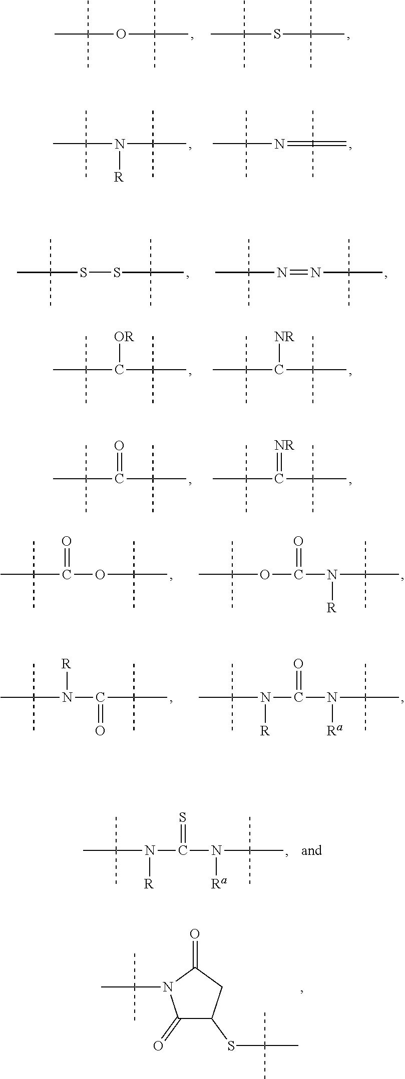

wherein
dashed lines indicate attachment to the remainder of the moiety or reagent; and —R and —R^{a} are independently of each other selected from the group consisting of —H, and methyl, ethyl, propyl, butyl, pentyl and hexyl.

As used herein, the term “C₃₋₁₀ cycloalkyl” means a cyclic alkyl chain having 3 to 10 carbon atoms, which may be saturated or unsaturated, e.g. cyclopropyl, cyclobutyl, cyclopentyl, cyclohexyl, cyclohexenyl, cycloheptyl, cyclooctyl, cyclononyl or cyclodecyl. Each hydrogen atom of a C₃0.10 cycloalkyl carbon may be replaced by a substituent as defined above. The term “C₃₋₁₀ cycloalkyl” also includes bridged bicycles like norbornane or norbornene.

The term “8- to 30-membered carbopolycyclyl” or “8- to 30-membered carbopolycycle” means a cyclic moiety of two or more rings with 8 to 30 ring atoms, where two neighboring rings share at least one ring atom and that may contain up to the maximum number of double bonds (aromatic or non-aromatic ring which is fully, partially or un-saturated). Preferably an 8- to 30-membered carbopolycyclyl means a cyclic moiety of two, three, four or five rings, more preferably of two, three or four rings.

As used herein, the term “3- to 10-membered heterocyclyl” or “3- to 10-membered heterocycle” means a ring with 3, 4, 5, 6, 7, 8, 9 or 10 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom.

Examples for 3- to 10-membered heterocycles include but are not limited to aziridine, oxirane, thiirane, azirine, oxirene, thiirene, azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine and homopiperazine. Each hydrogen atom of a 3- to 10-membered heterocyclyl or 3- to 10-membered heterocyclic group may be replaced by a substituent as defined below.

As used herein, the term “8- to 11-membered heterobicyclyl” or “8- to 11-membered heterobicycle” means a heterocyclic moiety of two rings with 8 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for an 8- to 11-membered heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquinoline, decahydroisoquinoline, tetrahydroisoquinoline, dihydroisoquinoline, benzazepine, purine and pteridine. The term 8- to 11-membered heterobicycle also includes spiro structures of two rings like 1,4-dioxa-8-azaspiro[4.5]decane or bridged heterocycles like 8-aza-bicyclo[3.2.1]octane. Each hydrogen atom of an 8- to 11-membered heterobicyclyl or 8- to 11-membered heterobicycle carbon may be replaced by a substituent as defined below.

Similarly, the term “8- to 30-membered heteropolycyclyl” or “8- to 30-membered heteropolycycle” means a heterocyclic moiety of more than two rings with 8 to 30 ring atoms, preferably of three, four or five rings, where two neighboring rings share at least one ring atom and that may contain up to the maximum number of double bonds (aromatic or non-aromatic ring which is fully, partially or unsaturated), wherein at least one ring atom up to 10 ring atoms are replaced by a heteroatom selected from the group of sulfur (including —S(O)—, —S(O)₂—), oxygen and nitrogen (including ═N(O)—) and wherein the ring is linked to the rest of a molecule via a carbon or nitrogen atom.

It is understood that the phrase “the pair R^{x}/R^{y} is joined together with the atom to which they are attached to form a C₃0.10 cycloalkyl or a 3- to 10-membered heterocyclyl” in relation with a moiety of the structure

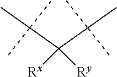

means that R^{x} and R^{y} form the following structure:

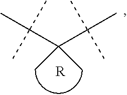

wherein R is C₃₋₁₀ cycloalkyl or 3- to 10-membered heterocyclyl.

It is also understood that the phrase “the pair R^{x}/R^{y} is joint together with the atoms to which they are attached to form a ring A” in relation with a moiety of the structure

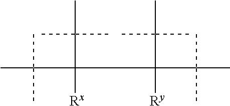

means that R^{x} and R^{y} form the following structure:

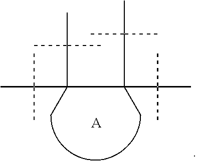

As used herein, “halogen” means fluoro, chloro, bromo or iodo. It is generally preferred that halogen is fluoro or chloro.

As used herein, the term “functional group” means a group of atoms which can react with other groups of atoms. Exemplary functional groups are, for example, carboxylic acid (—(C═O)OH), primary or secondary amine (—NH₂, —NH—), maleimide, thiol (—SH), sulfonic acid (—(O═S═O)OH), carbonate, carbamate (—O(C═O)N<), hydroxyl (—OH), aldehyde (—(C═O)H), ketone (—(C═O)—), hydrazine (>N—N<), isocyanate, isothiocyanate, phosphoric acid (—O(P═O)OHOH), phosphonic acid (—O(P═O)OHH), haloacetyl, alkyl halide, acryloyl, aryl fluoride, hydroxylamine, disulfide, sulfonamides, sulfuric acid, vinyl sulfone, vinyl ketone, diazoalkane, oxirane, and aziridine.

In case the IL-2 conjugates of the present invention comprise one or more acidic or basic groups, the invention also comprises their corresponding pharmaceutically or toxicologically acceptable salts, in particular their pharmaceutically utilizable salts. Thus, the IL-2 conjugates of the present invention comprising acidic groups can be used according to the invention, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine or amino acids. IL-2 conjugates of the present invention comprising one or more basic groups, i.e. groups which can be protonated, can be present and can be used according to the invention in the form of their addition salts with inorganic or organic acids. Examples for suitable acids include hydrogen chloride, hydrogen bromide, phosphoric acid, sulfuric acid, nitric acid, methanesulfonic acid, β-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. For the person skilled in the art further methods are known for converting the basic group into a cation like the alkylation of an amine group resulting in a positively-charge ammonium group and an appropriate counterion of the salt. If the IL-2 conjugates of the present invention simultaneously comprise acidic and basic groups, the invention also includes, in addition to the salt forms mentioned, inner salts or betaines (zwitterions). The respective salts can be obtained by customary methods, which are known to the person skilled in the art like, for example by contacting these conjugates with an organic or inorganic acid or base in a solvent or dispersant, or by anion exchange or cation exchange with other salts. The present invention also includes all salts of the IL-2 conjugates of the present invention which, owing to low physiological compatibility, are not directly suitable for use in pharmaceuticals but which can be used, for example, as intermediates for chemical reactions or for the preparation of pharmaceutically acceptable salts.

The term “pharmaceutically acceptable” means a substance that does not cause harm when administered to a patient and preferably means approved by a regulatory agency, such as the EMA (Europe) and/or the FDA (US) and/or any other national regulatory agency for use in animals, such as for use in humans.

As used herein, the term “excipient” refers to a diluent, adjuvant or vehicle with which the therapeutic, such as a drug or prodrug, is administered. Such pharmaceutical excipient can be sterile liquids, such as water and oils, including those of petroleum, animal, vegetable or synthetic origin, including but not limited to peanut oil, soybean oil, mineral oil, sesame oil and the like. Water is a preferred excipient when the pharmaceutical composition is administered orally. Saline and aqueous dextrose are preferred excipients when the pharmaceutical composition is administered intravenously. Saline solutions and aqueous dextrose and glycerol solutions are preferably employed as liquid excipients for injectable solutions. Suitable pharmaceutical excipients include starch, glucose, lactose, sucrose, mannitol, trehalose, gelatin, malt, rice, flour, chalk, silica gel, sodium stearate, glycerol monostearate, talc, sodium chloride, dried skim milk, glycerol, propylene, glycol, water, ethanol and the like. The pharmaceutical composition, if desired, can also contain minor amounts of wetting or emulsifying agents, pH buffering agents, like, for example, acetate, succinate, tris, carbonate, phosphate, HEPES (4-(2-hydroxyethyl)—1-piperazineethanesulfonic acid), MES (2-(N-morpholino)ethanesulfonic acid), or can contain detergents, like Tween, poloxamers, poloxamines, CHAPS, Igepal or amino acids like, for example, glycine, lysine or histidine. These pharmaceutical compositions can take the form of solutions, suspensions, emulsions, tablets, pills, capsules, powders, sustained-release formulations and the like. The pharmaceutical composition can be formulated as a suppository, with traditional binders and excipients such as triglycerides. Oral formulation can include standard excipients such as pharmaceutical grades of mannitol, lactose, starch, magnesium stearate, sodium saccharine, cellulose, magnesium carbonate, etc. Such compositions will contain a therapeutically effective amount of the drug or biologically active moiety, together with a suitable amount of excipient so as to provide the form for proper administration to the patient. The formulation should suit the mode of administration.

As used herein the term “liquid pharmaceutical formulation” refers to a mixture comprising a water-soluble compound, such as the IL-2 conjugate of the present invention, and one or more solvents, such as water.

As used herein, the term “dry pharmaceutical formulation” means that a pharmaceutical composition is provided in a dry form. Suitable methods for drying are spray-drying and lyophilization, i.e., freeze-drying. Such dry composition has a residual water content of a maximum of 10%, such as less than 5% or less than 2%, determined according to Karl Fischer.

In certain embodiments such dry pharmaceutical composition is dried by lyophilization.

As used herein, the term “percent (w/w)” or “% (w/w)” refers to a percentage expressed in terms of the weight of the compound, substance, ingredient or agent in a composition divided by the total weight of the composition multiplied by 100%.

As used herein, the term “percent (v/v)” or “% (v/v)” refers to a percentage expressed in terms of the liquid volume of the solvent, compound, substance, ingredient, or agent in a composition divided by the total liquid volume of the composition multiplied by 100%.

In general, the term “comprise” or “comprising” also encompasses “consist of” or “consisting of”.

The IL-2 conjugates of the formula (I) release a moiety D-H. Such moiety D-H may be a biased IL-2.

In certain embodiments the dose of the first to third aspects ranges from 100 μg IL-2/kg to 140 μg IL-2/kg. In certain embodiments the dose of the first to third aspects ranges from 110 μg IL-2/kg to 130 μg IL-2/kg. In certain embodiments the dose of the first to third aspects is about 120 μg IL-2/kg. In certain embodiments the dose of the first to third aspect is 120 μg IL-2/kg.

Doses can be administered once or multiple times. For multiple administrations, the interval between two consecutive administrations may range from two to eight weeks. In certain embodiments the IL-2 conjugate of formula (I) is administered to the patient every three weeks.

In certain embodiments the IL-2 conjugate of formula (I) is administered to the patient every six weeks. In certain embodiments the IL-2 conjugate of formula (I) is administered at a frequency dependent on disease progression. The IL-2 conjugate of formula (I) can be administered for at least six months, a year, two years or indefinitely. Treatment can start directly on diagnosis of a cancer or later. Treatment with the IL-2 conjugate of formula (I) can be the first line of treatment or can be the second, third or further line of treatment. The IL-2 conjugate of formula (I) may be administered via external application, injection or infusion, including intraarticular, periarticular, intradermal, subcutaneous, intramuscular, intravenous, intraosseous, intraperitoneal, intrathecal, intracapsular, intraorbital, intravitreal, intratympanic, intravesical, intracardiac, transtracheal, subcuticular, subcapsular, subarachnoid, intraspinal, intraventricular, intrasternal injection and infusion; direct delivery to the brain via an implanted device allowing delivery of the IL-2 conjugate to brain tissue or brain fluids (e.g., Ommaya Reservoir), direct intracerebroventricular injection or infusion, injection or infusion into brain or brain associated regions, injection into the subchoroidal space, retro-orbital injection or ocular instillation. In certain embodiments the IL-2 conjugate of formula (I) is administered intravenously, such as via intravenous infusion or intravenous injection.

In certain embodiments the IL-2 conjugate of formula (I) is administered to the patient in the form of a pharmaceutical formulation, such as a dry or liquid pharmaceutical formulation. Such pharmaceutical formulation comprises at least one IL-2 conjugate of formula (I) and one or more excipients. A dry formulation is resuspended prior to administration to a patient using a liquid such as sterile water or a sterile buffer.

The IL-2 conjugate of formula (I) may be administered to a patient once or multiple times. In certain embodiments the IL-2 conjugate is administered once to the patient. In certain embodiments the IL-2 conjugate is administered multiple times, such as twice, three times, four times, five times, eight times, ten times, twelve times, fourteen times or as often as necessary.

In certain embodiments the medicament of the third aspect is a unit dosage form, details are as provided elsewhere herein.

In certain embodiments the unit dosage form comprises from 100 μg IL-2/kg to 140 μg IL-2/kg. In certain embodiments the unit dosage form of the fourth aspect comprises from 110 μg IL-2/kg to 130 μg IL-2/kg. In certain embodiments the unit dosage form of the fourth aspect comprises about 120 μg IL-2/kg. In certain embodiments the unit dosage form of the fourth aspect comprises 120 μg IL-2/kg.

In certain embodiments the unit dosage form is a vial, such as a vial with a stopper. In certain embodiments the unit dosage form is a type 1 glass vial with a chloro-butyl rubber stopper and an aluminum seal cap. In certain embodiments the unit dosage form is a type 1 glass vial with a bromo-butyl rubber stopper and an aluminum seal cap.

In certain embodiments the unit dosage form of the fourth aspect is for use in the treatment of cancer. Embodiments of such a cancer are as described elsewhere herein.

If a dose is determined for a particular drug, such as a reversible conjugate of IL-2 of formula (I), then the dose can be used as a guide for other reversible conjugates of IL-2, such that the dose of the other reversible conjugates is the same by moles of IL-2 as that for the IL-2 conjugate of formula (I). Such guidance is particularly useful when the other conjugate releases an IL-2 moiety with a release half-life within plus or minus 20% of that of the IL-2 conjugate of formula (I).

The present methods can be used for treating or effecting prophylaxis of a patient having or at risk of (e.g., genetic risk) cancer. The present methods can also be used for treating a population of patients having or at risk of such a disease. Such a population can include at least 10, 100 or 1000 patients or may represent all patients at a particular institution.

Although clinical trials can be useful for determining doses and dosage regimens de novo, the present methods can also be performed not in the course of a clinical trial.

In certain embodiments the —D′— has at least 95% sequence identity to the amino acid sequence of SEQ ID NO:1. SEQ ID NO:1 has the following sequence:

APTSSSTKKTQLQLEHLLLDLQMILNGINNYKNPKLTRMLTFKFY
MPKKATELKHLQCLEEELKPLEEVLNLAQSKNFHLRPRDLISNIN
VIVLELKGSETTFMCEYADETATIVEFLNRWITFSQSIISTLT

In certain embodiments —D′— has at least 96% sequence identity to the amino acid sequence of SEQ ID NO:1. In certain embodiments —D′— has at least 97% sequence identity to the amino acid sequence of SEQ ID NO:1. In certain embodiments —D′— has at least 98% sequence identity to the amino acid sequence of SEQ ID NO:1. In certain embodiments —D′— has at least 99% sequence identity to the amino acid sequence of SEQ ID NO:1.

In certain embodiments —D′— comprises the amino acid sequence of SEQ ID NO:1, in which one amino acid residue selected from the group consisting of K35, R38, M39, T41, F42, K43, F44, Y45, E62, P65, E68, V69, and L72 is replaced with a proteinogenic or non-proteinogenic amino acid residue.

In certain embodiments —D′— is lacking the N-terminal alanine, i.e., —D′— comprises the Aldel mutation. In certain embodiments —D′— has the amino acid sequence of SEQ ID NO:2, which optionally comprises one or more mutations at positions selected from the group consisting of K34, R37, M38, T40, F41, K42, F43, E61, P64, E67, V68 and L71.

In certain embodiments —D′— comprises the amino acid sequence of SEQ ID NO:1, in which two amino acid residues selected from the group consisting of K35, R38, M39, T41, F42, K43, F44, Y45, E62, P65, E68, V69, and L72 are replaced with a proteinogenic or non-proteinogenic amino acid residue. A proteinogenic amino acid may be selected from the group consisting of cysteine, methionine, histidine, lysine, tryptophan, serine, threonine, tyrosine, aspartic acid, glutamic acid, asparagine, glutamine and arginine. In certain embodiments the proteinogenic amino acid is cysteine. In certain embodiments the proteinogenic amino acid is methionine. In certain embodiments the proteinogenic amino acid is histidine. In certain embodiments the proteinogenic amino acid is lysine. In certain embodiments the proteinogenic amino acid is tryptophan. In certain embodiments the proteinogenic amino acid is serine. In certain embodiments the proteinogenic amino acid is threonine. In certain embodiments the proteinogenic amino acid is tyrosine. In certain embodiments the proteinogenic amino acid is aspartic acid. In certain embodiments the proteinogenic amino acid is glutamic acid. In certain embodiments the proteinogenic amino acid is glutamine. In certain embodiments the proteinogenic amino acid is arginine.

In certain embodiments the non-proteinogenic amino acid comprises a functional group in its side chain selected from the group consisting of carbonyl (including a keto group and a dicarbonyl group); carbonyl derivatives, such as carbonyl-like, marked carbonyl and protected carbonyl groups; azide; oxime; hydrazine, hydrazide; semicarbazide; alkyne; and hydroxylamine.

In certain embodiments the non-proteinogenic amino acid is selected from the group consisting of lysine analogues, cysteine analogues and histidine analogues.

In certain embodiments the non-proteinogenic amino acid comprises an aromatic side chain, an azido group, an alkyne group, an aldehyde group or a ketone group. In certain embodiments the non-proteinogenic amino acid does not comprise an aromatic side chain.

In certain embodiments the non-proteinogenic amino acid is selected from the group consisting of D-stereoisomers of each of the proteinogenic amino acids, pyrrolysine (Pyl, 0), selenocysteine (Sec, U), 2-aminoadipic acid (2-AAA), 3-aminoadipic acid (bAad), beta-alanine (bAla), 2-aminobutyric acid (Abu), 4-aminobutyric acid (4Abu), 6-aminocaproic acid (Acp), 2-aminoheptanoic acid (Ahe), 2-aminoisobutyric acid (Aib), 3-aminoisobutyric acid (bAib), 2-aminopimelic acid (Apm), 2,4-diaminobutyric acid (Dbu), desmosine (Des), 2,2′-diaminopimelic acid (Dpm), 2,3-diaminoproprionic acid (Dpr), N-ethylglycine (EtGly), N-ethylasparagine (EtAsn), hydroxylysine (Hyl), allo-hydroxylysine (aHyl), 3-hydroxyproline (3Hyp), 4-hydroxyproline (4Hyp), isodesmosine (Ide), allo-isoleucine (alle), N-methylglycine (MeGly), N-methylisoleucine (Melle), 6-N-methyllysine (MeLys), N-methylvaline (MeVal), norvaline (Nva), norleucine (Nlle), and ornithine (Orn). The respective abbreviations are provided in brackets.

In certain embodiments the non-proteinogenic amino acid is selected from the group consisting of N6-((2-azidoethoxy)-carbonyl)-L-lysine, N6-azidoethoxy-L-lysine, N6-propargylethoxy-L-lysine, BCN-L-lysine, norbornene lysine, trans-cyclooctene (TCO)-lysine, methyltetrazine lysine, allyloxycarbonyllysine, 2-amino-8-oxononanoic acid, 2-amino-8-oxooctanoic acid, p-acetyl-L-phenylalanine, p-azidomethyl-L-phenylalanine, p-iodo-L-phenylalanine, m-acetylphenylalanine, 2-amino-8-oxononanoic acid, p-propargyloxyphenylalanine, p-propargyl-phenylalanine, 3-methyl-phenylalanine, L-Dopa, fluorinated phenylalanine, isopropyl-L-phenylalanine, p-azido-L-phenylalanine, p-acyl-L-phenylalanine, p-benzoyl-L-phenylalanine, p-bromophenylalanine, p-amino-L-phenylalanine, isopropyl-L-phenylalanine, 0-allyltyrosine, O-methyl-L-tyrosine, 0-4-allyl-L-tyrosine, 4-propyl-L-tyrosine, phosphonotyrosine, tri-O-acetyl-GlcNAcp-serine, L-phosphoserine, phosphonoserine, L-3-(2-naphthyl)alanine, 2-amino-3-((2-((3-(benzyloxy)—3-oxopropyl)amino)ethyl)selanyl)propanoic acid, 2-amino-3-(phenylselanyl)propanoic, and selenocysteine.

In certain embodiments such non-proteinogenic amino acid is a non-proteinogenic amino acid as described in WO2006/069246A2, which are incorporated by reference herewith. In certain embodiments the non-proteinogenic amino acid has a structure as described in formula (I) in [00265] to [00283], of formula (XXX) in [00284], of formula (XXX-A) in [00285], of formula (XXX-B) in [00286], of formula (XXXI) in [00287], of formula (XXXI-A) in [00288], of formula (XXXI-B) in [00289], of formula (XXXII) in [00290], of formula (XXXII-A) in [00291], of formula (XXXII-B) in [00292], of formula (XXXX) in [00293], of formula (XXXXI) in [00294], of formula (XXXXII) in erroneously labelled paragraph [0100], i.e. the paragraph between [00294] and [00295], of formula (XXXXIII) in [00295] and [00296], of formula (XIV) in [00302] to [00305], of formula (XV) in [00306] and [00307], of formula (XI) in [00310] to [00312], of formula (XII) in [00313], of formula (XII) in [00314] and [00315], of formula (XIV) in [00316], of formula (XVI) in [00317], of formula (XVI) in [00318] and [00319], of formula (XVIII) in [00320] and [00321], or of formula (XXIX) in [00530] of WO2006/069246A2, which non-proteinogenic amino acids are incorporated by reference herewith.

In certain embodiments —D′— has the amino acid sequence of SEQ ID NO:1, in which one amino acid residue selected from the group consisting of K35, R38, M39, T41, F42, K43, F44, Y45, E62, P65, E68, V69, and L72 is replaced with a cysteine residue.

In certain embodiments —D′— has the amino acid sequence of SEQ ID NO:1, in which two amino acid residues selected from the group consisting of K35, R38, M39, T41, F42, K43, F44, Y45, E62, P65, E68, V69, and L72 are replaced with a cysteine residue.

In certain embodiments —D′— has the amino acid sequence of SEQ ID NO:2, in which one amino acid residue selected from the group consisting of K34, R37, M38, T40, F41, K42, F43, E61, P64, E67, V68 and L71 is replaced with a cysteine residue.

In certain embodiments —D′— has the amino acid sequence of SEQ ID NO:2, in which two amino acid residues selected from the group consisting of K34, R37, M38, T40, F41, K42, F43, E61, P64, E67, V68 and L71 are replaced with a cysteine residue.

In certain embodiments —D′— has the amino acid sequence of SEQ ID NO:3:

APTSSSTKKTQLQLEHLLLDLQMILNGINNYKNPCLTRMLTFKFY
MPKKATELKHLQCLEEELKPLEEVLNLAQSKNFHLRPRDLISNIN
VIVLELKGSETTFMCEYADETATIVEFLNRWITFSQSIISTLT

In certain embodiments —D′— has the amino acid sequence of SEQ ID NO:4:

APTSSSTKKTQLQLEHLLLDLQMILNGINNYKNPKLTCMLTFKFY
MPKKATELKHLQCLEEELKPLEEVLNLAQSKNFHLRPRDLISNIN
VIVLELKGSETTFMCEYADETATIVEFLNRWITFSQSIISTLT

In certain embodiments —D′— has the amino acid sequence of SEQ ID NO:5:

APTSSSTKKTQLQLEHLLLDLQMILNGINNYKNPKLTRCLTFKFY
MPKKATELKHLQCLEEELKPLEEVLNLAQSKNFHLRPRDLISNIN
VIVLELKGSETTFMCEYADETATIVEFLNRWITFSQSIISTLT

In certain embodiments —D′— has the amino acid sequence of SEQ ID NO:6:

APTSSSTKKTQLQLEHLLLDLQMILNGINNYKNPKLTRMLCFKFY
MPKKATELKHLQCLEEELKPLEEVLNLAQSKNFHLRPRDLISNIN
VIVLELKGSETTFMCEYADETATIVEFLNRWITFSQSIISTLT

In certain embodiments —D′— has the amino acid sequence of SEQ ID NO:7:

APTSSSTKKTQLQLEHLLLDLQMILNGINNYKNPKLTRMLTCKFY
MPKKATELKHLQCLEEELKPLEEVLNLAQSKNFHLRPRDLISNIN
VIVLELKGSETTFMCEYADETATIVEFLNRWITFSQSIISTLT

In certain embodiments —D′— has the amino acid sequence of SEQ ID NO:8:

APTSSSTKKTQLQLEHLLLDLQMILNGINNYKNPKLTRMLTFCFY
MPKKATELKHLQCLEEELKPLEEVLNLAQSKNFHLRPRDLISNIN
VIVLELKGSETTFMCEYADETATIVEFLNRWITFSQSIISTLT

In certain embodiments —D′— has the amino acid sequence of SEQ ID NO:9:

APTSSSTKKTQLQLEHLLLDLQMILNGINNYKNPKLTRMLTFKCY
MPKKATELKHLQCLEEELKPLEEVLNLAQSKNFHLRPRDLISNIN
VIVLELKGSETTFMCEYADETATIVEFLNRWITFSQSIISTLT

In certain embodiments —D′— has the amino acid sequence of SEQ ID NO:10:

APTSSSTKKTQLQLEHLLLDLQMILNGINNYKNPKLTRMLTFKFC
MPKKATELKHLQCLEEELKPLEEVLNLAQSKNFHLRPRDLISNIN
VIVLELKGSETTFMCEYADETATIVEFLNRWITFSQSIISTLT

In certain embodiments —D′— has the amino acid sequence of SEQ ID NO:11:

APTSSSTKKTQLQLEHLLLDLQMILNGINNYKNPKLTRMLTFKFY
MPKKATELKHLQCLEECLKPLEEVLNLAQSKNFHLRPRDLISNIN
VIVLELKGSETTFMCEYADETATIVEFLNRWITFSQSIISTLT

In certain embodiments —D′— has the amino acid sequence of SEQ ID NO:12:

APTSSSTKKTQLQLEHLLLDLQMILNGINNYKNPKLTRMLTFKFY
MPKKATELKHLQCLEEELKCLEEVLNLAQSKNFHLRPRDLISNIN
VIVLELKGSETTFMCEYADETATIVEFLNRWITFSQSIISTLT

In certain embodiments —D′— has the amino acid sequence of SEQ ID NO:13:

APTSSSTKKTQLQLEHLLLDLQMILNGINNYKNPKLTRMLTFKFY
MPKKATELKHLQCLEEELKPLECVLNLAQSKNFHLRPRDLISNIN
VIVLELKGSETTFMCEYADETATIVEFLNRWITFSQSIISTLT

In certain embodiments —D′— has the amino acid sequence of SEQ ID NO:14:

APTSSSTKKTQLQLEHLLLDLQMILNGINNYKNPKLTRMLTFKFY
MPKKATELKHLQCLEEELKPLEECLNLAQSKNFHLRPRDLISNIN
VIVLELKGSETTFMCEYADETATIVEFLNRWITFSQSIISTLT

In certain embodiments —D′— has the amino acid sequence of SEQ ID NO:15:

APTSSSTKKTQLQLEHLLLDLQMILNGINNYKNPKLTRMLTFKFY
MPKKATELKHLQCLEEELKPLEEVLNCAQSKNFHLRPRDLISNIN
VIVLELKGSETTFMCEYADETATIVEFLNRWITFSQSIISTLT

In certain embodiments —D′— has the amino acid sequence of SEQ ID NO:16:

PTSSSTKKTQLQLEHLLLDLQMILNGINNYKNPCLTRMLTFKFYM
PKKATELKHLQCLEEELKPLEEVLNLAQSKNFHLRPRDLISNINV
IVLELKGSETTFMCEYADETATIVEFLNRWITFSQSIISTLT

In certain embodiments —D′— has the amino acid sequence of SEQ ID NO:17:

PTSSSTKKTQLQLEHLLLDLQMILNGINNYKNPKLTCMLTFKFYM
PKKATELKHLQCLEEELKPLEEVLNLAQSKNFHLRPRDLISNINV
IVLELKGSETTFMCEYADETATIVEFLNRWITFSQSIISTLT

In certain embodiments —D′— has the amino acid sequence of SEQ ID NO:18:

PTSSSTKKTQLQLEHLLLDLQMILNGINNYKNPKLTRCLTFKFYM
PKKATELKHLQCLEEELKPLEEVLNLAQSKNFHLRPRDLISNINV
IVLELKGSETTFMCEYADETATIVEFLNRWITFSQSIISTLT

In certain embodiments —D′— has the amino acid sequence of SEQ ID NO:19:

PTSSSTKKTQLQLEHLLLDLQMILNGINNYKNPKLTRMLCFKFYM
PKKATELKHLQCLEEELKPLEEVLNLAQSKNFHLRPRDLISNINV
IVLELKGSETTFMCEYADETATIVEFLNRWITFSQSIISTLT

In certain embodiments —D′— has the amino acid sequence of SEQ ID NO:20:

PTSSSTKKTQLQLEHLLLDLQMILNGINNYKNPKLTRMLTCKFYM
PKKATELKHLQCLEEELKPLEEVLNLAQSKNFHLRPRDLISNINV
IVLELKGSETTFMCEYADETATIVEFLNRWITFSQSIISTLT

In certain embodiments —D′— has the amino acid sequence of SEQ ID NO:21:

PTSSSTKKTQLQLEHLLLDLQMILNGINNYKNPKLTRMLTFCFYM
PKKATELKHLQCLEEELKPLEEVLNLAQSKNFHLRPRDLISNINV
IVLELKGSETTFMCEYADETATIVEFLNRWITFSQSIISTLT

In certain embodiments —D′— has the amino acid sequence of SEQ ID NO:22:

PTSSSTKKTQLQLEHLLLDLQMILNGINNYKNPKLTRMLTFKCYM
PKKATELKHLQCLEEELKPLEEVLNLAQSKNFHLRPRDLISNINV
IVLELKGSETTFMCEYADETATIVEFLNRWITFSQSIISTLT

In certain embodiments —D′— has the amino acid sequence of SEQ ID NO:23:

PTSSSTKKTQLQLEHLLLDLQMILNGINNYKNPKLTRMLTFKFCM
PKKATELKHLQCLEEELKPLEEVLNLAQSKNFHLRPRDLISNINV
IVLELKGSETTFMCEYADETATIVEFLNRWITFSQSIISTLT

In certain embodiments —D′— has the amino acid sequence of SEQ ID NO:24:

PTSSSTKKTQLQLEHLLLDLQMILNGINNYKNPKLTRMLTFKFYM
PKKATELKHLQCLEECLKPLEEVLNLAQSKNFHLRPRDLISNINV
IVLELKGSETTFMCEYADETATIVEFLNRWITFSQSIISTLT

In certain embodiments —D′— has the amino acid sequence of SEQ ID NO:25:

PTSSSTKKTQLQLEHLLLDLQMILNGINNYKNPKLTRMLTFKFYM
PKKATELKHLQCLEEELKCLEEVLNLAQSKNFHLRPRDLISNINV
IVLELKGSETTFMCEYADETATIVEFLNRWITFSQSIISTLT

In certain embodiments —D′— has the amino acid sequence of SEQ ID NO:26:

PTSSSTKKTQLQLEHLLLDLQMILNGINNYKNPKLTRMLTFKFYM
PKKATELKHLQCLEEELKPLECVLNLAQSKNFHLRPRDLISNINV
IVLELKGSETTFMCEYADETATIVEFLNRWITFSQSIISTLT

In certain embodiments —D′— has the amino acid sequence of SEQ ID NO:27:

PTSSSTKKTQLQLEHLLLDLQMILNGINNYKNPKLTRMLTFKFYM
PKKATELKHLQCLEEELKPLEECLNLAQSKNFHLRPRDLISNINV
IVLELKGSETTFMCEYADETATIVEFLNRWITFSQSIISTLT

In certain embodiments —D′— has the amino acid sequence of SEQ ID NO:28:

PTSSSTKKTQLQLEHLLLDLQMILNGINNYKNPKLTRMLTFKFYM
PKKATELKHLQCLEEELKPLEEVLNCAQSKNFHLRPRDLISNINV
IVLELKGSETTFMCEYADETATIVEFLNRWITFSQSIISTLT

—M_{mod} is a polymeric moiety. Such polymeric moiety may comprise a linear, branched or multi-arm polymer. In certain embodiments —M_{mod} is a linear polymer. In certain embodiments —M_{mod} is a branched polymer, such as a branched polymer with one, two, three, four or five branching points and from each branching point two, three or four polymeric arms may extend. In another embodiment —M_{mod} is a multi-arm polymer, such as a multi-arm polymer with 3, 4, 5, 6, 7 or 8 polymeric arms.

In certain embodiments —M_{mod} has a molecular weight ranging from 0.5 kDa to 20 kDa, such as from 1 kDa to 20 kDa, such as from 2 kDa to 10 kDa, from 3 kDa to 7 kDa or from 4 kDa to 6 kDa. In certain embodiments —M_{mod} has a molecular weight of about 0.5 kDa. In certain embodiments —M_{mod} has a molecular weight of about 1 kDa. In certain embodiments —M_{mod} has a molecular weight of about 2 kDa. In certain embodiments —M_{mod} has a molecular weight of about 3 kDa. In certain embodiments —M_{mod} has a molecular weight of about 4 kDa. In certain embodiments —M_{mod} has a molecular weight of about 5 kDa. In certain embodiments —M_{mod} has a molecular weight of about 6 kDa. In certain embodiments —M_{mod} has a molecular weight of about 7 kDa. In certain embodiments —M_{mod} has a molecular weight of about 8 kDa. In certain embodiments —M_{mod} has a molecular weight of about 9 kDa. In certain embodiments —M_{mod} has a molecular weight of about 10 kDa. In certain embodiments —M_{mod} has a molecular weight of about 15 kDa. In certain embodiments —M_{mod} has a molecular weight of about 20 kDa. In certain embodiments —M_{mod} has a molecular weight of 0.5 kDa. In certain embodiments —M_{mod} has a molecular weight of 1 kDa. In certain embodiments —M_{mod} has a molecular weight of 2 kDa. In certain embodiments —M_{mod} has a molecular weight of 3 kDa. In certain embodiments —M_{mod} has a molecular weight of 4 kDa. In certain embodiments —M_{mod} has a molecular weight of 5 kDa. In certain embodiments —M_{mod} has a molecular weight of 6 kDa. In certain embodiments —M_{mod} has a molecular weight of 7 kDa. In certain embodiments —M_{mod} has a molecular weight of 8 kDa. In certain embodiments —M_{mod} has a molecular weight of 9 kDa. In certain embodiments —M_{mod} has a molecular weight of 10 kDa. In certain embodiments —M_{mod} has a molecular weight of 15 kDa. In certain embodiments —M_{mod} has a molecular weight of 20 kDa.

In certain embodiments —M_{mod} has a molecular weight of 5±25% kDa. In certain embodiments —M_{mod} has a molecular weight of 5±20% kDa. In certain embodiments —M_{mod}has a molecular weight of 5 15% kDa.

In certain embodiments —M_{mod} is a polymer selected from the group consisting of poly(2-methacryloyl-oxyethyl phosphoyl cholins), poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, alginate, mannans, pectins, rhamnogalacturonans, starches, hydroxyalkyl starches, hydroxyethyl starches and other carbohydrate-based polymers, xylans, and copolymers thereof.

In certain embodiments —M_{mod} is a PEG-based polymer.

Attachment of —M_{mod} to —D′— is via a stable linkage, which in certain embodiments is an amide linkage. In certain embodiments the linkage between —D′— and —M_{mod} is the following linkage:

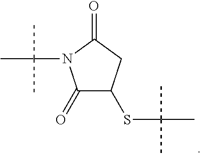

Attachment of —M_{mod} may be at a proteinogenic or non-proteinogenic amino acid residue of —D′—. In certain embodiments attachment of —M_{mod} occurs at a proteinogenic amino acid. Such proteinogenic amino acid residue is in certain embodiments selected from the group consisting of cysteine, methionine, histidine, lysine, tryptophan, serine, threonine, tyrosine, aspartic acid, glutamic acid, glutamine and arginine. In certain embodiments attachment of —M_{mod} occurs to a non-proteinogenic amino acid. In this case it is understood that such non-proteinogenic amino acid residue is artificially introduced into —D′—. Such non-proteinogenic amino acid residue may be any non-proteinogenic amino acid residue having a functional group available for conjugating —M_{mod} to —D′—. In certain embodiments such non-proteinogenic amino acid comprises a functional group in its side chain selected from the group consisting of carbonyl; carbonyl derivatives, such as carbonyl-like, marked carbonyl and protected carbonyl groups; azide; oxime; and hydroxylamine.

In certain embodiments such non-proteinogenic amino acid is a non-proteinogenic amino acid as described elsewhere herein.

In certain embodiments attachment of —M_{mod} occurs at a cysteine residue of —D′—. In certain embodiments attachment of —M_{mod} occurs at a lysine residue of —D′—. In certain embodiments attachment of —M_{mod} occurs at a threonine residue of —D′—. In certain embodiments attachment of —M_{mod} occurs at a serine residue of —D′—. In certain embodiments attachment of —M_{mod} occurs at a tyrosine residue of —D′—. In certain embodiments attachment of —M_{mod} occurs at a histidine residue of —D′—. In certain embodiments attachment of —M_{mod} occurs at a tryptophan residue of —D′—. In certain embodiments attachment of —M_{mod} occurs at an aspartic acid residue of —D′—.

In certain embodiments attachment of —M_{mod} occurs at a glutamic acid residue of —D′—. In certain embodiments attachment of —M_{mod} occurs at an arginine residue of —D′—. In certain embodiments attachment of —M_{mod} occurs at a methionine residue of —D′—. In certain embodiments attachment of —M_{mod} occurs at a glutamine residue of —D′—.

In certain embodiments attachment of —M_{mod} occurs at an amino acid position of the IL-2 protein known to be involved in binding to IL-2Rα. Thus, in certain embodiments attachment of —M_{mod} results in a reduced affinity of —D′— to IL-2Rα/p compared to aldesleukin (SEQ ID NO:2), i.e., results in a biased IL-2 moiety.

In certain embodiments attachment of —M_{mod} occurs at an amino acid position selected from the group consisting of K35, R38, M39, T41, F42, K43, F44, Y45, E62, P65, E68, V69, and L72, based on SEQ ID NO:1 or at the corresponding positions of homologs or variants thereof.

In certain embodiments attachment of —M_{mod} occurs at an amino acid position of —D′— selected from the group consisting of R38, F42, Y45, E62, P65, and L72 based on SEQ ID NO:1 or at the corresponding positions of homologs and variants thereof.

It is understood that not all of these amino acid positions comprise a functional group that would allow direct conjugation of —M_{mod} and that certain steps may be necessary prior to attaching —M_{mod} at these amino acid positions, such as for example replacing the naturally occurring amino acid with a different proteinogenic or non-proteinogenic amino acid or performing certain chemical modifications. Accordingly, attachment of —M_{mod} may occur in these positions either at the naturally occurring amino acid or at an amino acid that replaced the naturally occurring amino acid at that position and the attachment site may be a proteinogenic or non-proteinogenic amino acid, embodiments of both are as describe above.

In certain embodiments attachment of —M_{mod} occurs at amino acid position K35 of SEQ ID NO:1 or the corresponding position of homologs or variants thereof. In certain embodiments attachment of —M_{mod} occurs at amino acid position R38 of SEQ ID NO:1 or the corresponding position of homologs or variants thereof. In certain embodiments attachment of —M_{mod} occurs at amino acid position M39 of SEQ ID NO:1 or the corresponding position of homologs or variants thereof. In certain embodiments attachment of —M_{mod} occurs at amino acid position T41 of SEQ ID NO:1 or the corresponding position of homologs or variants thereof. In certain embodiments attachment of —M_{mod} occurs at amino acid position F42 of SEQ ID NO:1 or the corresponding position of homologs or variants thereof. In certain embodiments attachment of —M_{mod} occurs at amino acid position K43 of SEQ ID NO:1 or the corresponding position of homologs or variants thereof. In certain embodiments attachment of —M_{mod} occurs at amino acid position F44 of SEQ ID NO:1 or the corresponding position of homologs or variants thereof.

In certain embodiments attachment of —M_{mod} occurs at amino acid position Y45 of SEQ ID NO:1 or the corresponding position of homologs or variants thereof. In certain embodiments attachment of —M_{mod} occurs at amino acid position E62 of SEQ ID NO:1 or the corresponding position of homologs or variants thereof. In certain embodiments attachment of —M_{mod} occurs at amino acid position P65 of SEQ ID NO:1 or the corresponding position of homologs or variants thereof. In certain embodiments attachment of —M_{mod} occurs at amino acid position L72 of SEQ ID NO:1 or the corresponding position of homologs or variants thereof.

In certain embodiments —D′— has the amino acid sequence of SEQ ID NO:3 and —M_{mod} is stably conjugated to the sulfur atom of the side chain of the cysteine at position 35. In certain embodiments —D′— has the amino acid sequence of SEQ ID NO:4 and —M_{mod} is stably conjugated to the sulfur atom of the side chain of the cysteine at position 38. In certain embodiments —D′— has the amino acid sequence of SEQ ID NO:5 and —M_{mod} is stably conjugated to the sulfur atom of the side chain of the cysteine at position 39. In certain embodiments —D′— has the amino acid sequence of SEQ ID NO:6 and —M_{mod} is stably conjugated to the sulfur atom of the side chain of the cysteine at position 41. In certain embodiments —D′— has the amino acid sequence of SEQ ID NO:7 and —M_{mod} is stably conjugated to the sulfur atom of the side chain of the cysteine at position 42. In certain embodiments —D′— has the amino acid sequence of SEQ ID NO:8 and —M_{mod} is stably conjugated to the sulfur atom of the side chain of the cysteine at position 43. In certain embodiments —D′— has the amino acid sequence of SEQ ID NO:9 and —M_{mod} is stably conjugated to the sulfur atom of the side chain of the cysteine at position 44. In certain embodiments —D′— has the amino acid sequence of SEQ ID NO:10 and —M_{mod} is stably conjugated to the sulfur atom of the side chain of the cysteine at position 45. In certain embodiments —D′— has the amino acid sequence of SEQ ID NO:11 and —M_{mod} is stably conjugated to the sulfur atom of the side chain of the cysteine at position 62. In certain embodiments —D′— has the amino acid sequence of SEQ ID NO:12 and —M_{mod} is stably conjugated to the sulfur atom of the side chain of the cysteine at position 65. In certain embodiments —D′— has the amino acid sequence of SEQ ID NO:13 and —M_{mod} is stably conjugated to the sulfur atom of the side chain of the cysteine at position 68. In certain embodiments —D′— has the amino acid sequence of SEQ ID NO:14 and —M_{mod} is stably conjugated to the sulfur atom of the side chain of the cysteine at position 69. In certain embodiments —D′— has the amino acid sequence of SEQ ID NO:15 and —M_{mod} is stably conjugated to the sulfur atom of the side chain of the cysteine at position 72. In certain embodiments —D′— has the amino acid sequence of SEQ ID NO:16 and —M_{mod} is stably conjugated to the sulfur atom of the side chain of the cysteine at position 34. In certain embodiments —D′— has the amino acid sequence of SEQ ID NO:17 and —M_{mod} is stably conjugated to the sulfur atom of the side chain of the cysteine at position 37. In certain embodiments —D′— has the amino acid sequence of SEQ ID NO:18 and —M_{mod} is stably conjugated to the sulfur atom of the side chain of the cysteine at position 38. In certain embodiments —D′— has the amino acid sequence of SEQ ID NO:19 and —M_{mod} is stably conjugated to the sulfur atom of the side chain of the cysteine at position 40. In certain embodiments —D′— has the amino acid sequence of SEQ ID NO:20 and —M_{mod} is stably conjugated to the sulfur atom of the side chain of the cysteine at position 41. In certain embodiments —D′— has the amino acid sequence of SEQ ID NO:21 and —M_{mod} is stably conjugated to the sulfur atom of the side chain of the cysteine at position 42. In certain embodiments —D′— has the amino acid sequence of SEQ ID NO:22 and —M_{mod} is stably conjugated to the sulfur atom of the side chain of the cysteine at position 43. In certain embodiments —D′— has the amino acid sequence of SEQ ID NO:23 and —M_{mod} is stably conjugated to the sulfur atom of the side chain of the cysteine at position 44. In certain embodiments —D′— has the amino acid sequence of SEQ ID NO:24 and —M_{mod} is stably conjugated to the sulfur atom of the side chain of the cysteine at position 61. In certain embodiments —D′— has the amino acid sequence of SEQ ID NO:25 and —M_{mod} is stably conjugated to the sulfur atom of the side chain of the cysteine at position 64. In certain embodiments —D′— has the amino acid sequence of SEQ ID NO:26 and —M_{mod} is stably conjugated to the sulfur atom of the side chain of the cysteine at position 67. In certain embodiments —D′— has the amino acid sequence of SEQ ID NO:27 and —M_{mod} is stably conjugated to the sulfur atom of the side chain of the cysteine at position 68. In certain embodiments —D′— has the amino acid sequence of SEQ ID NO:28 and —M_{mod} is stably conjugated to the sulfur atom of the side chain of the cysteine at position 71.

Optionally, —D′— may comprise one or more further moieties —M_{mod} stably conjugated to —D, which may be the same or different moieties —M_{mod}. These one or more further moieties —M_{mod} are as described for —M_{mod} elsewhere herein.

In certain embodiments —M_{mod} is of formula (A-1)

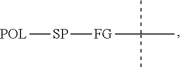

wherein
—FG— is a linkage;
—SP— is a spacer moiety; and
POL is a polymer.

—FG— of formula (A-1) may be any linkage used in chemistry, such as for example a linkage selected from the group consisting of

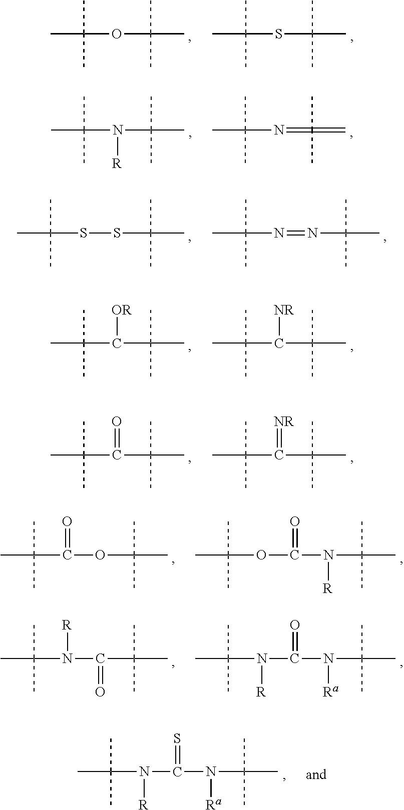

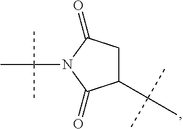

wherein
dashed lines indicate attachment to —D′— and —SP—, respectively, and
R and —R^{a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

In certain embodiments —FG— of formula (A-1) is of formula (FG-1a)

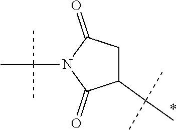

wherein
the dashed line marked with the asterisk indicates attachment to the sulfur atom of the side chain of a cysteine of —D′— and the unmarked dashed line indicates attachment to —SP—.

In certain embodiments IL-2 conjugates comprising a moiety —M_{mod} with a moiety —FG— of formula (FG-1a) are incubated at elevated pH, such a pH 9, and elevated temperature, such as 25° C., for several hours, such as at least 10 hours or at least 12 hours to facilitate opening of the ring.

Accordingly, in certain embodiments —FG— of formula (A-1) is of formula (FG-1d)

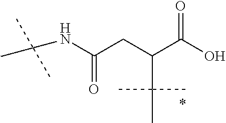

wherein the dashed line marked with the asterisk indicates attachment to the sulfur of the side chain of a cysteine residue of —D′— and the unmarked dashed line indicates attachment to —SP—.

Alternatively or additionally, in certain embodiments —FG— of formula (A-1) is of formula (FG— 1e)

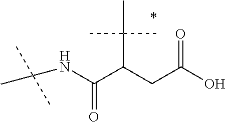

wherein the dashed line marked with the asterisk indicates attachment to the sulfur of the side chain of a cysteine residue of —D′— and the unmarked dashed line indicates attachment to —SP—.

When the IL-2 conjugate was incubated at elevated pH as described above, —FG— is present as a mixture of formula (FG-1d) and formula (FG-1e). Minor amounts of —FG— of formula (FG— 1a) may be present as a result of incomplete ring opening.

In certain embodiments —SP— of formula (A-1) is selected from the group consisting of C₁₋₅₀ alkyl, C₂₋₅₀ alkenyl, and C₂₋₅₀ alkynyl; wherein C₁₋₅₀ alkyl, C₂₋₅₀ alkenyl, and C₂₋₅₀ alkynyl are optionally substituted with one or more R⁹, which are the same or different and wherein C₁₋₅₀ alkyl, C₂₋₅₀ alkenyl, and C₂₋₅₀ alkynyl are optionally interrupted by one or more groups selected from the group consisting of —T—, —C(O)O—, —O—, —C(O)—, —C(O)N(R¹⁰)—, —S(O)₂N(R¹⁰)—, —S(O)N(R¹⁰)—, —S(O)₂—, —S(O)—, —N(R¹⁰)S(O)₂N(R^{10a})—, —S—, —N(R¹⁰)—, —OC(OR¹⁰)(R^{10a})—, —N(R¹⁰)C(O)N(R^{10a})—, and —OC(O)N(R¹⁰)—;

each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more R⁹, which are the same or different;

each —R⁹ is independently selected from the group consisting of halogen, —CN, oxo (═O), —COOR¹¹, —OR¹¹, —C(O)R¹¹, —C(O)N(R¹¹R^{11a}), —S(O)₂N(R¹¹R^{11a}), —S(O)N(R¹¹R^{11a}) —S(O)₂R¹¹, —S(O)R¹¹, —N(R¹¹)S(O)₂N(R^{11a}R^{11b}), —SR¹¹, —N(R¹¹R^{11a}), —NO₂, —OC(O)R¹¹ —N(R¹¹)C(O)R^{11a}, —N(R¹¹)S(O)₂R^{11a}, —N(R¹¹)S(O)R^{11a}, —N(R¹¹)C(O)OR^{11a} —N(R¹¹)C(O)N(R^{11a}R^{11b}), —OC(O)N(R¹¹R^{11a}), and C₁₋₆ alkyl; wherein C₁₋₆ alkyl is optionally substituted with one or more halogen, which are the same or different; and each —R¹⁰, —R^{10a}, —R¹¹, —R^{11a} and —R^{11b} is independently selected from the group consisting of —H, and C₁₋₆ alkyl, wherein C₁₋₆ alkyl is optionally substituted with one or more halogen, which are the same or different.

In certain embodiments —SP— of formula (A-1) is C₁₋₂₀ alkyl, which C₁₋₂₀ alkyl is optionally substituted with one or more —R⁹, and which C₁₋₂₀ alkyl is optionally interrupted by one or more groups selected from the group consisting of —O—, —C(O)N(R¹⁰)—, —S(O)₂—, —S(O)—, —S—, —N(R¹⁰)—, —OC(OR¹⁰)(R^{10a})—, —N(R¹⁰)C(O)N(R^{10a})—, and —OC(O)N(R¹⁰)—; wherein each —R⁹ is selected from the group consisting of C₁₋₆ alkyl; and each —R¹⁰ and —R^{10a} is independently selected from the group consisting of —H and C₁₋₆ alkyl.

In certain embodiments —SP— of formula (A-1) is C₁₋₁₀ alkyl, which C₁₋₁₀ alkyl is optionally substituted with one or more —R⁹, and which C₁₋₁₀ alkyl is optionally interrupted by one or more groups selected from the group consisting of —O—, —C(O)N(R¹⁰)—, —S(O)₂—, —S(O)—, —S—, —N(R¹⁰)—, —OC(OR¹⁰)(R^{10a})—, —N(R¹⁰)C(O)N(R^{10a})—, and —OC(O)N(R¹⁰)—; wherein each —R⁹ is selected from the group consisting of C₁₋₆ alkyl; and each —R¹⁰ and —R^{10a} is independently selected from the group consisting of —H and C₁₋₆ alkyl.

In certain embodiments—POL of formula (A-1) is a PEG-based polymer. In certain embodiments—POL is of formula (A-11)

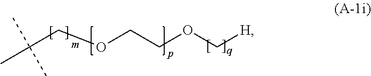

wherein
the dashed line indicates attachment to —SP—;
m is 0 or 1;
p is an integer ranging from about 10 to about 540; and
q is selected from the group consisting of 1, 2, 3, 4, 5, and 6.

In certain embodiments m of formula (A-11) is 0. In certain embodiments m of formula (A-11) is 1.

In certain embodiments p of formula (A-11) is an integer ranging from about 10 to about 450, such as from about 22 to about 340, from about 45 to about 230, from about 65 to about 160, from about 90 to about 140, such as from about 95 to about 135 or such as from about 100 to about 125.

In certain embodiments q of formula (A-11) is 1. In certain embodiments q of formula (A-11) is 2. In certain embodiments q of formula (A-11) is 3. In certain embodiments q of formula (A-11) is 4. In certain embodiments q of formula (A-11) is 5. In certain embodiments q of formula (A-li) is 6.

In certain embodiments —M_{mod} is of formula (A-1a)

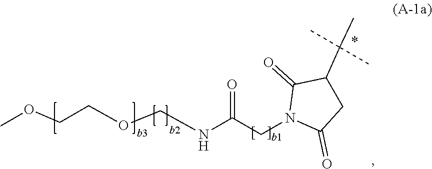

wherein
the dashed line marked with the asterisk indicates attachment to the sulfur of the side chain of a cysteine residue of —D′—;
b1 is an integer selected from the group consisting of 1, 2, 3, 4, 5, 6, 7, 8, 9, 10, 11, 12, 13, 14, 15, 16, 17, 18, 19 and 20;
b2 is an integer selected from the group consisting of 1, 2, 3, 4, 5, 6, 7, 8, 9, 10, 11, 12, 13, 14, 15, 16, 17, 18, 19 and 20; and
b3 is an integer ranging from about 10 to about 540.

In certain embodiments —M_{mod} is of formula (A-1a), wherein the dashed line marked with the asterisk indicates attachment to the sulfur of the side chain of the cysteine residue at position 38 of SEQ ID NO:4.

In certain embodiments b1 of formula (A-1a) is an integer ranging from 1 to 8. In certain embodiments b1 of formula (A-1a) is an integer ranging from 1 to 6. In certain embodiments b1 of formula (A-1a) is an integer ranging from 1 to 4. In certain embodiments b1 of formula (A-1a) is 1. In certain embodiments b1 of formula (A-1a) is 2. In certain embodiments b1 of formula (A-1a) is 3. In certain embodiments b1 of formula (A-1a) is 4. In certain embodiments b1 of formula (A-1a) is 5. In certain embodiments b1 of formula (A-1a) is 6.

In certain embodiments b2 of formula (A-1a) is an integer ranging from 1 to 8. In certain embodiments b2 of formula (A-1a) is an integer ranging from 1 to 6. In certain embodiments b2 of formula (A-1a) is an integer ranging from 1 to 4. In certain embodiments b2 of formula (A-1a) is 1. In certain embodiments b2 of formula (A-1a) is 2. In certain embodiments b2 of formula (A-1a) is 3. In certain embodiments b2 of formula (A-1a) is 4. In certain embodiments b2 of formula (A-1a) is 5. In certain embodiments b2 of formula (A-1a) is 6.

In certain embodiments b3 of formula (A-1a) is an integer ranging from about 10 to about 450, such as from about 22 to about 340, from about 45 to about 230, from about 65 to about 160, from about 90 to about 140, from about 95 to about 135 or such as from about 100 to about 125.

In certain embodiments b1 of formula (A-1a) is 2, b2 of formula (A-1a) is 2 and b3 of formula (A-1a) ranges from about 90 to about 140. In certain embodiments b1 of formula (A-1a) is 3, b2 of formula (A-1a) is 2 and b3 of formula (A-1a) ranges from about 90 to about 140. In certain embodiments b1 of formula (A-1a) is 2, b2 of formula (A-1a) is 3 and b3 of formula (A-1a) ranges from about 90 to about 140. In certain embodiments b1 of formula (A-1a) is 3, b2 of formula (A-1a) is 3 and b3 of formula (A-1a) ranges from about 90 to about 140.

In certain embodiments b1 of formula (A-1a) is 2, b2 of formula (A-1a) is 2 and b3 of formula (A-1a) ranges from about 95 to about 135. In certain embodiments b1 of formula (A-1a) is 3, b2 of formula (A-1a) is 2 and b3 of formula (A-1a) ranges from about 95 to about 135. In certain embodiments b1 of formula (A-1a) is 2, b2 of formula (A-1a) is 3 and b3 of formula (A-1a) ranges from about 95 to about 135. In certain embodiments b1 of formula (A-1a) is 3, b2 of formula (A-1a) is 3 and b3 of formula (A-1a) ranges from about 95 to about 135.

In certain embodiments b1 of formula (A-1a) is 2, b2 of formula (A-1a) is 2 and b3 of formula (A-1a) ranges from about 100 to about 125. In certain embodiments b1 of formula (A-1a) is 3, b2 of formula (A-1a) is 2 and b3 of formula (A-1a) ranges from about 100 to about 125. In certain embodiments b1 of formula (A-1a) is 2, b2 of formula (A-1a) is 3 and b3 of formula (A-1a) ranges from about 100 to about 125. In certain embodiments b1 of formula (A-1a) is 3, b2 of formula (A-1a) is 3 and b3 of formula (A-1a) ranges from about 100 to about 125.

In certain embodiments —M_{mod} is of formula (A-1d)

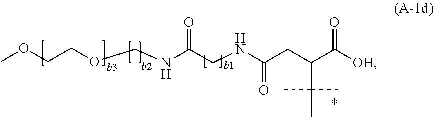

wherein
the dashed line marked with the asterisk indicates attachment to the sulfur of a cysteine residue of —D′—;
b1 is an integer selected from the group consisting of 1, 2, 3, 4, 5, 6, 7, 8, 9, 10, 11, 12, 13, 14, 15, 16, 17, 18, 19 and 20;
b2 is an integer selected from the group consisting of 1, 2, 3, 4, 5, 6, 7, 8, 9, 10, 11, 12, 13, 14, 15, 16, 17, 18, 19 and 20; and
b3 is an integer ranging from about 10 to about 540.

In certain embodiments —M_{mod} is of formula (A-1d), wherein the dashed line marked with the asterisk indicates attachment to the sulfur of the side chain of the cysteine residue at position 38 of SEQ ID NO:4.

In certain embodiments b1 of formula (A-1d) is an integer ranging from 1 to 8. In certain embodiments b1 of formula (A-1d) is an integer ranging from 1 to 6. In certain embodiments b1 of formula (A-1d) is an integer ranging from 1 to 4. In certain embodiments b1 of formula (A-1d) is 1. In certain embodiments b1 of formula (A-1d) is 2. In certain embodiments b1 of formula (A-1d) is 3. In certain embodiments b1 of formula (A-1d) is 4. In certain embodiments b1 of formula (A-1d) is 5. In certain embodiments b1 of formula (A-1d) is 6.

In certain embodiments b2 of formula (A-1d) is an integer ranging from 1 to 8. In certain embodiments b2 of formula (A-1d) is an integer ranging from 1 to 6. In certain embodiments b2 of formula (A-1d) is an integer ranging from 1 to 4. In certain embodiments b2 of formula (A-1d) is 1. In certain embodiments b2 of formula (A-1d) is 2. In certain embodiments b2 of formula (A-1d) is 3. In certain embodiments b2 of formula (A-1d) is 4. In certain embodiments b2 of formula (A-1d) is 5. In certain embodiments b2 of formula (A-1d) is 6.

In certain embodiments b3 of formula (A-1d) is an integer ranging from about 10 to about 450, such as from about 22 to about 340, from about 45 to about 230, from about 65 to about 160, from about 90 to about 140, from about 95 to about 135 or such as from about 100 to about 125.

In certain embodiments b1 of formula (A-1d) is 2, b2 of formula (A-1d) is 2 and b3 of formula (A-1d) ranges from about 90 to about 140. In certain embodiments b1 of formula (A-1d) is 3, b2 of formula (A-1d) is 2 and b3 of formula (A-1d) ranges from about 90 to about 140. In certain embodiments b1 of formula (A-1d) is 2, b2 of formula (A-1d) is 3 and b3 of formula (A-1d) ranges from about 90 to about 140. In certain embodiments b1 of formula (A-1d) is 3, b2 of formula (A-1d) is 3 and b3 of formula (A-1d) ranges from about 90 to about 140.

In certain embodiments b1 of formula (A-1d) is 2, b2 of formula (A-1d) is 2 and b3 of formula (A-1d) ranges from about 95 to about 135. In certain embodiments b1 of formula (A-1d) is 3, b2 of formula (A-1d) is 2 and b3 of formula (A-1d) ranges from about 95 to about 135. In certain embodiments b1 of formula (A-1d) is 2, b2 of formula (A-1d) is 3 and b3 of formula (A-1d) ranges from about 95 to about 135. In certain embodiments b1 of formula (A-1d) is 3, b2 of formula (A-1d) is 3 and b3 of formula (A-1d) ranges from about 95 to about 135.

In certain embodiments b1 of formula (A-1d) is 2, b2 of formula (A-1d) is 2 and b3 of formula (A-1d) ranges from about 100 to about 125. In certain embodiments b1 of formula (A-1d) is 3, b2 of formula (A-1d) is 2 and b3 of formula (A-1d) ranges from about 100 to about 125. In certain embodiments b1 of formula (A-1d) is 2, b2 of formula (A-1d) is 3 and b3 of formula (A-1d) ranges from about 100 to about 125. In certain embodiments b1 of formula (A-1d) is 3, b2 of formula (A-1d) is 3 and b3 of formula (A-1d) ranges from about 100 to about 125.

In certain embodiments —M_{mod} is of formula (A-1e)

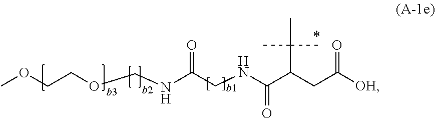

wherein
the dashed line marked with the asterisk indicates attachment to the sulfur of the side chain of a cysteine residue of —D′—;
b1 is an integer selected from the group consisting of 1, 2, 3, 4, 5, 6, 7, 8, 9, 10, 11, 12, 13, 14, 15, 16, 17, 18, 19 and 20;
b2 is an integer selected from the group consisting of 1, 2, 3, 4, 5, 6, 7, 8, 9, 10, 11, 12, 13, 14, 15, 16, 17, 18, 19 and 20; and
b3 is an integer ranging from 12 to 540.

In certain embodiments —M_{mod} is of formula (A-1e), wherein the dashed line marked with the asterisk indicates attachment to the sulfur of the side chain of the cysteine residue at position 38 of SEQ ID NO:4.

In certain embodiments b1 of formula (A-1e) is an integer ranging from 1 to 8. In certain embodiments b1 of formula (A-1e) is an integer ranging from 1 to 6. In certain embodiments b1 of formula (A-1e) is an integer ranging from 1 to 4. In certain embodiments b1 of formula (A-1e) is 1. In certain embodiments b1 of formula (A-1e) is 2. In certain embodiments b1 of formula (A-1e) is 3. In certain embodiments b1 of formula (A-1e) is 4. In certain embodiments b1 of formula (A-1e) is 5. In certain embodiments b1 of formula (A-1e) is 6.

In certain embodiments b2 of formula (A-1e) is an integer ranging from 1 to 8. In certain embodiments b2 of formula (A-1e) is an integer ranging from 1 to 6. In certain embodiments b2 of formula (A-1e) is an integer ranging from 1 to 4. In certain embodiments b2 of formula (A-1e) is 1. In certain embodiments b2 of formula (A-1e) is 2. In certain embodiments b2 of formula (A-1e) is 3. In certain embodiments b2 of formula (A-1e) is 4. In certain embodiments b2 of formula (A-1e) is 5. In certain embodiments b2 of formula (A-1e) is 6.

In certain embodiments b3 of formula (A-1e) is an integer ranging from 10 to 450, such as from 22 to 340, from 45 to 230, from 65 to 160, from 90 to 140, from 95 to 135 or such as from 100 to 125.

In certain embodiments b1 of formula (A-1e) is 2, b2 of formula (A-1e) is 2 and b3 of formula (A-1e) ranges from about 90 to about 140. In certain embodiments b1 of formula (A-1e) is 3, b2 of formula (A-1e) is 2 and b3 of formula (A-1e) ranges from about 90 to about 140. In certain embodiments b1 of formula (A-1e) is 2, b2 of formula (A-1e) is 3 and b3 of formula (A-1e) ranges from about 90 to about 140. In certain embodiments b1 of formula (A-1e) is 3, b2 of formula (A-1e) is 3 and b3 of formula (A-1e) ranges from about 90 to about 140.

In certain embodiments b1 of formula (A-1e) is 2, b2 of formula (A-1e) is 2 and b3 of formula (A-1e) ranges from about 95 to about 135. In certain embodiments b1 of formula (A-1e) is 3, b2 of formula (A-1e) is 2 and b3 of formula (A-1e) ranges from about 95 to about 135. In certain embodiments b1 of formula (A-1e) is 2, b2 of formula (A-1e) is 3 and b3 of formula (A-1e) ranges from about 95 to about 135. In certain embodiments b1 of formula (A-1e) is 3, b2 of formula (A-1e) is 3 and b3 of formula (A-1e) ranges from about 95 to about 135.

In certain embodiments b1 of formula (A-1e) is 2, b2 of formula (A-1e) is 2 and b3 of formula (A-1e) ranges from about 100 to about 125. In certain embodiments b1 of formula (A-1e) is 3, b2 of formula (A-1e) is 2 and b3 of formula (A-1e) ranges from about 100 to about 125. In certain embodiments b1 of formula (A-1e) is 2, b2 of formula (A-1e) is 3 and b3 of formula (A-1e) ranges from about 100 to about 125. In certain embodiments b1 of formula (A-1e) is 3, b2 of formula (A-1e) is 3 and b3 of formula (A-1e) ranges from about 100 to about 125.

In a plurality of IL-2 conjugates, some conjugates may comprise a moiety —M_{mod} of formula (A-1d) and some may comprise a moiety —M_{mod} of formula (A-1e), i.e., the plurality of IL-2 conjugates is a mixture. Optionally, a certain percentage of IL-2 conjugates may comprise a moiety —M_{mod} of formula (A-1a). It is understood that in such IL-2 conjugates with a moiety —M_{mod} of formula (A-1a) hydrolysis of the thiosuccinimide ring did not occur. A plurality of IL-2 conjugates may be present for example in a batch of IL-2 conjugates or in a pharmaceutical composition comprising such IL-2 conjugates.

In a plurality of IL-2 conjugates the sum of IL-2 conjugates with a moiety —M_{mod} of formula (A-1d) and IL-2 conjugates with a moiety —M_{mod} of formula (A-1e) is in certain embodiments at least 70% of all IL-2 conjugates. In a plurality of IL-2 conjugates the sum of IL-2 conjugates with a moiety —M_{mod} of formula (A-1d) and IL-2 conjugates with a moiety —M_{mod} of formula (A-1e) is in certain embodiments at least 80% of all IL-2 conjugates. In a plurality of IL-2 conjugates the sum of IL-2 conjugates with a moiety —M_{mod} of formula (A-1d) and IL-2 conjugates with a moiety —M_{mod} of formula (A-1e) is in certain embodiments at least 90% of all IL-2 conjugates. In a plurality of IL-2 conjugates the sum of IL-2 conjugates with a moiety —M_{mod} of formula (A-1d) and IL-2 conjugates with a moiety —M_{mod} of formula (A-1e) is in certain embodiments at least 95% of all IL-2 conjugates.

In a plurality of IL-2 conjugates some conjugates may comprise a moiety —M_{mod} of formula (A1-a), some conjugates may comprise a moiety —M_{mod} of formula (A-1d) and some conjugates may comprise a moiety —M_{mod} of formula (A-1e). In a plurality of IL-2 conjugates the sum of IL-2 conjugates with a moiety —M_{mod} of formula (A-1d) and IL-2 conjugates with a moiety —M_{mod} of formula (A-1e) is in certain embodiments at least 70% of all IL-2 conjugates and the remaining IL-2 conjugates comprise a moiety —M_{mod} of formula (A-1a). In a plurality of IL-2 conjugates the sum of IL-2 conjugates with a moiety —M_{mod} of formula (A-1d) and IL-2 conjugates with a moiety —M_{mod} of formula (A-1e) is in certain embodiments at least 80% of all IL-2 conjugates and the remaining IL-2 conjugates comprise a moiety —M_{mod} of formula (A-1a). In a plurality of IL-2 conjugates the sum of IL-2 conjugates with a moiety —M_{mod} of formula (A-1d) and IL-2 conjugates with a moiety —M_{mod} of formula (A-1e) is in certain embodiments at least 90% of all IL-2 conjugates and the remaining IL-2 conjugates comprise a moiety —M_{mod} of formula (A-1a). In a plurality of IL-2 conjugates the sum of IL-2 conjugates with a moiety —M_{mod} of formula (A-1d) and IL-2 conjugates with a moiety —M_{mod} of formula (A-1e) is in certain embodiments at least 95% of all IL-2 conjugates and the remaining IL-2 conjugates comprise a moiety —M_{mod} of formula (A-1a).

The IL-2 conjugate of formula (I) comprises at least one covalently and reversibly attached polymeric moiety —Z.

The addition of such at least one covalently and reversibly attached polymeric moiety results in an extension of the circulation half-life of the IL-2 moiety beyond the extension provided by an optionally present moiety —M_{mod}, while its reversible attachment ensures sufficient pharmaceutical activity.

In certain embodiments —Z has a molecular weight ranging from about 20 kDa to about 60 kDa, such as from about 25 kDa to about 55 kDa, from about 30 kDa to about 50 kDa or from about 35 kDa to about 45 kDa. In certain embodiments —Z has a molecular weight of about 40 kDa.

In certain embodiments —Z of formula (I) is a polymeric moiety comprising a polymer selected from the group consisting of poly(2-methacryloyl-oxyethyl phosphoyl cholins), poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, alginate, mannans, pectins, rhamnogalacturonans, starches, hydroxyalkyl starches, hydroxyethyl starches and other carbohydrate-based polymers, xylans, and copolymers thereof.

In certain embodiments —Z of formula (I) is a PEG-based moiety, such as a linear, branched or multi-arm PEG-based moiety. In certain embodiments —Z is a branched PEG-based moiety, such as a branched PEG-based moiety having one, two, three, four, five or six branching points.

In certain embodiments —Z is a branched PEG-based moiety having one, two or three branching points. In certain embodiments —Z is a branched PEG-based moiety having one branching point.

In certain embodiments —Z is a branched PEG-based moiety having two branching points. In certain embodiments —Z is a branched PEG-based moiety having three branching points.

Each branching point may be independently selected from the group consisting of —N<, —CH< and >C<.

In certain embodiments —Z of formula (I) comprises a moiety of formula (A)

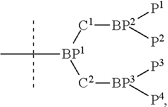

wherein
—BP¹<, —BP²<, —BP³< are independently of each other selected from the group consisting of —N< and —C(R⁸)K;
—R⁸ is selected from the group consisting of —H, C₁₋₆ alkyl, C₂₋₆ alkenyl and C₂₋₆ alkynyl; —P¹, —P² —P³ —P⁴ are independently of each other a PEG-based chain comprising at least 40% PEG and having a molecular weight ranging from about 5 to about 15 kDa; —C¹—, —C²— are independently of each other selected from the group consisting of C₁₋₅₀ alkyl, C₂₋₅₀ alkenyl, and C₂₋₅₀ alkynyl; wherein C₁₋₅₀ alkyl, C₂₋₅₀ alkenyl, and C₂₋₅₀ alkynyl are optionally substituted with one or more —R⁹, which are the same or different and wherein C₁₋₅₀ alkyl, C₂₋₅₀ alkenyl, and C₂₋₅₀ alkynyl are optionally interrupted by one or more groups selected from the group consisting of —T—, —C(O)O—, —O—, —C(O)—, —C(O)N(R¹⁰)—, —S(O)₂N(R¹⁰)—, —S(O)N(R¹⁰)—, —S(O)₂—, —S(O)—, —N(R¹⁰)S(O)₂N(R^{10a})—, —S—, —N(R¹⁰)—, —OC(ORO)(R^{10a})—, —N(R¹⁰)C(O)N(R^{10a})- and —OC(O)N(R¹⁰)—;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R⁹, which are the same or different;
each —R⁹ is independently selected from the group consisting of halogen, —CN, oxo (═O), —COOR¹¹, —OR¹, —C(O)R¹¹, —C(O)N(R¹¹R^{11a}), —S(O)₂N(R¹¹R^{11a}) —S(O)N(R¹¹R^{11a}), —S(O)₂R¹¹, —S(O)R¹¹, —N(R¹¹)S(O)₂N(R^{11a}R^{11b}), —SR¹¹, —N(R¹¹R^{11a}), —NO₂, —OC(O)R¹¹, —N(R¹¹)C(O)R^{11a}, —N(R¹¹)S(O)₂R^{11a}, —N(R¹¹)S(O)R^{11a} —N(R¹¹)C(O)OR^{11a}, —N(R¹¹)C(O)N(R^{11a}R^{11b}), —OC(O)N(R¹¹R^{11a}), and C₁₋₆ alkyl; wherein C₁₋₆ alkyl is optionally substituted with one or more halogen, which are the same or different; and
each —R¹⁰, —R^{10a}, —R¹¹, —R^{11a} and —R^{11b} is independently selected from the group consisting of —H, and C₁₋₆ alkyl, wherein C₁₋₆ alkyl is optionally substituted with one or more halogen, which are the same or different.

In certain embodiments —P¹, —P² —P³ —P⁴ of formula (A) are independently of each other a PEG-based chain comprising at least 50% PEG and having a molecular weight ranging from about 5 kDa to about 15 kDa, such as ranging from about 6.25 kDa to about 13.75 kDa, such as ranging from about 7.5 kDa to about 12.5 kDa or ranging from about 8.75 kDa to about 11.25 kDa. In certain embodiments —P¹, —P² —P³ —P⁴ of formula (A) are independently of each other a PEG-based chain comprising at least 50% PEG and having a molecular weight of about 10 kDa.

In certain embodiments —P¹, —P², —P³ and —P⁴ of formula (A) have the same structure.

In certain embodiments BP¹ of formula (A) is —N<.

In certain embodiments BP² and BP³ of formula (A) have the same structure. In certain embodiments BP² and BP³ of formula (A) are both —CH<.

In certain embodiments —C¹— and —C²— of formula (A) have the same structure. In certain embodiments —C¹— and —C²— of formula (A) are C₁₋₁₀ alkyl interrupted by one or more of the groups selected from the group consisting of —O—, —C(O)N(R¹⁰)— and 3- to 10-membered heterocyclyl; wherein the 3- to 10-membered heterocyclyl is substituted with at least one oxo (═O).

In certain embodiments —C¹— and —C²— of formula (A) are of formula (A-a)

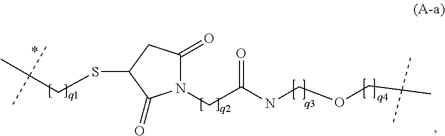

wherein
the dashed line marked with the asterisk indicates attachment to BP¹;
the unmarked dashed line indicates attachment to BP² or BP³, respectively;
q1 is selected from the group consisting of 1, 2, 3, 4, 5, 6, 7 and 8;
q2 is selected from the group consisting of 1, 2, 3, 4, and 5;
q3 is selected from the group consisting of 1, 2, 3, 4, 5, 6, 7 and 8; and
q4 is selected from the group consisting of 1, 2 and 3.

In certain embodiments q1 of formula (A-a) is selected from the group consisting of 4, 5, 6, 7, and 8. In certain embodiments q1 of formula (A-a) is selected from the group consisting of 5, 6 and 7. In certain embodiments q1 of formula (A-a) is 1. In certain embodiments q1 of formula (A-a) is 2. In certain embodiments q1 of formula (A-a) is 3. In certain embodiments q1 of formula (A-a) is 4. In certain embodiments q1 of formula (A-a) is 5. In certain embodiments q1 of formula (A-a) is 6. In certain embodiments q1 of formula (A-a) is 7. In certain embodiments q1 of formula (A-a) is 8.

In certain embodiments q2 of formula (A-a) is selected from the group consisting of 1, 2 and 3.

In certain embodiments q2 of formula (A-a) is 1. In certain embodiments q2 of formula (A-a) is 2. In certain embodiments q2 of formula (A-a) is 3. In certain embodiments q2 of formula (A-a) is 4. In certain embodiments q2 of formula (A-a) is 5.

In certain embodiments q3 of formula (A-a) is selected from the group consisting of 2, 3, 4, and 5. In certain embodiments q3 of formula (A-a) is selected from the group consisting of 2, 3 and 4. In certain embodiments q3 of formula (A-a) is 1. In certain embodiments q3 of formula (A-a) is 2. In certain embodiments q3 of formula (A-a) is 3. In certain embodiments q3 of formula (A-a) is 4. In certain embodiments q3 of formula (A-a) is 5. In certain embodiments q3 of formula (A-a) is 6. In certain embodiments q3 of formula (A-a) is 7. In certain embodiments q3 of formula (A-a) is 8.

In certain embodiments q4 of formula (A-a) is 1. In certain embodiments q4 of formula (A-a) is 2. In certain embodiments q4 of formula (A-a) is 3.

In certain embodiments —P¹, —P², —P³ and —P⁴ of formula (A) are independently of each other of formula (A-b)

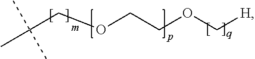

wherein
the dashed line indicates attachment to the remainder of —Z;
m is 0 or 1;
p is an integer ranging from 110 to 345; and
q is selected from the group consisting of 1, 2, 3, 4, 5, and 6.

In certain embodiments m of formula (A-b) is 0. In certain embodiments m of formula (A-b) is 1.

In certain embodiments p of formula (A-b) is an integer ranging from 140 to 315. In certain embodiments p of formula (A-b) is an integer ranging from 170 to 285. In certain embodiments p of formula (A-b) is an integer ranging from 195 to 255. In certain embodiments p of formula (A-b) is an integer ranging from 200 to 250. In certain embodiments p of formula (A-b) is an integer ranging from 210 to 250. In certain embodiments p of formula (A-b) is an integer ranging from 220 to 240.

In certain embodiments q of formula (A-b) is 1. In certain embodiments q of formula (A-b) is 2. In certain embodiments q of formula (A-b) is 3. In certain embodiments q of formula (A-b) is 4. In certain embodiments q of formula (A-b) is 5. In certain embodiments q of formula (A-b) is 6.

In certain embodiments —Z of formula (I) comprises a moiety of formula (A-c):

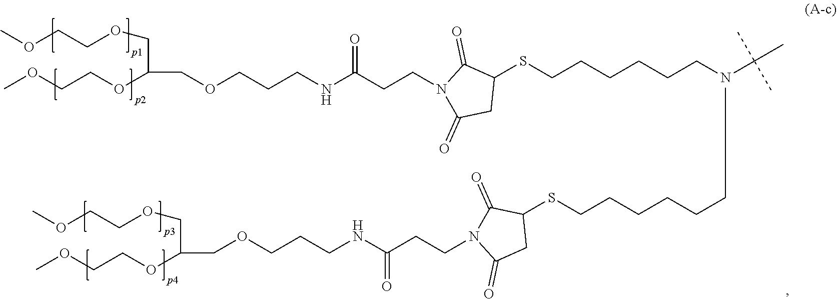

wherein
p1, p2, p3, p4 are independently of each other an integer ranging from 110 to 345 In certain embodiments —Z is a moiety of formula (A-c).

In certain embodiments p1, p2, p3, p4 of formula (A-c) are independently of each other an integer ranging from 140 to 315. In certain embodiments p1, p2, p3, p4 of formula (A-c) are independently of each other an integer ranging from 170 to 285. In certain embodiments p1, p2, p3, p4 of formula (A-c) are independently of each other an integer ranging from 195 to 255.

In certain embodiments p1, p2, p3, p4 of formula (A-c) are independently of each other an integer ranging from 200 to 250. In certain embodiments p1, p2, p3, p4 of formula (A-c) are independently of each other an integer ranging from 210 to 250. In certain embodiments p1, p2, p3, p4 of formula (A-c) are independently of each other an integer ranging from 220 to 240.

The moiety —M_{mod} is attached to a different amino acid residue of —D′— than the moiety —L¹—L²—Z. —L¹— may be attached to a proteinogenic or non-proteinogenic amino acid residue of —D′—. In certain embodiments —L¹— is attached to a proteinogenic amino acid residue of —D′—, such as to a proteinogenic amino acid residue selected from the group consisting of cysteine, methionine, histidine, lysine, tryptophan, serine, threonine, tyrosine, aspartic acid, glutamic acid, glutamine and arginine. In certain embodiments such proteinogenic amino acid residue is selected from the group consisting of cysteine, histidine, lysine, tryptophan, serine, threonine, tyrosine, aspartic acid, glutamic acid and arginine. In certain embodiments attachment of —L¹— is to a non-proteinogenic amino acid residue of —D′—. Examples for such non-proteinogenic amino acid residues are as described elsewhere herein.

In certain embodiments —L¹— is attached to a cysteine residue of —D′—. In certain embodiments —L¹— is attached to a histidine residue of —D′—. In certain embodiments —L¹— is attached to a lysine residue of —D′—. In certain embodiments —L¹— is attached to a tryptophan residue of —D′—. In certain embodiments —L¹—0 is attached to a serine residue of —D′—. In certain embodiments —L¹— is attached to a threonine residue of —D′—. In certain embodiments —L¹— is attached to a tyrosine residue of —D′—. In certain embodiments —L¹— is attached to an aspartic acid residue of —D′—. In certain embodiments —L¹— is attached to a glutamic acid residue of —D′—. In certain embodiments —L¹— is attached to an arginine residue of —D′—.

The moiety —L¹— may be connected to —D′— through any type of linkage, provided that it is reversible. In certain embodiments —L¹— is connected to —D′— through a linkage selected from the group consisting of amide, ester, carbamate, acetal, aminal, imine, oxime, hydrazone, disulfide and acylguanidine. In certain embodiments —L¹— is connected to —D′— through a linkage selected from the group consisting of amide, ester, carbamate and acylguanidine. It is understood that these linkages may not be reversible per se, but that reversibility may be an effect of certain groups of atoms or moieties present in —L¹—. In certain embodiments —L¹— is connected to —D′— through a carbamate linkage. In certain embodiments —L¹— is connected to —D′— through an amide linkage.

In certain embodiments —L¹— is connected to —D′— via the nitrogen of the ε-amino group of a lysine residue of —D′— or via the nitrogen of the N-terminal amine of —D′—. In certain embodiments —L¹— is connected to —D′— via the nitrogen of the ε-amino group of a lysine residue or via the nitrogen of the N-terminal amine and the linkage formed between —D′— and —L¹— is a carbamate.

In certain embodiments —L¹— is connected to —D′— via the nitrogen of the ε-amino group of a lysine residue of —D′—. In certain embodiments —L¹— is connected to —D′— via the nitrogen of the c-amino group of a lysine residue of —D′— and the linkage formed between —D′— and —L¹— is a carbamate.

In certain embodiments —L¹— is connected to —D′— via the nitrogen of the N-terminal amine of —D′—. In certain embodiments —L¹— is connected to —D′— via the nitrogen of the N-terminal amine of —D′— and the linkage formed between —D′— and —L¹— is a carbamate.

In certain embodiments —L¹— has a structure as disclosed in WO 2009/095479 A2. Accordingly, in certain embodiments the moiety —L¹— is of formula (II):

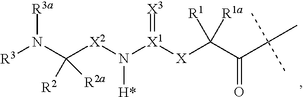

wherein the dashed line indicates attachment to a nitrogen of —D′— by forming an amide bond;
—X— is —C(R⁴R^{4a})—; —N(R⁴)—; —O—; —C(R⁴R^{4a})—C(R⁵R^{5a})—; —C(R⁵R^{5a})—C(R⁴R^{4a})—; —C(R⁴R^{4a})—N(R⁶)—; —N(R⁶)—C(R⁴R^{4a})—; —C(R⁴R^{4a})—O—; —O—C(R⁴R^{4a})—; or —C(R⁷R^{7a})—X¹ is C; or S(O);
—X²— is —C(R⁸R^{8a})—; or —C(R⁸R^{8a})—C(R⁹R^{9a})—; ═X³ is ═O; ═S; or ═N—CN;
—R¹, —R^{1a} —R², —R^{2a}, —R⁴, —R^{4a}, —R⁵, —R^{5a}, —R⁶, —R⁸, —R^{8a}, —R⁹, —R^{9a} are independently selected from the group consisting of —H; and C₁₋₆ alkyl;
—R³, —R^{3a} are independently selected from the group consisting of —H; and C₁₋₆ alkyl, provided that in case one of —R³, —R^{3a} or both are other than —H they are connected to N to which they are attached through an SP³-hybridized carbon atom;
—R⁷ is —N(R¹⁰R^{10a}); or —NR¹⁰—(C═O)—R¹¹;
R^{7a}—R¹⁰, —R^{10a}, —R¹¹ are independently of each other —H; or C₁₋₆ alkyl; optionally, one or more of the pairs —R^{1a}/—R^{4a}, —R^{1a}/—R^{5a}, —R^{1a}/—R^{7a}, —R^{4a}/—R^{5a}, —R^{8a}/—R^{9a} form a chemical bond;
optionally, one or more of the pairs —R¹/R^{1a}, —R²/—R^{2a}, —R⁴/—R^{4a}, —R⁵/—R^{5a}, —R⁸/—R^{8a}, —R⁹/—R^{9a} are joined together with the atom to which they are attached to form a C₃₋₁₀ cycloalkyl; or 3- to 10-membered heterocyclyl;
optionally, one or more of the pairs —R¹/—R⁴, —R¹/R⁵, —R¹/—R⁶, —R¹/—R^{7a}, —R⁴/—R⁵, —R⁴/—R⁶, —R¹/—R⁹, —R²/—R³ are joined together with the atoms to which they are attached to form a ring A;
optionally, R³/R^{3a} are joined together with the nitrogen atom to which they are attached to form a 3- to 10-membered heterocycle;
A is selected from the group consisting of phenyl; naphthyl; indenyl; indanyl; tetralinyl; C₃₋₁₀ cycloalkyl; 3- to 10-membered heterocyclyl; and 8- to 11-membered heterobicyclyl; and
wherein —L¹— is substituted with at least one —L²—Z and wherein —L¹— is optionally further substituted, provided that the hydrogen marked with the asterisk in formula (II) is not replaced by —L²—Z or a substituent.

Preferably —L¹— of formula (II) is substituted with one moiety —L²—Z.

In one embodiment —L¹— of formula (II) is not further substituted.

It is understood that if —R³/—R^{3a} of formula (II) are joined together with the nitrogen atom to which they are attached to form a 3- to 10-membered heterocycle, only such 3- to 10-membered heterocycles may be formed in which the atoms directly attached to the nitrogen are sp³—hybridized carbon atoms. In other words, such 3- to 10-membered heterocycle formed by —R³/—R^{3a} together with the nitrogen atom to which they are attached has the following structure:

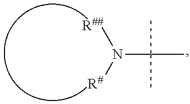

wherein
the dashed line indicates attachment to the rest of —L¹—;
the ring comprises 3 to 10 atoms comprising at least one nitrogen; and R^{#} and R^{##} represent an SP³-hydridized carbon atom.

It is also understood that the 3- to 10-membered heterocycle may be further substituted.

Exemplary embodiments of suitable 3- to 10-membered heterocycles formed by —R³/—R^{3a} of formula (II) together with the nitrogen atom to which they are attached are the following:

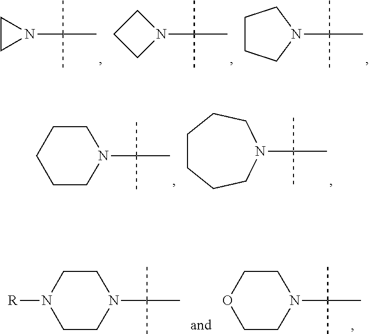

wherein
dashed lines indicate attachment to the rest of the molecule; and
—R is selected from the group consisting of —H and C₁₋₆ alkyl.

—L¹— of formula (II) may optionally be further substituted. In general, any substituent may be used as far as the cleavage principle is not affected, i.e. the hydrogen marked with the asterisk in formula (II) is not replaced and the nitrogen of the moiety

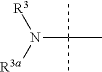

of formula (II) remains part of a primary, secondary or tertiary amine, i.e. —R³ and —R^{3a} are independently of each other —H or are connected to —N< through an sp³-hybridized carbon atom.

In certain embodiments —L¹— has a structure as disclosed in WO2016/020373A1. Accordingly, in certain embodiments the moiety —L¹— is of formula (III):

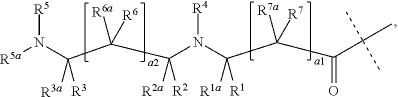

wherein
the dashed line indicates attachment to a primary or secondary amine or hydroxyl of —D′— by forming an amide or ester linkage, respectively;
—R¹, —R^{1a}, —R², —R^{2a}, —R³ and —R^{3a} are independently of each other selected from the group consisting of —H, —C(R⁸R^{8a}R^{8b}), —C(═O)R⁸, —C—N, —C(═NR⁸)R^{8a}, —CR⁸(═CR^{8a}R^{8b}),
—C≡CR⁸ and —T;
—R⁴, —R⁵ and —R^{5a} are independently of each other selected from the group consisting of —H, —C(R⁹R^{9a}R^{9b}) and —T;
a1 and a2 are independently of each other 0 or 1;
each —R⁶, —R^{6a}, —R⁷, —R^{7a}, —R⁸, —R^{8a}, —R^{8b}, —R⁹, —R^{9a}, —R^{9b} are independently of each other selected from the group consisting of —H, halogen, —CN, —COOR¹⁰, —OR¹⁰, —C(O)R¹⁰, —C(O)N(R¹⁰R^{10a}), —S(O)₂N(R¹⁰R^{10a}), —S(O)N(R¹⁰R^{10a}), —S(O)₂R¹⁰, —S(O)R¹⁰, —N(R¹⁰)S(O)₂N(R^{10a}R^{10b}), —SR¹⁰, —N(R¹⁰R^{10a}), —NO₂, —OC(O)R¹⁰, —N(R¹⁰)C(O)R^{10a}, —N(R¹⁰)S(O)₂R^{10a}, —N(R¹⁰)S(O)R^{10a}, —N(R¹⁰)C(O)OR^{10a} —N(R¹⁰)C(O)N(R^{10a}R^{10b}), —OC(O)N(R¹⁰R^{10a}), —T, C₁₋₂₀ alkyl, C₂₋₂₀ alkenyl, and C₂₋₂₀ alkynyl; wherein —T, C₁₋₂₀ alkyl, C₂₋₂₀ alkenyl, and C₂₋₂₀ alkynyl are optionally substituted with one or more —R¹¹, which are the same or different and wherein C₁₋₂₀ alkyl, C₂₋₂₀ alkenyl, and C₂₋₂₀ alkynyl are optionally interrupted by one or more groups selected from the group consisting of —T—, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S——N(R¹²)—, —OC(OR¹²)(R^{12a})—, —N(R¹²)C(O)N(R^{12a})—, and —OC(O)N(R¹²)—;
each —R¹⁰, —R^{10a}, —R^{10b} is independently selected from the group consisting of —H, —T, C₁-20 alkyl, C₂₋₂₀ alkenyl, and C₂₋₂₀ alkynyl; wherein —T, C₁₋₂₀ alkyl, C₂₋₂₀ alkenyl, and C₂₋₂₀ alkynyl are optionally substituted with one or more —R¹¹, which are the same or different and wherein C₁₋₂₀ alkyl, C₂₋₂₀ alkenyl, and C₂₋₂₀ alkynyl are optionally interrupted by one or more groups selected from the group consisting of —T—, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})——S—, —N(R¹²)—, —OC(OR¹²)(R^{12a})—, —N(R¹²)C(O)N(R^{12a})—, and —OC(O)N(R¹²)—;
each T is independently of each other selected from the group consisting of phenyl, naphthyl, indenyl, indanyl, tetralinyl, C₃₁₀ cycloalkyl, 3- to 10-membered heterocyclyl, and 8- to 11-membered heterobicyclyl; wherein each T is independently optionally substituted with one or more —R¹¹, which are the same or different;
each —R¹¹ is independently of each other selected from halogen, —CN, oxo (═O), —COOR¹³, —OR¹³, —C(O)R¹³, —C(O)N(R¹³R^{13a}), —S(O)₂N(R¹³R^{13a}), —S(O)N(R¹³R^{13a}), —S(O)₂R¹³, —S(O)R¹³, —N(R¹³)S(O)₂N(R^{13a}R^{13b}), —SR¹³, —N(R¹³R^{13a}), —NO₂, —OC(O)R¹³, —N(R¹³)C(O)R^{13a}, —N(R¹³)S(O)₂R^{13a}, —N(R¹³)S(O)R^{13a}, —N(R¹³)C(O)OR^{13a}, —N(R¹³)C(O)N(R^{13a}R^{13b}), —OC(O)N(R¹³R^{13a}), and C₁₋₆alkyl; wherein C₁₋₆ alkyl is optionally substituted with one or more halogen, which are the same or different;
each —R¹², —R^{12a}, —R¹³, —R^{13a}, —R^{3b} is independently selected from the group consisting of —H, and C₁₋₆ alkyl; wherein C₁₋₆ alkyl is optionally substituted with one or more halogen, which are the same or different;
optionally, one or more of the pairs —R¹/—R^{1a}, —R²/—R^{2a}, —R³/—R^{3a}, —R⁶/—R^{6a}, —R⁷/—R^{7a} are joined together with the atom to which they are attached to form a C₃₋₁₀ cycloalkyl or a 3- to 10-membered heterocyclyl;
optionally, one or more of the pairs —R¹/—R², —R¹/—R³, —R¹/—R⁴, —R¹/R⁵, —R¹/—R⁶, —R¹/—R⁷, —R²/—R³, —R²/—R⁴, —R²/—R⁵, —R²/—R⁶, —R²/—R⁷, —R³/—R⁴, —R³/—R⁵, —R³/—R⁶, —R³/—R⁷, —R⁴/—R⁵, —R⁴/—R⁶, —R⁴/—R⁷, —R⁵/—R⁶, —R⁵/—R⁷, —R⁶/—R⁷ are joint together with the atoms to which they are attached to form a ring A; A is selected from the group consisting of phenyl; naphthyl; indenyl; indanyl; tetralinyl; C₃₋₁₀ cycloalkyl; 3- to 10-membered heterocyclyl; and 8- to 11-membered heterobicyclyl;
wherein —L¹— is substituted with at least one —L²—Z and wherein —L¹— is optionally further substituted.

The optional further substituents of —L¹— of formula (III) are preferably as described above.

Preferably —L¹— of formula (III) is substituted with one moiety —L²—Z.

In one embodiment —L¹— of formula (III) is not further substituted.

In another embodiment —L¹— has a structure as disclosed in EP1536334B1, WO2009/009712A1, WO2008/034122A1, WO2009/143412A2, WO2011/082368A2, and U.S. Pat. No. 8,618,124B2, which are herewith incorporated by reference.

In certain embodiments —L¹— has a structure as disclosed in U.S. Pat. No. 8,946,405B2 and U.S. Pat. No. 8,754,190B2.

Accordingly, in certain embodiments —L¹— is of formula (IV):

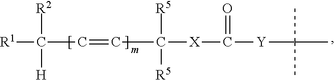

wherein
the dashed line indicates attachment to —D′— through a functional group of —D′— selected from the group consisting of —OH, —SH and —NH₂;
m is 0 or 1;
at least one or both of —R¹ and —R² is/are independently of each other selected from the group consisting of —CN, —NO₂, optionally substituted aryl, optionally substituted heteroaryl, optionally substituted alkenyl, optionally substituted alkynyl, —C(O)R³, —S(O)R³, —S(O)₂R³, and —SR⁴,
one and only one of —R¹ and —R² is selected from the group consisting of —H, optionally substituted alkyl, optionally substituted arylalkyl, and optionally substituted heteroarylalkyl;
—R³ is selected from the group consisting of —H, optionally substituted alkyl, optionally substituted aryl, optionally substituted arylalkyl, optionally substituted heteroaryl, optionally substituted heteroarylalkyl, —OR⁹ and —N(R⁹)₂;
—R⁴ is selected from the group consisting of optionally substituted alkyl, optionally substituted aryl, optionally substituted arylalkyl, optionally substituted heteroaryl, and optionally substituted heteroarylalkyl;
each —R⁵ is independently selected from the group consisting of —H, optionally substituted alkyl, optionally substituted alkenylalkyl, optionally substituted alkynylalkyl, optionally substituted aryl, optionally substituted arylalkyl, optionally substituted heteroaryl and optionally substituted heteroarylalkyl;
—R⁹ is selected from the group consisting of —H and optionally substituted alkyl;
—Y— is absent and —X— is —O— or —S—; or
—Y— is —N(Q)CH₂— and —X— is —O—;
Q is selected from the group consisting of optionally substituted alkyl, optionally substituted aryl, optionally substituted arylalkyl, optionally substituted heteroaryl and optionally substituted heteroarylalkyl;
optionally, —R¹ and —R² may be joined to form a 3 to 8-membered ring; and optionally, both —R⁹ together with the nitrogen to which they are attached form a heterocyclic ring;
wherein —L¹— is substituted with —L²—Z and wherein —L¹— is optionally further substituted.

Only in the context of formula (IV) the terms used have the following meaning: The term “alkyl” as used herein includes linear, branched or cyclic saturated hydrocarbon groups of 1 to 8 carbons, or in some embodiments 1 to 6 or 1 to 4 carbon atoms.

The term “alkoxy” includes alkyl groups bonded to oxygen, including methoxy, ethoxy, isopropoxy, cyclopropoxy, cyclobutoxy, and similar.

The term “alkenyl” includes non-aromatic unsaturated hydrocarbons with carbon-carbon double bonds.

The term “alkynyl” includes non-aromatic unsaturated hydrocarbons with carbon-carbon triple bonds.

The term “aryl” includes aromatic hydrocarbon groups of 6 to 18 carbons, preferably 6 to 10 carbons, including groups such as phenyl, naphthyl, and anthracenyl. The term “heteroaryl” includes aromatic rings comprising 3 to 15 carbons containing at least one N, O or S atom, preferably 3 to 7 carbons containing at least one N, O or S atom, including groups such as pyrrolyl, pyridyl, pyrimidinyl, imidazolyl, oxazolyl, isoxazolyl, thiazolyl, isothiazolyl, quinolyl, indolyl, indenyl, and similar.

In some instance, alkenyl, alkynyl, aryl or heteroaryl moieties may be coupled to the remainder of the molecule through an alkylene linkage. Under those circumstances, the substituent will be referred to as alkenylalkyl, alkynylalkyl, arylalkyl or heteroarylalkyl, indicating that an alkylene moiety is between the alkenyl, alkynyl, aryl or heteroaryl moiety and the molecule to which the alkenyl, alkynyl, aryl or heteroaryl is coupled.

The term “halogen” includes bromo, fluoro, chloro and iodo.

The term “heterocyclic ring” refers to a 4 to 8 membered aromatic or non-aromatic ring comprising 3 to 7 carbon atoms and at least one N, O or S atom. Examples are piperidinyl, piperazinyl, tetrahydropyranyl, pyrrolidine, and tetrahydrofuranyl, as well as the exemplary groups provided for the term “heteroaryl” above.

When a ring system is optionally substituted, suitable substituents are selected from the group consisting of alkyl, alkenyl, alkynyl or an additional ring, each optionally further substituted.

Optional substituents on any group, including the above, include halo, nitro, cyano, —OR, —SR, —NR₂, —OCOR, —NRCOR, —COOR, —CONR₂, —SOR, —SO₂R, —SONR₂, —SO₂N R₂, wherein each R is independently alkyl, alkenyl, alkynyl, aryl or heteroaryl, or two R groups taken together with the atoms to which they are attached form a ring.

Preferably —L¹— of formula (IV) is substituted with one moiety —L²—Z.

In certain embodiments —L¹— has a structure as disclosed in WO2013/036857A1. Accordingly, in certain embodiments —L¹— is of formula (V):

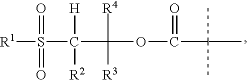

wherein
the dashed line indicates attachment to —D′— through an amine functional group of —D′—; —R¹ is selected from the group consisting of optionally substituted C₁-C₆ linear, branched, or cyclic alkyl; optionally substituted aryl; optionally substituted heteroaryl; alkoxy; and —NR⁵²;
—R² is selected from the group consisting of —H; optionally substituted C₁-C₆ alkyl; optionally substituted aryl; and optionally substituted heteroaryl;
—R³ is selected from the group consisting of —H; optionally substituted C₁-C₆ alkyl; optionally substituted aryl; and optionally substituted heteroaryl;
—R⁴ is selected from the group consisting of —H; optionally substituted C₁-C₆ alkyl; optionally substituted aryl; and optionally substituted heteroaryl;
each —R⁵ is independently of each other selected from the group consisting of —H; optionally substituted C₁-C₆ alkyl; optionally substituted aryl; and optionally substituted heteroaryl; or when taken together two —R⁵ can be cycloalkyl or cycloheteroalkyl;
wherein —L¹— is substituted with —L²—Z and wherein —L¹— is optionally further substituted.

Only in the context of formula (V) the terms used have the following meaning: “Alkyl”, “alkenyl”, and “alkynyl” include linear, branched or cyclic hydrocarbon groups of 1-8 carbons or 1-6 carbons or 1-4 carbons wherein alkyl is a saturated hydrocarbon, alkenyl includes one or more carbon-carbon double bonds and alkynyl includes one or more carbon-carbon triple bonds. Unless otherwise specified these contain 1-6 C. “Aryl” includes aromatic hydrocarbon groups of 6-18 carbons, preferably 6-10 carbons, including groups such as phenyl, naphthyl, and anthracene. “Heteroaryl” includes aromatic rings comprising 3-15 carbons containing at least one N, O or S atom, preferably 3-7 carbons containing at least one N, O or S atom, including groups such as pyrrolyl, pyridyl, pyrimidinyl, imidazolyl, oxazolyl, isoxazolyl, thiszolyl, isothiazolyl, quinolyl, indolyl, indenyl, and similar.

The term “substituted” means an alkyl, alkenyl, alkynyl, aryl or heteroaryl group comprising one or more substituent groups in place of one or more hydrogen atoms. Substituents may generally be selected from halogen including F, Cl, Br, and I; lower alkyl including linear, branched, and cyclic; lower haloalkyl including fluoroalkyl, chloroalkyl, bromoalkyl, and iodoalkyl; OH; lower alkoxy including linear, branched, and cyclic; SH; lower alkylthio including linear, branched and cyclic; amino, alkylamino, dialkylamino, silyl including alkylsilyl, alkoxysilyl, and arylsilyl; nitro; cyano; carbonyl; carboxylic acid, carboxylic ester, carboxylic amide, aminocarbonyl; aminoacyl; carbamate; urea; thiocarbamate; thiourea; ketne; sulfone; sulfonamide; aryl including phenyl, naphthyl, and anthracenyl; heteroaryl including 5-membered heteroaryls including as pyrrole, imidazole, furan, thiophene, oxazole, thiazole, isoxazole, isothiazole, thiadiazole, triazole, oxadiazole, and tetrazole, 6-membered heteroaryls including pyridine, pyrimidine, pyrazine, and fused heteroaryls including benzofuran, benzothiophene, benzoxazole, benzimidazole, indole, benzothiazole, benzisoxazole, and benzisothiazole.

In certain embodiments —L¹— of formula (V) is substituted with one moiety —L²—Z.

In certain embodiments —L¹— has a structure as disclosed in U.S. Pat. No. 7,585,837B2. Accordingly, in certain embodiments —L¹— is of formula (VI):

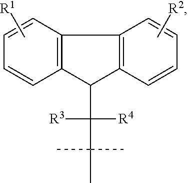

wherein
the dashed line indicates attachment to —D′— through an amine functional group of —D′—;
R¹ and R² are independently selected from the group consisting of hydrogen, alkyl, alkoxy, alkoxyalkyl, aryl, alkaryl, aralkyl, halogen, nitro, —SO₃H, —SO₂NHR⁵, amino, ammonium, carboxyl, PO₃H₂, and OPO₃H₂;
R³, R⁴, and R⁵ are independently selected from the group consisting of hydrogen, alkyl, and aryl;
wherein —L¹— is substituted with —L²—Z and wherein —L¹— is optionally further substituted.

Suitable substituents for formulas (VI) are alkyl (such as C₁₋₆ alkyl), alkenyl (such as C₂₋₆ alkenyl), alkynyl (such as C₂₋₆ alkynyl), aryl (such as phenyl), heteroalkyl, heteroalkenyl, heteroalkynyl, heteroaryl (such as aromatic 4 to 7 membered heterocycle) or halogen moieties.

Only in the context of formula (VI) the terms used have the following meaning: The terms “alkyl”, “alkoxy”, “alkoxyalkyl”, “aryl”, “alkaryl” and “aralkyl” mean alkyl radicals of 1-8, preferably 1-4 carbon atoms, e.g. methyl, ethyl, propyl, isopropyl and butyl, and aryl radicals of 6-10 carbon atoms, e.g. phenyl and naphthyl. The term “halogen” includes bromo, fluoro, chloro and iodo.

In certain embodiments —L¹— of formula (VI) is substituted with one moiety —L²—Z.

In certain embodiments —L¹— has a structure as disclosed in WO2002/089789A1. Accordingly, in certain embodiments —L¹— is of formula (VII):

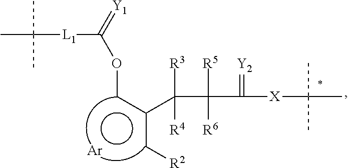

wherein
the dashed line indicates attachment to —D′— through an amine functional group of —D′—; Y₁ and Y₂ are independently O, S or NR⁷;
R², R³, R⁴, R⁵, R⁶ and R⁷ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy, and C₁₋₆ heteroalkoxy;
Ar is a moiety which when included in formula (VII) forms a multisubstituted aromatic hydrocarbon or a multi-substituted heterocyclic group;
X is a chemical bond or a moiety that is actively transported into a target cell, a hydrophobic moiety or a combination thereof,
y is 0 or 1;
wherein —L¹— is substituted with —L²—Z and wherein —L¹— is optionally further substituted.

Only in the context of formula (VII) the terms used have the following meaning: The term “alkyl” shall be understood to include, e.g. straight, branched, substituted C₁₋₁₂ alkyls, including alkoxy, C₃₋₈ cycloalkyls or substituted cycloalkyls, etc.

The term “substituted” shall be understood to include adding or replacing one or more atoms contained within a functional group or compounds with one or more different atoms.

Substituted alkyls include carboxyalkyls, aminoalkyls, dialkylaminos, hydroxyalkyls and mercaptoalkyls; substitued cycloalkyls include moieties such as 4-chlorocyclohexyl; aryls include moieties such as napthyl; substituted aryls include moieties such as 3-bromo-phenyl; aralkyls include moieties such as toluyl; heteroalkyls include moieties such as ethylthiophene; substituted heteroalkyls include moieties such as 3-methoxythiophone; alkoxy includes moieities such as methoxy; and phenoxy includes moieties such as 3-nitrophenoxy. Halo- shall be understood to include fluoro, chloro, iodo and bromo.

In certain embodiments —L¹— of formula (VII) is substituted with one moiety —L²—Z.

In certain embodiments —L¹— comprises a substructure of formula (VIII)

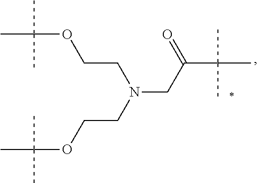

wherein
the dashed line marked with the asterisk indicates attachment to a nitrogen of —D′— by forming an amide bond;
the unmarked dashed lines indicate attachment to the remainder of —L¹—; and wherein —L¹— is substituted with —L²—Z and wherein —L¹— is optionally further substituted.

In certain embodiments —L¹— of formula (VIII) is substituted with one moiety —L²—Z.

In certain embodiments —L¹— of formula (VIII) is not further substituted.

In certain embodiments —L¹— comprises a substructure of formula (IX)

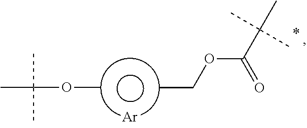

wherein
the dashed line marked with the asterisk indicates attachment to a nitrogen of —D′— by forming a carbamate bond;
the unmarked dashed lines indicate attachment to the remainder of —L¹—; and wherein —L¹— is substituted with —L²—Z and wherein —L¹— is optionally further substituted.

In certain embodiments —L¹— of formula (IX) is substituted with one moiety —L²—Z.

In certain embodiments —L¹— of formula (IX) is not further substituted.

In certain embodiments —L¹— is of formula (IX-a):

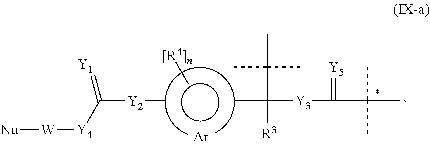

wherein
the dashed line marked with the asterisk indicates attachment to a nitrogen of —D′— and
the unmarked dashed line indicates attachment to —L²—Z;
n is 0, 1, 2, 3 or 4;
═Y₁ is selected from the group consisting of ═O and ═S;
—Y₂— is selected from the group consisting of —O— and —S—;
—Y₃— is selected from the group consisting of —O— and —S—;
—Y₄— is selected from the group consisting of —O—, —NR⁵— and —C(R⁶R^{6a})—;
═Y₅ is selected from the group consisting of ═O and ═S;
—R³, —R⁵, —R⁶, —R^{6a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; —R⁴ is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl;
—W— is selected from the group consisting of C₁₋₂₀ alkyl optionally interrupted by one or more groups selected from the group consisting of C₃₋₁₀ cycloalkyl, 8- to 30-membered carbopolycyclyl, 3- to 10-membered heterocyclyl, —C(O)—, —C(O)N(R⁷)—, —O—, —S— and —N(R⁷)—;
—Nu is a nucleophile selected from the group consisting of —N(R⁷R^{7a}), —N(R⁷OH), —N(R⁷)—N(R^{7a}R^{7b}), —S(R⁷), —COOH,

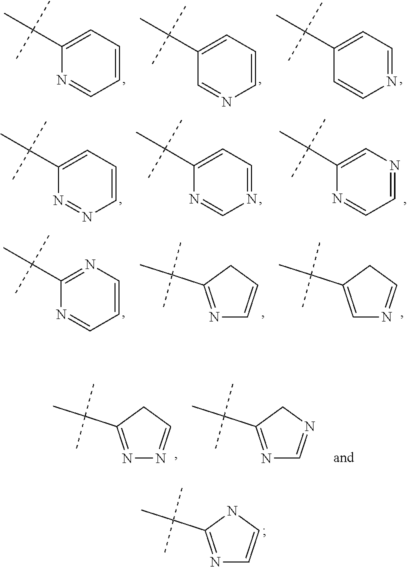

—Ar— is selected from the group consisting of

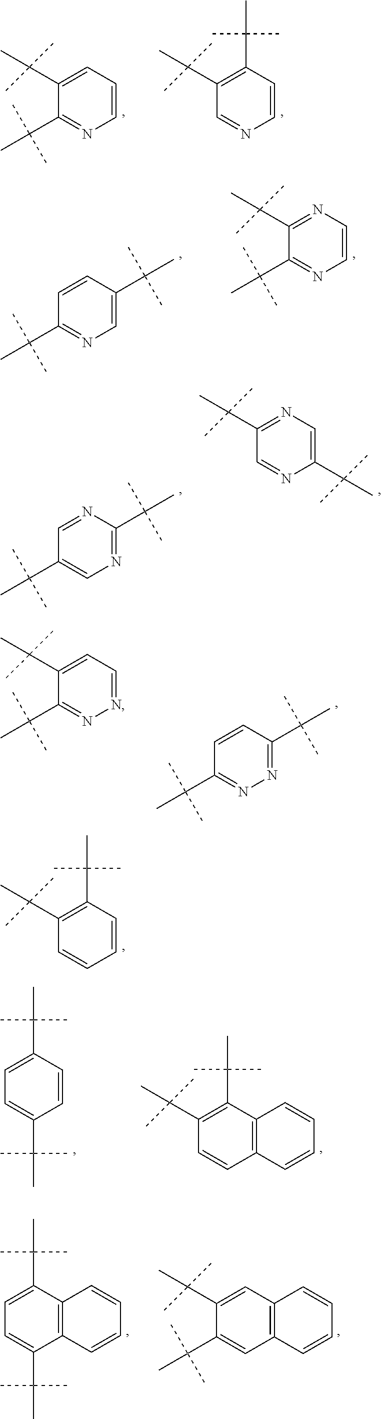

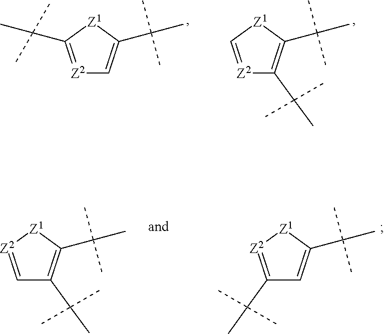

wherein
dashed lines indicate attachment to the remainder of —L¹—,
—Z¹— is selected from the group consisting of —O—, —S— and —N(R⁷)—, and
—Z²—0 is —N(R⁷)—; and
—R⁷, —R^{7a}, —R^{7b} are independently of each other selected from the group consisting of —H, C₁₋₆ alkyl, C₂₋₆ alkenyl and C₂₋₆ alkynyl;
wherein —L¹— is optionally further substituted.

In certain embodiments —L¹— is of formula (IX-a), wherein the dashed line marked with the asterisk indicates attachment to the nitrogen of the ε-amino group of a lysine residue of —D′— or to the nitrogen of the N-terminal amine of —D′—.

In certain embodiments —L¹— is of formula (IX-a), wherein the dashed line marked with the asterisk indicates attachment to the nitrogen of the ε-amino group of a lysine residue of —D′—.

In certain embodiments —L¹— is of formula (IX-a), wherein the dashed line marked with the asterisk indicates attachment to the nitrogen of the N-terminal amine of —D′—.

In certain embodiments —L¹— of formula (IX-a) is not further substituted.

In certain embodiments —L¹— is of formula (IX-b):

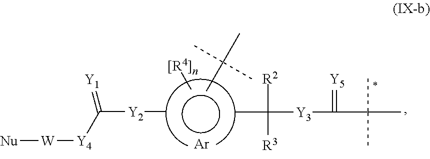

wherein
the dashed line marked with the asterisk indicates attachment to a nitrogen of —D′— and
the unmarked dashed line indicates attachment to —L²—Z;
n is 0, 1, 2, 3 or 4;
═Y₁ is selected from the group consisting of ═O and ═S;
—Y₂— is selected from the group consisting of —O— and —S—;
—Y₃— is selected from the group consisting of —O— and —S—;
—Y₄— is selected from the group consisting of —O—, —NR⁵— and —C(R⁶R^{6a})—;
═Y₅ is selected from the group consisting of ═O and ═S;
—R², —R³, —R⁵, —R⁶, —R^{6a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; —R⁴ is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl;
—W— is selected from the group consisting of C₁₋₂₀ alkyl optionally interrupted by one or more groups selected from the group consisting of C₃₋₁₀ cycloalkyl, 8- to 30-membered carbopolycyclyl, 3- to 10-membered heterocyclyl, —C(O)—,
—C(O)N(R⁷)—, —O—, —S— and —N(R⁷)—;
—Nu is a nucleophile selected from the group consisting of —N(R⁷R^{7a}), —N(R⁷OH), —N(R⁷)—N(R^{7a}R^{7b}), —S(R⁷), —COOH,

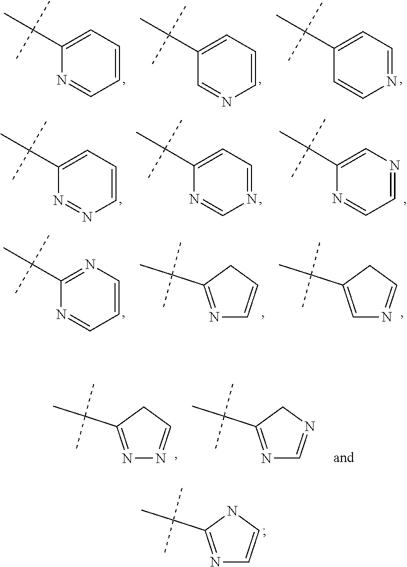

—Ar— is selected from the group consisting of

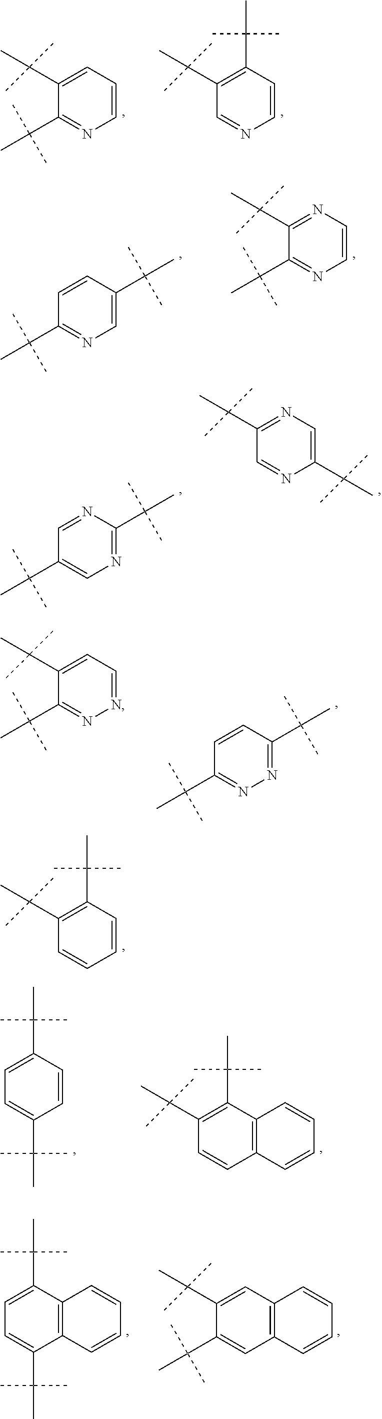

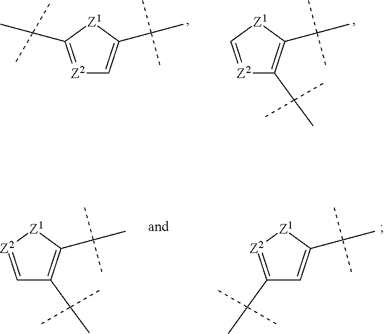

wherein
dashed lines indicate attachment to the remainder of —L¹—,
—Z¹— is selected from the group consisting of —O—, —S— and —N(R⁷)—, and
—Z²— is —N(R⁷)—; and
—R⁷, —R^{7a}, —R^{7b} are independently of each other selected from the group consisting of —H, C₁₋₆ alkyl, C₂₋₆ alkenyl and C₂₋₆ alkynyl;
wherein —L¹— is optionally further substituted.

In certain embodiments —L¹— is of formula (IX-b), wherein the dashed line marked with the asterisk indicates attachment to the nitrogen of the ε-amino group of a lysine residue of —D′— or to the nitrogen of the N-terminal amine of —D′—.

In certain embodiments —L¹— is of formula (IX-b), wherein the dashed line marked with the asterisk indicates attachment to the nitrogen of the ε-amino group of a lysine residue of —D′—.

In certain embodiments —L¹— is of formula (IX-b), wherein the dashed line marked with the asterisk indicates attachment to the nitrogen of the N-terminal amine of —D′—.

In certain embodiments —L¹— of formula (IX-b) is not further substituted.

In certain embodiments ═Y¹ of formula (IX-a) and (IX-b) is ═O.

In certain embodiments —Y²— of formula (IX-a) and (IX-b) is —O—.

In certain embodiments —Y³— of formula (IX-a) and (IX-b) is —O—.

In certain embodiments —Y⁴— of formula (IX-a) and (IX-b) is —NR—.

In certain embodiments ═Y⁵ of formula (IX-a) and (IX-b) is ═O.

In certain embodiments n of formula (IX-a) and (IX-b) is 0 or 1. In certain embodiments n of formula (IX-a) and (IX-b) is 0. In certain embodiments n of formula (IX-a) and (IX-b) is 1.

In certain embodiments —R² of formula (IX-b) is selected from the group consisting of —H, methyl, ethyl, n-propyl, isopropyl, n-butyl, isobutyl, sec-butyl and tert-butyl. In certain embodiments —R² of formula (IX-b) is selected from the group consisting of —H, methyl, ethyl, n-propyl and isopropyl. In certain embodiments —R² of formula (IX-b) is selected from —H, methyl and ethyl. In certain embodiments —R² of formula (IX-b) is —H.

In certain embodiments —R³ of formula (IX-a) and (IX-b) is selected from the group consisting of —H, methyl, ethyl, n-propyl, isopropyl, n-butyl, isobutyl, sec-butyl and tert-butyl. In certain embodiments —R³ of formula (IX-a) and (IX-b) is selected from the group consisting of —H, methyl, ethyl, n-propyl and isopropyl. In certain embodiments —R³ of formula (IX-a) and (IX-b) is selected from —H, methyl and ethyl. In certain embodiments —R³ of formula (IX-a) and (IX-b) is —H.

In certain embodiments each —R⁴ of formula (IX-a) and (IX-b) is independently selected from methyl, ethyl, n-propyl, isopropyl, n-butyl, isobutyl, sec-butyl and tert-butyl. In certain embodiments —R⁴ of formula (IX-a) and (IX-b) is selected from the group consisting of methyl, ethyl, n-propyl and isopropyl. In certain embodiments —R⁴ of formula (IX-a) and (IX-b) is selected from methyl and ethyl.

In certain embodiments —R⁵ of formula (IX-a) and (IX-b) is selected from the group consisting of —H, methyl, ethyl, n-propyl, isopropyl, n-butyl, isobutyl, sec-butyl and tert-butyl. In certain embodiments —R⁵ of formula (IX-a) and (IX-b) is selected from the group consisting of —H, methyl, ethyl, n-propyl and isopropyl. In certain embodiments —R⁵ of formula (IX-a) and (IX-b) is selected from methyl and ethyl. In certain embodiments —R⁵ of formula (IX-a) and (IX-b) is methyl.

In certain embodiments —R⁶ and —R^{6a} of formula (IX-a) and (IX-b) are independently selected from the group consisting of —H, methyl, ethyl, n-propyl, isopropyl, n-butyl, isobutyl, sec-butyl and tert-butyl. In certain embodiments —R⁶ and —R^{6a} of formula (IX-a) and (IX-b) are independently selected from the group consisting of —H, methyl, ethyl, n-propyl and isopropyl.

In certain embodiments —R⁶ and —R^{6a} of formula (IX-a) and (IX-b) are independently selected from —H, methyl and ethyl. In certain embodiments —R⁶ and —R^{6a} of formula (IX-a) and (IX-b) are both —H.

In certain embodiments —Ar— of formula (IX-a) and (IX-b) is phenyl. In certain embodiments —Ar— of formula (IX-a) and (IX-b) is

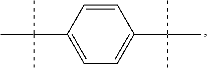

wherein the dashed lines indicate attachment to the remainder of the moiety of formula (IX-a) and (IX-b).

In certain embodiments —W— of formula (IX-a) and (IX-b) is C₁₋₂₀ alkyl, optionally interrupted with C₃₋₁₀ cycloalkyl, —C(O)—, —C(O)N(R⁷)—, —O—, —S— and —N(R⁷)—. In certain embodiments —W— of formula (IX-a) and (IX-b) is C₁₋₁₀ alkyl, optionally interrupted with C₃₋₁₀ cycloalkyl, —C(O)—, —C(O)N(R⁷)—, —O—, —S— and —N(R⁷)—. In certain embodiments —W— of formula (IX-a) and (IX-b) is C₁₋₆ alkyl, optionally interrupted with C₃₋₁₀ cycloalkyl, —C(O)—, —C(O)N(R⁷)—, —O—, —S— and —N(R⁷)—. In certain embodiments —W— of formula (IX-a) and (IX-b) is

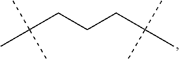

wherein
the dashed lines indicate attachment to the remainder of the moiety of formula (IX-a) or (IX-b), respectively.

In certain embodiments —Nu of formula (IX-a) and (IX-b) is —N(R⁷R^{7a}).

In certain embodiments —R⁷, —R^{7a} and —R^{7b} of formula (IX-a) and (IX-b) are independently of each other selected from the group consisting of —H, methyl, ethyl, n-propyl, isopropyl, n-butyl, isobutyl, sec-butyl and tert-butyl. In certain embodiments —R⁷, —R^{7a} and —R^{7b} of formula (IX-a) and (IX-b) are independently of each other selected from —H, methyl, ethyl, n-propyl and isopropyl. In certain embodiments —R⁷, —R^{7a} and —R⁷ of formula (IX-a) and (IX-b) are independently of each other selected from methyl or ethyl. In certain embodiments —R⁷, —R^{7a} and —R⁷ of formula (IX-a) and (IX-b) are both methyl.

In certain embodiments —L¹— is of formula (IX-c)

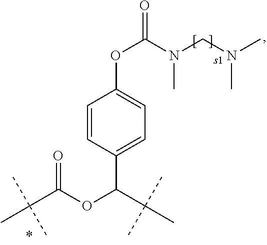

wherein
the dashed line marked with the asterisk indicates attachment to a nitrogen of —D′—;
the unmarked dashed line indicates attachment to —L²—Z; and
s1 is an integer selected from the group consisting of 1, 2, 3, 4, 5, 6, 7, 8, 9 and 10.

In certain embodiments —L¹— is of formula (IX-c), wherein the dashed line marked with the asterisk indicates attachment to the nitrogen of the ε-amino group of a lysine residue of —D′— or to the nitrogen of the N-terminal amine of —D′—.

In certain embodiments —L¹— is of formula (IX-c), wherein the dashed line marked with the asterisk indicates attachment to the nitrogen of the ε-amino group of a lysine residue of —D′—.

In certain embodiments —L¹— is of formula (IX-c), wherein the dashed line marked with the asterisk indicates attachment to the nitrogen of the N-terminal amine of —D′—.

In certain embodiments s1 of formula (IX-c) is an integer selected from the group consisting of 1, 2, 3, 4 and 5. In certain embodiments s1 of formula (IX-c) is 1. In certain embodiments s1 of formula (IX-c) is 2. In certain embodiments s1 of formula (IX-c) is 3. In certain embodiments s1 of formula (IX-c) is 4. In certain embodiments s1 of formula (IX-c) is 5.

In certain embodiments —L¹— is of formula (IX-d)

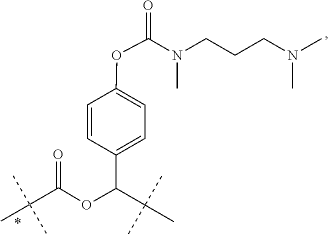

wherein
the dashed line marked with the asterisk indicates attachment to a nitrogen of —D′—; and
the unmarked dashed line indicates attachment to —L²—Z.

In certain embodiments —L¹— is of formula (IX-d), wherein the dashed line marked with the asterisk indicates attachment to the nitrogen of the ε-amino group of a lysine residue of —D′— or to the nitrogen of the N-terminal amine of —D′—.

In certain embodiments —L¹— is of formula (IX-d), wherein the dashed line marked with the asterisk indicates attachment to the nitrogen of the ε-amino group of a lysine residue of —D′—.

In certain embodiments —L¹— is of formula (IX-d), wherein the dashed line marked with the asterisk indicates attachment to the nitrogen of the N-terminal amine of —D′—.

In certain embodiments —L¹— has a structure as disclosed in WO2020/206358 A1. Accordingly, in certain embodiments the moiety —L¹— is of formula (X):

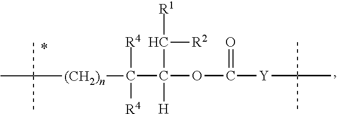

wherein
the unmarked dashed line indicates attachment to —D′—;
the dashed line marked with the asterisk indicates attachment to —L²—Z;
n is an integer selected from the group consisting of 0, 1, 2, 3, 4, 5 and 6;
—R¹ and —R² are independently an electron-withdrawing group, alkyl or —H, and wherein at least one of —R¹ or —R² is an electron-withdrawing group;
each —R⁴ is independently C₁-C₃ alkyl or the two —R⁴ are taken together with the carbon atom to which they are attached to form a 3- to 6-membered ring; and
—Y— is absent when —D′— is a drug moiety connected through an amine, or —Y— is —N(R⁶)CH₂— when —D′— is a drug moiety connected through a phenol, alcohol, thiol, thiophenol, imidazole or non-basic amine; wherein —R⁶ is optionally substituted C₁-C₆ alkyl, optionally substituted aryl, or optionally substituted heteroaryl.

Only in the context of formula (X) the terms used have the following meaning: The term “alkyl” refers to linear, branched or cyclic saturated hydrocarbon groups of 1 to 20, 1 to 12, 1 to 8, 1 to 6 or 1 to 4 carbon atoms. In certain embodiments an alkyl is linear or branched.

Examples of linear or branched alkyl groups include methyl, ethyl, n-propyl, isopropyl, n-butyl, t-butyl, isobutyl, sec-butyl, n-pentyl, n-hexyl, n-heptyl, n- octyl, n-nonyl, and n-decyl. In certain embodiments an alkyl is cyclic. Examples of cyclic alkyl groups include cyclopropyl, cyclobutyl, cyclopentyl, cyclopentadienyl, and cyclohexyl.

The term “alkoxy” refers to alkyl groups bonded to oxygen, including methoxy, ethoxy, isopropoxy, cyclopropoxy, and cyclobutoxy.

The term “alkenyl” refers to non-aromatic unsaturated hydrocarbons with carbon-carbon double bonds and 2 to 20, 2 to 12, 2 to 8, 2 to 6 or 2 to 4 carbon atoms.

The term “alkynyl” refers to non-aromatic unsaturated hydrocarbons with carbon-carbon triple bonds and 2 to 20, 2 to 12, 2 to 8, 2 to 6 or 2 to 4 carbon atoms.

The term “aryl” refers to aromatic hydrocarbon groups of 6 to 18 carbons, preferably 6 to 10 carbons, including groups such as phenyl, naphthyl, and anthracenyl. The term “heteroaryl” refers to aromatic rings comprising 3 to 15 carbons comprising at least one N, O or S atom, preferably 3 to 7 carbons comprising at least one N, O or S atom, including groups such as pyrrolyl, pyridyl, pyrimidinyl, imidazolyl, oxazolyl, isoxazolyl, thiazolyl, isothiazolyl, quinolyl, indolyl, and indenyl.

In certain embodiments alkenyl, alkynyl, aryl or heteroaryl moieties may be coupled to the remainder of the molecule through an alkyl linkage. Under those circumstances, the substituent will be referred to as alkenylalkyl, alkynylalkyl, arylalkyl or heteroarylalkyl, indicating that an alkylene moiety is between the alkenyl, alkynyl, aryl or heteroaryl moiety and the molecule to which the alkenyl, alkynyl, aryl or heteroaryl is coupled.

The term “halogen” or “halo” refers to bromo, fluoro, chloro and iodo.

The term “heterocyclic ring” or “heterocyclyl” refers to a 3- to 15-membered aromatic or non-aromatic ring comprising at least one N, O or S atom. Examples include piperidinyl, piperazinyl, tetrahydropyranyl, pyrrolidine, and tetrahydrofuranyl, as well as the exemplary groups provided for the term “heteroaryl” above. In certain embodiments a heterocyclic ring or heterocyclyl is non-aromatic. In certain embodiments a heterocyclic ring or heterocyclyl is aromatic.

The term “optionally substituted” refers to a group may be unsubstituted or substituted by one or more (e.g., 1, 2, 3, 4 or 5) of the substituents which may be the same or different. Examples of substituents include alkyl, alkenyl, alkynyl, halogen, —CN, —OR^{aa}, —SR^{aa}, —NR^{aa}R^{bb}, —NO₂, —C═NH(OR^{aa}), —C(O)R—, —OC(O)R^{aa}, —C(O)OR^{aa}, —C(O)NR^{aa}R^{bb}, —OC(O)NR^{aa}R^{bb}, —NR^{aa}C(O)R^{bb}, —NR^{aa}C(O)OR^{bb}, —S(O)R^{aa}, —S(O)₂R^{aa}, —NR^{aa}S(O)R^{bb}, —C(O)NR^{aa}S(O)R^{bb}, —NR^{aa}S(O)₂R^{bb}, —C(O)NR^{aa}S(O)₂R^{bb}, —S(O)NR^{aa}R^{bb}, —S(O)₂NR^{aa}R^{bb}, —P(O)(OR^{aa})(OR^{bb}), heterocyclyl, heteroaryl or aryl, wherein the alkyl, alkenyl, alkynyl, cycloalkyl, heterocyclyl, heteroaryl, and aryl are each independently optionally substituted by —R^{cc}, wherein —R^{aa} and —R^{bb} are each independently —H, alkyl, alkenyl, alkynyl, heterocyclyl, heteroaryl or aryl, or —R^{aa} and —R^{bb} are taken together with the nitrogen atom to which they attach to form a heterocyclyl, which is optionally substituted by alkyl, alkenyl, alkynyl, halogen, hydroxyl, alkoxy or —CN, and wherein: each —R^{cc} is independently alkyl, alkenyl, alkynyl, halogen, heterocyclyl, heteroaryl, aryl, —CN or —NO₂.

In certain embodiments —L²— is a spacer moiety. In certain embodiments —L²— is absent.

In certain embodiments —L²— is selected from the group consisting of —T—, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})——S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein —T—, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of —T—, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—; —R^{y1} and —R^{y1a} are independently of each other selected from the group consisting of —H, —T, C₁₋₅₀ alkyl, C₂₋₅₀ alkenyl, and C₂₋₅₀ alkynyl; wherein —T, C₁₋₅₀ alkyl, C₂₋₅₀ alkenyl, and C₂₋₅₀ alkynyl are optionally substituted with one or more —R^{y2}, which are the same or different, and wherein C₁₋₅₀ alkyl, C₂₋₅₀ alkenyl, and C₂₋₅₀ alkynyl are optionally interrupted by one or more groups selected from the group consisting of —T—, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;

each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;

each —R^{y2} is independently selected from the group consisting of halogen, —CN, oxo (═O), —COOR^{y5}, —OR^{y5}, —C(O)R^{y5}, —C(O)N(R⁵R^{y5a}), —S(O)₂N(R⁵R^{y5a}), —S(O)N(R⁵R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5}a —N(R^{y5})C(O)N(R^{y5a}R^{y5b}), —OC(O)N(R⁵R^{y5a}), and C₁₋₆ alkyl; wherein C₁₋₆ alkyl is optionally substituted with one or more halogen, which are the same or different; and each —R^{y3}, —R^{y3a}, —R^{y4}, —R^{y4a}, —R^{y5}, —R^{y5}a and —R^{y5b} is independently selected from the group consisting of —H, and C₁₋₆ alkyl, wherein C₁₋₆ alkyl is optionally substituted with one or more halogen, which are the same or different.

In certain embodiments —L²— is selected from the group consisting of —T—, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})——S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅o alkenyl, and C₂₋₅₀ alkynyl; wherein —T—, C₁₋₂₀ alkyl, C₂₋₂₀ alkenyl, and C₂₋₂₀ alkynyl are optionally substituted with one or more —R^{y2}, which are the same or different and wherein C₁₋₂₀ alkyl, C₂₋₂₀ alkenyl, and C₂₋₂₀ alkynyl are optionally interrupted by one or more groups selected from the group consisting of —T—, —C(O)O—, —O—, —C(O)—, —C(O)N(R^{y3})—, —S(O)₂N(R^{y}3)—, —S(O)N(R^{y}3)—, —S(O)₂—, —S(O)—, —N(R^{y}3)S(O)₂N(R^{y3a})—, —S—, —N(R^{y}3)—, —OC(OR^{y3})(R^{y3a})—, —N(R^{y3})C(O)N(R^{y3a})—, and —OC(O)N(R^{y3})—; —R^{y1} and —R^{y1a} are independently of each other selected from the group consisting of —H, —T, C₁₋₁₀ alkyl, C₂₋₁₀ alkenyl, and C₂₋₁₀ alkynyl; wherein —T, C₁₋₁₀ alkyl, C₂₋₁₀ alkenyl, and C₂₋₁₀ alkynyl are optionally substituted with one or more —R^{y2}, which are the same or different, and wherein C₁₋₁₀ alkyl, C₂₋₁₀ alkenyl, and C₂₋₁₀ alkynyl are optionally interrupted by one or more groups selected from the group consisting of —T—, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;

each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;

—R^{y2} is selected from the group consisting of halogen, —CN, oxo (═O), —COOR^{y5}, —OR^{y5}, —C(O)R^{y5}, —C(O)N(R⁵R^{y5a}), —S(O)₂N(R^{y5}R^{y5a}), —S(O)N(R⁵R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5}a —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}) —OC(O)N(R⁵R^{y5a}), and C₁₋₆ alkyl; wherein C₁₋₆ alkyl is optionally substituted with one or more halogen, which are the same or different; and

each —R^{y3}, —R^{y3a}, —R^{y4}, —R^{y4a}, —R^{y5}, —R^{y5}a and —R^{y5b} is independently of each other selected from the group consisting of —H, and C₁₋₆ alkyl; wherein C₁₋₆ alkyl is optionally substituted with one or more halogen, which are the same or different.

In certain embodiments —L²— is selected from the group consisting of —T—, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})——S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅o alkenyl, and C₂₋₅₀ alkynyl; wherein —T—, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of —T—, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;

—R^{y1} and —R^{y1a} are independently selected from the group consisting of —H, —T, C₁₋₁₀ alkyl, C₂₋₁₀ alkenyl, and C₂₋₁₀ alkynyl;

each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; each —R^{y2} is independently selected from the group consisting of halogen, and C₁₋₆ alkyl; and

each —R^{y3}, —R^{y3a}, —R^{y4}, —R^{y4a}, —R^{y5}, —R^{y1}a and —R^{y5b} is independently of each other selected from the group consisting of —H, and C₁₋₆ alkyl; wherein C₁₋₆ alkyl is optionally substituted with one or more halogen, which are the same or different.

In certain embodiments —L²— is a C₁₋₂₀ alkyl chain, which is optionally interrupted by one or more groups independently selected from —O—, —C(O)—, —T— and —C(O)N(R^{y1})—; and which C₁₋₂₀ alkyl chain is optionally substituted with one or more groups independently selected from —OH, —T and —C(O)N(R^{y6}R^{y6a}); wherein —R¹, —R^{y6}, —R^{y6a} are independently selected from the group consisting of H and C₁₋₄ alkyl and wherein T is selected from the group consisting of phenyl, naphthyl, indenyl, indanyl, tetralinyl, C₃₋₁₀cycloalkyl, 3- to 10-membered heterocyclyl, 8- to 11-membered heterobicyclyl, 8- to 30-membered carbopolycyclyl, and 8- to 30-membered heteropolycyclyl.

In certain embodiments —L²— has a molecular weight in the range of 14 g/mol to 750 g/mol.

In certain embodiments —L²— comprises a moiety selected from the group consisting of

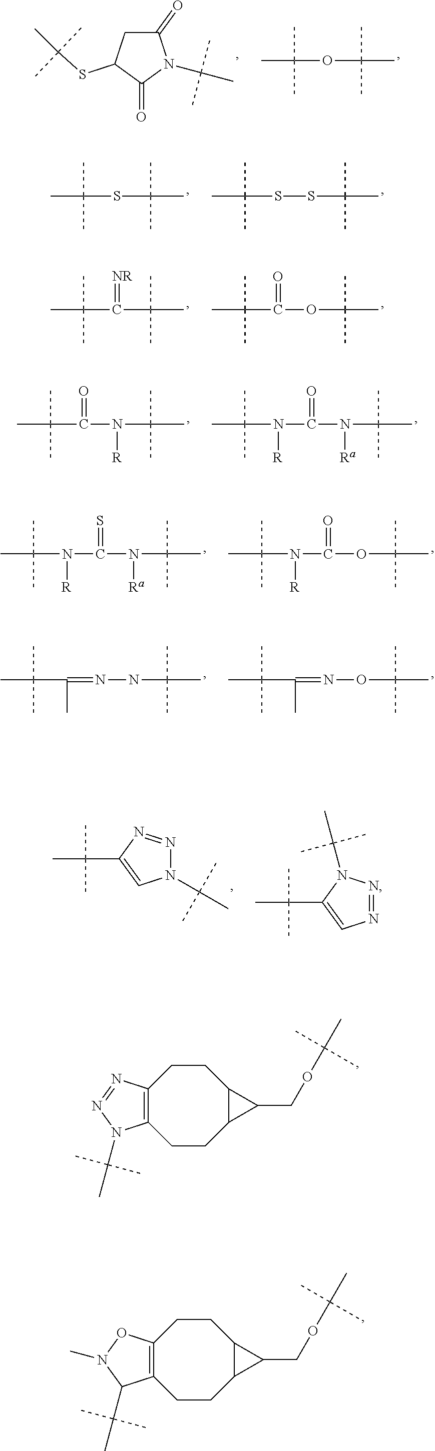

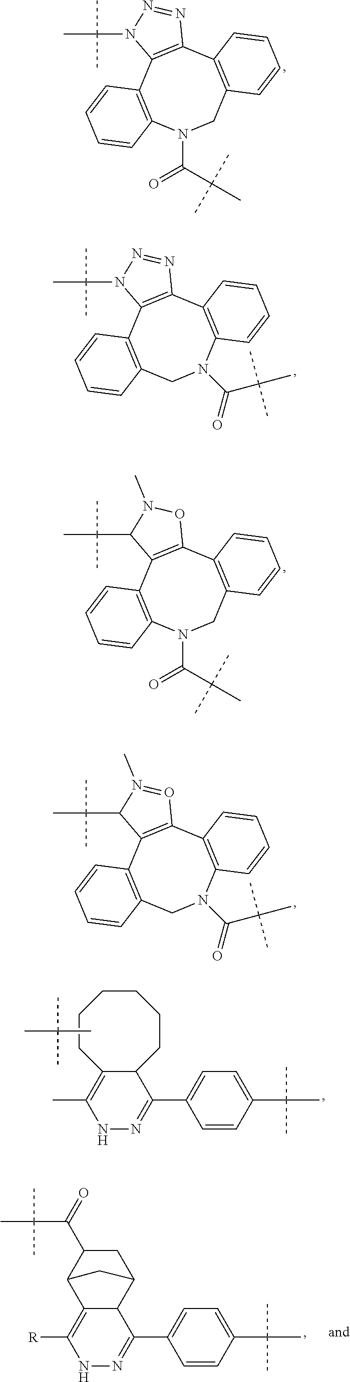

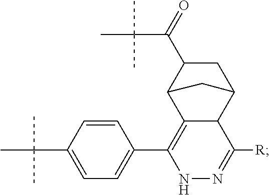

wherein
dashed lines indicate attachment to —L¹—, the remainder of —L²— or to —Z, respectively; and —R and —R^{a} are independently of each other selected from the group consisting of —H, methyl, ethyl, propyl, butyl, pentyl and hexyl.

In certain embodiments —L²— is of formula (IX-e)

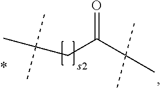

wherein
the dashed line marked with the asterisk indicates attachment to —L¹—;
the unmarked dashed line indicates attachment to —Z; and
s2 is an integer selected from the group consisting of 0, 1, 2, 3, 4, 5, 6, 7, 8, 9, 10, 11, 12, 13, 14, 15, 16, 17, 18, 19 and 20.

In certain embodiments s2 of formula (IX-e) is an integer selected from the group consisting of 1, 2, 3, 4, 5, 6, 7, 8, 9, 10, 11 and 12. In certain embodiments s2 of formula (IX-e) is an integer selected from the group consisting of 1, 2, 3, 4, 5, 6, 7 and 8. In certain embodiments s2 of formula (IX-e) is 1. In certain embodiments s2 of formula (IX-e) is 2. In certain embodiments s2 of formula (IX-e) is 3. In certain embodiments s2 of formula (IX-e) is 4. In certain embodiments s2 of formula (IX-e) is 5. In certain embodiments s2 of formula (IX-e) is 6. In certain embodiments s2 of formula (IX-e) is 7. In certain embodiments s2 of formula (IX-e) is 8.

In certain embodiments the moiety —L¹—L²— is of formula (IX-f)

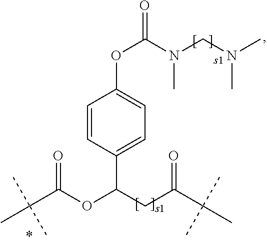

wherein
the dashed line marked with the asterisk indicates attachment to a nitrogen of —D′—;
the unmarked dashed line indicates attachment to —Z;
s1 is an integer selected from the group consisting of 1, 2, 3, 4, 5, 6, 7, 8, 9 and 10; and
s2 is an integer selected from the group consisting of 0, 1, 2, 3, 4, 5, 6, 7, 8, 9, 10, 11, 12, 13, 14, 15, 16, 17, 18, 19 and 20.

In certain embodiments —L¹— is of formula (IX-f), wherein the dashed line marked with the asterisk indicates attachment to the nitrogen of the ε-amino group of a lysine residue of —D′— or to the nitrogen of the N-terminal amine of —D′—.

In certain embodiments —L¹— is of formula (IX-f), wherein the dashed line marked with the asterisk indicates attachment to the nitrogen of the ε-amino group of a lysine residue of —D′—.

In certain embodiments —L¹— is of formula (IX-f), wherein the dashed line marked with the asterisk indicates attachment to the nitrogen of the N-terminal amine of —D′—.

Accordingly, the linkage between the moiety —L¹— and —D′— formed in the compound of formula (IX-f) is a carbamate.

In certain embodiments s1 of formula (IX-f) is an integer selected from the group consisting of 1, 2, 3, 4 and 5. In certain embodiments s1 of formula (IX-f) is 1. In certain embodiments s1 of formula (IX-f) is 2. In certain embodiments s1 of formula (IX-f) is 3. In certain embodiments s1 of formula (IX-f) is 4. In certain embodiments s1 of formula (IX-f) is 5.

In certain embodiments s2 of formula (IX-f) is an integer selected from the group consisting of 1, 2, 3, 4, 5, 6, 7, 8, 9, 10, 11 and 12. In certain embodiments s2 of formula (IX-f) is an integer selected from the group consisting of 1, 2, 3, 4, 5, 6, 7 and 8. In certain embodiments s2 of formula (IX-f) is 1. In certain embodiments s2 of formula (IX-f) is 2. In certain embodiments s2 of formula (IX-f) is 3. In certain embodiments s2 of formula (IX-f) is 4. In certain embodiments s2 of formula (IX-f) is 5. In certain embodiments s2 of formula (IX-f) is 6. In certain embodiments s2 of formula (IX-f) is 7. In certain embodiments s2 of formula (IX-f) is 8.

In certain embodiments s1 of formula (IX-f) is 3 and s2 of formula (IX-f) is 3.

In certain embodiments the moiety —L¹—L²—Z is of formula (XI)

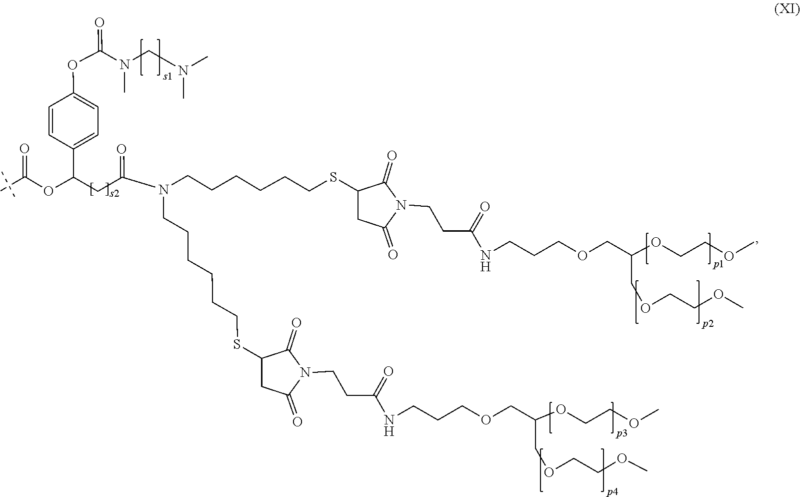

Wherein the dashed line indicates attachment to a nitrogen of —D′—;
s1 is an integer selected from the group consisting of 1, 2, 3, 4, 5, 6, 7, 8, 9 and 10;
s2 is an integer selected from the group consisting of 0, 1, 2, 3, 4, 5, 6, 7, 8, 9, 10, 11, 12, 13, 14, 15, 16, 17, 18, 19 and 20; and
p1, p2, p3, p4 are independently of each other an integer ranging from 110 to 345.

In certain embodiments —L¹—L²—Z is of formula (XI), wherein the dashed line indicates attachment to the nitrogen of the ε-amino group of a lysine residue of —D′— or to the nitrogen of the N-terminal amine of —D′—.

In certain embodiments —L¹—L²—Z is of formula (XI), wherein the dashed line indicates attachment to the nitrogen of the ε-amino group of a lysine residue of —D′—.

In certain embodiments —L¹—L²—Z is of formula (XI), wherein the dashed line indicates attachment to the nitrogen of the N-terminal amine of —D′—.

Accordingly, the linkage between the moiety —L¹—L²—Z and —D′— formed in the compound of formula (XI) is a carbamate.

In certain embodiments s1 of formula (XI) is an integer selected from the group consisting of 1, 2, 3, 4 and 5. In certain embodiments s1 of formula (XI) is 1. In certain embodiments s1 of formula (XI) is 2. In certain embodiments s1 of formula (XI) is 3. In certain embodiments s1 of formula (XI) is 4. In certain embodiments s1 of formula (XI) is 5.

In certain embodiments s2 of formula (XI) is an integer selected from the group consisting of 1, 2, 3, 4, 5, 6, 7, 8, 9, 10, 11 and 12. In certain embodiments s2 of formula (XI) is an integer selected from the group consisting of 1, 2, 3, 4, 5, 6, 7 and 8. In certain embodiments s2 of formula (XI) is 1. In certain embodiments s2 of formula (XI) is 2. In certain embodiments s2 of formula (XI) is 3. In certain embodiments s2 of formula (XI) is 4. In certain embodiments s2 of formula (XI) is 5. In certain embodiments s2 of formula (XI) is 6. In certain embodiments s2 of formula (XI) is 7. In certain embodiments s2 of formula (XI) is 8.

In certain embodiments s1 of formula (XI) is 3 and s2 of formula (XI) is 3.

In certain embodiments p1, p2, p3, p4 of formula (XI) are independently of each other an integer ranging from 140 to 315. In certain embodiments p1, p2, p3, p4 of formula (XI) are independently of each other an integer ranging from 170 to 285. In certain embodiments p1, p2, p3, p4 of formula (XI) are independently of each other an integer ranging from 195 to 255. In certain embodiments p 1, p2, p3, p4 of formula (XI) are independently of each other an integer ranging from 200 to 250. In certain embodiments p1, p2, p3, p4 of formula (XI) are independently of each other an integer ranging from 210 to 250. In certain embodiments p1, p2, p3, p4 of formula (XI) are independently of each other an integer ranging from 220 to 240.

In certain embodiments p1, p2, p3 and p4 of formula (XI) are identical and p1, p2, p3 and p4 range from 195 to 255. In certain embodiments p1, p2, p3 and p4 of formula (XI) are identical and p1, p2, p3 and p4 range from 200 to 250. In certain embodiments p1, p2, p3 and p4 of formula (XI) are identical and p1, p2, p3 and p4 range from 210 to 250. In certain embodiments p1, p2, p3 and p4 of formula (XI) are identical and p1, p2, p3 and p4 range from 220 to 240.

In certain embodiments p1, p2, p3 and p4 of formula (XI) are identical and are such that the molecular weight of a PEG arm ranges from 8 to 12 kDa. In certain embodiments p1, p2, p3 and p4 of formula (XI) are identical and are such that the molecular weight of a PEG arm ranges from 9 to 11 kDa. In certain embodiments p1, p2, p3 and p4 of formula (XI) are identical and are such that the molecular weight of a PEG arm is about 10 kDa.

The term “PEG arm” with regards to p1, p2, p3 and p4 of formula (XI) or (XI-a) refers to a moiety

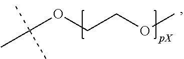

wherein pX is either p1, p2, p3 or p4.

In certain embodiments the moiety —L¹—L²—Z is of formula (XI-a)

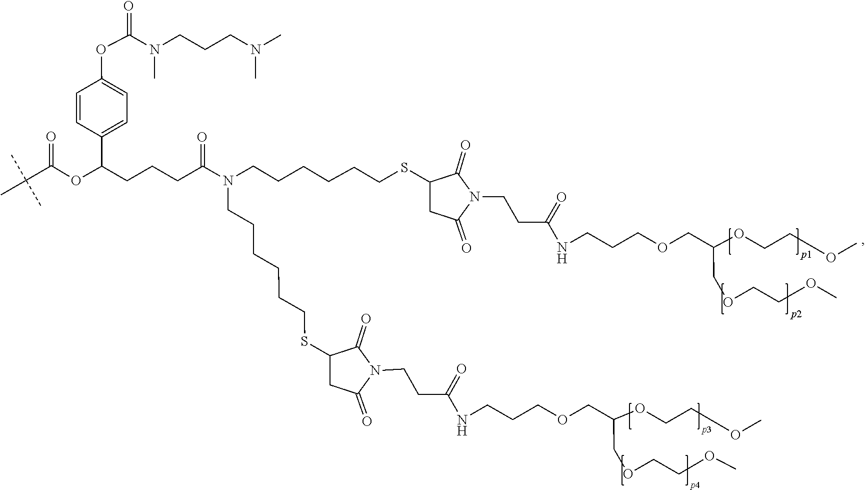

wherein the dashed line indicates attachment to a nitrogen of —D; and
p1, p2, p3, p4 are independently of each other an integer ranging from 110 to 345.

In certain embodiments —L¹—L²—Z is of formula (XI-a), wherein the dashed line indicates attachment to the nitrogen of the ε-amino group of a lysine residue of —D′— or to the nitrogen of the N-terminal amine of —D′—.

In certain embodiments —L¹—L²—Z is of formula (XI-a), wherein the dashed line attachment to the nitrogen of the ε-amino group of a lysine residue of —D′—.

In certain embodiments —L¹—L²—Z is of formula (XI-a), wherein the dashed line indicates attachment to the nitrogen of the N-terminal amine of —D′—.

In certain embodiments —L¹—L²—Z is of formula (XI-a), wherein the dashed line indicates attachment to the nitrogen of the ε-amino group of a lysine residue of —D′— or to the nitrogen of the N-terminal amine of —D′—, which has the amino acid sequence of SEQ ID NO. 4.

In certain embodiments —L¹—L²—Z is of formula (XI-a), wherein the dashed line indicates attachment to the nitrogen of the ε-amino group of a lysine residue of —D′—, which has the amino acid sequence of SEQ ID NO. 4.

In certain embodiments —L¹—L²—Z is of formula (XI-a), wherein the dashed line indicates attachment to the nitrogen of the N-terminal amine of —D′—, which has the amino acid sequence of SEQ ID NO. 4.

Accordingly, the linkage between the moiety —L¹—L²—Z and —D′— formed in the compound of formula (XI-a) is a carbamate.

In certain embodiments p1, p2, p3, p4 of formula (XI-a) are independently of each other an integer ranging from 140 to 315. In certain embodiments p1, p2, p3, p4 of formula (XI-a) are independently of each other an integer ranging from 170 to 285. In certain embodiments p1, p2, p3, p4 of formula (XI-a) are independently of each other an integer ranging from 195 to 255. In certain embodiments p1, p2, p3, p4 of formula (XI-a) are independently of each other an integer ranging from 200 to 250. In certain embodiments p1, p2, p3, p4 of formula (XI-a) are independently of each other an integer ranging from 210 to 250. In certain embodiments p1, p2, p3, p4 of formula (XI-a) are independently of each other an integer ranging from 220 to 240.

In certain embodiments p1, p2, p3 and p4 of formula (XI-a) are identical and p1, p2, p3 and p4 range from 195 to 255. In certain embodiments p1, p2, p3 of formula (XI-a) and p4 are identical and p1, p2, p3 and p4 range from 200 to 250. In certain embodiments p1, p2, p3 of formula (XI-a) and p4 are identical and p1, p2, p3 and p4 range from 210 to 250. In certain embodiments p1, p2, p3 of formula (XI-a) and p4 are identical and p1, p2, p3 and p4 range from 220 to 240.

In certain embodiments p1, p2, p3 and p4 of formula (XI-a) are identical and are such that the molecular weight of a PEG arm ranges from 8 to 12 kDa. In certain embodiments p1, p2, p3 and p4 of formula (XI-a) are identical and are such that the molecular weight of a PEG arm ranges from 9 to 11 kDa. In certain embodiments p1, p2, p3 and p4 of formula (XI-a) are identical and are such that the molecular weight of a PEG arm is about 10 kDa.

In certain embodiments the IL-2 conjugate is of formula (I), wherein —D- is —D′—M_{mod} and wherein —D′— has the amino acid sequence of SEQ ID NO. 4, —M_{mod} has the structure of (A-1d) or (A-1e)

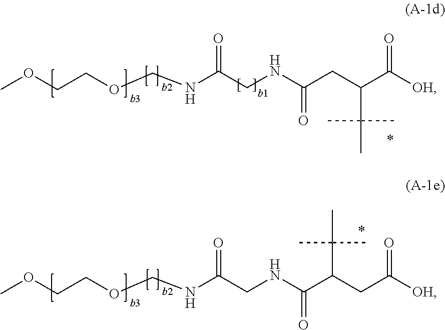

wherein
the dashed line marked with the asterisk indicates attachment to the sulfur atom of the side chain of the cysteine at position 38;
b1 is 2;
b2 is 2;
b3 ranges from 90 to 140, from 95 to 135 or from 100 to 125;
and wherein —L¹—L²—Z is of formula (XI-a)

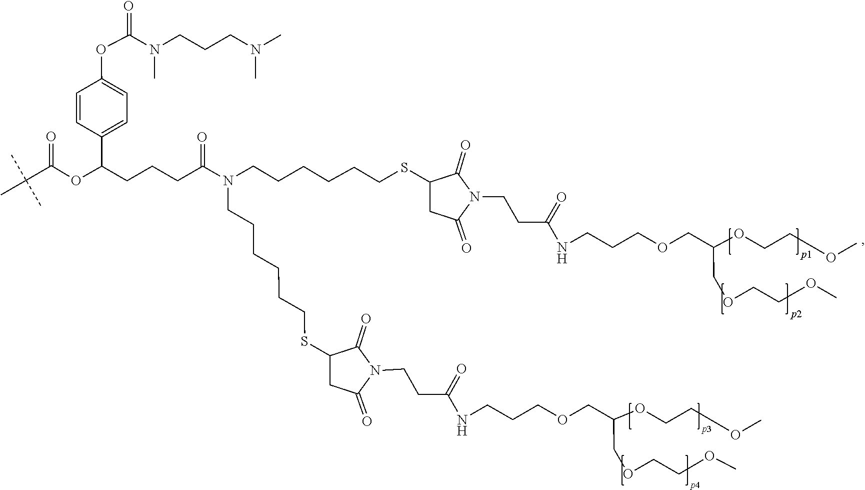

wherein the dashed line indicates attachment to the nitrogen of the ε-amino group of a lysine residue of —D′— or to the nitrogen of the N-terminal amine of —D′—; and
p1, p2, p3, p4 are independently of each other an integer ranging from 110 to 345, from 140 to 315, from 170 to 285, from 195 to 255, from 200 to 250, from 210 to 250 or from 220 to 240.

In certain embodiments the dashed line indicates attachment to the nitrogen of the ε-amino group of a lysine residue of —D′—.

In certain embodiments the IL-2 conjugate of formula (I), wherein —D is —D′—M_{mod} and wherein —D′— has the amino acid sequence of SEQ ID NO:4, —M_{mod} has the structure of (A-1d) or (A-1e)

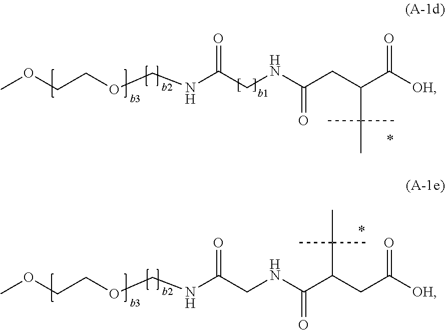

wherein
the dashed line marked with the asterisk indicates attachment to the sulfur atom of the side chain of the cysteine at position 38;
b1 is 3;
b2 is 2;
b3 ranges from 90 to 140, from 95 to 135 or from 100 to 125;
and wherein —L¹—L²—Z is of formula (XI-a)

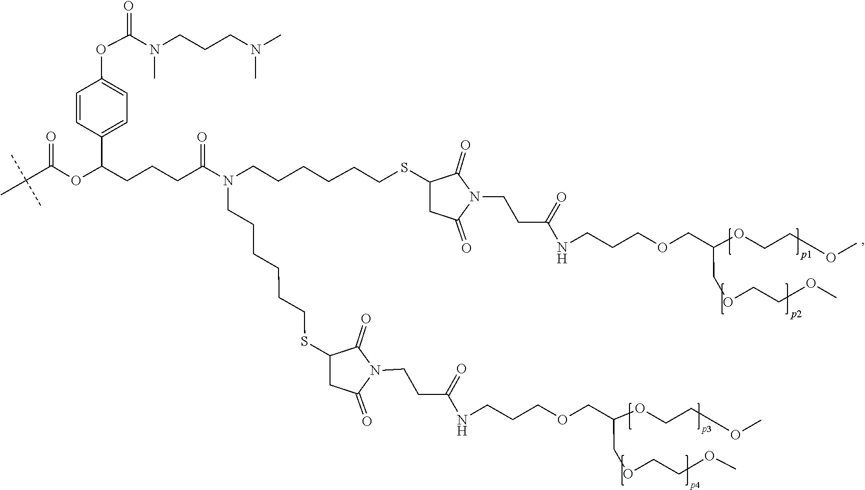

wherein the dashed line indicates attachment to the nitrogen of the ε-amino group of a lysine residue of —D′— or to the nitrogen of the N-terminal amine of —D′—; and
p1, p2, p3, p4 are independently of each other an integer ranging from 110 to 345, from 140 to 315, from 170 to 285, from 195 to 255, from 200 to 250, from 210 to 250 or from 220 to 240.

In certain embodiments the dashed line indicates attachment to the nitrogen of the ε-amino group of a lysine residue of —D′—.

In certain embodiments the IL-2 conjugate is of formula (I), wherein —D- is —D′—M_{mod} and 5 wherein —D′— has the amino acid sequence of SEQ ID NO:4, —M_{mod} has the structure of (A-1d) or (A-1e)

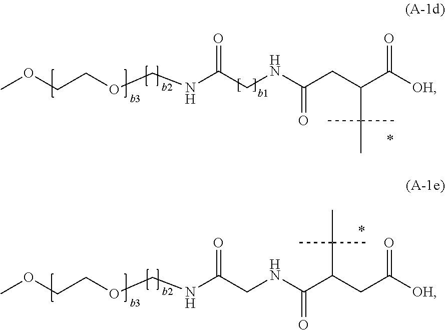

wherein
the dashed line marked with the asterisk indicates attachment to the sulfur atom of the side chain of the cysteine at position 38;
b1 is 2;
b2 is 3;
b3 ranges from 90 to 140, from 95 to 135 or from 100 to 125;
and wherein —L¹—L²—Z is of formula (XI-a)

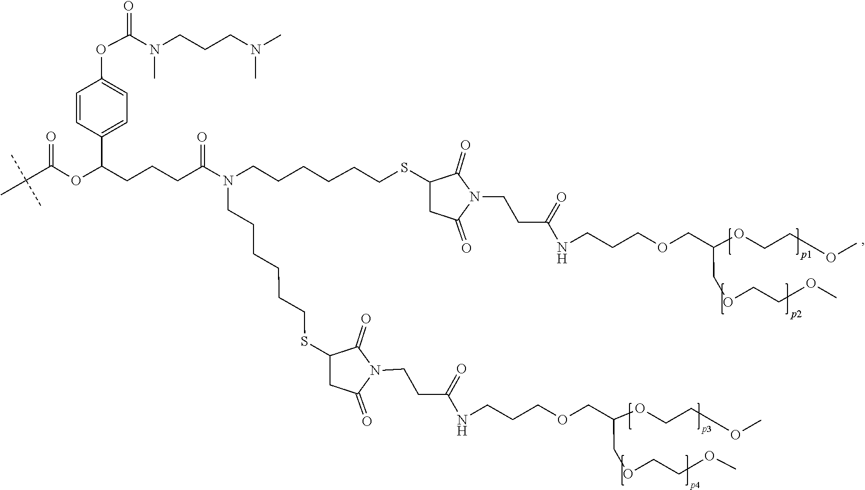

wherein the dashed line indicates attachment to the nitrogen of the ε-amino group of a lysine residue of —D′— or to the nitrogen of the N-terminal amine of —D′—; and
p1, p2, p3, p4 are independently of each other an integer ranging from 110 to 345, from 140 to 315, from 170 to 285, from 195 to 255, from 200 to 250, from 210 to 250 or from 220 to 240.

In certain embodiments the dashed line indicates attachment to the nitrogen of the ε-amino group of a lysine residue of —D′—.

In certain embodiments the IL-2 conjugate is of formula (I), wherein —D is —D′—M_{mod} and wherein —D′— has the amino acid sequence of SEQ ID NO. 4, —M_{mod} has the structure of (A-1d) or (A-1 e)

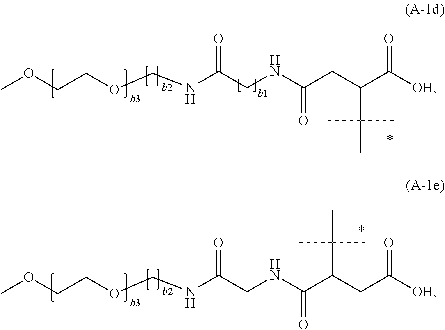

wherein
the dashed line marked with the asterisk indicates attachment to the sulfur atom of the side chain of the cysteine at position 38;
b1 is 3;
b2 is 3;
b3 ranges from 90 to 140, from 95 to 135 or from 100 to 125;

and wherein —L¹—L²—Z is of formula (XI-a)

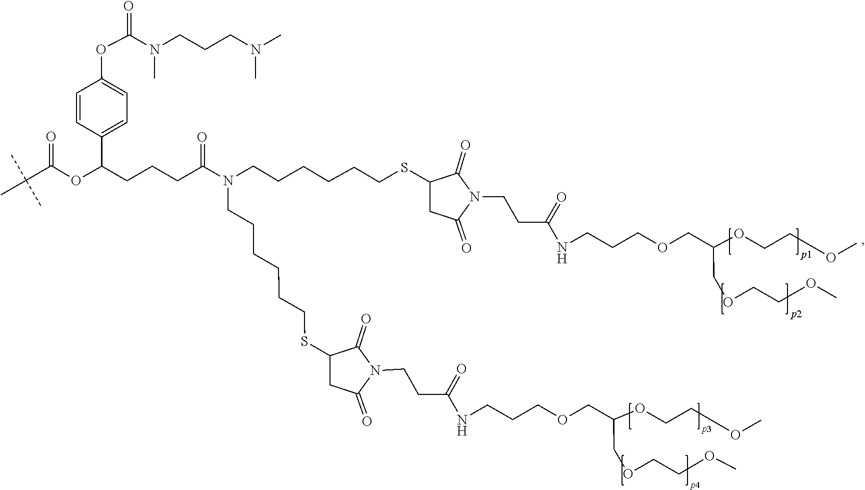

wherein the dashed line indicates attachment to the nitrogen of the ε-amino group of a lysine residue of —D′— or to the nitrogen of the N-terminal amine of —D′—; and
p1, p2, p3, p4 are independently of each other an integer ranging from 110 to 345, from 140 to 315, from 170 to 285, from 195 to 255, from 200 to 250, from 210 to 250 or from 220 to 240.

In certain embodiments the dashed line indicates attachment to the nitrogen of the ε-amino group of a lysine residue of —D′—.

The combination of a first stably attached polymeric moiety M_{mod} and a second reversibly attached polymeric moiety —Z provides the advantages of both ways of conjugation (stable and reversible). On the one hand, the first stably attached polymeric moiety M_{mod} may permanently alter functional properties of the IL-2-protein, e.g. by altering its receptor-binding affinity through steric hinderance. On the other hand, the second reversibly attached polymeric moiety —Z may change the bioavailability of IL-2, e.g. extend its half-life or shield its bioactivity in circulation while subsequently releasing the protein in its free form.

For all aspects of the present invention the cancer may in certain embodiments be selected from the group consisting of liquid tumors, solid tumors and lymphomas.

A liquid tumor may be a leukemia or myeloid neoplasm, such as chronic lymphocytic leukemia (CLL), chronic myelogenous leukemia (CML), hairy cell leukemia, lymphoblastic leukemia, myeloid leukemia, plasma cell leukemia, acute lymphoblastic leukemia (ALL), acute myeloid leukemia (AML), myelodysplastic syndrome (MDS), myeloproliferative neoplasm (MPN), post-MPN AML, post-MDS AML, del(5q)-associated high risk MDS or AML, blast-phase chronic myelogenous leukemia, multiple myeloma, myelodysplastic syndromes, chronic myeloproliferative disorders, plasma cell neoplasm and Waldenstrom's macroglobulinemia.

A solid tumor or lymphoma may be selected from the group consisting of lip and oral cavity cancer, oral cancer, liver cancer/hepatocellular cancer, primary liver cancer, lung cancer, lymphoma, malignant mesothelioma, malignant thymoma, skin cancer, intraocular melanoma, metastatic squamous neck cancer with occult primary, childhood multiple endocrine neoplasia syndrome, mycosis fungoides, nasal cavity and paranasal sinus cancer, nasopharyngeal cancer, neuroblastoma, oropharyngeal cancer, ovarian cancer, pancreatic cancer, parathyroid cancer, pheochromocytoma, pituitary tumor, adrenocortical carcinoma, AIDS-related malignancies, anal cancer, bile duct cancer, bladder cancer, brain and nervous system cancer, breast cancer, bronchial adenoma/carcinoid, gastrointestinal carcinoid tumor, carcinoma, colorectal cancer, endometrial cancer, esophageal cancer, extracranial germ cell tumor, extragonadal germ cell tumor, extrahepatic bile duct cancer, gallbladder cancer, gastric (stomach) cancer, gestational trophoblastic tumor, head and neck cancer, hypopharyngeal cancer, islet cell carcinoma (endocrine pancreas), kidney cancer/renal cell cancer, laryngeal cancer, pleuropulmonary blastoma, prostate cancer, transitional cell cancer of the renal pelvis and ureter, retinoblastoma, salivary gland cancer, sarcoma, Sezary syndrome, small intestine cancer, genitourinary cancer, malignant thymoma, thyroid cancer, Wilms' tumor, cholangiocarcinoma, and also their related earlier stages of aberrant cell growth such as dysplasias, adenomas, and carcinoma in situs.

In certain embodiments the cancer is a liver cancer/hepatocellular cancer. In certain embodiments the cancer is a lung cancer. In certain embodiments the cancer is a lymphoma. In certain embodiments the cancer is a malignant thymoma. In certain embodiments the cancer is a skin cancer. In certain embodiments the cancer is a metastatic squamous neck cancer with occult primary. In certain embodiments the cancer is a neuroblastoma. In certain embodiments the cancer is an ovarian cancer. In certain embodiments the cancer is a pancreatic cancer. In certain embodiments the cancer is a bile duct cancer. In certain embodiments the cancer is a bladder cancer. In certain embodiments the cancer is a brain and nervous system cancer. In certain embodiments the cancer is a breast cancer. In certain embodiments the cancer is a gastrointestinal carcinoid tumor. In certain embodiments the cancer is a carcinoma. In certain embodiments the cancer is a colorectal cancer. In certain embodiments the cancer is an extrahepatic bile duct cancer. In certain embodiments the cancer is a gallbladder cancer. In certain embodiments the cancer is a gastric (stomach) cancer. In certain embodiments the cancer is a head and neck cancer. In certain embodiments the cancer is a kidney cancer/renal cell cancer. In certain embodiments the cancer is a prostate cancer. In certain embodiments the cancer is a sarcoma. In certain embodiments the cancer is a small intestine cancer. In certain embodiments the cancer is a genitourinary cancer. In certain embodiments the cancer is a salivary gland cancer Examples for salivary gland cancer are mucoepidermoid carcinoma, acinic cell carcinoma, adenoid cystic carcinoma and polymorphous adenocarcinoma. In certain embodiments the cancer is a mucoepidermoid carcinoma. In certain embodiments the cancer is an acinic cell carcinoma. In certain embodiments the cancer is an adenoid cystic carcinoma. In certain embodiments the cancer is a polymorphous adenocarcinoma.

In certain embodiments the cancer is a PD-L1 positive acinic cell carcinoma.

Examples for endometrial cancers are endometrioid adenocarcinoma, clear cell carcinoma, uterine papillary serous carcinoma, uterine clear cell carcinoma, uterine carcinosarcoma and uterine sarcoma.

In certain embodiments the cancer is a mismatch repair deficient (dMMR) endometrial cancer.

Examples for lung cancer are non-small cell lung cancer and small cell lung cancer. In certain embodiments the cancer is a non-small cell lung cancer (NSCLC). In certain embodiment the cancer is a small cell lung cancer (SCLC).

Examples for lymphomas are AIDS-related lymphoma, primary central nervous system lymphoma, T-cell lymphoma, cutaneous T-cell lymphoma, Hodgkin's lymphoma, Hodgkin's lymphoma during pregnancy, non-Hodgkin's lymphoma, non-Hodgkin's lymphoma during pregnancy and angioimmunoblastic lymphoma.

Examples for skin cancer are melanoma and Merkel cell carcinoma. In certain embodiments the cancer is a skin cancer. In certain embodiments the cancer is a Merkel cell carcinoma. In certain embodiments the cancer is a melanoma.

An ovarian cancer may for example be an epithelial cancer, a germ cell tumor or a low malignant potential tumor. In certain embodiments the ovarian cancer is an epithelial cancer. In certain embodiments the ovarian cancer is a germ cell tumor. In certain embodiments the ovarian cancer is a low malignant potential tumor.

In certain embodiments the cancer is an ovarian cancer. In certain embodiments the ovarian cancer is a platinum-resistant ovarian cancer (PROC).

A pancreatic cancer may for example be a pancreatic exocrine tumor/adenocarcinoma, pancreatic endocrine tumor (PET) or pancreatic neuroendocrine tumor (NET). In certain embodiments the cancer is a pancreatic exocrine tumor/adenocarcinoma. In certain embodiments the cancer is a pancreatic endocrine tumor. In certain embodiments the cancer is a pancreatic neuroendocrine tumor.

In certain embodiments the cancer is a pancreatic cancer. In certain embodiments the cancer is a microsatellite stable pancreatic cancer. In certain embodiments the cancer is a microsatellite instability-high (MSI-H) pancreatic cancer. In certain embodiments the cancer is a microsatellite instability-low (MSI-L) pancreatic cancer. In certain embodiments the cancer is a microsatellite stable (MSS) pancreatic cancer.

Examples for brain and nervous system cancer are medulloblastoma, such as a childhood medulloblastoma, astrocytoma, ependymoma, neuroectodermal tumors, schwannoma, meningioma, pituitary adenoma and glioma. In certain embodiment the cancer is a medulloblastoma. In certain embodiments the cancer is a childhood medulloblastoma. In certain embodiments the cancer is an astrocytoma. In certain embodiments the cancer is an ependymoma. In certain embodiments the cancer is a neuroectodermal tumor. In certain embodiments the cancer is a schwannoma. In certain embodiments the cancer is a meningioma. In certain embodiments the cancer is a pituitary adenoma. In certain embodiments the cancer is a glioma.

An astrocytoma may be selected from the group consisting of giant cell glioblastoma, glioblastoma, secondary glioblastoma, primary adult glioblastoma, primary pediatric glioblastoma, oligodendroglial tumor, oligodendroglioma, anaplastic oligodendroglioma, oligoastrocytic tumor, oligoastrocytoma, anaplastic oligodendroglioma, oligoastrocytic tumor, oligoastrocytoma, anaplastic oligoastrocytoma, anaplastic astrocytoma, pilocytic astrocytoma, subependymal giant-cell astrocytoma, diffuse astrocytoma, pleomorphic xanthoastrocytoma and cerebellar astrocytoma.

Examples for a neuroectodermal tumor are a pineal primitive neuroectodermal tumor and a supratentorial primitive neuroectodermal tumor.

An ependymoma may be selected from the group consisting of subependymoma, ependymoma, myxopapillary ependymoma and anaplastic ependymoma.

A meningioma may be an atypical meningioma or an anaplastic meningioma.

A glioma may be selected from the group consisting of glioblastoma multiforme, paraganglioma, suprantentorial primordial neuroectodermal tumor (sPNET), brain stem glioma, childhood brain stem glioma, hypothalamic and visual pathway glioma, childhood hypothalamic and visual pathway glioma and malignant glioma.

Examples for breast cancer are breast cancer during pregnancy, triple negative breast cancer, ductal carcinoma in situ (DCIS), invasive ductal carcinoma (IDC), tubular carcinoma of the breast, medullary carcinoma of the breast, mucinous carcinoma of the breast, papillary carcinoma of the breast, cribriform carcinoma of the breast, invasive lobular carcinoma (ILC), inflammatory breast cancer, lobular carcinoma in situ (LCIS), male breast cancer, Paget's disease of the nipple, phyllodes tumors of the breast and metastatic breast cancer. In certain embodiments the cancer is a breast cancer during pregnancy. In certain embodiments the cancer is a triple negative breast cancer. In certain embodiments the cancer is a ductal carcinoma in situ. In certain embodiments the cancer is an invasive ductal carcinoma. In certain embodiments the cancer is a tubular carcinoma of the breast. In certain embodiments the cancer is a medullary carcinoma of the breast. In certain embodiments the cancer is a mucinous carcinoma of the breast. In certain embodiments the cancer is a papillary carcinoma of the breast. In certain embodiments the cancer is a cribriform carcinoma of the breast. In certain embodiments the cancer is an invasive lobular carcinoma. In certain embodiments the cancer is an inflammatory breast cancer. In certain embodiments the cancer is a lobular carcinoma in situ. In certain embodiments the cancer is a male breast cancer. In certain embodiments the cancer is a Paget's disease of the nipple. In certain embodiments the cancer is a phyllodes tumor of the breast. In certain embodiments the cancer is a metastatic breast cancer.

In certain embodiments the cancer is a triple negative breast cancer (TNBC).

Examples for a carcinoma are neuroendocrine carcinoma, adrenocortical carcinoma and Islet cell carcinoma. In certain embodiments the cancer is a neuroendocrine carcinoma. In certain embodiments the cancer is an adrenocortical carcinoma. In certain embodiments the cancer is an Islet cell carcinoma.

Examples for a colorectal cancer are colon cancer and rectal cancer. In certain embodiments the cancer is a colon cancer. In certain embodiments the cancer is a rectal cancer.

In certain embodiments the cancer is a microsatellite instability-high (MSI-H) colorectal cancer. In certain embodiments the cancer is a microsatellite instability-low (MSI-L) colorectal cancer. In certain embodiments the cancer is a microsatellite stable (MSS) colorectal cancer.

Examples for kidney cancer/renal cell cancer are clear cell renal carcinoma (ccRCC), papillary renal carcinoma (PRCC), chromophobe renal carcinoma (chRCC), clear cell papillary renal carcinoma (ccpRCC), collecting duct renal carcinoma (cdRCC) and medullary renal carcinoma (RMC). In certain embodiments the cancer is a clear cell renal carcinoma (ccRCC). In certain embodiments the cancer is a papillary renal carcinoma (PRCC). In certain embodiments the cancer is a chromophobe renal carcinoma (chRCC). In certain embodiments the cancer is a clear cell papillary renal carcinoma (ccpRCC). In certain embodiments the cancer is a collecting duct renal carcinoma (cdRCC). In certain embodiments the cancer is a medullary renal carcinoma (RMC).

In certain embodiments the cancer is a clear cell renal cell carcinoma (ccRCC) which is refractory to treatment with an inhibitor of PD-1 or PD-L1 therapy. In certain embodiments the cancer which is refractory to treatment with an inhibitor of PD-1 is a clear cell renal cell carcinoma (ccRCC). In certain embodiments the cancer which is refractory to treatment with an inhibitor of PD-L1 is a clear cell renal cell carcinoma (ccRCC).

A sarcoma may be selected from the group consisting of Kaposi's sarcoma, osteosarcoma/malignant fibrous histiocytoma of bone, soft tissue sarcoma, Ewing's family of tumors/sarcomas, rhabdomyosarcoma, clear cell sarcoma of tendon sheaths, central chondrosarcoma, central and periosteal chondroma, fibrosarcoma and uterine sarcoma. In certain embodiments the cancer may be a Kaposi's sarcoma. In certain embodiments the cancer may be an osteosarcoma/malignant fibrous histiocytoma of bone. In certain embodiments the cancer may be a soft tissue sarcoma. In certain embodiments the cancer may be an Ewing's family of tumors/sarcomas. In certain embodiments the cancer may be a rhabdomyosarcoma.

In certain embodiments the cancer may be a clear cell sarcoma of tendon sheaths. In certain embodiments the cancer may be a central chondrosarcoma. In certain embodiments the cancer may be a central and periosteal chondroma. In certain embodiments the cancer may be a fibrosarcoma. In certain embodiments the cancer may be a uterine sarcoma.

Examples for a genitourinary cancer are testicular cancer, urethral cancer, vaginal cancer, cervical cancer, penile cancer and vulvar cancer. In certain embodiments the cancer may be a testicular cancer. In certain embodiments the cancer may be a urethral cancer. In certain embodiments the cancer may be a vaginal cancer. In certain embodiments the cancer may be a cervical cancer. In certain embodiments the cancer may be a penile cancer. In certain embodiments the cancer may be a vaginal cancer.

In certain embodiments the solid tumor is selected from the group consisting of squamous cell carcinoma of the head and neck (SCCHN); HPV-associated cancers, such as anal, vulvar, cervical, penile and vaginal cancers; melanomas; pancreatic cancer and breast cancer, such as triple-negative breast cancer (TNBC).

In certain embodiments the solid tumor is selected from the group consisting of melanoma, cervical cancer, pancreatic cancer, renal cell cancer, colorectal cancer, ovarian cancer, breast cancer, salivary gland cancer, endometrial cancer and lung cancer.

In certain embodiments the solid tumor is selected from the group consisting of pancreatic cancer, renal cell cancer, colorectal cancer, ovarian cancer, breast cancer, acinic cell carcinoma, endometrial cancer and lung cancer.

In certain embodiments the solid tumor is selected from the group consisting of pancreatic cancer, renal cell cancer (RCC), colorectal cancer (CRC), ovarian cancer and breast cancer.

In certain embodiments the solid tumor is selected from the group consisting of colorectal cancer, lung cancer and ovarian cancer.

In certain embodiments the solid tumor is selected from the group consisting of melanoma, cervical cancer, colorectal cancer, lung cancer and ovarian cancer.

Early clinical data showed that hard to treat cancers, such as pancreatic cancer or small cell lung cancer, responded to the treatment with the IL-2 conjugate of formula (I) as monotherapy or in combination with pembrolizumab surprisingly well.

In certain embodiments the cancer was not subject to prior lines of treatment. In certain embodiments the cancer has previously been treated with standard of care treatment. In certain embodiments the cancer has previously been treated with chemotherapy, such as platinum-based chemotherapy.

In certain embodiments the cancer has previously been treated with an immune checkpoint inhibitor. In certain the embodiments the cancer has previously been treated with an inhibitor of PD-1 or PD-L1. In certain embodiments the cancer has previously been treated with an inhibitor of PD-1. In certain embodiments the cancer has previously been treated with an inhibitor of PD-L1. In certain embodiments the cancer has previously been treated with an anti-PD-1 antibody. In certain embodiments the cancer has previously been treated with an anti-PD-L1 antibody. In certain embodiments the cancer has previously been treated with pembrolizumab. In certain embodiments the cancer has previously been treated with nivolumab. In certain embodiments the cancer has previously been treated with atezolizumab.

In certain embodiments the cancer which has previously been treated with an inhibitor of PD-1 or PD-L1 is a lung cancer. In certain embodiments the cancer which has previously been treated with an inhibitor of PD-1 or PD-L1 is a small cell lung cancer (SCLC). In certain embodiments the cancer which has previously been treated with an inhibitor of PD-1 or PD-L1 is a non-small cell lung cancer (NSCLC).

In certain embodiments the cancer which has previously been treated with an inhibitor of PD-1 or PD-L1 is a melanoma.

It was surprisingly found that also cancers which are refractory to treatment with an inhibitor of PD-1 or PD-L1 respond to the treatment with the IL-2 conjugate of formula (I) as monotherapy or in combination with pembrolizumab.

In certain embodiments the cancer is treated with the IL-2 conjugate of formula (I) in a neoadjuvant setting. In certain embodiments the cancer is treated with the IL-2 conjugate of formula (I) in an adjuvant setting.

In certain embodiments the cancer to be treated in a neoadjuvant setting is melanoma. In certain embodiments the cancer to be treated in a neoadjuvant setting is non-small cell lung cancer.

In certain embodiments the cancer is a PD-1 positive cancer. In certain embodiments the cancer is a PD-1 negative cancer. In certain embodiments the cancer is a PD-L1 positive cancer. In certain embodiments the cancer is a PD-L1 negative cancer.

In certain embodiments the IL-2 conjugate or the pharmaceutical composition of the present invention is administered to the patient prior to, simultaneously with or after administration of one or more additional drug, which one or more additional drug is in certain embodiments selected from the group consisting of pattern recognition receptor agonists (PRRA), cytotoxic/chemotherapeutic agents, immune checkpoint inhibitors or antagonists, immune checkpoint agonists, immune activating receptor agonists, multi-specific drugs, antibody-drug conjugates (ADC), antibody-adjuvant conjugates (AAC), radionuclides or targeted radionuclide therapeutics, DNA damage repair inhibitors, tumor metabolism inhibitors, pattern recognition receptor agonists, protein kinase inhibitors, chemokine and chemoattractant receptor agonists, chemokine or chemokine receptor antagonists, cytokine receptor agonists, death receptor agonists, CD47 or SIRPα antagonists, oncolytic drugs, signal converter proteins, epigenetic modifiers, tumor peptides or tumor vaccines, heat shock protein (HSP) inhibitors, proteolytic enzymes, ubiquitin and proteasome inhibitors, adhesion molecule antagonists, hormones including hormone peptides and synthetic hormones, and adoptive cellular therapies such as Tumor Infiltrating Lymphocyte (TIL) therapy, Chimeric Antigen Receptor (CAR) therapy, T cell therapy, Natural Killer (NK) cell therapy, CAR-T therapy, CAR-NK therapy, CAR-76 therapy, CAR-Macrophage therapy or any other cellular therapy with a genetically modified or genetically unmodified immune cell type.

The PRRA may be selected from the group consisting of Toll-like receptor (TLR) agonists, NOD-like receptor agonists (NLRs), RIG-I-like receptor agonists, cytosolic DNA sensor agonists, STING agonists, and aryl hydrocarbon receptor agonists (AhR).

In certain embodiments the PRRA is a Toll-like receptor agonist, such as a Toll-like receptor agonists selected from the group consisting of agonists of TLR½, such as peptidoglycans, lipoproteins, Pam3CSK4, Amplivant, SLP-AMPLIVANT, HESPECTA, ISA101 and ISA201; agonists of TLR2, such as LAM-MS, LPS-PG, LTA-BS, LTA-SA, PGN-BS, PGN-EB, PGN-EK, PGN-SA, CL429, FSL-1, Pam2CSK4, Pam3CSK4, zymosan, CBLB612, SV-283, ISA204, SMP105, heat killed Listeria monocytogenes; agonists of TLR3, such as poly(A:U), poly(I.C) (poly-ICLC), rintatolimod, apoxxim, IPH3102, poly-ICR, PRV300, RGCL2, RGIC.1, Riboxxim (RGC100, RGIC100), Riboxxol (RGIC50)), synthetic natural or modified double stranded RNA, synthetic natural or modified nucleic acid oligomers and Riboxxon; agonists of TLR4, such as lipopolysaccharides (LPS), neoceptin-3, glucopyranosyl lipid adjuvant (GLA), GLA-SE, G100, GLA-AF, clinical center reference endotoxin (CCRE), monophosphoryl lipid A, grass MATA MPL, PEPA10, ONT—10 (PET-Lipid A, oncothyreon), G-305, ALD046, CRX527, CRX675 (RC527, RC590), GSK1795091, OM197MPAC, OM294DP, tumor targeted TLR4 agonists, and SAR439794; agonists of TLR2/4, such as lipid A, OM174 and PGN007; agonists of TLR5, such as flagellin, entolimod, mobilan, protectan CBLB501; agonists of TLR6/2, such as diacylated lipoproteins, diacylated lipopeptides, FSL-1, MALP-2 and CBLB613; agonists of TLR7, such as CL264, CL307, imiquimod (R837), TMX-101, TMX-201, TMX-202, TMX302, gardiquimod, S-27609, 851, UC-IV150, 852A (3M-001, PF-04878691), loxoribine, polyuridylic acid, GSK2245035, GS-9620, R06864018 (ANA773, RG7795), R07020531, isatoribine, AN0331, ANA245, ANA971, ANA975, DSP0509, DSP3025 (AZD8848), GS986, MBS2, MBS5, RG7863 (R06870868), sotirimod, SZU101, synthetic natural or modified single stranded RNA, synthetic nucleic acids, synthetic natural or modified nucleic acid oligomers, tumor targeted TLR7 agonists, and TQA3334; agonists of TLR8, such as ssPolyUridine, ssRNA40, TL8-506, XG-1-236, VTX-2337 (motolimod), VTX-1463, VTX378, VTX763, DN1508052, SBT6050, synthetic natural or modified single stranded RNA, synthetic nucleic acids, synthetic natural or modified nucleic acid oligomers, tumor targeted TLR8 agonists, and GS9688; agonists of TLR7/8, such as TransCon™ TLR7/8 agonist, CL075, CL097, poly(dT), resiquimod (R-848, VML600, S28463), MEDI9197 (3M-052), NKTR262, DV1001, IM04200, IPH3201, synthetic natural or modified single stranded RNA, synthetic nucleic acids, synthetic nucleic acid oligomers, BDC-1001, other tumor targeted TLR7/8 agonists and VTX1463; agonists of TLR9, such as CpG DNA, CpG ODN, lefitolimod (MGN1703), SD-101, QbG10, CYT003, CYT003-QbG10, DUK-CpG-001, CpG-7909 (PF-3512676), GNKG168, EMD 1201081, IMO—2125, IMO—2055, CpG10104, AZD1419, AST008, IM02134, MGN1706, IRS 954, 1018 ISS, actilon (CPG10101), ATP00001, AVE0675, AVE7279, CMP001, DIMS0001, DIMS9022, DIMS9054, DIMS9059, DV230, DV281, EnanDIM, heplisav (V270), kappaproct (DIMS0150), NJP834, NPI503, SAR21609, synthetic natural or modified nucleic acid oligomers and tolamba; and agonists of TLR7/9, such as DV1179. In certain embodiments the one or more additional drug is an agonist of TLR7/8, such as resiquimod.

In certain embodiments the one or more additional drug is of formula (A-3)

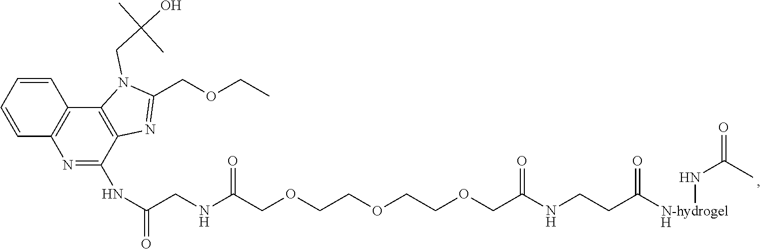

wherein “hydrogel” is a hydrogel comprising backbone moieties of formula (A-4):

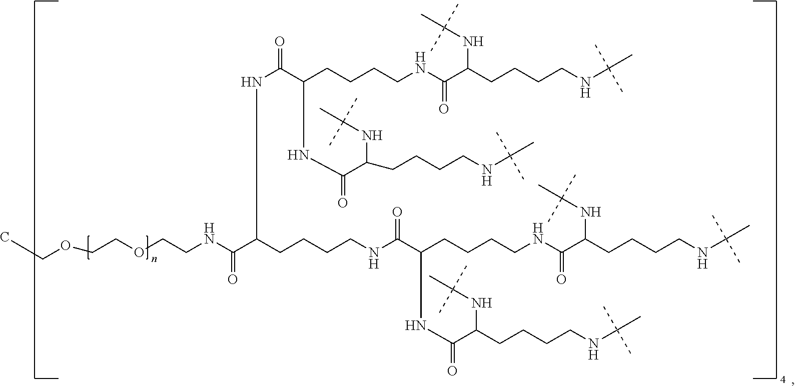

wherein n ranges from approx. 25 to 29 and dashed lines indicate attachment to either a moiety

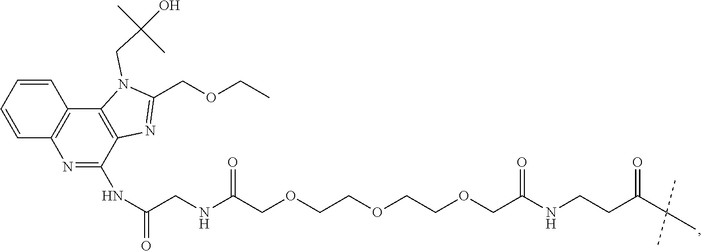

to a moiety

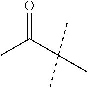

or to a crosslinker of formula (A-5):

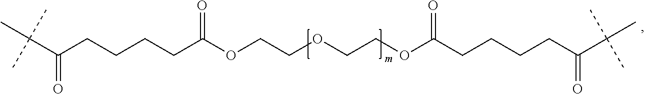

wherein m ranges from approx. 41 to 45 and the dashed lines in (A-5) indicate attachment to a backbone moiety of formula (A-4).

In certain embodiments the one or more additional drug is of formula (A-6)

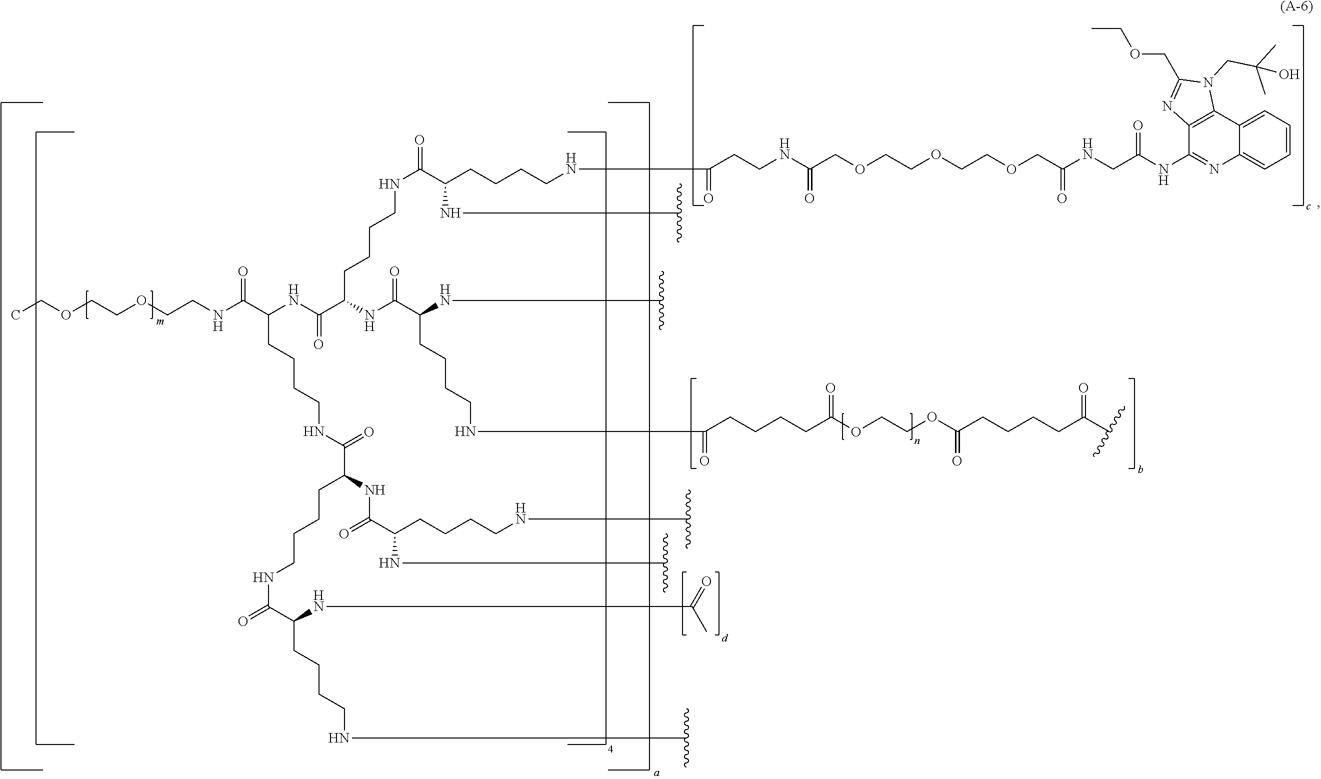

wherein
m ranges from approx. 25 to 29;
n ranges from approx. 41 to 45;
“a” denotes a backbone moiety, “b” denotes a crosslinker moiety, “c” denotes a reversibly conjugated resiquimod moiety and “d” denotes an acetamide moiety; and the ratio of moieties “a”:“b”:“c”:“d” in the conjugate is approx. 1:13:2:4 or is approx. 1:13:2.4:3.6.

In certain embodiments the one or more additional drug is of formula (Ai-6)

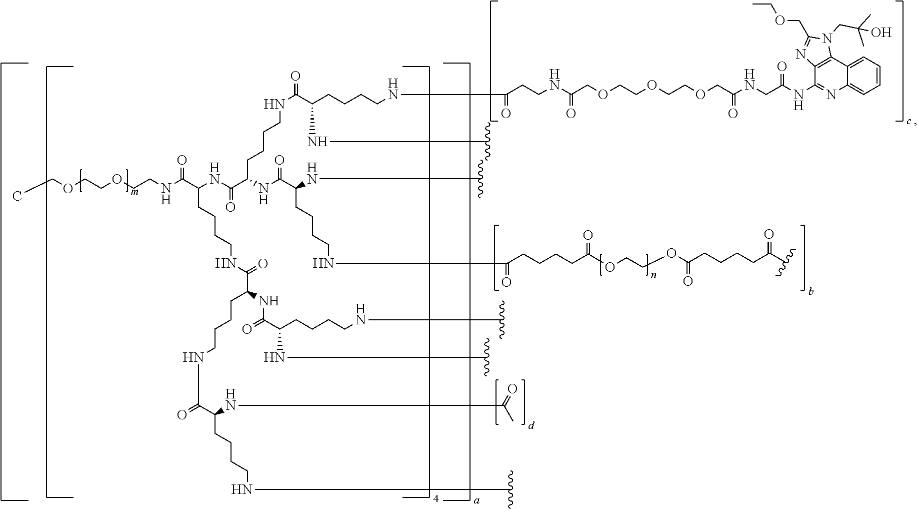

wherein
m ranges from approx. 25 to 29;
n ranges from approx. 41 to 45;
“a” denotes a backbone moiety, “b” denotes a crosslinker moiety, “c” denotes a reversibly conjugated resiquimod moiety and “d” denotes an acetamide moiety; and
the ratio of moieties “a”:“b”:“c”:“d” in the conjugate is approx. 1:13:c:d with c+d=6, such as a ratio of moieties “a”:“b”:“c”:“d” of approx. 1:13:3:3 or of approx. 1:13:2:4 or of approx. 1:13:4:2.

The drug of formula (A-3), (A-6) or (Ai-6) may be administered as 0.5 mg per tumor lesion by intratumoral administration, such as intratumoral injection, once or multiple times. For multiple administrations, the interval between two consecutive administrations may range from two to eight weeks.

In certain embodiments the one or more additional drug is of formula (A-3), (A-6) or (Ai-6), which is administered via intratumoral administration at a dose of 0.5 mg per lesion, either prior to, together with or after administration of the IL-2 conjugate. In certain embodiments the one or more additional drug is of formula (A-3), (A-6) or (Ai-6), which is administered via intratumoral administration at a dose of 0.5 mg per lesion prior to administration of the IL-2 conjugate. In certain embodiments the one or more additional drug is of formula (A-3), (A-6) or (Ai-6), which is administered via intratumoral administration at a dose of 0.5 mg per lesion together with administration of the IL-2 conjugate. In certain embodiments the one or more additional drug is of formula (A-3), (A-6) or (Ai-6), which is administered via intratumoral administration at a dose of 0.5 mg per lesion after administration of the IL-2 conjugate.

In certain embodiments the IL-2 conjugate or a pharmaceutically acceptable salt thereof is for use in the treatment of cancer, wherein the IL-2 conjugate is administered in a dose ranging from 80 μg IL-2/kg to 160 μg IL-2/kg as a cotreatment with the drug of formula (A-3), (A-6) or (Ai-6), wherein the drug of formula (A-3), (A-6) or (Ai-6) is administered via intratumoral administration at a dose of 0.5 mg per lesion, either prior to, together with or after administration of the IL-2 conjugate.

In certain embodiments the IL-2 conjugate or a pharmaceutically acceptable salt thereof is for use in the treatment of cancer, wherein the IL-2 conjugate is administered in a dose ranging from 100 μg IL-2/kg to 140 μg IL-2/kg as a cotreatment with the drug of formula (A-3), (A-6) or (Ai-6), wherein the drug of formula (A-3), (A-6) or (Ai-6) is administered via intratumoral administration at a dose of 0.5 mg per lesion, either prior to, together with or after administration of the IL-2 conjugate.

In certain embodiments the IL-2 conjugate or a pharmaceutically acceptable salt thereof is for use in the treatment of cancer, wherein the IL-2 conjugate is administered in a dose ranging from 110 μg IL-2/kg to 130 μg IL-2/kg as a cotreatment with the drug of formula (A-3), (A-6) or (Ai-6), wherein the drug of formula (A-3), (A-6) or (Ai-6) is administered via intratumoral administration at a dose of 0.5 mg per lesion, either prior to, together with or after administration of the IL-2 conjugate.

In certain embodiments the IL-2 conjugate or a pharmaceutically acceptable salt thereof is for use in the treatment of cancer, wherein the IL-2 conjugate is administered in a dose of about 120 μg IL-2/kg as a cotreatment with the drug of formula (A-3), (A-6) or (Ai-6), wherein the drug of formula (A-3), (A-6) or (Ai-6) is administered via intratumoral administration at a dose of 0.5 mg per lesion, either prior to, together with or after administration of the IL-2 conjugate.

In certain embodiments the IL-2 conjugate or a pharmaceutically acceptable salt thereof is for use in the treatment of cancer, wherein the IL-2 conjugate is administered in a dose of 120 μg IL-2/kg as a cotreatment with the drug of formula (A-3), (A-6) or (Ai-6), wherein the drug of formula (A-3), (A-6) or (Ai-6) is administered every three weeks via intratumoral administration at a dose of 0.5 mg per lesion, either prior to, together with or after administration of the IL-2 conjugate.

In certain embodiments the drug of formula (A-3), (A-6) or (Ai-6) is administered to the patient every three weeks. In certain embodiments the drug of formula (A-3), (A-6) or (Ai-6) is administered to the patient every six weeks. In certain embodiments the drug of formula (A-3), (A-6) or (Ai-6) is administered at a frequency dependent on disease progression.

In certain embodiments the IL-2 conjugate or a pharmaceutically acceptable salt thereof is for use in the treatment of cancer, wherein the IL-2 conjugate is administered in a dose of about 120 μg IL-2/kg as a cotreatment with the drug of formula (A-3), (A-6) or (Ai-6), wherein the drug of formula (A-3), (A-6) or (Ai-6) is administered via intratumoral injection every three weeks at a dose of 0.5 mg per lesion, either prior to, together with or after administration of the IL-2 conjugate.

In certain embodiments the IL-2 conjugate or a pharmaceutically acceptable salt thereof is for use in the treatment of cancer, wherein the IL-2 conjugate is administered in a dose of about 120 μg IL-2/kg as a cotreatment with the drug of formula (A-3), (A-6) or (Ai-6), wherein the drug of formula (A-3), (A-6) or (Ai-6) is administered via intratumoral injection every six weeks at a dose of 0.5 mg per lesion, either prior to, together with or after administration of the IL-2 conjugate.

In certain embodiments the IL-2 conjugate or a pharmaceutically acceptable salt thereof for use in the treatment of cancer is administered via intravenous infusion as a cotreatment with the drug of formula (A-3), (A-6) or (Ai-6).

Examples for CpG ODN are ODN 1585, ODN 2216, ODN 2336, ODN 1668, ODN 1826, ODN 2006, ODN 2007, ODN BWO06, ODN D-SLO1, ODN 2395, ODN M362 and ODN D-SLO3.

In certain embodiments the PRRA is a NOD-like receptor agonist. If the one or more additional drug is a NOD-like receptor agonist, such NOD-like receptor agonist may be selected from the group consisting of agonists of NOD1, such as C12-iE-DAP, C₁₋₄—Tri-LAN-Gly, iE-DAP, iE-Lys, and Tri-DAP; and agonists of NOD2, such as L18-MDP, MDP, M-TriLYS, murabutide and N-glycolyl-MDP. In certain embodiments the one or more additional drug is an agonist of NOD1. In certain embodiments the one or more additional drug is an agonist of NOD2.

In certain embodiments the PRRA is a RIG-I-like receptor agonist. If the one or more additional drug is a RIG-I-like receptor agonist, such RIG-I-like receptor agonist may be selected from the group consisting of 3p-hpRNA, 5′ppp-dsRNA, 5′ppp RNA (M8), 5′OH RNA with kink (CBS-13—BPS), 5′PPP SLR, KIN100, KIN 101, KIN1000, KIN1400, KIN1408, KIN1409, KIN1148, KIN131A, poly(dA:dT), SB9200, RGT100 and hiltonol.

In certain embodiments the PRRA is a cytosolic DNA sensor agonist. If the one or more additional drug is a cytosolic DNA sensor agonist, such cytosolic DNA sensor agonist may be selected from the group consisting of cGAS agonists, dsDNA-EC, G3-YSD, HSV-60, ISD, ODN TTAGGG (A151), poly(dG:dC) and VACV-70.

In certain embodiments the PRRA is a STING agonist. If the one or more additional drug is a STING agonist, such STING agonist may be selected from the group consisting of MK-1454, ADU-S100 (MIW815), 2′3′-cGAMP, 3′3′-cGAMP, c-di-AMP, c-di-GMP, cAIMP (CL592), cAIP difluor (CL614), cAIM(PS)2 difluor (Rp/Sp) (CL656), 2′2′-cGAMP, 2′3′-cGAM(PS)2 (Rp/Sp), 3′3′-cGAM fluorinated, c-di-AMP fluorinated, 2′3′-c-di-AMP, 2′3′-c-di-AM(PS)2 (Rp,Rp), c-di-GMP fluorinated, 2′3′-c-di-GMP, c-di-IMP, c-di-UMP and DMXAA (vadimezan, ASA404). In certain embodiments the one or more additional drug is MK-1454.

In certain embodiments the one or more additional drug is ADU-S100 (MIW815). In certain embodiments the one or more additional drug is 2′3′-cGAMP.

In certain embodiments the PRRA is an aryl hydrocarbon receptor agonist. If the one or more additional drug is an aryl hydrocarbon receptor (AhR) agonist, such AhR agonist may be selected from the group consisting of FICZ, ITE and L-kynurenine.

In certain embodiments the one or more additional drug is a cytotoxic/chemotherapeutic agent.

In certain embodiments the one or more additional drug is an immune checkpoint inhibitor or antagonist. In certain embodiments the one or more additional drug is an immune activating receptor agonist. In certain embodiments the one or more additional drug is a multi-specific drug. In certain embodiments the one or more additional drug is an antibody-drug conjugate (ADC). In certain embodiments the one or more additional drug is an antibody-adjuvant conjugate (AAC). In certain embodiments the one or more additional drug is a radionuclide or a targeted radionuclide therapeutic. In certain embodiments the one or more additional drug is DNA damage repair inhibitor. In certain embodiments the one or more additional drug is a tumor metabolism inhibitor. In certain embodiments the one or more additional drug is a pattern recognition receptor agonist. In certain embodiments the one or more additional drug is a protein kinase inhibitor. In certain embodiments the one or more additional drug is a chemokine and chemoattractant receptor agonist. In certain embodiments the one or more additional drug is a chemokine or chemokine receptor antagonist. In certain embodiments the one or more additional drug is a cytokine receptor agonist. In certain embodiments the one or more additional drug is a death receptor agonist. In certain embodiments the one or more additional drug is a CD47 antagonist. In certain embodiments the one or more additional drug is a SIRPα antagonist. In certain embodiments the one or more additional drug is an oncolytic drug. In certain embodiments the one or more additional drug is a signal converter protein. In certain embodiments the one or more additional drug is an epigenetic modifier. In certain embodiments the one or more additional drug is a tumor peptide or tumor vaccine. In certain embodiments the one or more additional drug is a heat shock protein (HSP) inhibitor. In certain embodiments the one or more additional drug is a proteolytic enzyme. In certain embodiments the one or more additional drug is a ubiquitin and proteasome inhibitor. In certain embodiments the one or more additional drug is an adhesion molecule antagonist. In certain embodiments the one or more additional drug is a hormone including hormone peptides and synthetic hormones.

The cytotoxic or chemotherapeutic agent may be selected from the group consisting of alkylating agents, anthracyclines, pyrrolobenzodiazepines, nitrogen mustards, platinum agents, anti-metabolites, anti-microtubule agents, topoisomerase inhibitors, cytotoxic antibiotics, auristatins, enediynes, lexitropsins, duocarmycins, cyclopropylpyrroloindoles, puromycin, dolastatins, maytansine derivatives, alkylsufonates, triazenes and piperazine.

The alkylating agent may be selected from the group consisting of nitrogen mustards, such as mechlorethamine, cyclophosphamide, melphalan, chlorambucil, ifosfamide and busulfan; nitrosoureas, such as N-nitroso-N-methylurea, carmustine, lomustine, semustine, fotemustine and streptozotocin; tetrazines, such as dacarbazine, mitozolomide and temozolomide; ethylenimines, such as altretamine; aziridines, such as thiotepa, mitomycin and diaziquone; cisplatin and derivatives, such as cisplatin, carboplatin, oxaliplatin; and non-classical alkylating agents, such as procarbazine and hexamethylmelamine.

The anti-metabolite may be selected from the group consisting of anti-folates, such as methotrexate and pemetrexed; fluoropyrimidines, such as fluorouracil and capecitabine; deoxynucleoside analogues, such as cytarabine, gemcitabine, decitabine, azacytidine, fludarabine, nelarabine, cladribine, clofarabine and pentostatin; and thiopurines, such as thioguanine and mercaptopurine.

In certain embodiments the one or more additional drug is pemetrexed.

The anti-microtubule agent may be selected from the group consisting of Vinca alkaloids, such as vincristine, vinblastine, vinorelbine, vindesine and vinflunine; taxanes, such as paclitaxel and docetaxel; podophyllotoxins and derivatives, such as podophyllotoxin, etoposide and teniposide; stilbenoid phenol and derivatives, such as zybrestat (CA4P); and BNC105.

In certain embodiments the one or more additional drug is paclitaxel.

In certain embodiments the one or more additional drug is docetaxel.

In certain embodiments paclitaxel is administered at a dose of 80 mg/m². In certain embodiments paclitaxel is administered every two weeks. In certain embodiments paclitaxel is administered every three weeks. In certain embodiments paclitaxel is administered intravenously. In certain embodiments paclitaxel is administered intravenously at a dose of 80 mg/m² every two or three weeks.

In certain embodiments docetaxel is administered at a dose of 75 mg/m². In certain embodiments docetaxel is administered every three weeks. In certain embodiments docetaxel is administered intravenously. In certain embodiments docetaxel is administered intravenously at a dose of 75 mg/m² every three weeks.

In certain embodiments pemetrexed is administered at a dose of 500 mg/m². In certain embodiments pemetrexed is administered every three weeks. In certain embodiments pemetrexed is administered intravenously. In certain embodiments pemetrexed is administered intravenously at a dose of 500 mg/m² every three weeks.

The topoisomerase inhibitor may be selected from the group consisting of topoisomerase I inhibitors, such as irinotecan, topotecan and camptothecin; and topoisomerase II inhibitors, such as etoposide, doxorubicin, mitoxantrone, teniposide, novobiocin, merbarone and aclarubicin.

In certain embodiments the one or more additional drug is doxorubicin.

The cytotoxic antibiotic may be selected from the group consisting of anthracyclines, such as doxorubicin, daunorubicin, epirubicin and idarubicin; pirarubicin, aclarubicin, bleomycin, mitomycin C, mitoxantrone, actinomycin, dactinomycin, adriamycin, mithramycin and tirapazamine.

The auristatin may be selected from the group consisting of monomethyl auristatin E (MMAE) and monomethyl auristatin F (MMAF).

The enediyne may be selected from the group consisting of neocarzinostatin, lidamycin (C-1027), calicheamicins, esperamicins, dynemicins and golfomycin A.

The maytansine derivative may be selected from the group consisting of ansamitocin, mertansine (emtansine, DM1) and ravtansine (soravtansine, DM4).

The immune checkpoint inhibitor or antagonist may be selected from the group consisting of inhibitors of CTLA-4 (cytotoxic T-lymphocyte-associated protein 4), such as ipilimumab, tremelimumab, MK-1308, FPT155, PRSO10, BMS-986249, BPI-002, CBT509, JS007, ONC392, TE1254, IB1I310, BR02001, CG0161, KN044, PBI5D3H5, BCD145, ADU1604, AGEN1884, AGEN1181, CS1002 and CP675206; inhibitors of PD-1 (programmed cell death protein 1), such as pembrolizumab, nivolumab, pidilizumab, AMP-224, BMS-936559, cemiplimab and PDR001; inhibitors of PD-L1 (programmed cell death ligand 1), such as MDX—1105, MEDI4736, atezolizumab, avelumab, BMS-936559 and durvalumab; inhibitors of PD-L2 (programmed cell death-ligand 2); inhibitors of KIR (killer-cell immunoglobulin-like receptor), such as lirlumab (IPH2102) and IPH2101; inhibitors of B7—H3, such as MGA271; inhibitors of B7—H4, such as FPA150; inhibitors of BTLA (B- and T-lymphocyte attenuator); inhibitors of LAG3 (lymphocyte-activation gene 3), such as IMP321 (eftilagimod alpha), relatlimab, MK-4280, AVA017, BI754111, ENUM006, GSK2831781, INCAGN2385, LAG3Ig, LAG525, REGN3767, Sym016, Sym022, TSR033, TSR075 and XmAb22841; inhibitors of TIM-3 (T-cell immunoglobulin and mucin-domain containing-3), such as LY3321367, MBG453, and TSR-022; inhibitors of VISTA (V-domain Ig suppressor of T cell activation), such as JNJ-61610588; inhibitors of ILT2/LILRB1 (Ig-like transcript 2/leukocyte Ig-like receptor 1); inhibitor of ILT3/LILRB4 (Ig-like transcript 3/leukocyte Ig-like receptor 4); inhibitors of ILT4/LILRB2 (Ig-like transcript 4/leukocyte Ig-like receptor 2), such as MK-4830; inhibitors of TIGIT (T cell immunoreceptor with Ig and ITIM domains), such as MK-7684, PTZ-201, RG6058 and COM902; inhibitors of NKG2A, such as IPH-2201; and inhibitors of PVRIG, such as COM701.

In certain embodiments the one or more additional drug is an inhibitor of PD-1 or PD-L1. In certain embodiments the one or more additional drug is an inhibitor of PD-1. In certain embodiments the one or more additional drug is an inhibitor of PD-L1. In certain embodiments the one or more additional drug is an anti-PD-1 antibody. In certain embodiments the one or more additional drug is an anti-PD-L1 antibody. In certain embodiments the one or more additional drug is pembrolizumab. While in development, pembrolizumab was known as MK3475 and as lambrolizumab and is marketed as KEYTRUDA®.

Pembrolizumab may be given as per the prescription information, such as 200 mg pembrolizumab per intravenous infusion, for example every three weeks. The infusion time may range from 15 minutes to 4 hours, such as from 30 minutes to one hour and is typically about 30 minutes. In general, pembrolizumab is administered with the dose, administration frequency and form of administration approved for a given indication.

In certain embodiments the one or more additional drug is pembrolizumab, which is administered every three weeks via intravenous infusion at a dose of 200 mg, either prior to, together with or after administration of the IL-2 conjugate. In certain embodiments pembrolizumab is administered every three weeks via intravenous infusion at a dose of 200 mg prior to administration of the IL-2 conjugate. In certain embodiments pembrolizumab is administered every three weeks via intravenous infusion at a dose of 200 mg together with the administration of the IL-2 conjugate. In certain embodiments pembrolizumab is administered every three weeks via intravenous infusion at a dose of 200 mg after administration of the IL-2 conjugate.

In certain embodiments the IL-2 conjugate or a pharmaceutically acceptable salt thereof is for use in the treatment of cancer, wherein the IL-2 conjugate is administered in a dose ranging from 80 μg IL-2/kg to 160 μg IL-2/kg as a cotreatment with pembrolizumab, wherein pembrolizumab is administered every three weeks at a dose of 200 mg, either prior to, together with or after administration of the IL-2 conjugate.

In certain embodiments the IL-2 conjugate or a pharmaceutically acceptable salt thereof is for use in the treatment of cancer, wherein the IL-2 conjugate is administered in a dose ranging from 100 μg IL-2/kg to 140 μg IL-2/kg as a cotreatment with pembrolizumab, wherein pembrolizumab is administered every three weeks at a dose of 200 mg, either prior to, together with or after administration of the IL-2 conjugate.

In certain embodiments the IL-2 conjugate or a pharmaceutically acceptable salt thereof is for use in the treatment of cancer, wherein the IL-2 conjugate is administered in a dose ranging from 110 μg IL-2/kg to 130 μg IL-2/kg as a cotreatment with pembrolizumab, wherein pembrolizumab is administered every three weeks at a dose of 200 mg, either prior to, together with or after administration of the IL-2 conjugate.

In certain embodiments the IL-2 conjugate or a pharmaceutically acceptable salt thereof is for use in the treatment of cancer, wherein the IL-2 conjugate is administered in a dose of about 120 μg IL-2/kg as a cotreatment with pembrolizumab, wherein pembrolizumab is administered every three weeks at a dose of 200 mg, either prior to, together with or after administration of the IL-2 conjugate.

In certain embodiments the IL-2 conjugate or a pharmaceutically acceptable salt thereof is for use in the treatment of cancer, wherein the IL-2 conjugate is administered in a dose of 120 μg IL-2/kg as a cotreatment with pembrolizumab, wherein pembrolizumab is administered every three weeks at a dose of 200 mg, either prior to, together with or after administration of the IL-2 conjugate.

In certain embodiments the IL-2 conjugate or a pharmaceutically acceptable salt thereof for use in the treatment of cancer is administered via intravenous infusion as a cotreatment with pembrolizumab.

In certain embodiments pembrolizumab is administered every three weeks via intravenous infusion at a dose of 200 mg, either prior to, together with or after administration of the IL-2 conjugate. In certain embodiments pembrolizumab is administered via intravenous infusion at a dose of 200 mg prior to administration of the IL-2 conjugate. In certain embodiments pembrolizumab is administered via intravenous infusion at a dose of 200 mg together with administration of the IL-2 conjugate. In certain embodiments pembrolizumab is administered via intravenous infusion at a dose of 200 mg after administration of the IL-2 conjugate.

The immune activating receptor agonist may be selected from the group consisting of agonists of CD27, such as recombinant CD70, such as HERA-CD27L, and varlilumab (CDX-1127); agonists of CD28, such as recombinant CD80, recombinant CD86, TGN1412 and FPT155; agonists of CD40, such as recombinant CD40L, CP-870,893, dacetuzumab (SGN-40), Chi Lob 7/4, ADC-1013 and CDX1140; agonists of 4-1BB (CD137), such as recombinant 4-1BBL, urelumab, utomilumab and ATOR-1017; agonists of OX40, such as recombinant OX40L, MEDI0562, GSK3174998, MOXR0916 and PF-04548600; agonists of GITR, such as recombinant GITRL, TRX518, MEDI1873, INCAGN01876, MK-1248, MK-4166, GWN323 and BMS-986156; and agonists of ICOS, such as recombinant ICOSL, JTX-2011 and GSK3359609.

The multi-specific drug may be selected from the group consisting of biologics and small molecule immune checkpoint inhibitors. Examples for biologics are multi-specific immune checkpoint inhibitors, such as CD137/HER2 multispecifics, PD-(L)1/LAG3 antagonists (for example FS118, MGD013), CTLA4/LAG3 antagonists (for example XmAb22841) and CTLA4/PD-(L)1 antagonists (for example XmAb20717, MGD019); multispecific immune activating receptor agonists, immunocytokines and multi-specific immune checkpoint agonists.

Such multi-specific immune checkpoint agonists may be selected from the group consisting of Ig superfamily agonists, such as ALPN-202, FPT155, TGN1412, GSK3359609, JTX-2011; TNF superfamily agonists, such as FAP-4-1BBL (RG7826), OX40-41BB (FS120) ATOR-1015, ATOR-1144, ALG.APV-527, lipocalin/PRS-343, PRS344/ONC0055, FAP-CD40 DARPin, MP0310 DARPin, FAP-0X40 DARPin, EGFR-CD40 DARPin, EGFR41BB/CD137 DARPin, EGFR-0X40/DARFPin, HER2-CD40 DARPin, HER2-41BB/CD137 DARPin, HER2-0X40 DARPin, FIBRONECTIN ED-B-CD40 DARPin, FIBRONECTIN ED-B-41BB/CD137 and FIBRONECTIN ED-B-0X40 DARPin; CD3 multispecific agonists, such as blinatumomab, solitomab, MEDI-565, ertumaxomab, anti-HER2/CD3, 1Fab-immunoblobulin G TDB, GBR 1302, MGD009, MGD007, EGFRBi, EGFR-CD Probody, RG7802, PF-06863135, PF-06671008, AMG212/BAY2010112, CD3-5T4, XmAb14045, XmAb13676, XmAbl8087, S80880, REGN1979, REGN5458, REGN4018, RG6026, Mosunetuzumab, EM801, ERY974, RG6194, AMG420, AMG330, AMG 212, AMG 596, AMG 160, AMG 427, AMG 562, AMG 673, AMG 701, AMG 757, AFM13, AMF24, AFM26, AFM11. TNB-486, TNB-383B, GEN3013, JNJ-63709178, JNJ-63898081, JNJ-64007957, JNJ-64407564, JNJ-67571244, AMV564, APVO414 (MOR209, ES414), APV0436, HPN424, HPN536, HPN217, HPN 328 and other multispecific CD3 agonists or T cell receptor (TCR) agonists including T⁶ TCR agonists targeting T cell activity towards a tumor cell antigen or viral antigen or expressing cell; Natural Killer (NK) cell receptor multispecific agonists targeting an activating NK receptor and a target tumor cell antigen, such as NKG2D multispecific agonists, NKp30 multispecific agonists, NKp44 multispecific agonists, NKp46 multispecific agonists, NKp80 multispecific agonists, NKG2C multispecific agonists, 2B4 (CD244) multispecific agonists, CD32a multispecific agonists, CD64 multispecific agonists, multispecific agonists that bind to a tumor antigen as well as activating receptors such as NKG2D or NKp30 or other NK receptors listed above as well as binding to Fc receptors such as TriNKeTs, and CD16 multispecific agonists, such as 1633 BiKE, 161533 TriKE, OXS-3550, OXS-C3550, AFM13 and AFM24; and other therapeutic antibodies capable of binding a target antigen as well as Fc receptors such as CD16, CD32a, CD64.

Other examples of immune activating receptor agonists include Dectin agonists (Imprime PGG), recombinant NKG2D ligands, ligand or modifiers of T6 TCR signaling such as anti-BTN3A1 mAbs or anti- BTN2A1 mAbs or Vy9/VS2 TCR activating ligand such as phospho antigens and pyrophosphate antigens, or agents which increase endogenous Vy9/V62 ligands such as bisphosphonates like pamidronate and zoledronate.

An example for a small molecule immune checkpoint inhibitor is CA-327 (TIM3/PD-L1 antagonist).

The antibody-drug conjugate may be selected from the group consisting of ADCs targeting hematopoietic cancers, such as gemtuzumab ozogamicin, brentuximab vedotin, inotuzumab ozogamicin, SAR3419, BT062, SGN-CD19A, IMGN529, MDX—1203, polatuzumab vedotin (RG7596), pinatuzumab vedotin (RG7593), RG7598, milatuzumab-doxorubicin and OXS-1550; and ADCs targeting solid tumor antigens, such as trastuzumab emtansine, glembatumomab vedotin, SAR56658, AMG-172, AMG-595, BAY-94-9343, BIIB015, vorsetuzumab mafodotin (SGN-75), ABT—414, ASG-5ME, enfortumab vedotin (ASG-22ME), ASG-16M8F, IMGN853, indusatumab vedotin (MLN-0264), vadortuzumab vedotin (RG7450), sofituzumab vedotin (RG7458), lifastuzumab vedotin (RG7599), RG7600, DEDN6526A (RG7636), PSMA TTC, 1095 from Progenics Pharmaceuticals, lorvotuzumab mertansine, lorvotuzumab emtansine, IMMU-130, sacituzumab govitecan (IMMU-132), PF-06263507 and MEDI0641.

The antibody-adjuvant conjugate may be a boltbody, such as the boltbodies described in WO2018112108A1 and WO2018009916A1, which are incorporated by reference herewith. In certain embodiments the boltbody is selected from the group consisting of BDC-1001 and BDC-2034. In certain embodiments the boltbody is BDC-1001. In certain embodiments the boltbody is BDC-2034.

In certain embodiments the one or more additional drug is a radionuclide which may be selected from the group consisting of β-emitters, such as ¹⁷⁷Lutetium, ¹⁶⁶Holmium, ¹⁸⁶ Rhenium ¹⁸⁸Rhenium, ⁶⁷Copper, ¹⁴⁹Promethium, ¹⁹⁹Gold, ⁷⁷Bromine, ¹⁵³Samarium, ¹⁰⁵Rhodium, ⁸⁹Strontium, ⁹⁰Yttrium, ¹³¹Iodine; α-emitters, such as ²¹³Bismuth, ²²³Radium, ²²⁵Actinium, ²¹¹Astatine; and Auger electron-emitters, such as ⁷⁷Bromine, ¹¹¹Indium, ¹²³Iodine and ¹²⁵Iodine.

The targeted radionuclide therapeutics may be selected from the group consisting of zevalin (⁹⁰Y-ibritumomab tiuxetan), bexxar (¹³¹I-tositumomab), oncolym (¹³¹I-Lym 1), lymphocide (⁹⁰Y-epratuzumab), cotara (¹³¹I-chTNT—1/B), labetuzumab (⁹⁰Y or ¹³¹I-CEA), theragyn (⁹⁰Y-pemtumomab), licartin (¹³¹I-metuximab), radretumab (¹³¹I-L19) PAM4 (⁹⁰Y-clivatuzumab tetraxetan), xofigo (²²³Ra dichloride), lutathera (¹⁷⁷Lu-DOTA-Tyr³-Octreotate) and ¹³¹I-MIBG.

The DNA damage repair inhibitor may be selected from the group consisting of poly (ADP-ribose) polymerase (PARP) inhibitors, such as olaparib, rucaparib, niraparib, veliparib, CEP 9722 and E7016; CHK1/CHK2 dual inhibitors, such as AZD7762, V158411, CBP501 and XL844; CHK1 selective inhibitors, such as PF477736, MK8776/SCH900776, CCT244747, CCT245737, LY2603618, LY2606368/prexasertib, AB-IsoG, ARRY575, AZD7762, CBP93872, ESPO1, GDC0425, SAR020106, SRA737, V158411 and VER250840; CHK2 inhibitors, such as CCT241533 and PV1019; ATM inhibitors, such as AZD0156, AZD1390, KU55933, M3541 and SX-RDS1; ATR inhibitors, such as AZD6738, BAY1895344, M4344 and M6620 (VX-970); and DNA-PK inhibitors, such as M3814.

The tumor metabolism inhibitor may be selected from the group consisting of inhibitors of the adenosine pathway, inhibitors of the tryptophan metabolism and inhibitors of the arginine pathway.

Examples for an inhibitor of the adenosine pathway are inhibitors of A2AR (adenosine A2A receptor), such as ATL-444, istradefylline (KW-6002), MSX-3, preladenant (SCH-420,814), SCH-58261, SCH412,348, SCH-442,416, ST—1535, caffeine, VER-6623, VER-6947, VER-7835, vipadenant (BIIB-014), ZM-241,385, PBF-509 and V81444; inhibitors of CD73, such as IPH53 and SRF373; and inhibitors of CD39, such as IPH52.

Examples for an inhibitor of the tryptophane metabolism are inhibitors of IDO, such as indoximod (NLG8189), epacadostat, navoximod, BMS-986205 and MK-7162; inhibitors of TDO, such as 680C91; and IDO/TDO dual inhibitors.

Examples for inhibitors of the arginine pathway are inhibitors of arginase, such as INCB001158.

The protein kinase inhibitor may be selected from the group consisting of receptor tyrosine kinase inhibitors, intracellular kinase inhibitors, cyclin dependent kinase inhibitors, phosphoinositide-3-kinase inhibitors, mitogen-activated protein kinase inhibitors, inhibitors of nuclear factor kappa-β kinase (IKK), and Wee-1 inhibitors.

Examples for receptor tyrosine kinase inhibitors are EGF receptor inhibitors, such as afatinib, cetuximab, erlotinib, gefitinib, pertuzumab and margetuximab; VEGF receptor inhibitors, such as axitinib, lenvatinib, pegaptanib and linifanib (ABT—869); C-KIT Receptor inhibitors, such as CDX0158 (KTN0158); ERBB2 (HER2) inhibiors, such as herceptin (trastuzumab); ERBB3 receptor inhibitors, such as CDX3379 (MEDI3379, KTN3379) and AZD8931 (sapitinib); FGF receptor inhibitors, such as erdafitinib; AXL receptor inhibitors, such as BGB324 (BGB 324, R 428, R428, bemcentinib) and SLC391; and MET receptor inhibitors, such as CGEN241.

Examples for intracellular kinase inhibitors are Bruton's tyrosine kinase (BTK) inhibitors, such as ibrutinib, acalabrutinib, GS-4059, spebrutinib, BGB-3111, HM71224, zanubrutinib, ARQ531, BI-BTK1 and vecabrutinib; spleen tyrosine kinase inhibitors, such as fostamatinib; Bcr-Abl tyrosine kinase inhibitors, such as imatinib and nilotinib; Janus kinase inhibitors, such as ruxolitinib, tofacitinib and fedratinib; and multi-specific tyrosine kinase inhibitors, such as bosutinib, crizotinib, cabozantinib, dasatinib, entrectinib, lapatinib, mubritinib, pazopanib, sorafenib, sunitinib, SU6656 and vandetanib.

Examples for cyclin dependent kinase inhibitors are ribociclib, palbociclib, abemaciclib, trilaciclib, purvalanol A, olomucine II and MK-7965.

Examples for phophoinositide-3-kinase inhibitors are IPI549, GDc-0326, pictilisib, serabelisib, IC-87114, AMG319, seletalisib, idealisib and CUDC907.

Examples for mitogen-activated protein kinase inhibitors are Ras/farnesyl transferase inhibitors, such as tipirafinib and LB42708; Raf inhibitors, such as regorafenib, encorafenib, vemurafenib, dabrafenib, sorafenib, PLX-4720, GDC-0879, AZ628, lifirafenib, PLX7904 and RO5126766; MEK inhibitors, such as cobimetinib, trametinib, binimetinib, selumetinib, pimasertib, refametinib and PD0325901; ERK inhibitors, such as MK-8353, GDC-0994, ulixertinib and SCH772984.

Examples for inhibitors of nuclear factor kappa-β kinase (IKK) are BPI-003 and AS602868.

An example of a Wee-1 inhibitor is adavosertib.

The chemokine receptor and chemoattractant receptor agonist may be selected from the group consisting of CXC chemokine receptors, CC chemokine receptors, C chemokine receptors, CX3C chemokine receptors and chemoattractant receptors.

The CXC chemokine receptor may be selected from the group consisting of CXCR1 agonists, such as recombinant CXCL8 and recombinant CXCL6; CXCR2 agonists, such as recombinant CXCL8, recombinant CXCL1, recombinant CXCL2, recombinant CXCL3, recombinant CXCL5, recombinant CXCL6, MGTA 145 and SB251353; CXCR3 agonists, such as recombinant CXCL9, recombinant CXCL10, recombinant CXCL11 and recombinant CXCL4; CXCR4 agonists, such as recombinant CXCL12, ATI2341, CTCE0214, CTCE0324 and NNZ4921; CXCR5 agonists, such as recombinant CXCL13; CXCR6 agonists, such as recombinant CXCL16; and CXCL7 agonists, such as recombinant CXCL11.

The CC chemokine receptor may be selected from the group consisting of CCR1 agonists, such as recombinant CCL3, ECI301, recombinant CCL4, recombinant CCL5, recombinant CCL6, recombinant CCL8, recombinant CCL9/10, recombinant CCL14, recombinant CCL15, recombinant CCL16, recombinant CCL23, PB103, PB105 and MPIF1; CCR2 agonists, such as recombinant CCL2, recombinant CCL8, recombinant CCL16, PB103 and PB105; CCR3 agonists, such as recombinant CCL11, recombinant CCL26, recombinant CCL7, recombinant CCL13, recombinant CCL15, recombinant CCL24, recombinant CCL5, recombinant CCL28 and recombinant CCL18; CCR4 agonists, such as recombinant CCL3, ECI301, recombinant CCL5, recombinant CCL17 and recombinant CCL22; CCR5 agonists, such as recombinant CCL3, ECI301, recombinant CCL5, recombinant CCL8, recombinant CCL11, recombinant CCL13, recombinant CCL14, recombinant CCL16, PB103 and PB105; CCR6 agonists, such as recombinant CCL20; CCR7 agonists, such as recombinant CCL19 and recombinant CCL21; CCR8 agonists, such as recombinant CCL1, recombinant CCL16, PB103 and PB105; CCR9 agonists, such as recombinant CCL25; CCR10 agonists, such as recombinant CCL27 and recombinant CCL28; and CCR11 agonists, such as recombinant CCL19, recombinant CCL21 and recombinant CCL25.

The C chemokine receptors may be a XCR1 agonist, such as recombinant XCL1 or recombinant XCL2.

The CX3C chemokine receptors may be a CX3CR1 agonist, such as recombinant CX3CL1.

The chemoattractant receptors may be selected from the group consisting of formyl peptide receptor agonists, such as N-formyl peptides, N-formylmethionine-leucyl-phenylalanine, enfuvirtide, T21/DP107, annexin A1, Ac2-26 and Ac9-25; C5a receptor agonists; and chemokine-like receptor 1 agonists, such as chemerin.

The chemokine antagonists may be selected from the group consisting of inhibitors of CXCL chemokines, such as UNBS5162; inhibitors of CXCL8, such as BMS986253 and PA620; inhibitors of CXCL10, such as TM110, eldelumab and NI0801; inhibitors of CXCL12, such as NOX-A12 and JVS100; inhibitors of CXCL13, such as VX5; inhibitors of CCL2, such as PA508, ABN912, AF2838, BN83250, BN83470, C243, CGEN54, CNT0888, NOXE36, VT224 and SSR150106; inhibitors of CCL5, such as HGS1025 and NI0701; inhibitors of CCL2/CCL5, such as BKTP46; inhibitors of CCL5/FMLP receptor, such as RAP160; inhibitors of CCL11, such as bertilimumab and RAP701; inhibitors of CCL5/CXCL4, such as CT2008 and CT2009; inhibitors of CCL20, such as GSK3050002; and inhibitors of CX3CL1, such as quetmolimab.

The chemokine receptor antagonists may be selected from the group consisting of inhibitors of CXCR1, such as repertaxin, CCX832, FX68 and KB03; inhibitors of CXCR2, such as AZD5069, AZD5122, AZD8309, GSK1325756, GSK1325756H, PS291822, SB332235 and SB656933; inhibitors of CXCR1/CXCR2, such as DF1970, DF2156A, DF2162, DF2755A, reparixin, SX576, SX682, PACG31P, AZD4721 and PA401; inhibitors of CXCR3; inhibitors of CXCR4, such as BL8040; inhibitors of CXCR4/E-selectin, such as GMI1359; inhibitors of CXCR6, such as CCX5224; inhibitors of CCR1, such as AZD4818, BAY865047, BMS817399, CCX354, CCX634, CCX9588, CP481715, MLN3701, MLN3897, PS031291, PS375179 and PS386113; inhibitors of CCR2, such as AZD2423, BL2030, BMS741672, CCX140, CCX598, CCX872, CCX915, CNTX6970, INCB3284, INCB3344, INCB8696, JNJ17166864, JNJ27141491, MK0812, OPLCCL2LPM, PF4136309, serocion, STIB0201, STIB0211, STIB0221, STIB0232, STIB0234, TAK202, TPI526; inhibitors of CCR2/CCR5, such as PF04634817, RAP103 and TBR652; inhibitors of CCR2/CCR5/CCR8, such as RAP310; inhibitors of CCR3, such as ASM8, AXP1275, BMS639623, CM101, DPC168, GW766994, GW824575, MT0814, OPLCCL11LPM and QAP642; inhibitors of CCR4, such as AT008, AZD2098, CCX6239, FLX193, FLX475, GBV3019, GSK2239633, IC487892 and poteligeo; inhibitors of CCR5, such as 5P12—RANTES, AZD5672, AZD8566, CMPD167, ESN196, GSK706769, GW873140, HGS004, INCB15050, INCB9471, L872, microbicide, PF232798, PRO140, RAP101, SAR113244, SCH350634, SCH351125, SCH417690, selzentry, TAK779, TBR220, TD0232 and VX286; inhibitors of CCR5/CXCR4, such as AMD887, ND401 and SPO1A; inhibitors of CCR6, such as CCX507, CCX9664 and STIB100X; inhibitors of CCR6, such as CCX025, CCX507, CCX807, eut22, MLN3126, POL7085, traficet-EN; inhibitors of CXCR3, such as AMG487, ATO10, STIA120X; inhibitors of CXCR4, such as AD114, AD214, ALX0651, ALX40-4C, AMDO70, AT007, AT009, BKT170, BMS936564, celixafor, CTCE9908, GBV4086, GSK812397, KRH2731, KRH3140, LY2510924, LY2624587, mozobil, OPLCXCL12LPM, PF06747143, POL6326, Q122, revixil, TG0054, USL311, X4P001 and X4P002; and inhibitors of CXCR7, such as CCX650 and CCX662.

The cytokine receptor agonist may be selected from the group consisting of mRNAs, DNAs or plasmids encoding the genes for IL-2, IL-15, IL-7, IL-10, IL-12, IL-21, IFNα IL-17, IFNβ, IFNγ, IL-18, IL-27, TNFα, GM-CSF, FLT3L, LTα, LTβ and TRAIL and recombinant proteins, such as agonists of IL-2/IL-15 β/γ receptors, agonists of IL-10 receptor, agonists of IL-12 receptor, agonists of IL-18 receptor, agonists of IL-21 receptor, agonists of IL-7 receptor, agonists of IFNα/β receptor, agonists of IFN γ receptor, agonists of FLT3 receptor, agonists of GM-CSF receptor, agonists of LTα receptor, agonists of LTβ receptor, and agonists of TNFα receptor.

Examples for agonists of IL-10 receptor are AGO 11, dekavil, EG10, IL1ONanocap, Ilodecakin, AM0010, tenovil and VT310 VIRON.

Examples for agonists of IL-12 receptor are recombinant IL-12 p70, recombinant IL-12 p35, AM0012, AS1409, dodekin, HemaMax, LipoVIL12, MSB0010360N, Ad-RTS-hIL-12, tavokinogene telseplasmid, exoIL-12 and NHS-IL12.

An example for an agonist of IL-18 receptor is SB485232.

An example for an agonist of IL-21 receptor is BMS982470 (denenicokin).

Examples for agonists of IL-7 receptor are CYT107, CYT99007 and GX-I7.

An example for an agonist of FLT3R is CDX-301.

Examples for agonist of TNFα receptor are L19—TNFα, aurimune, beromun, BreMel/TNFα, fibromun, refnot and TNFPEG20.

The death receptor agonists may be selected from the group consisting of TRAILR1/DR4 agonists, such as AMG951 (dulanermin), APG350, APG880, HGSETR1 (mapatumumab) and SL231; and TRAILR2/DR5 agonists, such as AMG655, DS8273, HGSETR2 (lexatumumab), HGSTR2J, IDD004/GEN1029, INBRX109, LBY135, MEDI3039, PR095780, RG7386 and TAS266.

The CD47 antagonists may be selected from the group consisting of ALX148, CC-90002, Hu5F9G4, SRF231, TI061, TTI-621, TTI-622, A0176, IBI188, IMC002, recombinant SIRPα and LYN00301.

An example for a SIRPα antagonist is FSI89 or recombinant CD47.

Examples for oncolytic drugs are CAVATAK, BCG, mobilan, TG4010, Pexa-Vec (JX-594), JX-900, JX-929 and JX-970.

Examples for signal converter proteins are Fn14—TRAIL (KAHR101), CD80-Fc (FTP155), CTLA4-FasL (KAHR102), PD1-41BBL (DSP 105), PD-L1-41BB (PRS-344, NM21-1480, FS222), PD1-CD70 (DSP 106) and SIRPa-41BBL (DSP 107).

The epigenetic modifiers may be selected from the group consisting of DNA methyltransferase inhibitors, lysine-specific demethylase 1 inhibitors, Zeste homolog 2 inhibitors, bromodomain and extra-terminal motif (BET) protein inhibitors such as GSK525762, and histone deacetylase (HDAC) inhibitors such as beleodaq, SNDX275 and CKD-M808.

Examples for tumor peptides/vaccines are NY-ESO, WT1, MART—1, 10102 and PF-06753512 and personalized cancer vaccines using patient derived tumor sequences or neoantigens.

Examples for heat shock protein (HSP) inhibitors are inhibitors of HSP90, such as PF-04929113 (SNX-5422).

Examples of proteolytic enzymes are recombinant hyaluronidase, such as rHuPH20 and PEGPH20.

The ubiquitin and proteasome inhibitors may be selected from the group consisting ofubiquitin-specific protease (USP) inhibitors, such as P005091; 20S proteasome inhibitors, such as bortezimib, carfilzomib, ixazomib, oprozomib, delanzomib and celastrol; and immunoproteasome inhibitors, such as ONX-0914.

The adhesion molecule antagonists may be selected from the group consisting of 02-integrin antagonists and selectin antagonists.

The hormones may be selected from the group consisting of hormone receptor agonists and hormone receptor antagonists.

Examples for a hormone receptor agonist are somatostatin receptor agonists, such as somatostatin, lanreotide, octreotide, FX125L, FX141L and FX87L.

Examples for hormone receptor antagonists are anti-androgens, anti-estrogens and anti-progestogens. Examples for anti-androgens are steroidal antiandrogens, such as cyproterone acetate, megestrol acetate, chlormadinone acetate, spironolactone, oxendolone and osaterone acetate; nonsteroidal anti-androgens, such as flutamide, bicalutamide, nilutamide, topilutamide, enzalutamide and apalutamide; androgen synthesis inhibitors, such as ketoconazole, abiraterone acetate, seviteronel, aminoglutethimide, finasteride, dutasteride, epristeride and alfatradiol.

Examples for anti-estrogens are selective estrogen receptor modulators (SERMs), such as tamoxifen, clomifene, Fareston and raloxifene; ER silent antagonists and selective estrogen receptor degrader (SERD), such as fulvestrant; aromatase inhibitors, such as anastrozole, letrozole, exemestane, vorozole, formestane and fadrozole; and anti-gonadotropins, such as testosterone, progestogens and GnRH analogues. Examples for anti-progestogens are mifepristone, lilopristone and onapristone.

Examples of cellular therapy include CAR therapies such as CAR-T therapies such as tisagenlecleucel, axicabtagene ciloleucel, bb21217, LCAR-B38M, JCARH125, MCARH171, JNJ-4528, idecabtagene vicleucel (bb2121), SCRI-CAR19x22; CAR therapies targeting tumor antigens such as CAR therapies targeting CD19 expressing cells, CAR therapies targeting CD22 expressing cells, CAR therapies targeting BCMA expressing cells, CAR therapies targeting HER2 expressing cells, CAR therapies targeting CD138 expressing cells, CAR therapies targeting CD133 expressing cells, CAR therapies targeting BCMA expressing cells, CAR therapies targeting CEA expressing cells, CAR therapies targeting Claudin 18.2 expressing cells, CAR therapies targeting EGFR expressing cells, CAR therapies targeting EGFRvIII expressing cells, CAR therapies targeting Eph2A expressing cells, CAR therapies targeting EpCAM expressing cells, CAR therapies targeting GD2 expressing cells, CAR therapies targeting GPC3 expressing cells, CAR therapies targeting MSLN expressing cells, CAR therapies targeting 5T4 expressing cells, CAR therapies targeting LMP1 expressing cells, CAR therapies targeting PD-L1 expressing cells, CAR therapies targeting PSMA expressing cells, CAR therapies targeting FRa expressing cells, and CAR therapies targeting MUC1 expressing cells. Examples of cellular therapy include TIL therapy, NK therapy, Cytokine induced memory NK cell therapy, NK cell therapy with ex vivo expanded cells. Examples of cellular therapy include therapy with Δβ or γδ T cells which may be engineered to express a tumor antigen or tumor neoantigen specific T Cell Receptor or which may have been expanded in the context of tumor antigen or tumor neoantigens.

In certain embodiments the one or more additional drug is given in the approved dose.

In certain embodiments the one or more additional drug is the standard of care treatment for a certain cancer, such as standard of care chemotherapy.

In a fifth aspect the invention relates to a kit-of-parts comprising the IL-2 conjugate or a pharmaceutically acceptable salt thereof. In certain embodiments the kit-of-parts comprises the IL-2 conjugate or a pharmaceutically acceptable salt thereof and at least one container.

In certain embodiments the at least one container of the kit-of-parts is a sterile container for holding the IL-2 conjugate or a pharmaceutically acceptable salt thereof. In certain embodiments the sterile container is selected from the group consisting of ampoules, bottles, vials, tubes, bags, pouches, blister-packs, syringes, dual-chamber syringes and other suitable container forms known in the art. Such sterile containers can be made of plastic, glass, laminated paper, metal foil or other materials suitable for holding medicaments. In certain embodiments the sterile container is a glass vial.

In certain embodiments the kit-of-parts further comprises instructions in a readable medium for dosing and/or administration of the IL-2 conjugate or a pharmaceutically acceptable salt thereof. In certain embodiments the readable medium can be read by a human or by a machine.

In certain embodiments the human-readable medium is selected from the group consisting of pamphlets, inserts, and other written forms. In certain embodiments the machine-readable medium is selected from the group consisting of read-only memory (ROM), such as a CD-ROM; random access memory (RAM); magnetic disk storage media; a Quick Response Code (QR Code) or other matrix barcode; optical storage media; and flash memory devices, such as a USB drive.

In certain embodiments the kit-of-parts comprises the IL-2 conjugate or a pharmaceutically acceptable salt thereof provided in a glass vial and instructions in a readable medium for dosing and/or administration of the IL-2 conjugate or a pharmaceutically acceptable salt thereof.

In certain embodiments the kit-of-parts optionally further comprises parts and/or devices necessary or suitable for the administration of IL-2 conjugate or a pharmaceutically acceptable salt thereof, including needles, applicators, and injection- devices, such as syringes.

In certain embodiments the kit-of-parts optionally further comprises a reconstitution solution.

In certain embodiments the reconstitution solution is a sterile liquid, such as phosphate buffered saline, isotonic saline, water for injection, or other buffers, which may contain further excipients, such as preservatives and/or antimicrobials, such as, for example, benzylalcohol and cresol.

In certain embodiments the kit-of-parts comprises a unit dosage form of the IL-2 conjugate or a pharmaceutically acceptable salt thereof as described elsewhere herein. In certain embodiments the kit-of-parts comprises a unit dosage form of the IL-2 conjugate or a pharmaceutically acceptable salt thereof as described elsewhere herein and instructions in a readable medium for dosing and/or administration of the IL-2 conjugate or a pharmaceutically acceptable salt thereof.

In certain embodiments the kit-of-parts comprises the IL-2 conjugate or a pharmaceutically acceptable salt thereof and any of the one or more additional drug described above. In certain embodiments the kit-of-parts comprises the IL-2 conjugate or a pharmaceutically acceptable salt thereof and the drug of formula (A-3), (A-6) or (Ai-6). In certain embodiments the kit-of-parts comprises the IL-2 conjugate or a pharmaceutically acceptable salt thereof and pembrolizumab.

In certain embodiments the IL-2 conjugate or a pharmaceutically acceptable salt thereof and the one or more additional drug are comprised in one container. In certain embodiments the IL-2 conjugate or a pharmaceutically acceptable salt thereof and the drug of formula (A-3), (A-6) or (Ai-6) are comprised in one container. In certain embodiments the IL-2 conjugate or a pharmaceutically acceptable salt thereof and pembrolizumab are comprised in one container.

In certain embodiments the kit-of-parts comprises a first container comprising the IL-2 conjugate or a pharmaceutically acceptable salt thereof and a second container comprising the one or more additional drug. In certain embodiments the kit-of-parts comprises a first container comprising the IL-2 conjugate or a pharmaceutically acceptable salt thereof and a second container comprising the drug of formula (A-3), (A-6) or (Ai-6). In certain embodiments the kit-of-parts comprises a first container comprising the IL-2 conjugate or a pharmaceutically acceptable salt thereof and a second container comprising pembrolizumab.

In certain embodiments the IL-2 conjugate or a pharmaceutically acceptable salt thereof and the one or more additional drug provided in the kit-of-parts are to be administered simultaneously, separately or sequentially. In certain embodiments the IL-2 conjugate or a pharmaceutically acceptable salt thereof and the drug of formula (A-3), (A-6) or (Ai-6) provided in the kit-of-parts are to be administered simultaneously, separately or sequentially.

In certain embodiments the IL-2 conjugate or a pharmaceutically acceptable salt thereof and pembrolizumab provided in the kit-of-parts are to be administered simultaneously, separately or sequentially.

In certain embodiments the kit-of-parts further comprises instructions in a readable medium for dosing and/or administration of the IL-2 conjugate or a pharmaceutically acceptable salt thereof and the one or more additional drug. In certain embodiments the kit-of-parts further comprises instructions in a readable medium for dosing and/or administration of the IL-2 conjugate or a pharmaceutically acceptable salt thereof and the drug of formula (A-3), (A-6) or (Ai-6). In certain embodiments the kit-of-parts further comprises instructions in a readable medium for dosing and/or administration of the IL-2 conjugate or a pharmaceutically acceptable salt thereof and pembrolizumab.

In a sixth aspect the invention relates to a kit-of-parts as described elsewhere herein for use in the treatment of cancer comprising the IL-2 conjugate or a pharmaceutically acceptable salt thereof or the unit dosage form of an IL-2 conjugate or a pharmaceutically acceptable salt thereof as described elsewhere herein.

In a seventh aspect the invention relates to a method of synthesizing the IL-2 conjugate or a pharmaceutically acceptable salt thereof. In certain embodiments the IL-2 conjugate is synthesized as described in WO2021/245130, example 5, compound 6. In certain embodiments the IL-2 conjugate is synthesized as described in example 1 herein.

In certain embodiments the IL-2 conjugate of formula (I)

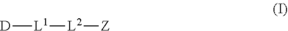

is synthesized by a method comprising the steps of
a) providing
a)i. at least one reagent Z—L²—L¹—FG;
a)ii. a liquid comprising at least one organic solvent; and
a)iii. one or more drug D—H;

b) combining and incubating the at least one reagent Z—L²—L¹—FG from step a)i., the liquid from step a)ii. and the one or more drug D—H from step a)iii. to obtain the IL-2 conjugate of formula (I); and

c) optionally, purifying the IL-2 conjugate of formula (I) obtained in step b), wherein —D, —L¹—, —L²— and —Z are defined as described elsewhere herein;

D—H is the free form of —D; and

—FG is a functional group moiety.

In certain embodiments the liquid of step a)ii. comprises at least about 5% (v/v) of the organic solvent, such as ranging from about 5% to about 80% (v/v), ranging from about 10% to about 60% (v/v), or ranging from about 20% to about 50% (v/v). In certain embodiments the liquid of step a)ii. comprises at least 5% (v/v) of the organic solvent, such as ranging from 5% to 80% (v/v), ranging from 10% to 60% (v/v), or ranging from 20% to 50% (v/v).

In certain embodiments the organic solvent of step a)ii. is selected from the group consisting of acetaldehyde, acetic acid, acetone, acetonitrile, 1,2-butanediol, 1,3-butanediol, 1,4-butanediol, 2-butoxyethanol, butyric acid, dimethoxyethane, dimethyl sulfoxide, dimethyl formamide, 1,4-dioxane, ethanol, ethylene glycol, formic acid, furfuryl alcohol, glycerol, 2-methyl-2,4-pentanediol, methanol, methyl isocyanide, n-methyl-2-pyrrolidone, 1-propanol, 1,3-propanediol, 1,5-pentanediol, 2-propanol, propanoic acid, propylene glycol, pyridine, tetrahydrofuran and triethylene glycol.

In certain embodiments the organic solvent of step a)ii. is selected from the group consisting of acetonitrile, dimethyl sulfoxide, 1,4-dioxane and 2-methyl-2,4-pentanediol.

In certain embodiments the liquid of step a)ii. comprises about 20% (v/v), about 30% (v/v), about 40% (v/v) or about 50% (v/v) acetonitrile, dimethyl sulfoxide, 1,4-dioxane or 2-methyl-2,4-pentanediol. In certain embodiments the liquid of step a)ii. comprises 20% (v/v), 30% (v/v), 40% (v/v) or 50% (v/v) acetonitrile, dimethyl sulfoxide, 1,4-dioxane or 2-methyl-2,4-pentanediol. In certain embodiments the liquid of step a)ii. comprises about 20% (v/v), about 30% (v/v), about 40% (v/v) or about 50% (v/v) acetonitrile. In certain embodiments the liquid of step a)ii. comprises 20% (v/v), 30% (v/v), 40% (v/v) or 50% (v/v) acetonitrile. In certain embodiments the liquid of step a)ii. comprises about 30% (v/v) acetonitrile. In certain embodiments the liquid of step a)ii. comprises 30% (v/v) acetonitrile.

In certain embodiments the liquid of step a)ii. is a mixture of the at least one organic solvent with water or a mixture of the at least one organic solvent with an aqueous solution, such as a buffer.

In certain embodiments the aqueous solution is an aqueous buffer. In certain embodiments the aqueous buffer comprises a borate; such as orthoborate, metaborate or tetraborate; phosphate; bicarbonate; N,N-bis(2-hydroxyethyl)—2-aminoethanesulfonic acid; 3-(N-morpholino)propanesulfonic acid; (4-2-hydroxyethyl)—1-piperazineethanesulfonic acid; 3-N-bis(hydroxyethyl)-amino-2-hydroxypropane sulfonic acid; 3-(N-morpholino) propanesulfonic acid; (2-hydroxyethyl)-piperazine-N-2-hydroxypropanesulfonic acid; piperazine-1,4-bis(2-hydroxypropanesulfonic acid); 3-[4-(2-hydroxyethyl)piperazin-1-yl]propane-1-sulfonic acid; bicine; N-(2-hydroxyethyl)piperazine-N′-(4-butanesulfonic acid); or any combination thereof.

In certain embodiments the aqueous buffer has a pH in the range of about 7 to about 11, such as of about 7.5 to about 10.5, about 8 to about 10, about 8.5 to about 9.5 or the pH is about 9.

In certain embodiments the mixture of the at least one organic solvent with water or a mixture of the at least one organic solvent an aqueous solution comprises about 20% (v/v), about 30% (v/v), about 40% (v/v) or about 50% (v/v) acetonitrile, dimethyl sulfoxide, 1,4-dioxane or 2-methyl-2,4-pentanediol. In certain embodiments the mixture comprises 20% (v/v), 30% (v/v), 40% (v/v) or 50% (v/v) acetonitrile, dimethyl sulfoxide, 1,4-dioxane or 2-methyl-2,4-pentanediol. In certain embodiments the mixture comprises about 20% (v/v), about 30% (v/v), about 40% (v/v) or about 50% (v/v) acetonitrile. In certain embodiments the mixture comprises 20% (v/v), 30% (v/v), 40% (v/v) or 50% (v/v) acetonitrile.

In certain embodiments the mixture is about 30% (v/v) acetonitrile in water. In certain embodiments the mixture is 30% (v/v) acetonitrile in water.

In certain embodiments the at least one reagent Z—L²—L¹—FG and the one or more moiety D—H in step b) are combined in a molar ratio of ranging from 10:1 to 1:10, such as 9:1 to 1:9, 8:1 to 1:8, 7:1 to 1:7, 6:1 to 1:6, 5:1 to 1:5, 4:1 to 1:4, 3:1 to 1:3, 2:1 to 1:2 or 1:1. In certain embodiments the molar ratio of Z—L²—L¹—FG to D—H is ranging from 4:1 to 1:1. In certain embodiments the molar ratio of Z—L²—L¹—FG to D—H is 3:1.

In certain embodiments step b) comprises steps b)i. to b)iii:
b)i. mixing the at least one reagent Z—L²—L¹—FG from step a)i. with the liquid from step a)ii;
b)ii. adding the one or more drug D—H from step a)iii. to the mixture of step b)i.; and
b)iii. incubating the mixture of step b)ii. for at least 10 min

In certain embodiments step b)iii. is performed for about 10 min to about 24 hours, such as about 30 min to about 6 hours or about 2 to about 5 hours. In certain embodiments step b)iii. is performed for 10 min to 24 hours, such as 30 min to 6 hours or 2 to 5 hours.

In certain embodiments step b)iii. is performed at a temperature ranging from about 0° C. to about 40° C., such as ranging from about 5° C. to about 30° C., ranging from about 10° C. to about 20° C., ranging from about 11° C. to about 17° C. or at a temperature of about 14° C. In certain embodiments step b)iii. is performed at a temperature ranging from 0° C. to 40° C., such as ranging from 5° C. to 30° C., ranging from 10° C. to 20° C., ranging from about 11° C. to about 17° C. or at a temperature of 14° C.

In certain embodiments the IL-2 conjugate of formula (I) in step c) is purified via sterile filtration, chromatography or ultrafiltration. In certain embodiments the IL-2 conjugate of formula (I) in step c) is purified via mixed mode chromatography

In certain embodiments —FG is selected from the group consisting of (methylsulfonyl)oxy, [(nonafluorobutyl)sulfonyl]oxy, [(trifluoromethyl) sulfonyl]oxy, [2-(trimethylsilyl)ethoxy]methyl acetal, 1-hydroxybenzotriazole, 4-methoxybenzenesulfonamide, 4-methoxybenzyl ether, 4-nitrophenolate, 9-fluorenylmethyl carbamate, azide, benzyloxy, benzyloxycarbonyl, brosylate, dichlorophenolate, difluorophenolate, dihalophosphinoyloxy, ethanesulfonyloxy, ethoxyethyl ether, hydroxysuccinimide, imidazole, methanesulfonylate/mesylate, methanesulfonyloxy, methoxymethyl ether, monofluorophenolate, N-hydroxysuccinimide, N-hydroxysulphosuccinimide, nitrophenolate, pentafluorophenolate, phenyl, β-toluenesulfonylate (tosylate), tert-butyldimethylsilyl ether, tetrachlorophenolate, tetrafluorophenolate, tetrahydropyranyl ether, thienyloxy, triflate, trifluoroacetoxy, trifluoromethylmethanesulfonylate and trityl(triphenylmethyl)ether.

In certain embodiments —FG is selected from the group consisting of 4-nitrophenolate, pentafluorophenolate and N-hydroxysuccinimide. In certain embodiments —FG is pentafluorophenolate.

In certain embodiments the IL-2 conjugate of formula (I) is synthesized by a method comprising the steps of
a) providing
   a)i. at least one reagent of formula (XII)

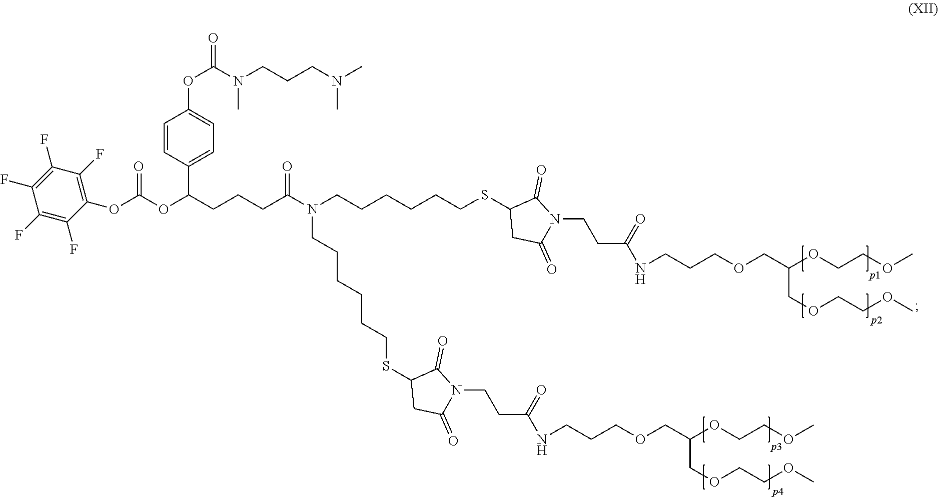

wherein p 1, p2, p3, p4 are independently of each other an integer ranging from 110 to 345, from 140 to 315, from 170 to 285, from 195 to 255, from 200 to 250, from 210 to 250 or from 220 to 240;
a)ii. a mixture of about 30% (v/v) acetonitrile in water; and
a)iii. a solution comprising D—H;
b) mixing the at least one reagent of step a)i. with the mixture of step a)ii.;
c) mixing the mixture of step b) with the solution of step a)iii., wherein the molar ratio of the reagent of formula (XII) and D—H is ranging from 4:1 to 1:1;
d) incubating the mixture of step c) for 1 to 5 hours at a temperature ranging from 10° C. to 20° C. to obtain the IL-2 conjugate of formula (I); and
e) optionally, purifying the IL-2 conjugate of formula (I) from the incubated mixture of step d).

D—H is the free form of —D, which is further described elsewhere herein.

In certain embodiments D—H of the method is D′—M_{mod} and wherein —D′— has the amino acid sequence of SEQ ID NO:4, —M_{mod} has the structure of (A-1d) or (A-1e)

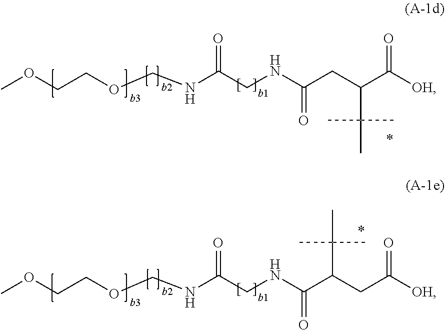

wherein
the dashed line marked with the asterisk indicates attachment to the sulfur atom of the side chain of the cysteine at position 38;
b1 is 2 or 3;
b2 is 2 or 3; and
b3 ranges from 90 to 140, from 95 to 135 or from 100 to 125.

It was surprisingly found that dissolution of the reagent of step a)i. in an aqueous mixture of an organic solvent can reduce the required amount of the reagent of step a)i. for synthesizing the IL-2 conjugate of formula (I) with a similar yield and purity compared to dissolution in pure water. In addition, it was surprisingly found that dissolution of the reagent of step a)i. in an aqueous mixture of an organic solvent reduced dissolution time and reduced hydrolysis of the reagent of step a)i. compared to dissolution in pure water.

It was surprisingly found that the IL-2 conjugate of formula (I) effectively increases the level of soluble CD25 (sCD25) and/or the level of CD8+ T cells in patients having cancer and furthermore that an increase in the levels of sCD25 and/or in the levels of CD8+ T cells correlates with a positive treatment outcome, such as a stable disease for at least 6 months, partial response or complete response. To the best of the applicant's knowledge, this is the first IL-2 conjugate for which such correlations are observed in patients having cancer at the time of filing.

In an eighth aspect the invention relates to a method of treating or delaying progression of cancer in a patient, comprising:
(a) measuring the level of sCD25 in a biological sample obtained from the patient after administration of at least one dose ranging from 80 μg IL2/kg to 160 μg IL-2/kg of the IL-2 conjugate of formula (I) to the patient; and/or
(b) measuring the level and/or proliferation of CD8+ T cells in a biological sample obtained from the patient after administration of at least one dose ranging from 80 μg IL2/kg to 160 μg IL-2/kg of the IL-2 conjugate of formula (I) to the patient; (c) comparing the level from step (a) and/or the level and/or proliferation from step (b) to a reference; wherein an increased level of sCD25 and/or increased level and/or proliferation of CD8+ T cells compared to the reference indicates that the patient is likely to benefit from the treatment with the IL-2 conjugate of formula (I); and
(d) if the patient has
   i) an increased level of sCD25 compared to the reference, administering to the patient one or more additional dose ranging from 80 μg IL2/kg to 160 μg IL-2/kg of the IL-2 conjugate of formula (I); and/or
   ii) an increased level and/or proliferation of CD8+ T cells compared to the reference, administering to the patient one or more additional dose ranging from 80 μg IL2/kg to 160 μg IL-2/kg of the IL-2 conjugate of formula (I).

The IL-2 conjugate of formula (I) of the eighth aspect is defined as described elsewhere herein.

In certain embodiments the dose in steps (a), (b) and (d) of the eighth aspects ranges from 100 μg IL-2/kg to 140 μg IL-2/kg. In certain embodiments the dose in steps (a), (b) and (d) of the eighth aspect ranges from 110 μg IL-2/kg to 130 μg IL-2/kg. In certain embodiments the dose in steps (a), (b) and (d) of the eighth aspect is about 120 μg IL-2/kg. In certain embodiments the dose in steps (a), (b) and (d) of the eighth aspect is 120 μg IL-2/kg.

In certain embodiments the biological sample in step (a) and/or step (b) is obtained after administration of the first dose of the IL-2 conjugate of formula (I). In certain embodiments the biological sample in step (a) and/or step (b) is obtained after administration of the second dose of the IL-2 conjugate of formula (I). In certain embodiments the biological sample in step (a) and/or step (b) is obtained after administration of the third dose of the IL-2 conjugate of formula (I). In certain embodiments the biological sample in step (a) and/or step (b) is obtained after administration of the fourth dose of the IL-2 conjugate of formula (I). In certain embodiments the biological sample in step (a) and/or step (b) is obtained after administration of the fifth dose of the IL-2 conjugate of formula (I). In certain embodiments the biological sample in step (a) and/or step (b) is obtained after administration of any dose administered after the fifth dose of the IL-2 conjugate of formula (I).

In certain embodiments the biological sample in step (a) and/or step (b) is obtained between 1 day to 6 weeks after administration of the IL-2 conjugate of formula (I). In certain embodiments the biological sample in step (a) and/or step (b) is obtained 1 day after administration of the IL-2 conjugate of formula (I). In certain embodiments the biological sample in step (a) and/or step (b) is obtained 2 days after administration of the IL-2 conjugate of formula (I). In certain embodiments the biological sample in step (a) and/or step (b) is obtained 3 days after administration of the IL-2 conjugate of formula (I). In certain embodiments the biological sample in step (a) and/or step (b) is obtained 4 days after administration of the IL-2 conjugate of formula (I). In certain embodiments the biological sample in step (a) and/or step (b) is obtained 5 days after administration of the IL-2 conjugate of formula (I). In certain embodiments the biological sample in step (a) and/or step (b) is obtained 6 days after administration of the IL-2 conjugate of formula (I). In certain embodiments the biological sample in step (a) and/or step (b) is obtained 1 week after administration of the IL-2 conjugate of formula (I). In certain embodiments the biological sample in step (a) and/or step (b) is obtained 2 weeks after administration of the IL-2 conjugate of formula (I). In certain embodiments the biological sample in step (a) and/or step (b) is obtained 3 weeks after administration of the IL-2 conjugate of formula (I). In certain embodiments the biological sample in step (a) and/or step (b) is obtained 4 weeks after administration of the IL-2 conjugate of formula (I). In certain embodiments the biological sample in step (a) and/or step (b) is obtained 5 weeks after administration of the IL-2 conjugate of formula (I). In certain embodiments the biological sample in step (a) and/or step (b) is obtained 6 weeks after administration of the IL-2 conjugate of formula (I).

In certain embodiments the biological sample in step (a) and step (b) is the same biological sample. In certain embodiments the biological samples in step (a) and step (b) are different biological samples.

In certain embodiments the biological sample in step (a) and/or step (b) is a blood sample or a tumor sample. In certain embodiments the biological sample in step (a) and/or step (b) is a blood sample. In certain embodiments the biological sample in step (a) and/or step (b) is a tumor sample.

In certain embodiments the blood sample comprises peripheral blood mononuclear cells (PBMCs).

In certain embodiments the level of sCD25 is measured. In certain embodiments the level of CD8+ T cells is measured. In certain embodiments the proliferation of CD8+ T cells is measured. In certain embodiments the level of sCD25 and the level of CD8+ T cells are measured. In certain embodiments the level of sCD25 and the proliferation of CD8+ T cells are measured. In certain embodiments the level of CD8+ T cells and the proliferation of CD8+ T cells are measured. In certain embodiments the level of sCD25, the level of CD8+ T cells and the proliferation of CD8+ T cells are measured.

In certain embodiments the level and/or proliferation of CD8+ T cells is measured by flow cytometry. In certain embodiments the proliferation of CD8+ T cells is measured by detecting expression of a cellular or nuclear proliferation-associated antigen on a CD8+ T cell. In certain embodiments the proliferation of CD8+ T cells is measured by detecting Ki-67.

In certain embodiments the reference comprises a biological sample obtained from the patient prior to administration of the IL-2 conjugate of formula (I). In certain embodiments the reference comprises a standard derived from a population of known non-responders to treatment with the IL-2 conjugate of formula (I). In certain embodiments the reference comprises a standard derived from a population of known responders to treatment with the IL-2 conjugate of formula (I). In certain embodiments the reference is data compiled across a plurality of patients. In certain embodiments the reference is a healthy subject.

In certain embodiments the one or more additional dose in step (d) is administered if the absolute level of sCD25 in the biological sample, such as measured in pg/ml, is increased compared to the reference. In certain embodiments the one or more additional dose in step (d) is administered if the relative level of sCD25 in the biological sample, such as indicated by a fold-change, is increased compared to the reference.

In certain embodiments the one or more additional dose in step (d) is administered if the absolute level of CD8+ T cells in the biological sample, such as measured in cells/β1, is increased compared to the reference. In certain embodiments the one or more additional dose in step (d) is administered if the relative level of CD8+ T cells in the biological sample, such as indicated by a fold-change, is increased compared to the reference.

In certain embodiments the one or more additional dose in step (d) is administered if the absolute number of proliferating CD8+ T cells in the biological sample, such as measured in cells/β1, is increased compared to the reference. In certain embodiments the one or more additional dose in step (d) is administered if the relative proliferation of CD8+ T cells in the biological sample, such as indicated by a fold-change, is increased compared to the reference. Steps (a), (b) and (c) may be repeated until an increased level sCD25 and/or the level and/or proliferation of CD8+ T cells is observed.

The invention is further described by the following non-limiting items. 1. An IL-2 conjugate or a pharmaceutically acceptable salt thereof for use in the treatment of cancer, wherein the IL-2 conjugate is administered in a dose ranging from 80 μg IL-2/kg to 160 μg IL-2/kg and wherein the IL-2 conjugate is of formula (I)

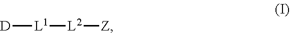

wherein
—D is —D′—M_{mod},
   wherein
   —D′— is an IL-2 moiety;
   —M_{mod} is a first polymeric moiety that is stably conjugated to a side chain of an amino acid residue of —D′—;
—L¹— is a linker moiety covalently and reversibly attached to —D′—;
—L²— is absent or a spacer moiety; and
—Z is a second polymeric moiety.
2. The IL-2 conjugate for use of item 1, wherein the dose ranges from 100 μg IL-2/kg to 140 μg IL-2/kg.
3. The IL-2 conjugate for use of item 1 or 2, wherein the dose ranges from 110 μg IL-2/kg to 130 μg IL-2/kg.
4. The IL-2 conjugate for use of any one of items 1 to 3, wherein the dose is about 120 μg IL-2/kg.
5. The IL-2 conjugate for use of any one of items 1 to 4, wherein the dose is 120 μg IL-2/kg.
6. The IL-2 conjugate for use of any one of items 1 to 5, wherein the dose is administered multiple times.
7. The IL-2 conjugate for use of any one of items 1 to 6, wherein the interval between two consecutive administrations ranges from two to eight weeks.
8. The IL-2 conjugate for use of any one of items 1 to 7, wherein the interval between two consecutive administrations is three weeks.
9. The IL-2 conjugate for use of any one of items 1 to 7, wherein the interval between two consecutive administrations is six weeks.
10. The IL-2 conjugate for use of any one of items 1 to 6, wherein the IL-2 conjugate is administered at a frequency dependent on disease progression.
11. The IL-2 conjugate for use of any one of items 1 to 5, wherein the dose is administered once.
12. The IL-2 conjugate for use of any one of items 1 to 11, wherein the treatment starts directly on diagnosis of the cancer.
13. The IL-2 conjugate for use of any one of items 1 to 12, wherein the treatment is a first line of treatment.
14. The IL-2 conjugate for use of any one of items 1 to 12, wherein the treatment is the second, third or further line of treatment.
15. The IL-2 conjugate for use of any one of items 1 to 14, wherein the IL-2 conjugate is administered via external application, injection or infusion, including intraarticular, periarticular, intradermal, subcutaneous, intramuscular, intravenous, intraosseous, intraperitoneal, intrathecal, intracapsular, intraorbital, intravitreal, intratympanic, intravesical, intracardiac, transtracheal, subcuticular, subcapsular, subarachnoid, intraspinal, intraventricular, intrasternal injection and infusion; direct delivery to the brain via an implanted device allowing delivery of the IL-2 conjugate to brain tissue or brain fluids (e.g., Ommaya Reservoir), direct intracerebroventricular injection or infusion, injection or infusion into brain or brain associated regions, injection into the subchoroidal space, retro-orbital injection or ocular instillation.
16. The IL-2 conjugate for use of any one of items 1 to 15, wherein the IL-2 conjugate is administered intravenously.
17. The IL-2 conjugate for use of any one of items 1 to 15, wherein the IL-2 conjugate is administered subcutaneously.
18. The IL-2 conjugate for use of any one of items 1 to 17, wherein —D′— has at least 95% sequence identity to the amino acid sequence of SEQ ID NO:1.
19. The IL-2 conjugate for use of any one of items 1 to 18, wherein —D′— has at least 96% sequence identity to the amino acid sequence of SEQ ID NO:1.
20. The IL-2 conjugate for use of any one of items 1 to 19, wherein —D′— has at least 97% sequence identity to the amino acid sequence of SEQ ID NO:1.
21. The IL-2 conjugate for use of any one of items 1 to 20, wherein —D′— has at least 98% sequence identity to the amino acid sequence of SEQ ID NO:1.
22. The IL-2 conjugate for use of any one of items 1 to 21, wherein —D′— has at least 99% sequence identity to the amino acid sequence of SEQ ID NO:1.
23. The IL-2 conjugate for use of any one of items 1 to 22, wherein —D′— comprises the amino acid sequence of SEQ ID NO:1, in which one amino acid residue selected from the group consisting of K35, R38, M39, T41, F42, K43, F44, Y45, E62, P65, E68, V69, and L72 is replaced with a proteinogenic or non-proteinogenic amino acid residue.
24. The IL-2 conjugate for use of any one of items 1 to 23, wherein —D′— comprises the Aldel mutation.
25. The IL-2 conjugate for use of any one of items 1 to 23, wherein —D′— comprises the amino acid sequence of SEQ ID NO:1, in which one amino acid residue selected from the group consisting of K35, R38, M39, T41, F42, K43, F44, Y45, E62, P65, E68, V69, and L72 is replaced with a cysteine residue.
26. The IL-2 conjugate for use of any one of items 1 to 23 or 25, wherein —D′— has the amino acid sequence of SEQ ID NO:1, in which one amino acid residue selected from the group consisting of K35, R38, M39, T41, F42, K43, F44, Y45, E62, P65, E68, V69, and L72 is replaced with a cysteine residue.
27. The IL-2 conjugate for use of any one of items 1 to 23, 25 or 26, wherein —D′— has the amino acid sequence of SEQ ID NO:4.
28. The IL-2 conjugate for use of any one of items 1 to 27, wherein —M_{mod} has a molecular weight ranging from 0.5 kDa to 20 kDa.
29. The IL-2 conjugate for use of any one of items 1 to 28, wherein —M_{mod} has a molecular weight ranging from 1 kDa to 20 kDa.
30. The IL-2 conjugate for use of any one of items 1 to 29, wherein —M_{mod} has a molecular weight ranging from 2 kDa to 10 kDa.
31. The IL-2 conjugate for use of any one of items 1 to 30, wherein —M_{mod} has a molecular weight ranging from 3 kDa to 7 kDa.
32. The IL-2 conjugate for use of any one of items 1 to 31, wherein —M_{mod} has a molecular weight of 5±25% kDa.
33. The IL-2 conjugate for use of any one of items 1 to 32, wherein —M_{mod} has a molecular weight ranging from 4 kDa to 6 kDa.
34. The IL-2 conjugate for use of any one of items 1 to 32, wherein —M_{mod} has a molecular weight of 5 20% kDa.
35. The IL-2 conjugate for use of any one of items 1 to 34, wherein —M_{mod} has a molecular weight of 5±15% kDa.
36. The IL-2 conjugate for use of any one of items 1 to 35, wherein —M_{mod} has a molecular weight of about 5 kDa.
37. The IL-2 conjugate for use of any one of items 1 to 36, wherein —M_{mod} is a PEG-based polymer.
38. The IL-2 conjugate for use of any one of items 1 to 37, wherein attachment of —M_{mod} occurs at an amino acid position selected from the group consisting of K35, R38, M39, T41, F42, K43, F44, Y45, E62, P65, E68, V69, and L72.
39. The IL-2 conjugate for use of any one of items 1 to 23 or 25 to 38, wherein —D′— has the amino acid sequence of SEQ ID NO. 4 and —M_{mod} is stably conjugated to the sulfur atom of the side chain of the cysteine at position 38.
40. The IL-2 conjugate for use of any one of items 1 to 39, wherein —M_{mod} is of formula (A-1)

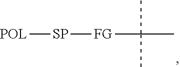

wherein
   —FG— is a linkage;
   —SP— is a spacer moiety; and
   —POL is a polymer.
41. The IL-2 conjugate for use of item 40, wherein —FG— of formula (A-1) is of formula (FG— 1a)

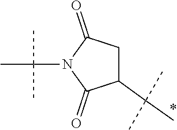

wherein
   the dashed line marked with the asterisk indicates attachment to the sulfur of a cysteine side chain of —D′— and the unmarked dashed line indicates attachment to —SP—.
42. The IL-2 conjugate for use of item 40, wherein —FG— of formula (A-1) is of formula (FG— 1d) or (FG-1e)

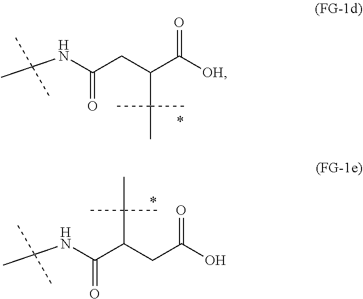

wherein the dashed line marked with the asterisk indicates attachment to the sulfur of a cysteine residue of —D′— and the unmarked dashed line indicates attachment to —SP—.
43. The IL-2 conjugate for use of any one of items 40 to 42, wherein —SP— is C₁₋₁₀ alkyl, which C₁₋₁₀ alkyl is optionally substituted with one or more —R⁹, and which C₁₋₁₀ alkyl is optionally interrupted by one or more groups selected from the group consisting of —O—, —C(O)N(R¹⁰)—, —S(O)₂—, —S(O)—, —S—, —N(R¹⁰)——OC(OR¹⁰)(R^{10a})—, —N(R¹⁰)C(O)N(R^{10a})—, and —OC(O)N(R¹⁰)—; wherein each —R⁹ is selected from the group consisting of C₁₋₆ alkyl; and each —R¹⁰ and —R^{10a} is independently selected from the group consisting of —H and C₁₋₆ alkyl.
44. The IL-2 conjugate for use of any one of items 40 to 43, wherein —POL is of formula (A-1i)

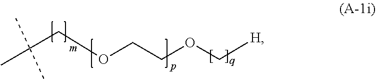

wherein
   the dashed line indicates attachment to —SP—;
   m is 0 or 1;
   p is an integer ranging from about 10 to about 540; and
   q is selected from the group consisting of 1, 2, 3, 4, 5, and 6.
45. The IL-2 conjugate for use of any one of items 1 to 41, 43 or 44, wherein —M_{mod} is of formula (A-1a)

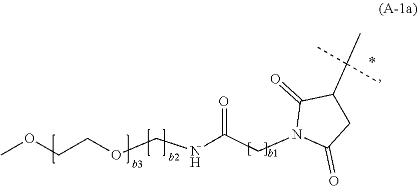

wherein
   the dashed line marked with the asterisk indicates attachment to the sulfur of a cysteine residue of —D′—;
   b1 is an integer selected from the group consisting of 1, 2, 3, 4, 5, 6, 7, 8, 9, 10, 11, 12, 13, 14, 15, 16, 17, 18, 19 and 20;
   b2 is an integer selected from the group consisting of 1, 2, 3, 4, 5, 6, 7, 8, 9, 10, 11, 12, 13, 14, 15, 16, 17, 18, 19 and 20; and
   b3 is an integer ranging from about 10 to about 540.
46. The IL-2 conjugate for use of any one of items 1 to 40 or 42 to 44, wherein —M_{mod} is of formula (A-1 d) or (A-1e)

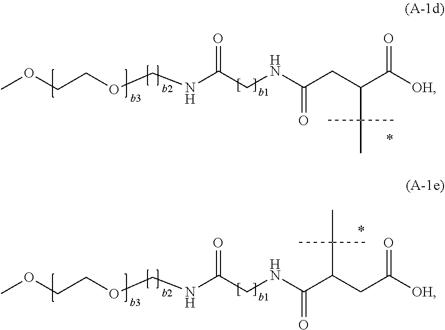

wherein
   the dashed line marked with the asterisk indicates attachment to the sulfur of a cysteine residue of —D′—;
   b1 is an integer selected from the group consisting of 1, 2, 3, 4, 5, 6, 7, 8, 9, 10, 11, 12, 13, 14, 15, 16, 17, 18, 19 and 20;
   b2 is an integer selected from the group consisting of 1, 2, 3, 4, 5, 6, 7, 8, 9, 10, 11, 12, 13, 14, 15, 16, 17, 18, 19 and 20; and
   b3 is an integer ranging from about 10 to about 540.
47. The IL-2 conjugate for use of item 45 or 46, wherein b1 is an integer ranging from 1 to 4.
48. The IL-2 conjugate for use of any one of items 45 to 47, wherein b1 is 2.
49. The IL-2 conjugate for use of any one of items 45 to 47, wherein b1 is 3.
50. The IL-2 conjugate for use of any one of items 45 to 49, wherein b2 is 2.
51. The IL-2 conjugate for use of any one of items 45 to 49, wherein b2 is 3.
52. The TL-2 conjugate for use of any one of items 45 to 51, wherein b3 is an integer ranging from 10 to 450.
53. The IL-2 conjugate for use of any one of items 45 to 52, wherein b3 is an integer ranging from 22 to 340.
54. The IL-2 conjugate for use of any one of items 45 to 53, wherein b3 is an integer ranging from 45 to 230.
55. The IL-2 conjugate for use of any one of items 45 to 54, wherein b3 is an integer ranging from 65 to 160.
56. The IL-2 conjugate for use of any one of items 45 to 55, wherein b3 is selected such that —M_{mod} has a molecular weight of 5±25% kDa.
57. The IL-2 conjugate for use of any one of items 45 to 56, wherein b3 is an integer ranging from 90 to 140.
58. The IL-2 conjugate for use of any one of items 45 to 57, wherein b3 is selected such that —M_{mod} has a molecular weight ranging from about 4 kDa to about 6 kDa.
59. The IL-2 conjugate for use of any one of items 45 to 58, wherein b3 is selected such that —M_{mod} has a molecular weight of 5 20% kDa.
60. The IL-2 conjugate for use of any one of items 45 to 59, wherein b3 is an integer ranging from 95 to 135.
61. The IL-2 conjugate for use of any one of items 45 to 60, wherein b3 is selected such that —M_{mod} has a molecular weight of 5 15% kDa.
62. The IL-2 conjugate for use of any one of items 45 to 61, wherein b3 is an integer ranging from 100 to 125.
63. The IL-2 conjugate for use of any one of items 45 to 62, wherein b3 is selected such that —M_{mod} has a molecular weight of about 5 kDa.
64. The IL-2 conjugate for use of any one of items 1 to 63, wherein —Z has a molecular weight ranging from about 20 kDa to about 60 kDa.
65. The IL-2 conjugate for use of any one of items 1 to 64, wherein —Z has a molecular weight ranging from about 25 kDa to about 55 kDa.
66. The IL-2 conjugate for use of any one of items 1 to 65, wherein —Z has a molecular weight ranging from about 30 kDa to about 50 kDa.
67. The IL-2 conjugate for use of any one of items 1 to 66, wherein —Z has a molecular weight ranging from about 35 kDa to about 45 kDa.
68. The IL-2 conjugate for use of any one of items 1 to 67, wherein —Z has a molecular weight ranging of about 40 kDa.
69. The IL-2 conjugate for use of any one of items 1 to 68, wherein —Z comprises a moiety of formula (A-c):

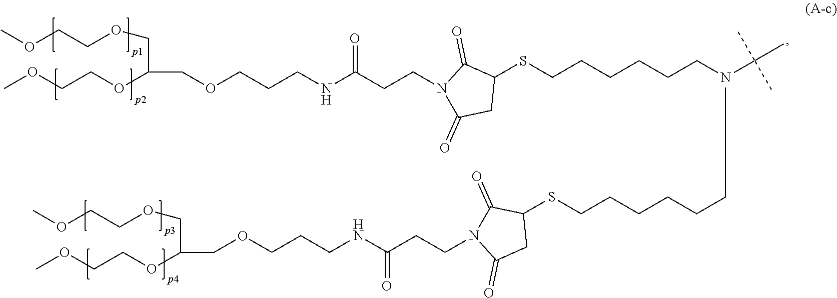

wherein
   p1, p2, p3, p4 are independently of each other an integer ranging from 110 to 345
70. The IL-2 conjugate for use of item 69, wherein p1, p2, p3, p4 of formula (A-c) are independently of each other an integer ranging from 140 to 315.
71. The IL-2 conjugate for use of any one of items 69 to 70, wherein p 1, p2, p3, p4 of formula (A-c) are independently of each other an integer ranging from 170 to 285.
72. The IL-2 conjugate for use of any one of items 69 to 71, wherein p 1, p2, p3, p4 of formula (A-c) are independently of each other an integer ranging from 195 to 255.
73. The IL-2 conjugate for use of any one of items 69 to 72, wherein p 1, p2, p3, p4 of formula (A-c) are independently of each other an integer ranging from 200 to 250.
74. The IL-2 conjugate for use of any one of items 69 to 73, wherein p 1, p2, p3, p4 of formula (A-c) are independently of each other an integer ranging from 210 to 250.
75. The IL-2 conjugate for use of any one of items 69 to 74, wherein p 1, p2, p3, p4 of formula (A-c) are independently of each other an integer ranging from 220 to 240.
76. The IL-2 conjugate for use of any one of items 1 to 75, wherein —M_{mod} is attached to a different amino acid residue of —D′— than the moiety —L¹—L²—Z.
77. The IL-2 conjugate for use of any one of items 1 to 76, wherein —L¹— is attached to a lysine residue of —D′—.
78. The IL-2 conjugate for use of any one of items 1 to 77, wherein the linkage between —D′— and —L¹— is a carbamate.
79. The IL-2 conjugate for use of any one of items 1 to 78, wherein —L¹— is of formula (IX-a):

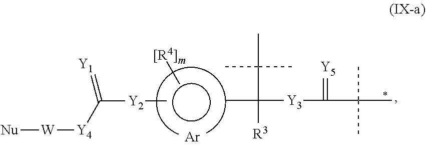

wherein
   the dashed line marked with the asterisk indicates attachment to a nitrogen of —D′— and
   the unmarked dashed line indicates attachment to —L²—Z;
   n is 0, 1, 2, 3 or 4;
   ═Y₁ is selected from the group consisting of ═O and ═S;
   —Y₂— is selected from the group consisting of —O— and —S—;
   —Y₃— is selected from the group consisting of —O— and —S—;
   —Y₄— is selected from the group consisting of —O—, —NR⁵— and —C(R⁶R^{6a})—;
   ═Y₅ is selected from the group consisting of ═O and ═S; —R³, —R⁵, —R⁶, —R^{6a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; —R⁴ is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl;
   —W— is selected from the group consisting of C₁₋₂₀ alkyl optionally interrupted by one or more groups selected from the group consisting of C₃₋₁₀ cycloalkyl, 8- to 30-membered carbopolycyclyl, 3- to 10-membered heterocyclyl, —C(O)—, —C(O)N(R⁷)—, —O—, —S— and —N(R⁷)—;
   —Nu is a nucleophile selected from the group consisting of —N(R⁷R^{7a}), —N(R⁷OH), —N(R⁷)—N(R^{7a}R^{7b}), —S(R⁷),—COOH

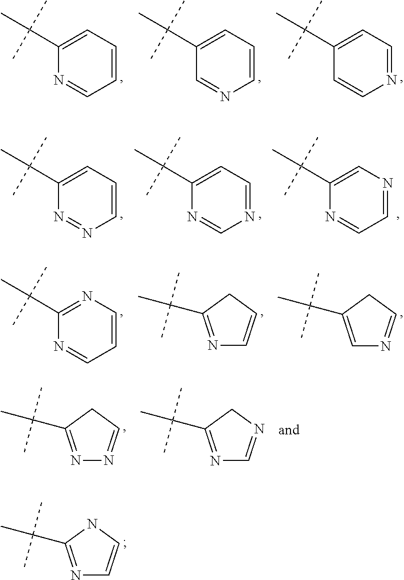

—Ar— is selected from the group consisting of

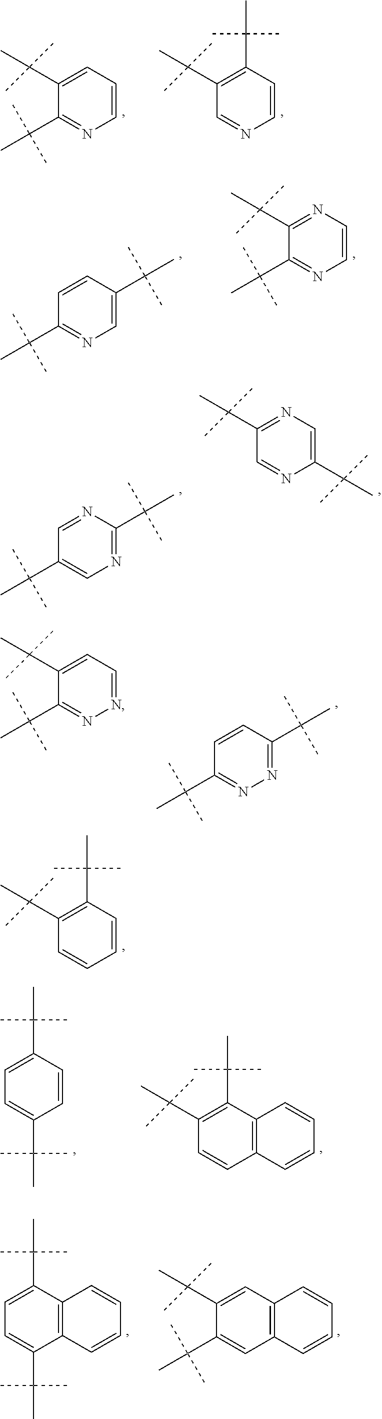

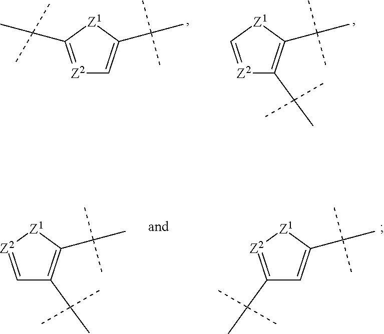

wherein
   dashed lines indicate attachment to the remainder of —L¹—,
   —Z¹— is selected from the group consisting of —O—, —S— and —N(R⁷)—, and —Z²—0 is —N(R⁷)—; and
   —R⁷, —R^{7a}, —R^{7b} are independently of each other selected from the group consisting of —H, C₁₋₆ alkyl, C₂₋₆ alkenyl and C₂₋₆ alkynyl;
   wherein —L¹— is optionally further substituted.
80. The IL-2 conjugate for use of any one of items 1 to 79, wherein —L¹— is of formula (IX-d)

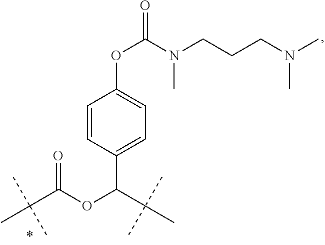

wherein
   the dashed line marked with the asterisk indicates attachment to a nitrogen of —D′—; and
   the unmarked dashed line indicates attachment to —L²—Z.
81. The IL-2 conjugate for use of item 80, wherein the dashed line marked with the asterisk indicates attachment to the nitrogen of the ε-amino group of a lysine residue of —D′— or to the nitrogen of the N-terminal amine of —D′—.
82. The IL-2 conjugate for use of item 80 or 81, wherein the dashed line marked with the asterisk indicates attachment to the nitrogen of the ε-amino group of a lysine residue of —D′—.
83. The IL-2 conjugate for use of any one of items 1 to 82, wherein —L²— is a C₁₋₂₀ alkyl chain, which is optionally interrupted by one or more groups independently selected from —O—, —C(O)—, —T— and —C(O)N(R^{y1})—; and which C₁₋₂₀ alkyl chain is optionally substituted with one or more groups independently selected from —OH, —T and —C(O)N(R⁶R^{y6a}); wherein —R¹, —R^{y6}, —R^{y6a} are independently selected from the group consisting of H and C₁₋₄ alkyl and wherein T is selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl.
84. The IL-2 conjugate for use of any one of items 1 to 83, wherein —L¹—L²—Z is of formula (XI)

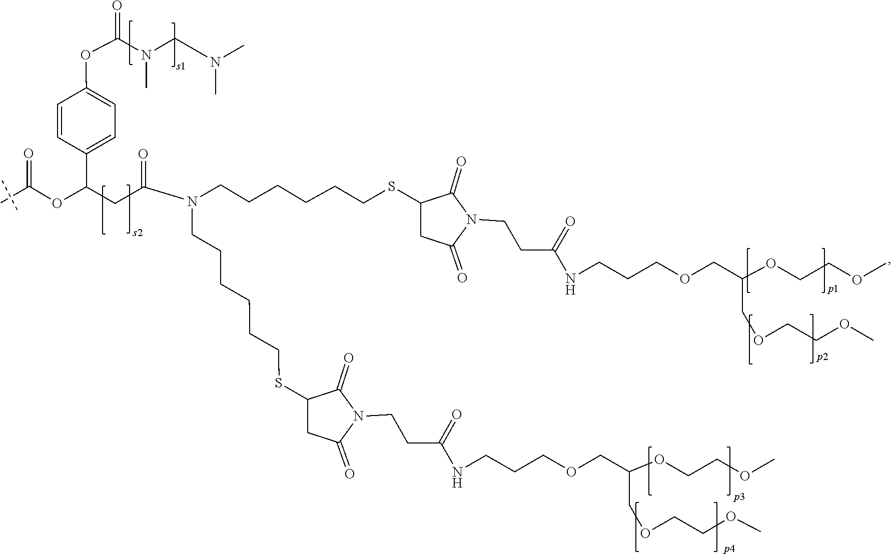

wherein the dashed line indicates attachment to a nitrogen of —D′—;
   s1 is an integer selected from the group consisting of 1, 2, 3, 4, 5, 6, 7, 8, 9 and 10;
   s2 is an integer selected from the group consisting of 0, 1, 2, 3, 4, 5, 6, 7, 8, 9, 10, 11, 12, 13, 14, 15, 16, 17, 18, 19 and 20; and
   p1, p2, p3, p4 are independently of each other an integer ranging from 110 to 345.
85. The IL-2 conjugate for use of item 84, wherein the dashed line indicates attachment to the nitrogen of the ε-amino group of a lysine residue of —D′— or to the nitrogen of the N-terminal amine of —D′—.
86. The IL-2 conjugate for use of item 84 or 85, wherein the dashed line indicates attachment to the nitrogen of the ε-amino group of a lysine residue of —D′—.
87. The IL-2 conjugate for use of any one of items 1 to 86, wherein the IL-2 conjugate is of formula (I), wherein —D is —D′—M_{mod} and wherein —D′— has the amino acid sequence of SEQ ID NO:4, —M_{mod} has the structure of (A-1d) or (A-1e)

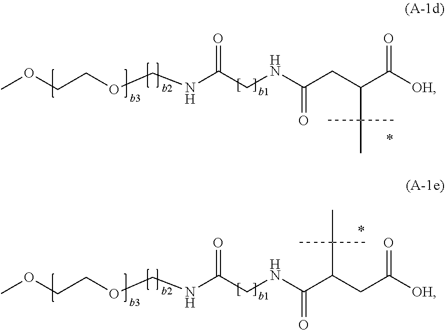

wherein
   the dashed line marked with the asterisk indicates attachment to the sulfur atom of the side chain of the cysteine at position 38;
   b1 is 2;
   b2 is 2;
   b3 ranges from 90 to 140, from 95 to 135 or from 100 to 125;
   and wherein —L¹—L²—Z is of formula (XI-a)

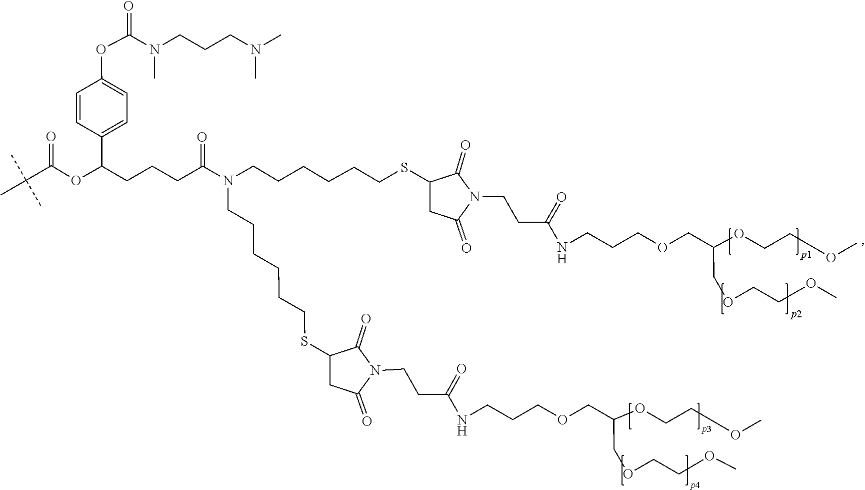

wherein the dashed line indicates attachment to the nitrogen of the ε-amino group of a lysine residue of —D′— or to the nitrogen of the N-terminal amine of —D′—; and p1, p2, p3, p4 are independently of each other an integer ranging from 110 to 345, from 140 to 315, from 170 to 285, from 195 to 255, from 200 to 250, from 210 to 250 or from 220 to 240.
88. The IL-2 conjugate for use of any one of items 1 to 87, wherein the cancer is selected from the group consisting of liquid tumors, solid tumors and lymphomas.
89. The IL-2 conjugate for use of item 88, wherein the liquid tumor may be a leukemia or myeloid neoplasm, such as chronic lymphocytic leukemia (CLL), chronic myelogenous leukemia (CMIL), hairy cell leukemia, lymphoblastic leukemia, myeloid leukemia, plasma cell leukemia, acute lymphoblastic leukemia (ALL), acute myeloid leukemia (AML), myelodysplastic syndrome (MDS), myeloproliferative neoplasm (MPN), post-MPN AML, post-MDS AML, del(5q)-associated high risk MDS or AML, blast-phase chronic myelogenous leukemia, multiple myeloma, myelodysplastic syndromes, chronic myeloproliferative disorders, plasma cell neoplasm and Waldenstrom's macroglobulinemia.
90. The IL-2 conjugate for use of item 88, wherein the solid tumor or lymphoma may be selected from the group consisting of lip and oral cavity cancer, oral cancer, liver cancer/hepatocellular cancer, primary liver cancer, lung cancer, lymphoma, malignant mesothelioma, malignant thymoma, skin cancer, intraocular melanoma, metastatic squamous neck cancer with occult primary, childhood multiple endocrine neoplasia syndrome, mycosis fungoides, nasal cavity and paranasal sinus cancer, nasopharyngeal cancer, neuroblastoma, oropharyngeal cancer, ovarian cancer, pancreatic cancer, parathyroid cancer, pheochromocytoma, pituitary tumor, adrenocortical carcinoma, AIDS-related malignancies, anal cancer, bile duct cancer, bladder cancer, brain and nervous system cancer, breast cancer, bronchial adenoma/carcinoid, gastrointestinal carcinoid tumor, carcinoma, colorectal cancer, endometrial cancer, esophageal cancer, extracranial germ cell tumor, extragonadal germ cell tumor, extrahepatic bile duct cancer, gallbladder cancer, gastric (stomach) cancer, gestational trophoblastic tumor, head and neck cancer, hypopharyngeal cancer, islet cell carcinoma (endocrine pancreas), kidney cancer/renal cell cancer, laryngeal cancer, pleuropulmonary blastoma, prostate cancer, transitional cell cancer of the renal pelvis and ureter, retinoblastoma, salivary gland cancer, sarcoma, Sezary syndrome, small intestine cancer, genitourinary cancer, malignant thymoma, thyroid cancer, Wilms' tumor, cholangiocarcinoma, and also their related earlier stages of aberrant cell growth such as dysplasias, adenomas, and carcinoma in situs.
91. The IL-2 conjugate for use of any one of items 1 to 90, 316 to 348 and 386 to 391, wherein the IL-2 conjugate is administered to the patient prior to, simultaneously with or after administration of one or more additional drug, which one or more additional drug is selected from the group consisting of pattern recognition receptor agonists (PRRA), cytotoxic/chemotherapeutic agents, immune checkpoint inhibitors or antagonists, immune checkpoint agonists, immune activating receptor agonists, multi-specific drugs, antibody-drug conjugates (ADC), antibody-adjuvant conjugates (AAC), radionuclides or targeted radionuclide therapeutics, DNA damage repair inhibitors, tumor metabolism inhibitors, pattern recognition receptor agonists, protein kinase inhibitors, chemokine and chemoattractant receptor agonists, chemokine or chemokine receptor antagonists, cytokine receptor agonists, death receptor agonists, CD47 or SIRPα antagonists, oncolytic drugs, signal converter proteins, epigenetic modifiers, tumor peptides or tumor vaccines, heat shock protein (HSP) inhibitors, proteolytic enzymes, ubiquitin and proteasome inhibitors, adhesion molecule antagonists, hormones including hormone peptides and synthetic hormones, and adoptive cellular therapies such as Tumor Infiltrating Lymphocyte (TIL) therapy, Chimeric Antigen Receptor (CAR) therapy, T cell therapy, Natural Killer (NK) cell therapy, CAR-T therapy, CAR-NK therapy, CAR-76 therapy, CAR-Macrophage therapy or any other cellular therapy with a genetically modified or genetically unmodified immune cell type.
92. The IL-2 conjugate for use of item 91, wherein the one or more additional drug is pembrolizumab.
93. The IL-2 conjugate for use of item 92 or 388, wherein pembrolizumab is administered every three weeks at a dose of 200 mg either prior to, together with or after administration of the IL-2 conjugate.
94. The IL-2 conjugate for use of item 92, 93 or 388, wherein pembrolizumab is administered every three weeks at a dose of 200 mg prior to administration of the IL-2 conjugate.
95. The IL-2 conjugate for use of item 92, 93 or 388, wherein pembrolizumab is administered every three weeks at a dose of 200 mg together with administration of the IL-2 conjugate.
96. The IL-2 conjugate for use of item 92, 93 or 388, wherein pembrolizumab is administered every three weeks at a dose of 200 mg after administration of the IL-2 conjugate.
97. The IL-2 conjugate for use of any one of items 92 to 96 and 388, wherein pembrolizumab is administered via intravenous infusion.
98. The IL-2 conjugate for use of item 91, wherein the one or more additional drug is of formula (A-3)

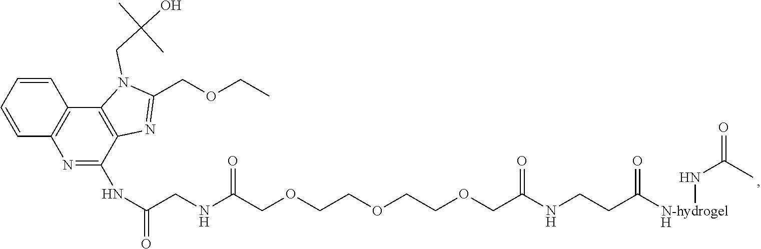

wherein “hydrogel” is a hydrogel comprising backbone moieties of formula (A-4):

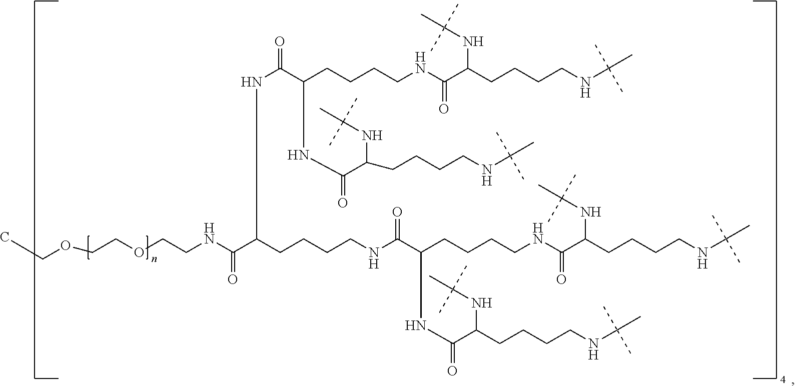

wherein n ranges from approx. 25 to 29 and dashed lines indicate attachment to either a moiety

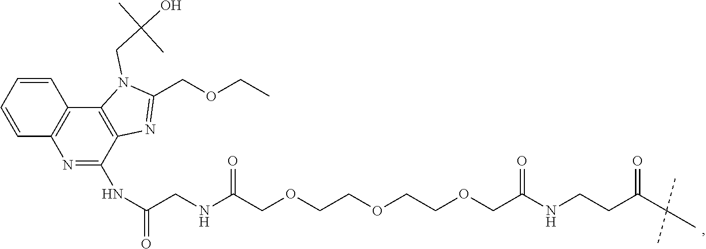

to a moiety

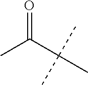

or to a crosslinker of formula (A-5):

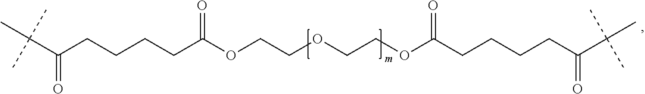

wherein m ranges from approx. 41 to 45 and the dashed lines in (A-5) indicate
attachment to a backbone moiety of formula (A-4).
99. The IL-2 conjugate for use of item 91, wherein the one or more additional drug is of formula (A-6)

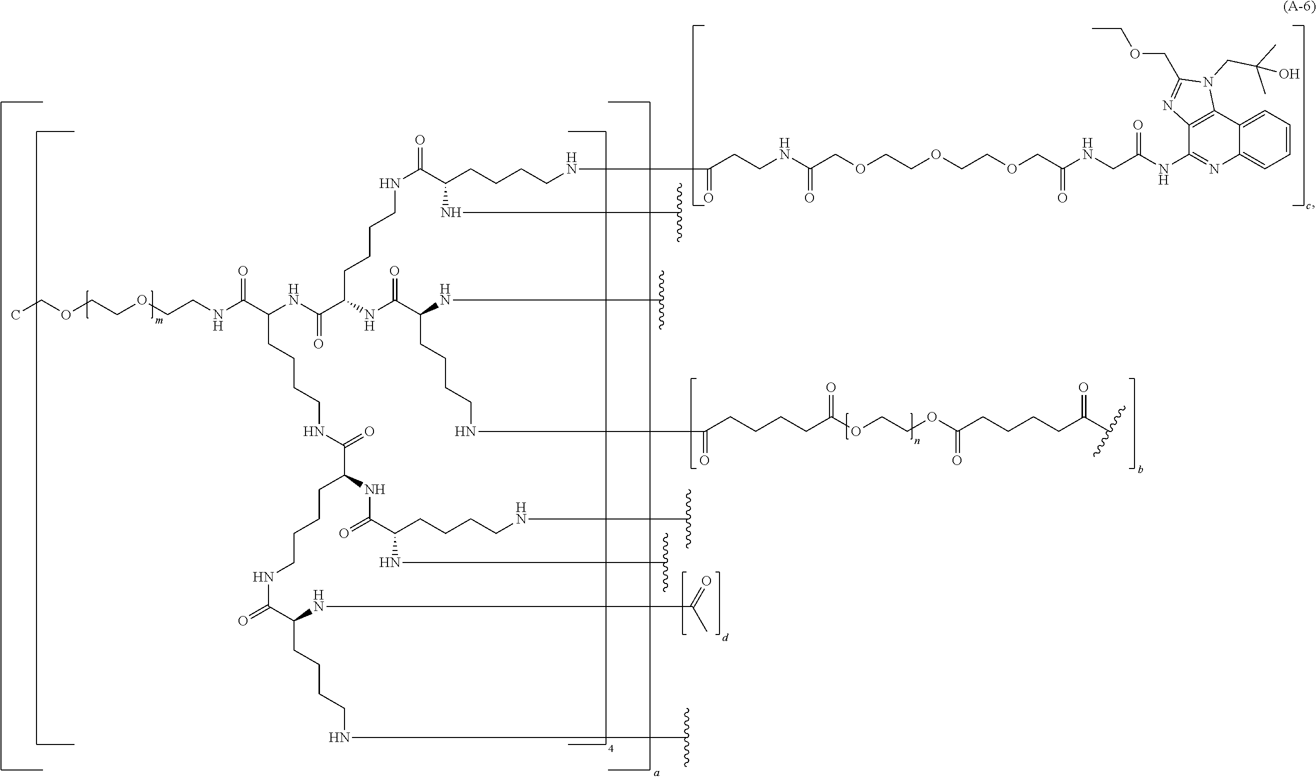

wherein
   m ranges from approx. 25 to 29;
   n ranges from approx. 41 to 45;
   “a” denotes a backbone moiety, “b” denotes a crosslinker moiety, “c” denotes a reversibly conjugated resiquimod moiety and “d” denotes an acetamide moiety; and the ratio of moieties “a”:“b”:“c”:“d” in the conjugate is approx. 1:13:2:4 or is approx. 1:13:2.4:3.6.
100. The IL-2 conjugate for use of item 91, wherein the one or more additional drug is of formula (Ai-6)

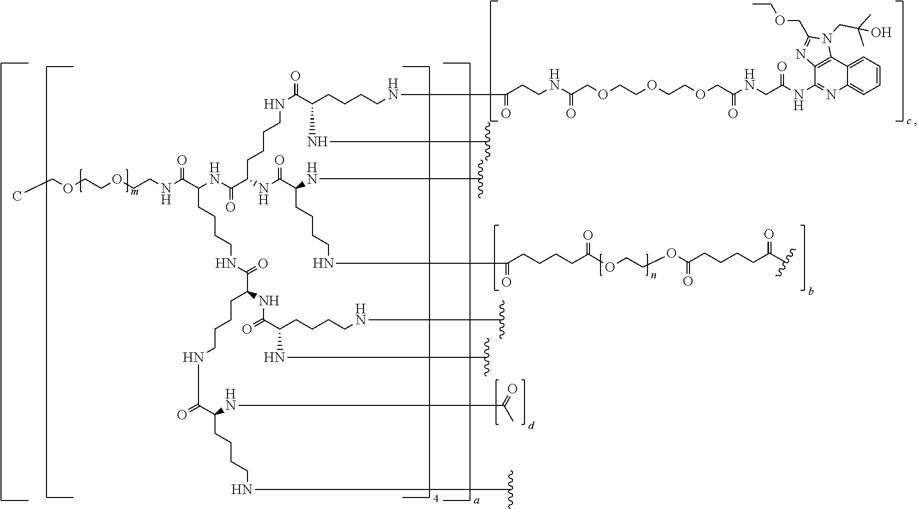

wherein
   m ranges from approx. 25 to 29;
   n ranges from approx. 41 to 45;
   “a” denotes a backbone moiety, “b” denotes a crosslinker moiety, “c” denotes a reversibly conjugated resiquimod moiety and “d” denotes an acetamide moiety; and the ratio of moieties “a”:“b”:“c”:“d” in the conjugate is approx. 1:13:c:d with c+d=6, such as a ratio of moieties “a”:“b”:“c”:“d” of approx. 1:13:3:3 or of approx. 1:13:2:4 or of approx. 1:13:4:2.
101. The IL-2 conjugate for use of any one of items 98 to 100, wherein the one or more additional drug is administered via intratumoral administration at a dose of 0.5 mg per lesion either prior to, together with or after administration of the IL-2 conjugate.
102. The IL-2 conjugate for use of any one of items 98 to 101, wherein the one or more additional drug is administered via intratumoral administration at a dose of 0.5 mg per lesion prior to administration of the IL-2 conjugate.
103. The IL-2 conjugate for use of any one of items 98 to 101, wherein the one or more additional drug is administered via intratumoral administration at a dose of 0.5 mg per lesion together with administration of the IL-2 conjugate.
104. The IL-2 conjugate for use of any one of items 98 to 101, wherein the one or more additional drug is administered via intratumoral administration at a dose of 0.5 mg per lesion after administration of the IL-2 conjugate.
105. The IL-2 conjugate for use of any one of items 98 to 104, wherein the one or more additional drug is administered every three weeks.
106. The IL-2 conjugate for use of any one of items 98 to 104, wherein the one or more additional drug is administered every six weeks.
107. The IL-2 conjugate for use of any one of items 98 to 104, wherein the one or more additional drug is administered at a frequency dependent on disease progression.
108. The IL-2 conjugate for use of any one of items 98 to 107, wherein the one or more additional drug is administered via intratumoral injection.
109. A unit dosage form comprising a unit dose of an IL-2 conjugate or a pharmaceutically acceptable salt thereof, wherein the unit dosage form comprises 80 μg/kg to 160 μg/kg IL-2 and wherein the IL-2 conjugate is of formula (I)

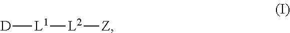

wherein
   —D is —D′—M_{mod},
      wherein
      —D′— is an IL-2 moiety;
      —M_{mod} is a first polymeric moiety that is stably conjugated to a side chain of an amino acid residue of —D′—;
   —L¹— is a linker moiety covalently and reversibly attached to —D′—;
   —L²— is absent or a spacer moiety; and
   —Z is a second polymeric moiety.
110. The unit dosage form of item 109, wherein the unit dosage form comprises 100 μg IL-2/kg to 140 μg IL-2/kg.
111. The unit dosage form of item 109 or 110, wherein the unit dosage form comprises 110 μg IL-2/kg to 130 μg IL-2/kg.
112. The unit dosage form of any one of items 109 to 111, wherein the unit dosage form comprises about 120 μg IL-2/kg.
113. The unit dosage form of any one of items 109 to 112, wherein the unit dosage form comprises 120 μg IL-2/kg.
114. The unit dosage form of any one of items 109 to 113, wherein the unit dosage form is for the treatment of cancer.
115. The unit dosage form of any one of items 109 to 114, wherein the unit dosage form is a vial.
116. The unit dosage form of any one of items 109 to 115, wherein the unit dosage form is for multiple administration.
117. The unit dosage form of item 116, wherein the interval between two consecutive administrations ranges from two to eight weeks.
118. The unit dosage form of item 116 or 117, wherein the interval between two consecutive administrations is three weeks.
119. The unit dosage form of item 116 or 117, wherein the interval between two consecutive administrations is six weeks.
120. The unit dosage form of any one of items 114 to 116, wherein administration occurs with a frequency dependent on disease progression.
121. The unit dosage form of item 114 or 115, wherein the unit dosage form is administered once.
122. The unit dosage form of any one of items 114 to 121, wherein the treatment starts directly on diagnosis of the cancer.
123. The unit dosage form of any one of items 114 to 122, wherein the treatment is a first line of treatment.
124. The unit dosage form of any one of items 114 to 122, wherein the treatment is the second, third or further line of treatment.
125. The unit dosage form of any one of items 114 to 124, wherein the unit dose of the unit dosage form is administered via external application, injection or infusion, including intraarticular, periarticular, intradermal, subcutaneous, intramuscular, intravenous, intraosseous, intraperitoneal, intrathecal, intracapsular, intraorbital, intravitreal, intratympanic, intravesical, intracardiac, transtracheal, subcuticular, subcapsular, subarachnoid, intraspinal, intraventricular, intrasternal injection and infusion; direct delivery to the brain via an implanted device allowing delivery of the IL-2 conjugate to brain tissue or brain fluids (e.g., Ommaya Reservoir), direct intracerebroventricular injection or infusion, injection or infusion into brain or brain associated regions, injection into the subchoroidal space, retro-orbital injection or ocular instillation.
126. The unit dosage form of any one of items 114 to 125, wherein the unit dose of the unit dosage form is administered intravenously.
127. The unit dosage form of any one of items 114 to 125, wherein the unit dose of the unit dosage form is administered subcutaneously.
128. The unit dosage form of any one of items 109 to 127, wherein —D′— has at least 95% sequence identity to the amino acid sequence of SEQ ID NO:1.
129. The unit dosage form of any one of items 109 to 128, wherein —D′— has at least 96% sequence identity to the amino acid sequence of SEQ ID NO:1.
130. The unit dosage form of any one of items 109 to 129, wherein —D′— has at least 97% sequence identity to the amino acid sequence of SEQ ID NO:1.
131. The unit dosage form of any one of items 109 to 130, wherein —D′— has at least 98% sequence identity to the amino acid sequence of SEQ ID NO:1.
132. The unit dosage form of any one of items 109 to 131, wherein —D′— has at least 99% sequence identity to the amino acid sequence of SEQ ID NO:1.
133. The unit dosage form of any one of items 109 to 132, wherein —D′— comprises the amino acid sequence of SEQ ID NO:1, in which one amino acid residue selected from the group consisting of K35, R38, M39, T41, F42, K43, F44, Y45, E62, P65, E68, V69, and L72 is replaced with a proteinogenic or non-proteinogenic amino acid residue.
134. The unit dosage form of any one of items 109 to 133, wherein —D′— comprises the Aldel mutation.
135. The unit dosage form of any one of items 109 to 134, wherein —D′— comprises the amino acid sequence of SEQ ID NO:1 in which one amino acid residue selected from the group consisting of K35, R38, M39, T41, F42, K43, F44, Y45, E62, P65, E68, V69, and L72 is replaced with a cysteine residue.
136. The unit dosage form of any one of items 109 to 133 or 135, wherein —D′— has the amino acid sequence of SEQ ID NO:1 in which one amino acid residue selected from the group consisting of K35, R38, M39, T41, F42, K43, F44, Y45, E62, P65, E68, V69, and L72 is replaced with a cysteine residue.
137. The unit dosage form of any one of items 109 to 133, 135 or 136, wherein —D′— has the amino acid sequence of SEQ ID NO:4.
138. The unit dosage form of any one of items 109 to 137, wherein —M_{mod} has a molecular weight ranging from 0.5 kDa to 20 kDa.
139. The unit dosage form of any one of items 109 to 138, wherein —M_{mod} has a molecular weight ranging from 1 kDa to 20 kDa.
140. The unit dosage form of any one of items 109 to 139, wherein —M_{mod} has a molecular weight ranging from 2 kDa to 10 kDa.
141. The unit dosage form of any one of items 109 to 140, wherein —M_{mod} has a molecular weight ranging from 3 kDa to 7 kDa.
142. The unit dosage form of any one of items 109 to 141, wherein —M_{mod} has a molecular weight of 5±25% kDa.
143. The unit dosage form of any one of items 109 to 142, wherein —M_{mod} has a molecular weight ranging from 4 kDa to 6 kDa.
144. The unit dosage form of any one of items 109 to 142, wherein —M_{mod} has a molecular weight of 5±20% kDa.
145. The unit dosage form of any one of items 109 to 144, wherein —M_{mod} has a molecular weight of 5±15% kDa.
146. The unit dosage form of any one of items 109 to 145, wherein —M_{mod} has a molecular weight of about 5 kDa.
147. The unit dosage form of any one of items 109 to 146, wherein —M_{mod} is a PEG-based polymer.
148. The unit dosage form of any one of items 109 to 147, wherein attachment of —M_{mod} occurs at an amino acid position selected from the group consisting of K35, R38, M39, T41, F42, K43, F44, Y45, E62, P65, E68, V69, and L72.
149. The unit dosage form of any one of items 109 to 133 or 135 to 148, wherein —D′— has the amino acid sequence of SEQ ID NO. 4 and —M_{mod} is stably conjugated to the sulfur atom of the side chain of the cysteine at position 38. 150. The unit dosage form of any one of items 109 to 149, wherein —M_{mod} is of formula (A-1)

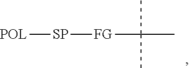

wherein
   —FG— is a linkage;
   —SP— is a spacer moiety; and
   —POL is a polymer.
151. The unit dosage form of item 150, wherein —FG— of formula (A-1) is of formula (FG-1a)

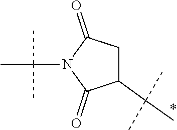

wherein
   the dashed line marked with the asterisk indicates attachment to the sulfur of a cysteine side chain of —D′— and the unmarked dashed line indicates attachment to —SP—.
152. The unit dosage form of item 150, wherein embodiments —FG— of formula (A-1) is of formula (FG-1d) or (FG-1e)

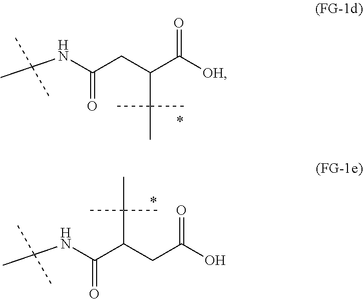

wherein the dashed line marked with the asterisk indicates attachment to the sulfur of a cysteine residue of —D′— and the unmarked dashed line indicates attachment to —SP—.
153. The unit dosage form of any one of items 150 to 152, wherein —SP— is C₁₋₁₀ alkyl, which C₁₋₁₀ alkyl is optionally substituted with one or more —R⁹, and which C₁₋₁₀ alkyl is optionally interrupted by one or more groups selected from the group consisting of —O—, —C(O)N(R¹⁰)—, —S(O)₂—, —S(O)—, —S—, —N(R¹⁰)—, —OC(OR¹⁰)(R^{10a})—, —N(R¹⁰)C(O)N(R^{10a})—, and —OC(O)N(R¹⁰)—; wherein each —R⁹ is selected from the group consisting of C₁₋₆ alkyl; and each —R¹⁰ and —R^{10a} is independently selected from the group consisting of —H and C₁₋₆ alkyl.
154. The unit dosage form of any one of items 150 to 153, wherein —POL is of formula (A-11)

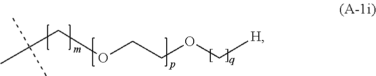

wherein
   the dashed line indicates attachment to —SP—;
   m is 0 or 1;
   p is an integer ranging from about 10 to about 540; and
   q is selected from the group consisting of 1, 2, 3, 4, 5, and 6.
155. The unit dosage form of any one of items 109 to 151, 153 or 154, wherein —M_{mod} is of formula (A-1a)

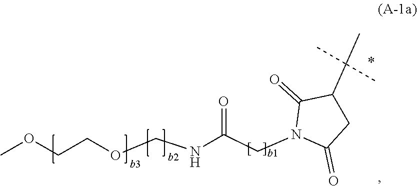

wherein
   the dashed line marked with the asterisk indicates attachment to the sulfur of a cysteine residue of —D′—;
   b1 is an integer selected from the group consisting of 1, 2, 3, 4, 5, 6, 7, 8, 9, 10, 11, 12, 13, 14, 15, 16, 17, 18, 19 and 20;
   b2 is an integer selected from the group consisting of 1, 2, 3, 4, 5, 6, 7, 8, 9, 10, 11, 12, 13, 14, 15, 16, 17, 18, 19 and 20; and
   b3 is an integer ranging from about 10 to about 540.
156. The unit dosage form of any one of items 109 to 150 or 152 to 154, wherein —M_{mod} is of formula (A-1d) or (A-1e)

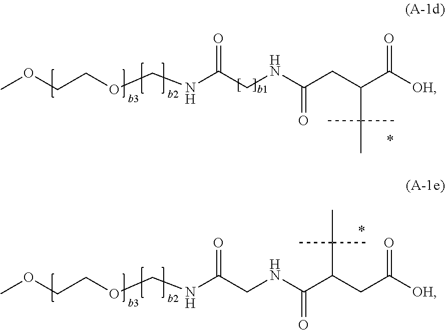

wherein
   the dashed line marked with the asterisk indicates attachment to the sulfur of a cysteine residue of —D′—;
   b1 is an integer selected from the group consisting of 1, 2, 3, 4, 5, 6, 7, 8, 9, 10, 11, 12, 13, 14, 15, 16, 17, 18, 19 and 20;
   b2 is an integer selected from the group consisting of 1, 2, 3, 4, 5, 6, 7, 8, 9, 10, 11, 12, 13, 14, 15, 16, 17, 18, 19 and 20; and b3 is an integer ranging from about 10 to about 540.
157. The unit dosage form of items 155 or 156, wherein b1 is an integer ranging from 1 to 4.
158. The unit dosage form of any one of items 155 to 157, wherein b1 is 2.
159. The unit dosage form of any one of items 155 to 157, wherein b1 is 3.
160. The unit dosage form of any one of items 155 to 159, wherein b2 is 2.
161. The unit dosage form of any one of items 155 to 159, wherein b2 is 3.
162. The unit dosage form of any one of items 155 to 161, wherein b3 is an integer ranging from 10 to 450.
163. The unit dosage form of any one of items 155 to 162, wherein b3 is an integer ranging from 22 to 340.
164. The unit dosage form of any one of items 155 to 163, wherein b3 is an integer ranging from 45 to 230.
165. The unit dosage form of any one of items 155 to 164, wherein b3 is an integer ranging from 65 to 160.
166. The unit dosage form of any one of items 155 to 165, wherein b3 is selected such that —M_{mod} has a molecular weight of 5±25% kDa.
167. The unit dosage form of any one of items 155 to 166, wherein b3 is an integer ranging from 90 to 140.
168. The unit dosage form of any one of items 155 to 167, wherein b3 is selected such that —M_{mod} has a molecular weight ranging from about 4 kDa to about 6 kDa.
169. The unit dosage form of any one of items 155 to 168, wherein b3 is selected such that —M_{mod} has a molecular weight of 5 20% kDa.
170. The unit dosage form of any one of items 155 to 169, wherein b3 is an integer ranging from 95 to 135.
171. The unit dosage form of any one of items 155 to 170, wherein b3 is selected such that —M_{mod} has a molecular weight of 5 15% kDa.
172. The unit dosage form of any one of items 155 to 171, wherein b3 is an integer ranging from 100 to 125.
173. The unit dosage form of any one of items 155 to 172, wherein b3 is selected such that —M_{mod} has a molecular weight of about 5 kDa.
174. The unit dosage form of any one of items 109 to 173, wherein —Z has a molecular weight ranging from about 20 kDa to about 60 kDa.
175. The unit dosage form of any one of items 109 to 174, wherein —Z has a molecular weight ranging from about 25 kDa to about 55 kDa.
176. The unit dosage form of any one of items 109 to 175, wherein —Z has a molecular weight ranging from about 30 kDa to about 50 kDa.
177. The unit dosage form of any one of items 109 to 176, wherein —Z has a molecular weight ranging from about 35 kDa to about 45 kDa.
178. The unit dosage form of any one of items 109 to 177, wherein —Z has a molecular weight ranging of about 40 kDa.
179. The unit dosage form of any one of items 109 to 178, wherein —Z comprises a moiety of formula (A-c):

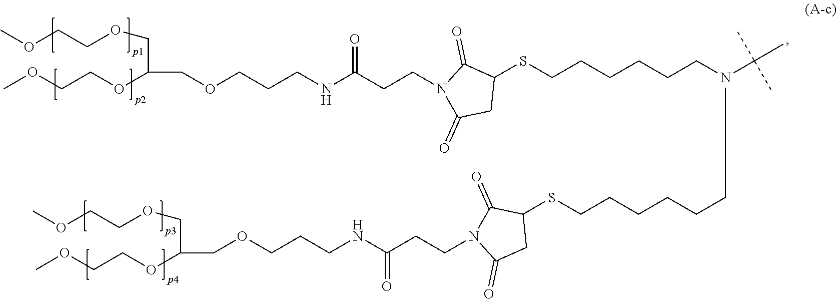

wherein
   p1, p2, p3, p4 are independently of each other an integer ranging from 110 to 345
180. The unit dosage form of item 179, wherein p1, p2, p3, p4 of formula (A-c) are independently of each other an integer ranging from 140 to 315.
181. The unit dosage form of any one of items 179 to 180, wherein p1, p2, p3, p4 of formula (A-c) are independently of each other an integer ranging from 170 to 285.
182. The unit dosage form of any one of items 179 to 181, wherein p1, p2, p3, p4 of formula (A-c) are independently of each other an integer ranging from 195 to 255.
183. The unit dosage form of any one of items 179 to 182, wherein p1, p2, p3, p4 of formula (A-c) are independently of each other an integer ranging from 200 to 250.
184. The unit dosage form of any one of items 179 to 183, wherein p1, p2, p3, p4 of formula (A-c) are independently of each other an integer ranging from 210 to 250.
185. The unit dosage form of any one of items 179 to 184, wherein p1, p2, p3, p4 of formula (A-c) are independently of each other an integer ranging from 220 to 240.
186. The unit dosage form of any one of items 109 to 185, wherein —M_{mod} is attached to a different amino acid residue of —D′— than the moiety —L¹—L²—Z.
187. The unit dosage form of any one of items 109 to 186, wherein —L¹— is attached to a lysine residue of —D′—.
188. The unit dosage form of any one of items 109 to 187, wherein the linkage between —D′— and —L¹— is a carbamate.
189. The unit dosage form of any one of items 109 to 188, wherein —L¹— is of formula (IX-a):

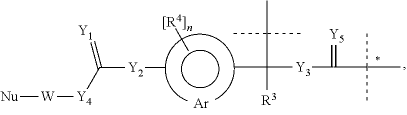

wherein
   the dashed line marked with the asterisk indicates attachment to a nitrogen of —D′— and the unmarked dashed line indicates attachment to —L²—Z;
   n is 0,1,2,3 or 4;
   ═Y₁ is selected from the group consisting of ═O and ═S;
   —Y₂— is selected from the group consisting of —O— and —S—;
   —Y₃— is selected from the group consisting of —O— and —S—;
   —Y₄— is selected from the group consisting of —O—, —NR⁵— and —C(R⁶R^{6a})—;
   ═Y₅ is selected from the group consisting of ═O and ═S;
   —R³, —R⁵, —R⁶, —R^{6a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl;
   —R⁴ is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl;
   —W— is selected from the group consisting of C₁₋₂₀ alkyl optionally interrupted by one or more groups selected from the group consisting of C₃₋₁₀ cycloalkyl, 8- to 30-membered carbopolycyclyl, 3- to 10-membered heterocyclyl, —C(O)—, —C(O)N(R⁷)—, —O—, —S— and —N(R⁷)—;
   —Nu is a nucleophile selected from the group consisting of —N(R⁷R^{7a}), —N(R⁷OH), —N(R⁷)—N(R^{7a}R^{7b}), —S(R⁷),—COOH,

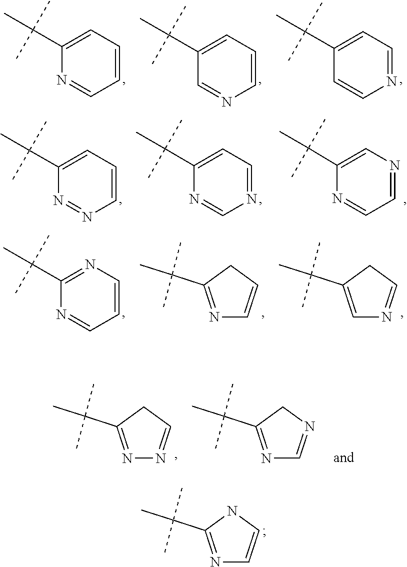

—Ar— is selected from the group consisting of

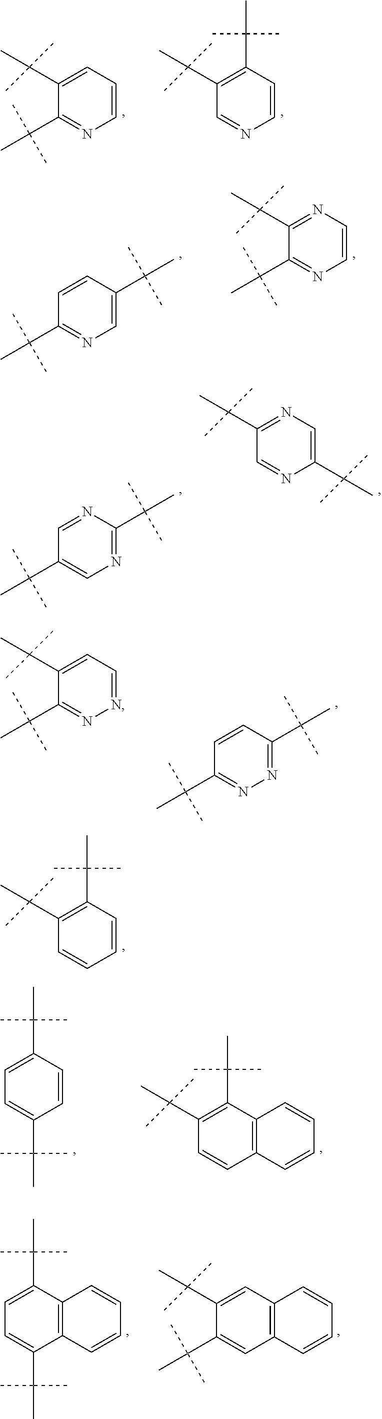

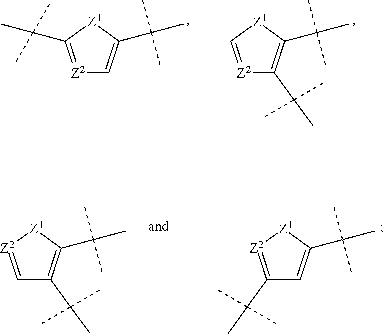

wherein
   dashed lines indicate attachment to the remainder of —L¹—,
   —Z¹— is selected from the group consisting of —O—, —S— and —N(R⁷)—, and
   —Z²—0 is —N(R⁷)—; and
   —R⁷, —R^{7a}, —R^{7b} are independently of each other selected from the group consisting of —H, C₁₋₆ alkyl, C₂₋₆ alkenyl and C₂₋₆ alkynyl;
wherein —L¹— is optionally further substituted.
190. The unit dosage form of any one of items 109 to 189, wherein —L¹— is of formula (IX-d)

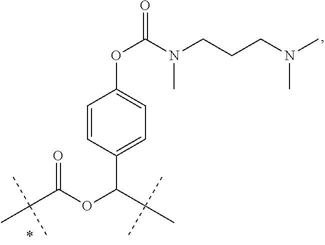

wherein
   the dashed line marked with the asterisk indicates attachment to a nitrogen of —D′—; and
   the unmarked dashed line indicates attachment to —L²—Z.
191. The unit dosage form of item 190, wherein the dashed line marked with the asterisk indicates attachment to the nitrogen of the ε-amino group of a lysine residue of —D′— or to the nitrogen of the N-terminal amine of —D′—.
192. The unit dosage form of item 190 or 191, wherein the dashed line marked with the asterisk indicates attachment to the nitrogen of the ε-amino group of a lysine residue of —D′—.
193. The unit dosage form of any one of items 109 to 192, wherein —L²— is a C₁₋₂₀ alkyl chain, which is optionally interrupted by one or more groups independently selected from —O—, —C(O)—, —T— and —C(O)N(R^{y1})—; and which C₁₋₂₀ alkyl chain is optionally substituted with one or more groups independently selected from —OH, —T and —C(O)N(R⁶R^{y6a}); wherein —R¹, —R^{y6}, —R^{y6a} are independently selected from the group consisting of H and C₁₋₄ alkyl and wherein T is selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl.
194. The unit dosage form of any one of items 109 to 193, wherein —L¹—L²—Z is of formula (XI)

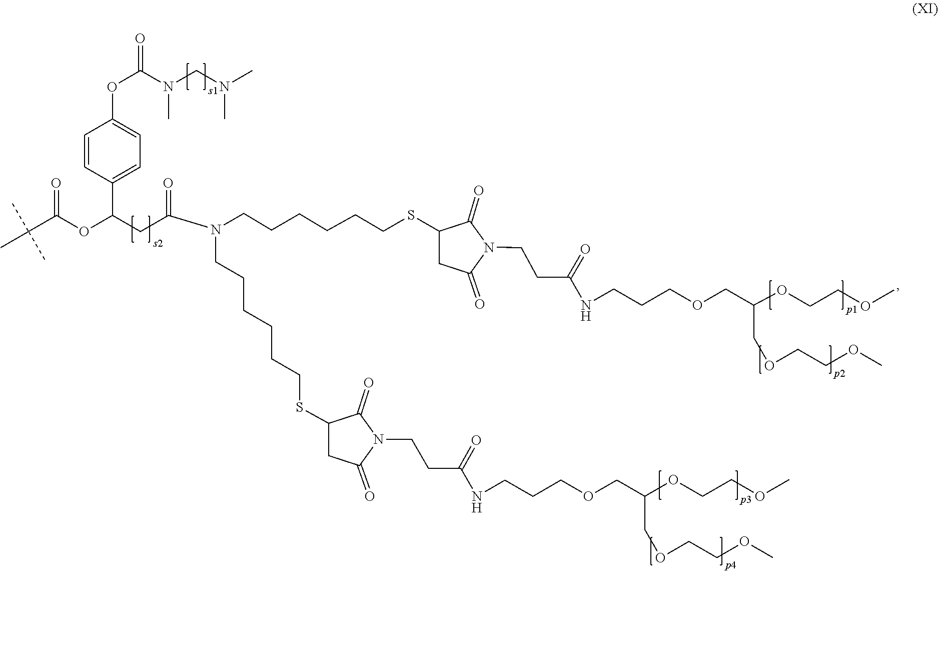

wherein the dashed line indicates attachment to a nitrogen of —D′—;
   s1 is an integer selected from the group consisting of 1, 2, 3, 4, 5, 6, 7, 8, 9 and 10;
   s2 is an integer selected from the group consisting of 0, 1, 2, 3, 4, 5, 6, 7, 8, 9, 10, 11, 12, 13, 14, 15, 16, 17, 18, 19 and 20; and
   p1, p2, p3, p4 are independently of each other an integer ranging from 110 to 345.
195. The unit dosage form of item 194, wherein the dashed line indicates attachment to the nitrogen of the ε-amino group of a lysine residue of —D′— or to the nitrogen of the N-terminal amine of —D′—.
196. The unit dosage form of item 194 or 195, wherein the dashed line indicates attachment to the nitrogen of the ε-amino group of a lysine residue of —D′—.
197. The unit dosage form of any one of items 109 to 196, wherein the IL-2 conjugate is of formula (I), wherein —D is —D′—M_{mod} and wherein —D′— has the amino acid sequence of SEQ ID NO:4, —M_{mod} has the structure of (A-1d) or (A-1e)

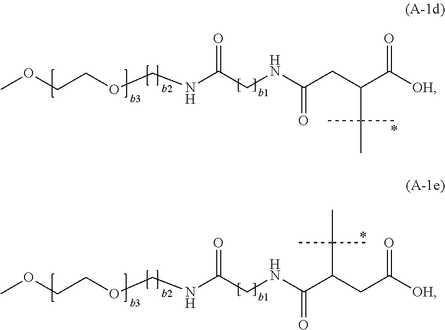

wherein
   the dashed line marked with the asterisk indicates attachment to the sulfur atom of the
   side chain of the cysteine at position 38;
   b1 is 2;
   b2 is 2;
   b3 ranges from 90 to 140, from 95 to 135 or from 100 to 125;
   and wherein —L¹—L²—Z is of formula (XI-a)

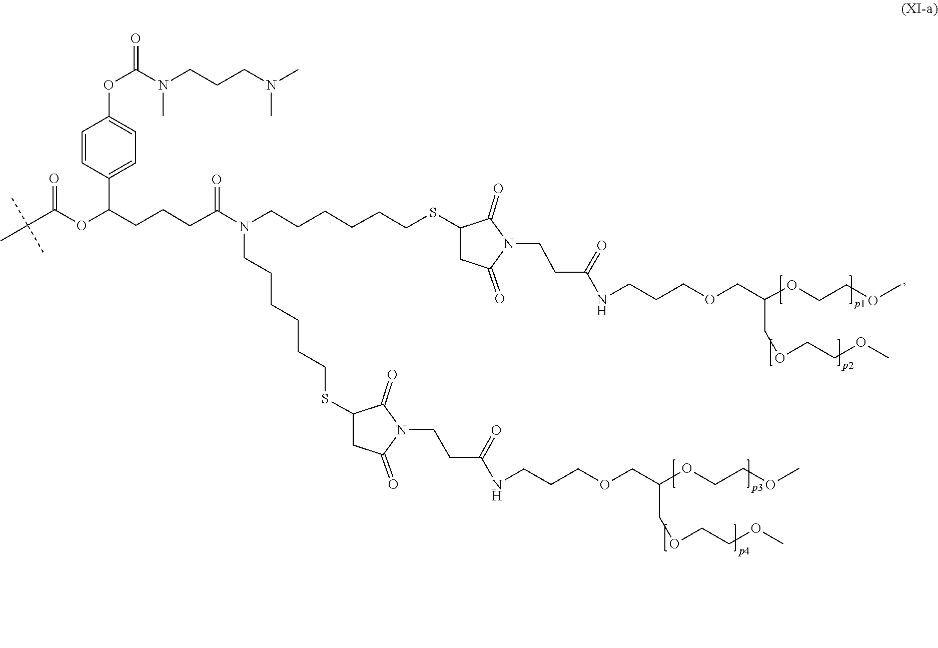

Wherein the dashed line indicates attachment to the nitrogen of the ε-amino group of
   a lysine residue of —D′— or to the nitrogen of the N-terminal amine of —D′—; and p1, p2, p3, p4 are independently of each other an integer ranging from 110 to 345, from 140 to 315, from 170 to 285, from 195 to 255, from 200 to 250, from 210 to 250 or from 220 to 240.
198. The unit dosage form of any one of items 114 to 197, wherein the cancer is selected from the group consisting of liquid tumors, solid tumors and lymphomas.
199. The unit dosage form of item 198, wherein the liquid tumor is a leukemia or myeloid neoplasm, such as chronic lymphocytic leukemia (CLL), chronic myelogenous leukemia (CML), hairy cell leukemia, lymphoblastic leukemia, myeloid leukemia, plasma cell leukemia, acute lymphoblastic leukemia (ALL), acute myeloid leukemia (AML), myelodysplastic syndrome (MDS), myeloproliferative neoplasm (MPN), post-MPN AML, post-MDS AML, del(5q)-associated high risk MDS or AIL, blast-phase chronic myelogenous leukemia, multiple myeloma, myelodysplastic syndromes, chronic myeloproliferative disorders, plasma cell neoplasm and Waldenstrom's macroglobulinemia.
200. The unit dosage form of item 198, wherein the solid tumor or lymphoma is selected from the group consisting of lip and oral cavity cancer, oral cancer, liver cancer/hepatocellular cancer, primary liver cancer, lung cancer, lymphoma, malignant mesothelioma, malignant thymoma, skin cancer, intraocular melanoma, metastatic squamous neck cancer with occult primary, childhood multiple endocrine neoplasia syndrome, mycosis fungoides, nasal cavity and paranasal sinus cancer, nasopharyngeal cancer, neuroblastoma, oropharyngeal cancer, ovarian cancer, pancreatic cancer, parathyroid cancer, pheochromocytoma, pituitary tumor, adrenocortical carcinoma, AIDS-related malignancies, anal cancer, bile duct cancer, bladder cancer, brain and nervous system cancer, breast cancer, bronchial adenoma/carcinoid, gastrointestinal carcinoid tumor, carcinoma, colorectal cancer, endometrial cancer, esophageal cancer, extracranial germ cell tumor, extragonadal germ cell tumor, extrahepatic bile duct cancer, gallbladder cancer, gastric (stomach) cancer, gestational trophoblastic tumor, head and neck cancer, hypopharyngeal cancer, islet cell carcinoma (endocrine pancreas), kidney cancer/renal cell cancer, laryngeal cancer, pleuropulmonary blastoma, prostate cancer, transitional cell cancer of the renal pelvis and ureter, retinoblastoma, salivary gland cancer, sarcoma, Sezary syndrome, small intestine cancer, genitourinary cancer, malignant thymoma, thyroid cancer, Wilms' tumor, cholangiocarcinoma, and also their related earlier stages of aberrant cell growth such as dysplasias, adenomas, and carcinoma in situs.
201. The unit dosage form of any one of items 114 to 200, 351 to 383, and 392 to 397, wherein the IL-2 conjugate is administered to the patient prior to, simultaneously with or after administration of one or more additional drug, which one or more additional drug is selected from the group consisting of pattern recognition receptor agonists (PRRA), cytotoxic/chemotherapeutic agents, immune checkpoint inhibitors or antagonists, immune checkpoint agonists, immune activating receptor agonists, multi-specific drugs, antibody-drug conjugates (ADC), antibody-adjuvant conjugates (AAC), radionuclides or targeted radionuclide therapeutics, DNA damage repair inhibitors, tumor metabolism inhibitors, pattern recognition receptor agonists, protein kinase inhibitors, chemokine and chemoattractant receptor agonists, chemokine or chemokine receptor antagonists, cytokine receptor agonists, death receptor agonists, CD47 or SIRPα antagonists, oncolytic drugs, signal converter proteins, epigenetic modifiers, tumor peptides or tumor vaccines, heat shock protein (HSP) inhibitors, proteolytic enzymes, ubiquitin and proteasome inhibitors, adhesion molecule antagonists, hormones including hormone peptides and synthetic hormones, and adoptive cellular therapies such as Tumor Infiltrating Lymphocyte (TIL) therapy, Chimeric Antigen Receptor (CAR) therapy, T cell therapy, Natural Killer (NK) cell therapy, CAR-T therapy, CAR-NK therapy, CAR-76 therapy, CAR-Macrophage therapy or any other cellular therapy with a genetically modified or genetically unmodified immune cell type.
202. The unit dosage form of item 201, wherein the one or more additional drug is pembrolizumab.
203. The unit dosage form of item 202 or 392, wherein pembrolizumab is administered every three weeks at a dose of 200 mg either prior to, together with or after administration of the IL-2 conjugate.
204. The unit dosage form of item 202, 203 or 392, wherein pembrolizumab is administered every three weeks at a dose of 200 mg prior to administration of the IL-2 conjugate.
205. The unit dosage form of item 202, 203 or 392, wherein pembrolizumab is administered every three weeks at a dose of 200 mg together with administration of the IL-2 conjugate.
206. The unit dosage form of item 202, 203 or 392, wherein pembrolizumab is administered every three weeks at a dose of 200 mg after administration of the IL-2 conjugate.
207. The unit dosage form of any one of items 202 to 206 and 392, wherein pembrolizumab is administered via intravenous infusion.
208. The unit dosage form of item 201, wherein the one or more additional drug is of formula (A-3)

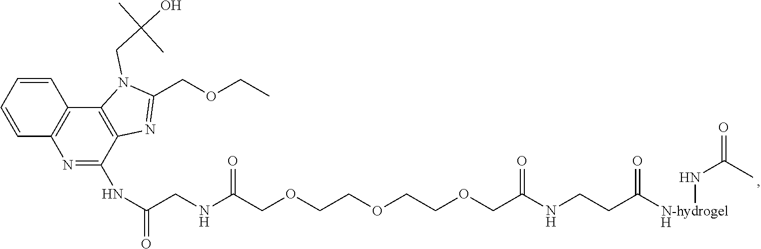

wherein “hydrogel” is a hydrogel comprising backbone moieties of formula (A-4):

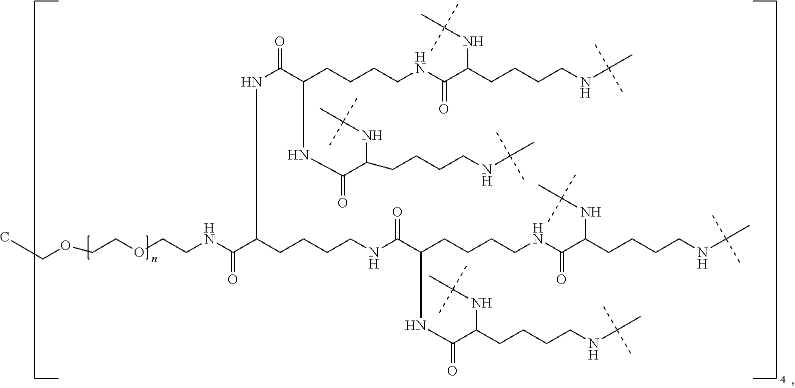

wherein n ranges from approx. 25 to 29 and dashed lines indicate attachment to either a moiety

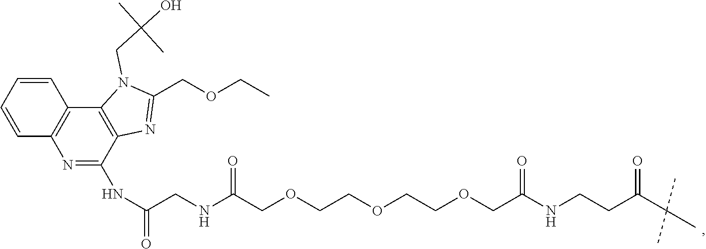

to a moiety

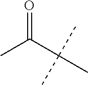

or to a crosslinker of formula (A-5):

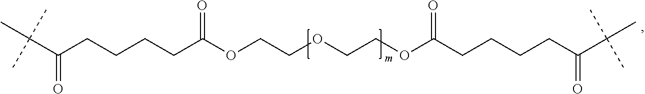

wherein m ranges from approx. 41 to 45 and the dashed lines in (A-5) indicate attachment to a backbone moiety of formula (A-4).
209. The unit dosage form of item 201, wherein the one or more additional is of formula (A-6)

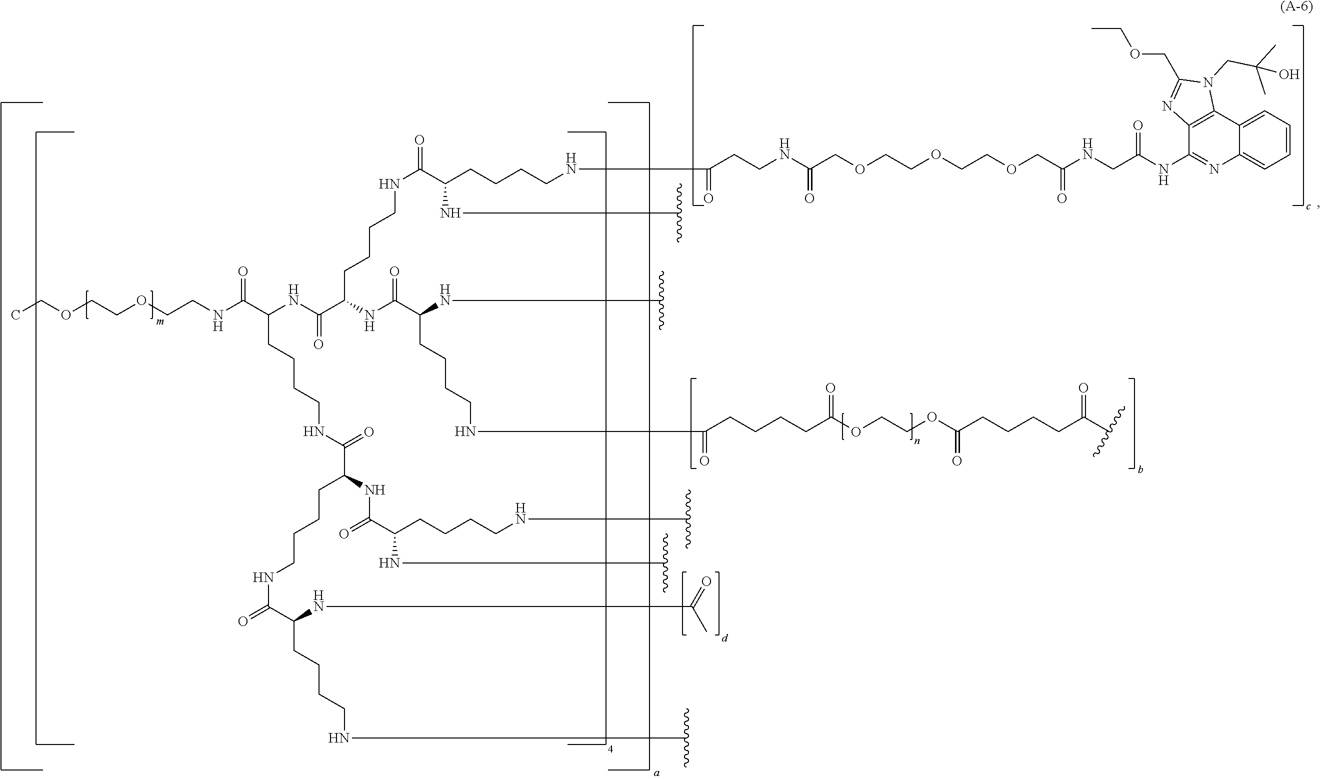

wherein
   m ranges from approx. 25 to 29;
   n ranges from approx. 41 to 45;
   “a” denotes a backbone moiety, “b” denotes a crosslinker moiety, “c” denotes a reversibly conjugated resiquimod moiety and “d” denotes an acetamide moiety; and the ratio of moieties “a”:“b”:“c”:“d” in the conjugate is approx. 1:13:2:4 or is approx. 1:13:2.4:3.6.
210. The unit dosage form of item 201, wherein the one or more additional drug is of formula (Ai-6)

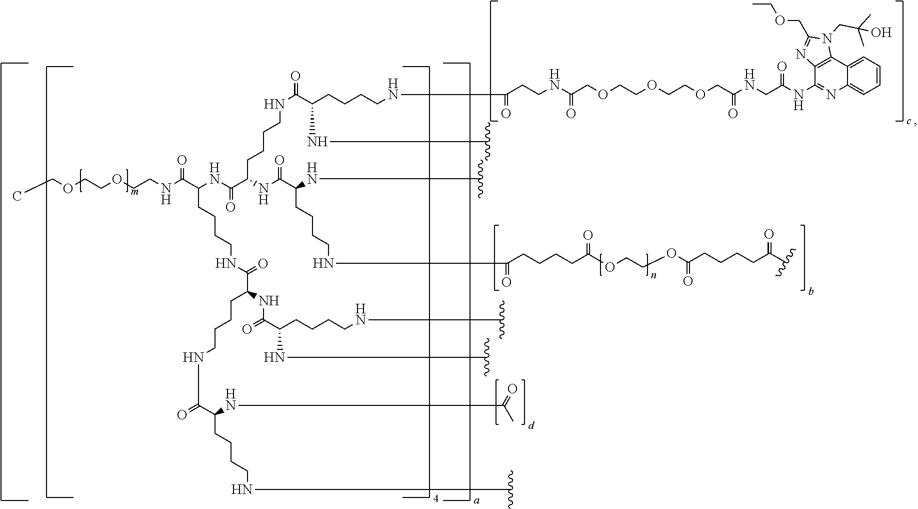

wherein
   m ranges from approx. 25 to 29;
   n ranges from approx. 41 to 45;
   “a” denotes a backbone moiety, “b” denotes a crosslinker moiety, “c” denotes a reversibly conjugated resiquimod moiety and “d” denotes an acetamide moiety; and the ratio of moieties “a”:“b”:“c”:“d” in the conjugate is approx. 1:13:c:d with c+d=6, such as the ratio of moieties “a”:“b”:“c”:“d” is approx. 1:13:3:3 or is approx. 1:13:2:4 or is approx. 1:13:4:2.
211. The unit dosage form of any one of items 208 to 210, wherein the one or more additional drug is administered via intratumoral administration at a dose of 0.5 mg per lesion either prior to, together with or after administration of the IL-2 conjugate.
212. The unit dosage form of any one of items 208 to 211, wherein the one or more additional drug is administered via intratumoral administration at a dose of 0.5 mg per lesion prior to administration of the IL-2 conjugate.
213. The unit dosage form of any one of items 208 to 211, wherein the one or more additional drug is administered via intratumoral administration at a dose of 0.5 mg per lesion together with administration of the IL-2 conjugate.
214. The unit dosage form of any one of items 208 to 211, wherein the one or more additional drug is administered via intratumoral administration at a dose of 0.5 mg per lesion after administration of the IL-2 conjugate.
215. The unit dosage form of any one of items 208 to 214, wherein the one or more additional drug is administered every three weeks.
216. The unit dosage form of any one of items 208 to 214, wherein the one or more additional drug is administered every six weeks.
217. The unit dosage form of any one of items 208 to 214, wherein the one or more additional drug is administered at a frequency dependent on disease progression.
218. The unit dosage form of any one of items 208 to 217, wherein the drug of formula (A-3), (A-6) or (Ai-6) is administered via intratumoral injection.
219. A kit-of-parts comprising the IL-2 conjugate or a pharmaceutically acceptable salt thereof of any one of items 1 to 108 or the unit dosage form of any one of items 109 to 218.
220. The kit-of-parts of item 219, wherein the kit-of-parts further comprises at least one container.
221. The kit-of-parts of item 219 or 220, wherein the container is a sterile container selected from the group consisting of boxes, ampoules, bottles, vials, tubes, bags, pouches, blister-packs, syringes, dual-chamber syringes and other suitable container forms for holding a medicament.
222. The kit-of-parts of any one of items 219 to 221, wherein the kit-of-parts further comprises instructions in a readable medium for dosing and/or administration of the IL-2 conjugate or a pharmaceutically acceptable salt thereof or the unit dosage form.
223. The kit-of-parts of any one of items 219 to 222, wherein the kit-of-parts comprises the IL-2 conjugate or a pharmaceutically acceptable salt thereof provided in a glass vial and instructions in a readable medium for dosing and/or administration of the IL-2 conjugate or a pharmaceutically acceptable salt thereof.
224. The kit-of-parts of any one of items 219 to 223, wherein the kit-of-parts comprises a first container comprising the IL-2 conjugate or a pharmaceutically acceptable salt thereof and a second container comprising one or more additional drug.
225. The kit-of-parts of any one of items 219 to 224, wherein the kit-of-parts comprises a first container comprising the IL-2 conjugate or a pharmaceutically acceptable salt thereof and a second container comprising pembrolizumab.
226. The kit-of-parts of any one of items 219 to 225 for use in the treatment of cancer.
227. The kit-of-parts for use of item 226, wherein the cancer is selected from the group consisting of liquid tumors, solid tumors and lymphomas.
228. The kit-of-parts for use of item 227, wherein the liquid tumor is a leukemia or myeloid neoplasm, such as chronic lymphocytic leukemia (CLL), chronic myelogenous leukemia (CMIL), hairy cell leukemia, lymphoblastic leukemia, myeloid leukemia, plasma cell leukemia, acute lymphoblastic leukemia (ALL), acute myeloid leukemia (AML), myelodysplastic syndrome (MDS), myeloproliferative neoplasm (MPN), post-MPN AML, post-MDS AML, del(5q)-associated high risk MDS or AML, blast-phase chronic myelogenous leukemia, multiple myeloma, myelodysplastic syndromes, chronic myeloproliferative disorders, plasma cell neoplasm and Waldenstrom's macroglobulinemia.
229. The kit-of-parts for use of item 227, wherein the solid tumor or lymphoma is selected from the group consisting of lip and oral cavity cancer, oral cancer, liver cancer/hepatocellular cancer, primary liver cancer, lung cancer, lymphoma, malignant mesothelioma, malignant thymoma, skin cancer, intraocular melanoma, metastatic squamous neck cancer with occult primary, childhood multiple endocrine neoplasia syndrome, mycosis fungoides, nasal cavity and paranasal sinus cancer, nasopharyngeal cancer, neuroblastoma, oropharyngeal cancer, ovarian cancer, pancreatic cancer, parathyroid cancer, pheochromocytoma, pituitary tumor, adrenocortical carcinoma, AIDS-related malignancies, anal cancer, bile duct cancer, bladder cancer, brain and nervous system cancer, breast cancer, bronchial adenoma/carcinoid, gastrointestinal carcinoid tumor, carcinoma, colorectal cancer, endometrial cancer, esophageal cancer, extracranial germ cell tumor, extragonadal germ cell tumor, extrahepatic bile duct cancer, gallbladder cancer, gastric (stomach) cancer, gestational trophoblastic tumor, head and neck cancer, hypopharyngeal cancer, islet cell carcinoma (endocrine pancreas), kidney cancer/renal cell cancer, laryngeal cancer, pleuropulmonary blastoma, prostate cancer, transitional cell cancer of the renal pelvis and ureter, retinoblastoma, salivary gland cancer, sarcoma, Sezary syndrome, small intestine cancer, genitourinary cancer, malignant thymoma, thyroid cancer, Wilms' tumor, cholangiocarcinoma, and also their related earlier stages of aberrant cell growth such as dysplasias, adenomas, and carcinoma in situs.
230. A method of synthesizing the IL-2 conjugate of formula (I) of any one of items 1 to 108, wherein the method comprises the steps of
   a) providing
      a)i. at least one reagent Z—L²—L¹—FG;
      a)ii. a liquid comprising at least one organic solvent; and
      a)iii. one or more drug D—H;
   b) combining and incubating the at least one reagent Z—L²—L¹—FG from step a)i., the liquid from step a)ii. and the one or more drug D—H from step a)iii. to obtain the IL-2 conjugate of formula (I); and
   c) optionally, purifying the IL-2 conjugate of formula (I) obtained in step b),
wherein
—D, —L¹—, —L²— and —Z are defined as in any one of items 1 to 108;
D—H is the free form of —D; and
—FG is a functional group moiety.
231. The method of item 230, wherein the liquid of step a)ii. comprises at least about 5% (v/v) of the organic solvent, such as ranging from about 5% to about 80% (v/v), ranging from about 10% to about 60% (v/v), or ranging from about 20% to about 50% (v/v).
232. The method of item 230 or 231, wherein the liquid of step a)ii. comprises at least 5% (v/v) of the organic solvent, such as ranging from 5% to 80% (v/v), ranging from 10% to 60% (v/v), or ranging from 20% to 50% (v/v).
233. The method of any one of items 230 to 232, wherein the organic solvent of step a)ii. is selected from the group consisting of acetaldehyde, acetic acid, acetone, acetonitrile, 1,2-butanediol, 1,3-butanediol, 1,4-butanediol, 2-butoxyethanol, butyric acid, dimethoxyethane, dimethyl sulfoxide, dimethyl formamide, 1,4-dioxane, ethanol, ethylene glycol, formic acid, furfuryl alcohol, glycerol, 2-methyl-2,4-pentanediol, methanol, methyl isocyanide, n-methyl-2-pyrrolidone, 1-propanol, 1,3-propanediol, 1,5-pentanediol, 2-propanol, propanoic acid, propylene glycol, pyridine, tetrahydrofuran and triethylene glycol.
234. The method of any one of items 230 to 233, wherein the organic solvent of step a)ii. is selected from the group consisting of acetonitrile, dimethyl sulfoxide, 1,4-dioxane or 2-methyl-2,4-pentanediol.
235. The method of any one of items 230 to 234, wherein the organic solvent of step a)ii. is acetonitrile, dimethyl sulfoxide or 1,4-dioxane.
236. The method of any one of items 230 to 235, wherein the organic solvent of step a)ii. is acetonitrile.
237. The method of any one of items 230 to 236, wherein liquid of step a)ii. comprises about 20% (v/v), about 30% (v/v), about 40% (v/v) or about 50% (v/v) acetonitrile, dimethyl sulfoxide, 1,4-dioxane or 2-methyl-2,4-pentanediol.
238. The method of any one of items 230 to 237, wherein liquid of step a)ii. comprises 20% (v/v), 30% (v/v), 40% (v/v) or 50% (v/v) acetonitrile, dimethyl sulfoxide, 1,4-dioxane or 2-methyl-2,4-pentanediol.
239. The method of any one of items 230 to 238, wherein liquid of step a)ii. comprises about 20% (v/v), about 30% (v/v), about 40% (v/v) or about 50% (v/v) acetonitrile.
240. The method of any one of items 230 to 239, wherein liquid of step a)ii. comprises 20% (v/v), 30% (v/v), 40% (v/v) or 50% (v/v) acetonitrile.
241. The method of any one of items 230 to 240, wherein the liquid of step a)ii. is a mixture of the at least one organic solvent with water or a mixture of the at least one organic solvent with an aqueous solution.
242. The method of any one of items 230 to 241, wherein the liquid of step a)ii. is a mixture of the at least one organic solvent with water.
243. The method of any one of items 230 to 241, wherein the liquid of step a)ii. is a mixture of the at least one organic solvent with an aqueous solution.
244. The method of item 243, wherein the aqueous solution is an aqueous buffer.
245. The method of item 244, wherein the aqueous buffer comprises borates; such as orthoborate, metaborate or tetraborate; phosphate; bicarbonate; N,N-bis(2-hydroxyethyl)—2-aminoethanesulfonic acid; 3-(N-morpholino)propanesulfonic acid; (4-2-hydroxyethyl)—1-piperazineethanesulfonic acid; 3—N-bis(hydroxyethyl)-amino-2-hydroxypropane sulfonic acid; 3-(N-morpholino) propanesulfonic acid; (2-hydroxyethyl)-piperazine-N-2-hydroxypropanesulfonic acid; piperazine-1,4-bis(2-hydroxypropanesulfonic acid); 3-[4-(2-hydroxyethyl)piperazin-1-yl]propane-1-sulfonic acid; bicine; N-(2-hydroxyethyl)piperazine-N′-(4-butanesulfonic acid); or any combination thereof.
246. The method of any one of items 244 or 245, wherein the aqueous buffer has a pH in the range of about 7 to about 11, such as of about 7.5 to about 10.5, about 8 to about 10, about 8.5 to about 9.5 or the pH is about 9.
247. The method of any one of items 244 to 246, wherein the aqueous buffer has a pH of about 9.0.
248. The method of item 242, wherein the mixture is about 20% (v/v), about 30% (v/v), about 40% (v/v) or about 50% (v/v) acetonitrile, dimethyl sulfoxide, 1,4-dioxane or 2-methyl-2,4-pentanediol in water.
249. The method of item 242 or 248, wherein the mixture is 20% (v/v), 30% (v/v), 40% (v/v) or 50% (v/v) acetonitrile, dimethyl sulfoxide, 1,4-dioxane or 2-methyl-2,4-pentanediol in water.
250. The method of any one of items 242, 248 or 249, wherein the mixture is about 20% (v/v), about 30% (v/v), about 40% (v/v) or about 50% (v/v) acetonitrile in water.
251. The method of any one of items 242 or 248 to 250, wherein the mixture is 20% (v/v), 30% (v/v), 40% (v/v) or 50% (v/v) acetonitrile in water.
252. The method of item any one of items 242 or 248 to 251, wherein the mixture is about 30% (v/v) acetonitrile in water.
253. The method of any one of items 242 or 248 to 252, wherein the mixture is 30% (v/v) acetonitrile in water.
254. The method of any one of items 230 to 253, wherein in step b) the at least one reagent Z—L²—L¹—FG and the one or more drug D—H are combined in a molar ratio of ranging from 10:1 to 1:10, such as 9:1 to 1:9, 8:1 to 1:8, 7:1 to 1:7, 6:1 to 1:6, 5:1 to 1:5, 4:1 to 1:4, 3:1 to 1:3, 2:1 to 1:2 or 1:1.
255. The method of any one of items 230 to 254, wherein the molar ratio of Z—L²—L¹—FG and D—H ranges from 4:1 to 1:1.
256. The method of any one of items 230 to 255, wherein the molar ratio of Z—L²—L¹—FG and D—H is 3:1.
257. The method of any one of items 230 to 256, wherein step b) comprises steps b)i. to b)iii.:
   b)i. mixing the at least one reagent Z—L²—L¹—FG from step a)i. with the liquid from step a)ii.;
   b)ii. adding the one or more drug D—H from step a)iii. to the mixture of step b)i.; and
   b)iii. incubating the mixture of step b)ii. for at least 10 min.
258. The method of item 257, wherein the incubation is performed for about 10 min to about 24 hours, such as about 30 min to about 6 hours or about 2 to about 5 hours.
259. The method of item 257 or 258, wherein the incubation is performed for 10 min to 24 hours, such as 30 min to 6 hours or 2 hours to 5 hours.
260. The method of any one of items 257 to 259, wherein step b)iii. is performed at a temperature ranging from about 0° C. to about 40° C., such as ranging from about 5° C. to about 30° C., ranging from about 10° C. to about 20° C., ranging from about 11° C. to about 17° C. or at a temperature of about 14° C.
261. The method of any one of items 257 to 260, wherein the incubation of step b)iii. is performed at a temperature ranging from 0° C. to 40° C., such as ranging from 5° C. to 25° C., ranging from 10° C. to 20° C., ranging from 11° C. to 17° C. or at a temperature of 14° C.
262. The method of any one of items 230 to 261, wherein Z—L²—L¹—D in step c) is purified via sterile filtration, chromatography or ultrafiltration.
263. The method of any one of items 230 to 262, wherein Z—L²—L¹—D in step c) is purified via chromatography.
264. The method of item 262 or 263, wherein the chromatography is selected from the group consisting of mixed mode chromatography, size exclusion chromatography, adsorption chromatography, thin layer chromatography, column chromatography or partition chromatography.
265. The method of any one of items 230 to 264, wherein —FG is selected from the group consisting of (methylsulfonyl)oxy, [(nonafluorobutyl)sulfonyl]oxy, [(trifluoromethyl) sulfonyl]oxy, [2-(trimethylsilyl)ethoxy]methyl acetal, 1-hydroxybenzotriazole, 4-methoxybenzenesulfonamide, 4-methoxybenzyl ether, 4-nitrophenolate, 9-fluorenylmethyl carbamate, azide, benzyloxy, benzyloxycarbonyl, brosylate, dichlorophenolate, difluorophenolate, dihalophosphinoyloxy, ethanesulfonyloxy, ethoxyethyl ether, hydroxysuccinimide, imidazole, methanesulfonylate/mesylate, methanesulfonyloxy, methoxymethyl ether, monofluorophenolate, N-hydroxysuccinimide, N-hydroxysulphosuccinimide, nitrophenolate, pentafluorophenolate, phenyl, β-toluenesulfonylate (tosylate), tert-butyldimethylsilyl ether, tetrachlorophenolate, tetrafluorophenolate, tetrahydropyranyl ether, thienyloxy, triflate, trifluoroacetoxy, trifluoromethylmethanesulfonylate and trityl(triphenylmethyl)ether.
266. The method of any one of items 230 to 265, wherein —FG is selected from the group consisting of 4-nitrophenolate, pentafluorophenolate and N-hydroxysuccinimide.
267. The method of any one of items 230 to 266, wherein —FG is pentafluorophenolate.
268. The method of any one of items 230 to 242 or 248 to 267, wherein the method comprises the steps of
   a) providing
      a)i. at least one reagent of formula (XII)

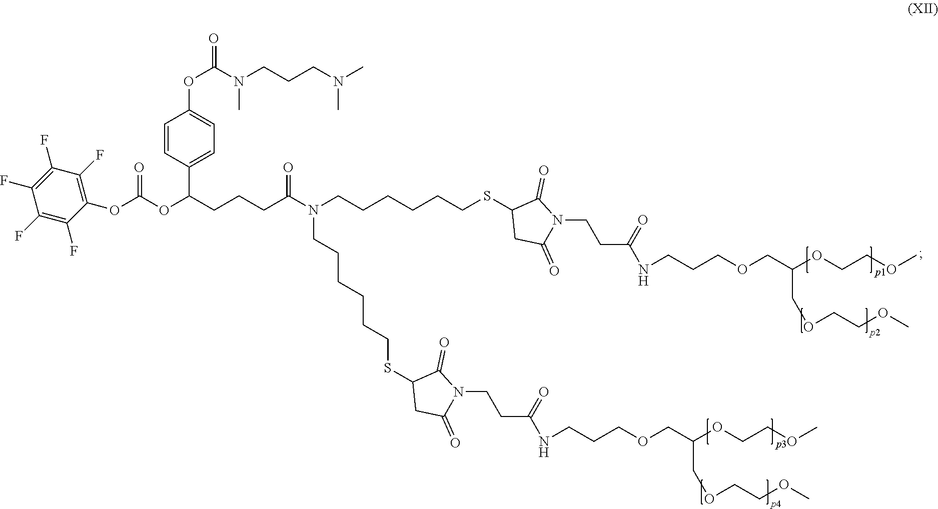

wherein p 1, p2, p3, p4 are independently of each other an integer ranging from 110 to 345, from 140 to 315, from 170 to 285, from 195 to 255, from 200 to 250, from 210 to 250 or from 220 to 240;
   a)ii. a mixture of about 30% (v/v) acetonitrile in water; and
   a)iii. a solution comprising D—H;
b) mixing the at least one reagent of step a)i. with the mixture of step a)ii.;
c) mixing the mixture of step b) with the solution of step a)iii., wherein the molar ratio of the reagent of formula (XII) and D—H is ranging from 4:1 to 1:1;
d) incubating the mixture of step c) for 1 to 5 hours at a temperature ranging from 10° C. to 20° C. to obtain the IL-2 conjugate of formula (I); and
e) optionally, purifying the IL-2 conjugate of formula (I) from the incubated mixture of step d).
269. The method of any one of items 230 to 268, wherein D—H is the free form of —D,
   wherein
   —D is —D′—M_{mod},
   wherein
   —D′— is an IL-2 moiety; and
   —M_{mod} is a first polymeric moiety that is stably conjugated to a side chain of an amino acid residue of —D′—.
270. The method of item 269, wherein —D′— has at least 95% sequence identity to the amino acid sequence of SEQ ID NO:1.
271. The method of item 269 or 270, wherein —D′— has at least 96% sequence identity to the amino acid sequence of SEQ ID NO:1.
272. The method of any one of items 269 to 271, wherein —D′— has at least 97% sequence identity to the amino acid sequence of SEQ ID NO:1.
273. The method of any one of items 269 to 272, wherein —D′— has at least 98% sequence identity to the amino acid sequence of SEQ ID NO:1.
274. The method of any one of items 269 to 273, wherein —D′— has at least 99% sequence identity to the amino acid sequence of SEQ ID NO:1.
275. The method of any one of items 269 to 274, wherein —D′— comprises the amino acid sequence of SEQ ID NO:1, in which one amino acid residue selected from the group consisting of K35, R38, M39, T41, F42, K43, F44, Y45, E62, P65, E68, V69, and L72 is replaced with a proteinogenic or non-proteinogenic amino acid residue.
276. The method of any one of items 269 to 275, wherein —D′— comprises the A1 del mutation.
277. The method of any one of items 269 to 276, wherein —D′— comprises the amino acid sequence of SEQ ID NO:1, in which one amino acid residue selected from the group consisting of K35, R38, M39, T41, F42, K43, F44, Y45, E62, P65, E68, V69, and L72 is replaced with a cysteine residue.
278. The method of any one of items 269 to 275 or 277, wherein —D′— has the amino acid sequence of SEQ ID NO:1, in which one amino acid residue selected from the group consisting of K35, R38, M39, T41, F42, K43, F44, Y45, E62, P65, E68, V69, and L72 is replaced with a cysteine residue.
279. The method of any one of items 269 to 275, 277 or 278, wherein —D′— has the amino acid sequence of SEQ ID NO:4.
280. The method of any one of items 269 to 279, wherein —M_{mod} has a molecular weight ranging from 0.5 kDa to 20 kDa.
281. The method of any one of items 269 to 280, wherein —M_{mod} has a molecular weight ranging from 1 kDa to 20 kDa.
282. The method of any one of items 269 to 281, wherein —M_{mod} has a molecular weight ranging from 2 kDa to 10 kDa.
283. The method of any one of items 269 to 282, wherein —M_{mod} has a molecular weight ranging from 3 kDa to 7 kDa.
284. The method of any one of items 269 to 283, wherein —M_{mod} has a molecular weight of 5±25% kDa.
285. The method of any one of items 269 to 284, wherein —M_{mod} has a molecular weight ranging from 4 kDa to 6 kDa.
286. The method of any one of items 269 to 284, wherein —M_{mod} has a molecular weight of 5±20% kDa.
287. The method of any one of items 269 to 286, wherein —M_{mod} has a molecular weight of 5±15% kDa.
288. The method of any one of items 269 to 287, wherein —M_{mod} has a molecular weight of about 5 kDa.
289. The method of any one of items 269 to 288, wherein —M_{mod} is a PEG-based polymer.
290. The method of any one of items 269 to 289, wherein attachment of —M_{mod} occurs at an amino acid position selected from the group consisting of K35, R38, M39, T41, F42, K43, F44, Y45, E62, P65, E68, V69, and L72.
291. The method of any one of items 269 to 275 or 277 to 290, wherein —D′— has the amino acid sequence of SEQ ID NO. 4 and —M_{mod} is stably conjugated to the sulfur atom of the side chain of the cysteine at position 38.
292. The method of any one of items 269 to 291, wherein —M_{mod} is of formula (A-1)

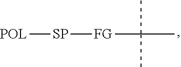

wherein
   —FG— is a linkage;
   —SP— is a spacer moiety; and
   —POL is a polymer.
293. The method of item 292, wherein —FG— of formula (A-1) is of formula (FG-1a)

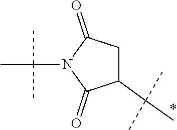

wherein
   the dashed line marked with the asterisk indicates attachment to the sulfur of a cysteine
   side chain of —D′— and the unmarked dashed line indicates attachment to —SP—.
294. The method of item 292, wherein —FG— of formula (A-1) is of formula (FG-1d) or (FG— 1e)

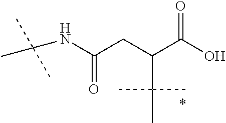

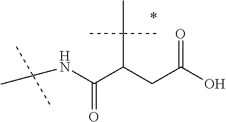

wherein the dashed line marked with the asterisk indicates attachment to the sulfur of
a cysteine residue of —D′— and the unmarked dashed line indicates attachment to —SP—.
295. The method of any one of items 292 to 294, wherein —SP— is C₁₋₁₀ alkyl, which C₁₋₁₀ alkyl is optionally substituted with one or more —R⁹, and which C₁₋₁₀ alkyl is optionally interrupted by one or more groups selected from the group consisting of —O—, —C(O)N(R¹⁰)—, —S(O)₂—, —S(O)—, —S—, —N(R¹⁰)—, —OC(OR¹⁰)(R^{10a})—, —N(R¹⁰)C(O)N(R^{10a})—, and —OC(O)N(R¹⁰)—; wherein each —R⁹ is selected from the group consisting of C₁₋₆ alkyl; and each —R¹⁰ and —R^{10a} is independently selected from the group consisting of —H and C₁₋₆ alkyl.
296. The method of any one of items 292 to 295, wherein —POL is of formula (A-11)

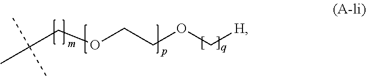

wherein
   the dashed line indicates attachment to —SP—;
   m is 0 or 1;
   p is an integer ranging from about 10 to about 540; and
q is selected from the group consisting of 1, 2, 3, 4, 5, and 6. 297. The method of any one of items 269 to 293, 295 or 296, wherein —M_{mod} is of formula (A-1a)

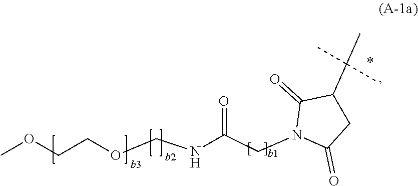

wherein
   the dashed line marked with the asterisk indicates attachment to the sulfur of a cysteine residue of —D′—;
   b1 is an integer selected from the group consisting of 1, 2, 3, 4, 5, 6, 7, 8, 9, 10, 11, 12, 13, 14, 15, 16, 17, 18, 19 and 20;
   b2 is an integer selected from the group consisting of 1, 2, 3, 4, 5, 6, 7, 8, 9, 10, 11, 12, 13, 14, 15, 16, 17, 18, 19 and 20; and
   b3 is an integer ranging from about 10 to about 540.
298. The method of any one of items 269 to 292 or 294 to 296, wherein —M_{mod} is of formula (A-1d) or (A-1e)

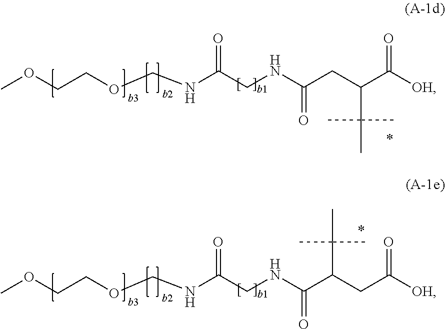

wherein
   the dashed line marked with the asterisk indicates attachment to the sulfur of a cysteine residue of —D′—;
   b1 is an integer selected from the group consisting of 1, 2, 3, 4, 5, 6, 7, 8, 9, 10, 11, 12, 13, 14, 15, 16, 17, 18, 19 and 20;
   b2 is an integer selected from the group consisting of 1, 2, 3, 4, 5, 6, 7, 8, 9, 10, 11, 12, 13, 14, 15, 16, 17, 18, 19 and 20; and
   b3 is an integer ranging from about 10 to about 540.
299. The method of item 297 or 298, wherein b1 is an integer ranging from 1 to 4.
300. The method of any one of items 297 to 299, wherein b1 is 2.
301. The method of any one of items 297 to 299, wherein b1 is 3.
302. The method of any one of items 297 to 301, wherein b2 is 2.
303. The method of any one of items 297 to 301, wherein b2 is 3.
304. The method of any one of items 297 to 303, wherein b3 is an integer ranging from 10 to 450.
305. The method of any one of items 297 to 304, wherein b3 is an integer ranging from 22 to 340.
306. The method of any one of items 297 to 305, wherein b3 is an integer ranging from 45 to 230.
307. The method of any one of items 297 to 306, wherein b3 is an integer ranging from 65 to 160.
308. The method of any one of items 297 to 307, wherein b3 is selected such that —M_{mod} has a molecular weight of 5±25% kDa.
309. The method of any one of items 297 to 308, wherein b3 is an integer ranging from 90 to 140.
310. The method of any one of items 297 to 309, wherein b3 is selected such that —M_{mod} has a molecular weight ranging from about 4 kDa to about 6 kDa.
311. The method of any one of items 297 to 310, wherein b3 is selected such that —M_{mod} has a molecular weight of 5 20% kDa.
312. The method of any one of items 297 to 311, wherein b3 is an integer ranging from 95 to 135.
313. The method of any one of items 297 to 312, wherein b3 is selected such that —M_{mod} has a molecular weight of 5 15% kDa.
314. The method of any one of items 297 to 313, wherein b3 is an integer ranging from 100 to 125.
315. The method of any one of items 230 to 314, wherein D—H is D′—M_{mod} and wherein —D′— has the amino acid sequence of SEQ ID NO. 4, —M_{mod} has the structure of (A-1d) or (A-1e)

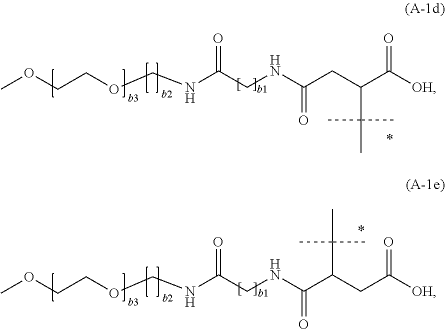

wherein
the dashed line marked with the asterisk indicates attachment to the sulfur atom of the side chain of the cysteine at position 38;
b1 is 2;
b2 is 2; and
b3 ranges from 90 to 140, from 95 to 135 or from 100 to 125.
316. The IL-2 conjugate for use of item 90, wherein the cancer is selected from the group consisting of melanoma, cervical cancer, pancreatic cancer, renal cell cancer, colorectal cancer, ovarian cancer, breast cancer, salivary gland cancer, endometrial cancer and lung cancer.
317. The IL-2 conjugate for use of item 90 or 316, wherein the cancer is selected from the group consisting of melanoma, cervical cancer, colorectal cancer, lung cancer and ovarian cancer.
318. The IL-2 conjugate for use of any one of items 90 or 316, wherein the cancer is pancreatic cancer.
319. The IL-2 conjugate for use of item 90 or 316, wherein the cancer is renal cell cancer.
320. The IL-2 conjugate for use of item 319, wherein the renal cell cancer is clear cell renal cell carcinoma (ccRCC).
321. The IL-2 conjugate for use of any one of items 90, 316 or 317, wherein the cancer is colorectal cancer.
322. The IL-2 conjugate for use of any one of items 90, 316 or 317, wherein the cancer is ovarian cancer.
323. The IL-2 conjugate for use of any one of items 322, wherein the ovarian cancer is platinum-resistant ovarian cancer (PROC).
324. The IL-2 conjugate for use of item 90 or 316, wherein the cancer is breast cancer.
325. The IL-2 conjugate for use of item 324, wherein the breast cancer is triple negative breast cancer (TNBC).
326. The IL-2 conjugate for use of item 88 or 316, wherein the cancer is salivary gland cancer.
327. The IL-2 conjugate for use of item 326, wherein the salivary gland cancer is acinic cell carcinoma.
328. The IL-2 conjugate for use of item 90 or 316, wherein the cancer is endometrial cancer.
329. The IL-2 conjugate for use of any one of items 90, 316 or 317, wherein the cancer is lung cancer.
330. The IL-2 conjugate for use of item 329, wherein the lung cancer is small cell lung cancer (SCLC).
331. The IL-2 conjugate for use of item 329, wherein the lung cancer is non-small cell lung cancer (NSCLC).
332. The IL-2 conjugate for use of any one of items 90 or 316 to 331, wherein the cancer has previously been treated with an inhibitor of PD-1 or PD-L1.
333. The IL-2 conjugate for use of any one of items 90 or 316 to 332, wherein the cancer has previously been treated with an inhibitor of PD-1.
334. The IL-2 conjugate for use of any one of items 90 or 316 to 332, wherein the cancer has previously been treated with an inhibitor of PD-L1.
335. The IL-2 conjugate for use of any one of items 90 or 316 to 334, wherein the cancer is refractory to treatment with an inhibitor of PD-1 or PD-L1.
336. The IL-2 conjugate for use of any one of items 90 or 316 to 335, wherein the cancer is refractory to treatment with an inhibitor of PD-1.
337. The IL-2 conjugate for use of any one of items 90 or 316 to 335, wherein the cancer is refractory to treatment with an inhibitor of PD-L1.
338. The IL-2 conjugate for use of any one of items 90 or 316 or 337, wherein the cancer is mismatch repair deficient (dMMR).
339. The IL-2 conjugate for use of any one of items 90 or 316 to 338, wherein the cancer is microsatellite instability-high (MSI-H).
340. The IL-2 conjugate for use of any one of items 90, 316 to 337, wherein the cancer is microsatellite instability-low (MSI-L).
341. The IL-2 conjugate for use of any one of items 90, 316 to 337, wherein the cancer is microsatellite stable (MSS).
342. The IL-2 conjugate for use of any one of items 90 or 316 to 341, wherein the cancer is PD-1 positive or PD-L1 positive.
343. The IL-2 conjugate for use of any one of items 90 or 316 to 342, wherein the cancer is PD-1 positive.
344. The IL-2 conjugate for use of any one of items 90 or 316 to 342, wherein the cancer is PD-L1 positive.
345. The IL-2 conjugate for use of any one of items 90 or 316 to 341, wherein the cancer is PD-1 negative or PD-L1 negative.
346. The IL-2 conjugate for use of any one of items 90, 316 to 341 or 345, wherein the cancer is PD-1 negative.
347. The IL-2 conjugate for use of any one of items 90, 316 to 341 or 345, wherein the cancer is PD-L1 negative.
348. The IL-2 conjugate for use of any one of items 90 or 316 to 347, wherein the cancer is treated in a neoadjuvant setting.
349. The IL-2 conjugate for use of item 91, wherein the one or more additional drug is pemetrexed.
350. The IL-2 conjugate for use of item 91, wherein the one or more additional drug is paclitaxel.
351. The unit dosage form of item 200, wherein the cancer is selected from the group consisting of melanoma, cervical cancer, pancreatic cancer, renal cell cancer, colorectal cancer, ovarian cancer, breast cancer, salivary gland cancer, endometrial cancer and lung cancer.
352. The unit dosage form of item 200 or 351, wherein the cancer is selected from the group consisting of melanoma, cervical cancer, colorectal cancer, lung cancer and ovarian cancer.
353. The unit dosage form of any one of items 200 or 351, wherein the cancer is pancreatic cancer.
354. The unit dosage form of item 200 or 351, wherein the cancer is renal cell cancer.
355. The unit dosage form of item 354, wherein the renal cell cancer is clear cell renal cell carcinoma (ccRCC).
356. The unit dosage form of any one of items 200, 351 or 352, wherein the cancer is colorectal cancer.
357. The unit dosage form of any one of items 200, 351 or 352, wherein the cancer is ovarian cancer.
358. The unit dosage form of any one of items 357, wherein the ovarian cancer is platinum-resistant ovarian cancer (PROC).
359. The unit dosage form of item 200 or 351, wherein the cancer is breast cancer.
360. The unit dosage form of item 359, wherein the breast cancer is triple negative breast cancer (TNBC).
361. The unit dosage form of item 88 or 351, wherein the cancer is salivary gland cancer.
362. The unit dosage form of item 361, wherein the salivary gland cancer is acinic cell
carcinoma.
363. The unit dosage form of item 200 or 351, wherein the cancer is endometrial cancer.
364. The unit dosage form of any one of items 200, 351 or 352, wherein the cancer is lung cancer.
365. The unit dosage form of item 364, wherein the lung cancer is small cell lung cancer (SCLC).
366. The unit dosage form of item 364, wherein the lung cancer is non-small cell lung cancer (NSCLC).
367. The unit dosage form of any one of items 200 or 351 to 366, wherein the cancer has previously been treated with an inhibitor of PD-1 or PD-L1.
368. The unit dosage form of any one of items 200 or 351 to 367, wherein the cancer has previously been treated with an inhibitor of PD-1.
369. The unit dosage form of any one of items 200 or 351 to 367, wherein the cancer has previously been treated with an inhibitor of PD-L1.
370. The unit dosage form of any one of items 200 or 351 to 369, wherein the cancer is refractory to treatment with an inhibitor of PD-1 or PD-L1.
371. The unit dosage form of any one of items 200 or 351 to 370, wherein the cancer is refractory to treatment with an inhibitor of PD-1.
372. The unit dosage form of any one of items 200 or 351 to 370, wherein the cancer is refractory to treatment with an inhibitor of PD-L1.
373. The unit dosage form of any one of items 200 or 351 or 372, wherein the cancer is mismatch repair deficient (dMMR).
374. The unit dosage form of any one of items 200 or 351 to 373, wherein the cancer is microsatellite instability-high (MSI-H).
375. The unit dosage form of any one of items 200, 351 to 372, wherein the cancer is microsatellite instability-low (MSI-L).
376. The unit dosage form of any one of items 200, 351 to 372, wherein the cancer is microsatellite stable (MSS).
377. The unit dosage form of any one of items 200 or 351 to 376, wherein the cancer is PD-1 positive or PD-L1 positive.
378. The unit dosage form of any one of items 200 or 351 to 377, wherein the cancer is PD-1 positive.
379. The unit dosage form of any one of items 200 or 351 to 377, wherein the cancer is PD-L1 positive.
380. The unit dosage form of any one of items 200 or 351 to 376, wherein the cancer is PD-1 negative or PD-L1 negative.
381. The unit dosage form of any one of items 200, 351 to 377 or 380, wherein the cancer is PD-1 negative.
382. The unit dosage form of any one of items 200, 351 to 377 or 380, wherein the cancer is PD-L1 negative.
383. The unit dosage form of any one of items 200 or 351 to 382, wherein the cancer is treated in a neoadjuvant setting.
384. The unit dosage form of item 201, wherein the one or more additional drug is pemetrexed.
385. The unit dosage form of item 201, wherein the one or more additional drug is paclitaxel.
386. The IL-2 conjugate for use of item 91, wherein the one or more additional drug is docetaxel.
387. The IL-2 conjugate for use of item 91, wherein the IL-2 conjugate is administered to the patient prior to, simultaneously with or after administration of two additional drugs.
388. The IL-2 conjugate for use of item 387, wherein the two additional drugs are a chemotherapeutic agent and pembrolizumab.
389. The IL-2 conjugate for use of item 388, wherein the chemotherapeutic agent is pemetrexed, paclitaxel or docetaxel.
390. The IL-2 conjugate for use of any one of items 90, 316 or 317, wherein the cancer is melanoma.
391. The IL-2 conjugate for use of any one of items 90, 316 or 317, wherein the cancer is cervical cancer.
392. The unit dosage form of item 201, wherein the one or more additional drug is docetaxel.
393. The unit dosage form of item 201, wherein the IL-2 conjugate is administered to the patient prior to, simultaneously with or after administration of two additional drugs.
394. The unit dosage form of item 393, wherein the additional drugs are a chemotherapeutic agent and pembrolizumab.
395. The unit dosage form of item 394, wherein the chemotherapeutic agent is pemetrexed, paclitaxel or docetaxel.
396. The unit dosage form of any one of items 200, 351 or 352, wherein the cancer is melanoma.
397. The unit dosage form of any one of items 200, 351 or 352, wherein the cancer is cervical cancer.
398. A method of treating or delaying progression of cancer in a patient, comprising:
   (a) measuring the level of sCD25 in a biological sample obtained from the patient after administration of at least one dose ranging from 80 μg IL2/kg to 160 μg IL-2/kg of the IL-2 conjugate of formula (I) to the patient; and/or
   (b) measuring the level and/or proliferation of CD8+ T cells in a biological sample obtained from the patient after administration of at least one dose ranging from 80 μg IL2/kg to 160 μg IL-2/kg of the IL-2 conjugate of formula (I) to the patient;
   (c) comparing the level from step (a) and/or the level and/or proliferation from step (b) to a reference; wherein an increased level of sCD25 and/or increased level and/or proliferation of CD8+ T cells compared to the reference indicates that the patient is likely to benefit from the treatment with the IL-2 conjugate of formula (I); and
   (d) if the patient has
      i) an increased level of sCD25 compared to the reference, administering to the patient one or more additional dose ranging from 80 μg IL2/kg to 160 μg IL-2/kg of the IL-2 conjugate of formula (I); and/or
      ii) an increased level and/or proliferation of CD8+ T cells compared to the reference, administering to the patient one or more additional dose ranging from 80 μg IL2/kg to 160 μg IL-2/kg of the IL-2 conjugate of formula (I).
399. The method of item 398, wherein the dose in steps (a), (b) and (d) ranges from 100 μg IL-2/kg to 140 μg IL 2/kg.
400. The method of item 398 or 399, wherein the dose in steps (a), (b) and (d) ranges from 110 μg IL-2/kg to 130 μg IL 2/kg.
401. The method of any one of items 398 to 400, wherein the dose in steps (a), (b) and (d) is about 120 μg IL 2/kg.
402. The method of any one of items 398 to 401, wherein the dose in steps (a), (b) and (d) is 120 μg IL 2/kg.
403. The method of any one of items 398 to 402, wherein the biological sample in step (a) and/or step (b) is obtained after administration of the first, second, third, fourth, fifth or any subsequent dose of the IL-2 conjugate of formula (I).
404. The method of any one of items 398 to 403, wherein the biological sample in step (a) and/or step (b) is obtained between 1 day to 6 weeks after administration of the IL-2 conjugate of formula (I), such as 1 day, 2 days, 3 days, 4 days, 5 days, 6 days, 1 week, 2 weeks, 3 weeks, 4 weeks, 5 weeks or 6 weeks.
405. The method of any one of items 398 to 404, wherein the biological sample in step (a) and step (b) is the same biological sample.
406. The method of any one of items 398 to 404, wherein the biological samples in step (a) and step (b) are different biological samples.
407. The method of any one of items 398 to 406, wherein the biological sample in step (a) and/or step (b) is a blood sample.
408. The method of item 407, wherein the blood sample comprises peripheral blood mononuclear cells (PBMCs).
409. The method of any one of items 398 to 406, wherein said biological sample in step (a) and/or step (b) is a tumor sample.
410. The method of any one of items 398 to 409, wherein the level of sCD25 is measured.
411. The method of any one of items 398 to 409, wherein the level of CD8+ T cells is measured.
412. The method of any one of items 398 to 409, wherein the proliferation of CD8+ T cells is measured.
413. The method of any one of items 398 to 409, wherein the level of sCD25 and the level of CD8+ T cells are measured.
414. The method of any one of items 398 to 409, wherein the level of sCD25 and the proliferation of CD8+ T cells are measured.
415. The method of any one of items 398 to 409, wherein the level of CD8+ T cells and the proliferation of CD8+ T cells are measured.
416. The method of any one of items 398 to 409, wherein the level of sCD25, the level of CD8+ T cells and the proliferation of CD8+ T cells are measured.
417. The method of any one of items 398 to 409 and 411 to 417, wherein the level and/or proliferation of CD8+ T cells is measured by flow cytometry, such as by detecting expression of a cellular or nuclear proliferation-associated antigen on a CD8+ T cell, such as Ki-67.
418. The method of any one of items 398 to 417, wherein the reference comprises a biological sample obtained from the patient prior to administration of the IL-2 conjugate of formula (I).
419. The method of any one of items 398 to 417, wherein the reference comprises a standard derived from a population of known non-responders to treatment with the IL-2 conjugate of formula (I).
420. The method of any one of items 398 to 417, wherein the reference comprises a standard derived from a population of known responders to treatment with the IL-2 conjugate of formula (I).
421. The method of any one of items 398 to 417, wherein the reference is data compiled across a plurality of patients.
422. The method of any one of items 398 to 417, wherein the reference is a healthy subject.
423. The method of any one of items 398 to 422, wherein the one or more additional dose in step (d) is administered if the absolute level of sCD25 in the biological sample, such as measured in pg/ml, is increased compared to the reference.
424. The method of any one of items 398 to 423, wherein the one or more additional dose in step (d) is administered if the relative level of sCD25 in the biological sample, such as indicated by a fold-change, is increased compared to the reference.
425. The method of any one of items 398 to 423, wherein the one or more additional dose in step (d) is administered if the absolute level of CD8+ T cells in the biological sample, such as measured in cells/β1, is increased compared to the reference.
426. The method of any one of items 398 to 423, wherein the one or more additional dose in step (d) is administered if the relative level of CD8+ T cells in the biological sample, such as indicated by a fold-change, is increased compared to the reference.
427. The method of any one of items 398 to 423, wherein the one or more additional dose in step (d) is administered if the absolute number of proliferating CD8+ T cells in the biological sample, such as measured in cells/β1, is increased compared to the reference.
428. The method of any one of items 398 to 423, wherein the one or more additional dose in step (d) is administered if the relative proliferation of CD8+ T cells in the biological sample, such as indicated by a fold-change, is increased compared to the reference.
429. The method of any one of items 398 to 428, wherein steps (a), (b) and (c) may be repeated until an increased level of sCD25 and/or the level and/or proliferation of CD8+ T cells is observed.

### EXAMPLES

### Example 1

### Synthesis of Compound 1

### Compound 1 has the Following Structure:

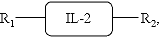

—R¹ is attached to the nitrogen of the N-terminal amine functional group or to a nitrogen of an amine functional group from a lysine side chain of the IL-2 moiety and has the following structure:

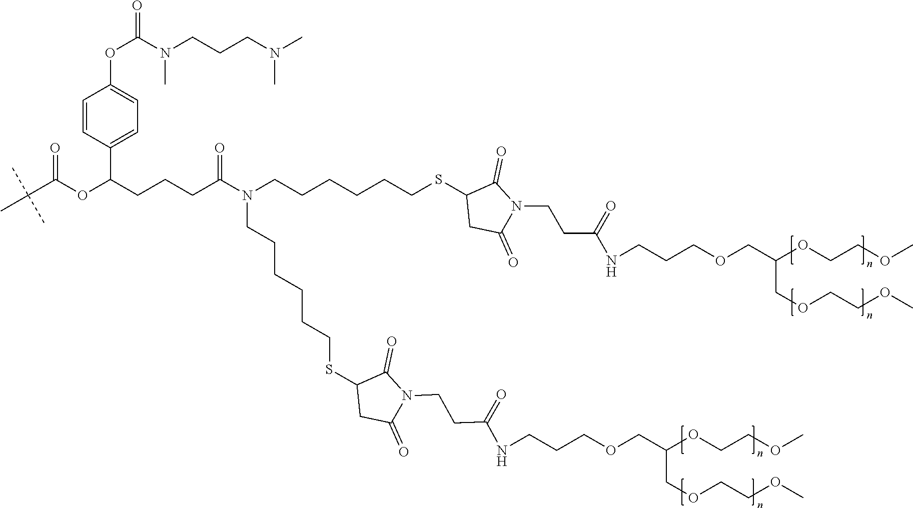

with n ranging from 200 to 250;

—R₂ is attached to the thiol of the cysteine at position 38 of the IL-2 moiety and has the following structure:

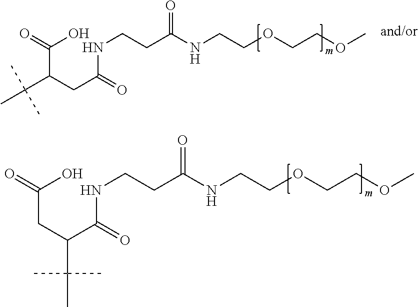

with m ranging from 100 to 125;

and

IL-2 has the sequence:

APTSSSTKKTQLQLEHLLLDLQMILNGINNYKNPKLTCMLTFKFY
MPKKATELKHLQCLEEELKPLEEVLNLAQSKNFHLRPRDLISNIN
VIVLELKGSETTFMCEYADETATIVEFLNRWITFSQSIISTLT.

Compound 1 can be synthesized as described in WO2021/245130, example 5, compound 6.

Compound 1 can alternatively be synthesized as follows: 40 mL of purified IL-2 protein 2 (SEQ ID NO:4) formulated at 2.46 mg/mL in 20 mM sodium phosphate, 140 mM sodium chloride, pH 7.4 were used as starting protein solution. TCEP HCl was dissolved in PBS, pH 7.4 to give a 50 mM solution. No adjustment of the pH was performed. 326 μL of the TCEP solution were added to the protein solution. The sample was incubated at room temperature for approximately 30 min. Subsequently, 5 mM 5 kDa PEG maleimide (M-Mal-5000, CAS 99126-64-4, Jenkem Technology Co., Beijing, China) in PBS, pH 7.4 (3.5 mol. eq.) were added to the reaction solution. After incubation at room temperature for 10 min, the buffer of the conjugation mixture was exchanged to pH 9 buffer (0.1 M boric acid adjusted to pH 9 with NaOH) using an Aekta system equipped with two HiPrep Desalting 26/10 columns connected in series. The sample was then concentrated to 6.2 mg/mL using Amicon Ultra-15, Ultracel 3 K centrifugation filters (Merck Millipore) and incubated at 25° C. overnight to give unpurified IL-2 conjugate 4.

mPEG-linker reagent 3 (synthesis can be performed as described for the compound 17ca in WO2009/133137, example 7, using compound 16c and 1A) were dissolved in a cold mixture of 30% (v/v) acetonitrile in water to give a stock solution of 1.96 mmol/L. The solution was stored at 4-8° C. for 60 min. The solution of conjugate 4 was diluted to 5 mg/mL by addition of pH 9 buffer (0.1 M boric acid adjusted to pH 9 with NaOH), then the cooled stock solution of reagent 3 was added (corresponding to 3 mol. eq. with respect to the protein).

The conjugation mixture was placed in a thermoshaker at 14° C., 300 rpm for 2 h. After conjugation, the pH was shifted to pH 4 by addition of water and 2 M sodium acetate, pH 3.6. After incubation at 25° C. for 27 h, the conjugate with one single mPEG linker attached (compound 1) was isolated from the reaction mixture using a HiScreen Capto MMC ImpRes column (column dimensions: 0.77×10 cm) connected to an Aekta system. A flow rate of 1.2 mL/min and a linear gradient from 10 mM succinic acid, pH 5.0 to 80% of 10 mM succinic acid, 1 M sodium chloride, pH 5.0 in 12 column volumes was applied. The peak containing mainly compound 1 eluting during the gradient was identified by analytical size exclusion chromatography.

The main difference between the above-described alternative process and the process in WO2021/245130, example 5, compound 6 is that reagent 3 is dissolved in a mixture of 30% (v/v) acetonitrile in water instead of in pure water. Surprisingly, it was observed that dissolution of reagent 3 in a mixture of 30% (v/v) acetonitrile in water compared to pure water:
required around 25% less reagent 3 as starting material in the conjugation process to obtain compound 1 in a similar yield and purity;
reduced hydrolysis of reagent 3 to approx. 23% hydrolysis compared to >85% in pure water after 24 hours incubation at 5° C. and to approx. 53% hydrolysis compared to approx. 95% in pure water after approx. 3 hours at 25° C.;
was approx. 4 times faster; and
resulted in less or basically no foaming or persistent floating of residual reagent 3 on the liquid surface after dissolution.

Similar effects were observed for dissolution of reagent 3 in mixtures of 30% (v/v) DMSO in water (e.g. reduced hydrolysis of 57.0% at 5° C.) or 30% (v/v) and mixtures of 90% (v/v) 1.4-dioxane in water (e.g. reduced hydrolysis of 35.5% or 13.5% at 5° C., respectively).

### Synthesis of Compound 2

Compound 2 is disclosed as the drug of formula (A-3) herein. Compound 2 can be manufactured as described for hydrogel 14 in WO2020/141221.

### Example 2—Study Design

Note: Experiments and resulting data are reported in past tense, even though at the time of filing the clinical trial was ongoing.

Data shown herein is from Part 1, Part 2 and Part 3 of a study to investigate safety and tolerability of compound 1 alone or in combination with pembrolizumab, standard of care chemotherapy, compound 2, or in combination with pembrolizumab and standard of care chemotherapy, in adult participants with locally advanced or metastatic solid tumor malignancies (NCT05081609). The study uses a standard 3±3 dose range finding design to investigate the safety and tolerability, and to establish the maximum tolerated dose (MTD) and the recommended Phase 2 dose (RP2D), of compound 1 alone (Part 1) or in combination with pembrolizumab (Part 2), or in combination with standard of care chemotherapy, or compound 2, or in combination with pembrolizumab and standard of care chemotherapy in Part 3 (Phase 2) in multiple solid tumors. Secondary objectives were to assess the anti-tumor activity and pharmacokinetics (PK) of compound 1 in Part 1, Part 2 and Part 3. Biomarkers in peripheral blood was included as exploratory endpoints. Effects on eosinophil counts and interleukin 5 (IL-5) were analyzed as surrogate markers for safety, and lymphocyte counts and concentrations and proliferation of lymphocyte subsets were used as markers of pharmacodynamic (PD) activity and clinical response.

### Part 1: Monotherapy Dose Escalation (Phase 1)

Part 1 enrolls participants with advanced cancers that have progressed on available standard-of-care (SOC) treatment options and have disease for which there is no SOC therapy. The starting dose of compound 1 will be Dose Level 1, administered intravenously (IV) every 3 weeks (21 days, Q3W). Upon disease progression, the addition of pembrolizumab every Q3W was allowed if deemed appropriate by the investigator and after discussion with the Medical Monitor (Table 1).

Part 2: Combination Dose Escalation with Pembrolizumab (Phase 1)

Part 2 Combination Dose Escalation with pembrolizumab start after Dose Level 2 from Part 1 has passed safety evaluation. Compound 1 is given IV starting with Dose Level 1 in combination with pembrolizumab IV at 200 mg Q3W. For combination dose escalation in Part 2, pembrolizumab is administered with a dose of compound 1 that is one dose level lower to ensure adequate monotherapy dose-limiting toxicity (DLT) evaluation before moving to subsequent dose levels (Table 1). Combination dose escalation (Part 2) with pembrolizumab enrolls participants with tumor types where there may be expected clinical benefit from pembrolizumab (e.g., indications with regulatory approval or available data demonstrating clinical activity in the advanced setting) as deemed by the treating investigator. Participants who will also have received no more than 2 lines of therapy or treatment regimens for locally advanced, unresectable, recurrent, or metastatic disease.

TABLE 1
Dose Levels of Parts 1 and 2

| Compound 1 (Parts 1 and 2)¹ | | | Pembrolizumab (for Part 2 or optional |
|---|---|---|---|
| Dose Level | Dose | | for Part 1 after disease progression)² |
| 1 | 20 | μg/kg | 200 mg Q3W |
| 2 | 40 | μg/kg | |
| 3 | 80 | μg/kg | |
| 3.5 | 120 | μg/kg | |
| 4³ | 160 | μg/kg | |

Abbreviations: DLT = dose-limiting toxicity; PD = pharmacodynamic; PK = pharmacokinetic; μg = microgram; mg = milligram; Q3W = every 3 weeks; RP2D = recommended phase 2 dose.
¹Dose levels refer to IL-2 equivalents contained in compound 1. With the exception of Dose Level 1, dose levels for subsequent cohorts may have been adjusted (escalated or de-escalated) based on clinical and available PK/PD observations. Each level was evaluated by the Safety Review Committee (SRC) after safety and available PK/PD data from the DLT period of a minimum of 3 participants were enrolled to receive a dose level.
²If DLTs were observed at any one dose level, dose may have been de-escalated in the same cohort or the previous dose level may have been reopened to enrollment to ensure the availability of safety and PK/PD data for a minimum of 6 participants for RP2D decision. Upon disease progression in Part 1, the addition of pembrolizumab was permitted at the discretion of the Investigator and after discussion with and approval from the Medical Monitor.
³Dose level 4 was only used in Part 1.

### Part 3: Indication Specific Dose Expansion Cohorts (Phase 2)

Part 3 enrolls participants with platinum resistant ovarian cancer (PROC) (Cohort 3), post anti-PD-1 melanoma (Cohort 4), second line or later cervical cancer (Cohort 5), neoadjuvant melanoma (Cohort 6), neoadjuvant non-small cell lung cancer (NSCLC) (Cohort 7), post anti-PD-(L)1 NSCLC (Cohort 8) and post anti- PD-(L)1 small cell lung cancer (SCLC) (Cohort 9).

The dose of compound 1 is administered at 120 μg/kg (the RP2D determined in Part 1 and Part 2 (see Example 9)), administered (IV, Q3W) in combination with pembrolizumab (200 mg, IV, Q3W), standard of care chemotherapy, or compound 2 (0.5 mg/lesion, intratumoral injection, Q3W), or in combination with pembrolizumab and standard of care chemotherapy depending on the cohort.

### Example 3—Patient Demographics

Part 1: Monotherapy Dose Escalation (Phase 1) As of 28 Apr. 2023, 25 participants enrolled in the dose escalation Part 1. Patient demographics are outlined in Table 2.1.

TABLE 2.1
Demographics for dose escalation Part 1

| | Compound 1 Dose (μg/kg) | | | | | |
|---|---|---|---|---|---|---|
| | 20 | 40 | 80 | 120 | 160 | Total |
| Demographics | (n = 4) | (n = 3) | (n = 5) | (n = 8) | (n = 5) | (n = 25) |
| Age (years), median | 65.5 | 74 | 67 | 64.5 | 62 | 64 |
| (min, max) | (60, 74) | (59, 82) | (37, 72) | (55, 72) | (45, 73) | (37, 82) |
| Sex | | | | | | |
| Male | 2 | 1 | 1 | 3 | 1 | 8 |
| Female | 2 | 2 | 4 | 5 | 4 | 17 |
| Prior Anti-PD1 | 2 | 1 | 0 | 4 | 2 | 9 |
| Therapy (Yes), n (%) | (50.0) | (33.3) | | (50.0) | (40.0) | (36.0) |
| Prior Lines of | 3 | 2 | 4 | 4.5 | 4 | 4 |
| Systemic Therapy, | (2, 3) | (2, 3) | (3, 9) | (4, 15) | (1, 11) | (1, 15) |
| median (min, max) | | | | | | |
| Tumor Types | | | | | | |
| HNSCC | 1 | 1 | 0 | 2 | 1 | 5 |
| CRC | 2 | 0 | 0 | 1 | 0 | 3 |
| Ovarian | 0 | 1 | 1 | 1 | 0 | 3 |
| Pancreatic | 0 | 0 | 1 | 1 | 1 | 3 |
| Triple Neg Breast | 0 | 0 | 0 | 1 | 1 | 2 |
| Endometrial | 0 | 0 | 0 | 0 | 1 | 1 |
| SCLC | 1 | 0 | 0 | 0 | 0 | 1 |
| Esophageal | 0 | 1 | 0 | 0 | 0 | 1 |
| Cholangiocarcinoma | 0 | 0 | 1 | 0 | 0 | 1 |
| IDC (breast) | 0 | 0 | 1 | 0 | 0 | 1 |
| Leiomyosarcoma | 0 | 0 | 1 | 0 | 0 | 1 |
| Renal | 0 | 0 | 0 | 1 | 0 | 1 |
| Uterine | 0 | 0 | 0 | 1 | 0 | 1 |
| Leiomyosarcoma | | | | | | |
| Rectal | 0 | 0 | 0 | 0 | 1 | 1 |

Abbreviations:
HNSCC: Head and Neck Squamous-Cell Carcinoma;
CRC: Colorectal cancer;
SCLC: Small Cell Lung Cancer;
IDC: Invasive ductal carcinoma

Part 2: Combination Dose Escalation with Pembrolizumab (Phase 1)

As of 3 Aug. 2023, 21 participants enrolled in the dose escalation Part 2. Patient demographics are outlined in Table 2.2.

TABLE 2.2
Demographics for dose escalation Part 2

| | Compound 1 Dose (μg/kg) | | | | |
|---|---|---|---|---|---|
| | 20 | 40 | 80 | 120 | Total |
| Demographics | (n = 4) | (n = 5) | (n = 5) | (n = 7) | (n = 21) |
| Age (years), median | 67.5 | 62 | 56 | 61 | 61 |
| (min, max) | (59, 79) | (35, 78) | (49, 61) | (52, 77) | (35, 79) |
| Sex | | | | | |
| Male | 2 | 2 | 1 | 3 | 8 |
| Female | 2 | 3 | 4 | 4 | 13 |
| Prior Anti-PD1 | 3 | 2 | 1 | 2 | 8 |
| Therapy (Yes), n (%) | (75.0) | (40.0) | (20.0) | (28.5) | (38.0) |
| Prior Lines of | 2 | 1.5 | 1.5 | 2 | 2 |
| Systemic Therapy, | (1, 3) | (1, 2) | (1, 2) | (1, 3) | (1, 3) |
| median (min, max) | | | | | |
| Tumor Types | | | | | |
| SCLC | 0 | 0 | 1 | 2 | 3 |
| Cervical | 0 | 1 | 1 | 1 | 3 |
| NSCLC | 2 | 0 | 0 | 0 | 2 |
| HNSCC | 0 | 1 | 0 | 1 | 2 |
| Endometrial | 0 | 2 | 0 | 0 | 2 |
| Cholangiocarcinoma | 0 | 0 | 0 | 2 | 2 |
| CRC | 1 | 0 | 0 | 0 | 1 |
| Acinic Cell | 0 | 1 | 0 | 0 | 1 |
| Esophageal | 1 | 0 | 0 | 0 | 1 |
| Adenoid Cystic | 0 | 0 | 1 | 0 | 1 |
| Vulvar | 0 | 0 | 1 | 0 | 1 |
| Nasopharyngeal | 0 | 0 | 1 | 0 | 1 |
| Adrenocortical | 0 | 0 | 0 | 1 | 1 |

Abbreviations:
SCLC: Small cell lung cancer;
NSCLC: Non-small-cell lung cancer;
HNSCC: Head and neck squamous-cell carcinoma;
CRC: Colorectal cancer

### Part 3: Indication Specific Dose Expansion (Phase 2), Cohort 3 and Cohort 4

As of 16 Apr. 12, 2024, participants enrolled in Part 3, Cohort 3 PROC and 6 participants enrolled in Part 3, Cohort 4, post anti-PD-1 melanoma. Patient demographics are outlined in Table 2.3 and 2.4.

TABLE 2.3
Demographics for dose expansion, Part 3, Cohort 3

| | Cohort 3: Compound 1 | |
|---|---|---|
| | (dose: 120 μg/kg) | |
| | in combination with | |
| | Standard of Care Chemotherapy | |
| Demographics | n = 12 | |
| Standard of Care Chemotherapy | | |
| Pemetrexed | n = 7 | |
| Paclitaxel | n = 4 | |
| Docetaxel | n = 1 | |
| Age (years), median (min, max) | 65 | (38, 77) |
| Sex | | |
| Male | 0 | |
| Female | 12 | |
| Prior Anti-PD1 Therapy (Yes), n (%) | 3 | (25) |
| Prior Lines of Systemic | 5 | (2, 7) |

| Therapy, median (min, max) | |
|---|---|
| Tumor Type | |
| Ovarian Cancer | 12 |

TABLE 2.4
Demographics for dose expansion, Part 3, Cohort 4

| | Cohort 4: Compound 1 | |
|---|---|---|
| | (dose: 120 μg/kg) | |
| | in combination with compound | |
| | 2 (dose: 0.5 mg/lesion) | |
| Demographics | n = 6 | |
| Age (years), median (min, max) | 67 | (53, 77) |
| Sex | | |
| Male | 4 | |
| Female | 2 | |
| Prior Anti-PD1 Therapy (Yes), n (%) | 6 | (100) |
| Prior Lines of Systemic | 1.5 | (1, 2) |

| | |
|---|---|
| Therapy, median (min, max) | |
| Tumor Type | |
| Melanoma | 6 |

### Example 4—Clinical Safety Summary

### Part 1: Monotherapy Dose Escalation (Phase 1)

Data as of 28 Apr. 2023 are presented for treatment-emergent adverse events (TEALs) related to compound 1 monotherapy (Table 3.1). Five Grade 3 TEALs related to compound 1 were reported, all at 160 μg/kg. They were cytokine release syndrome, neutrophil count decreased, anaemia, hypoxia and thrombocytopaenia. There was one Grade 4 TEAL (lymphopaenia) related to compound 1 at 20 μg/kg (this event was deemed non-clinically significant by the treating physician). There were no fatal TEALs (Grade 5) related to compound 1. The most common TEALs related to compound 1 (>15%) were: pyrexia, cytokine release syndrome, nausea, fatigue, vomiting, influenza like illness, neutrophil count decreased and tachycardia (Table 4.1).

TABLE 3.1
Summary of Participant Status from Part 1 Dose Escalation

| | Number of participants, n (%) | | | | | |
|---|---|---|---|---|---|---|
| | Compound 1 Dose (μg/kg) | | | | | |
| | 20 | 40 | 80 | 120 | 160 | Total |
| | (N = 4) | (N = 3) | (N = 5) | (N = 8) | (N = 5) | (N = 25) |
| ≥Grade 3 TEAE | 2 | 3 | 2 | 3 | 4 | 14 |
| | (50.0) | (100.0) | (40.0) | (37.5) | (80.0) | (56.0) |
| ≥Grade 3 TEAE related to | 1 | 0 | 0 | 0 | 4 | 5 |
| compound 1 | (25.0) | | | | (80.0) | (20.0) |
| Serious TEAE | 1 | 3 | 3 | 3 | 4 | 14 |
| | (25.0) | (100.0) | (60.0) | (37.5) | (80.0) | (56.0) |
| Serious TEAE related to | 0 | 0 | 2 | 1 | 3 | 6 |
| compound 1 | | | (40.0) | (12.5) | (60.0) | (24.0) |
| TEAE leading to | 1 | 0 | 1 | 2 | 1 | 5 |
| discontinuation of compound 1 | (25.0) | | (20.0) | (25.0) | (20.0) | (20.0) |
| TEAE related leading to | 0 | 0 | 1 | 1 | 0 | 2 |
| discontinuation of compound 1 | | | (20.0) | (12.5) | | (8.0) |
| TEAE leading to | 3 | 2 | 1 | 2 | 2 | 10 |
| interruption/reduction of | (75.0) | (66.7) | (20.0) | (25.0) | (40.0) | (40.0) |
| compound 1 | | | | | | |
| TEAE related leading to | 0 | 0 | 2 | 0 | 1 | 3 |
| interruption/reduction of | | | (40.0) | | (20.0) | (12.0) |
| compound 1 | | | | | | |

TABLE 4.1
Dose Escalation Part 1: TEAEs related to compound 1

| | Number of participants, n (%) | | | | | |
|---|---|---|---|---|---|---|
| | Compound 1 Dose (μg/kg) | | | | | |
| Preferred Term | 20 | 40 | 80 | 120 | 160 | Total |
| Highest Grade⁴ | (N = 4) | (N = 3) | (N = 5) | (N = 8) | (N = 5) | (N = 25) |
| Subjects with any TEAEs | 4 | 2 | 4 | 7 | 5 | 22 |
| related to compound 1 | (100.0) | (66.7) | (80.0) | (87.5) | (100.0) | (88.0) |
| Pyrexia | 1 (25.0) | 0 | 2 (40.0) | 2 (25.0) | 3 (60.0) | 8 (32.0) |
| Cytokine release syndrome | 0 | 0 | 2 (40.0) | 3 (37.5) | 2 (40.0) | 7 (28.0) |
| Grade 3 | 0 | 0 | 0 | 0 | 1 (20.0) | 1 (4.0) |
| Nausea | 1 (25.0) | 1 (33.3) | 1 (20.0) | 3 (37.5) | 1 (20.0) | 7 (28.0) |
| Fatigue | 1 (25.0) | 2 (66.7) | 1 (20.0) | 0 | 1 (20.0) | 5 (20.0) |
| Vomiting | 0 | 1 (33.3) | 1 (20.0) | 3 (37.5) | 0 | 5 (20.0) |
| Influenza like illness | 1 (25.0) | 0 | 2 (40.0) | 0 | 1 (20.0) | 4 (16.0) |
| Neutrophil count | 0 | 0 | 1 (20.0) | 2 (25.0) | 1 (20.0) | 4 (16.0) |
| decreased | | | | | | |
| Grade 3 | 0 | 0 | 0 | 0 | 1 (20.0) | 1 (4.0) |
| Tachycardia | 0 | 0 | 2 (40.0) | 0 | 2 (40.0) | 4 (16.0) |
| Anaemia | 1 (25.0) | 1 (33.3) | 0 | 0 | 1 (20.0) | 3 (12.0) |
| Grade 3 | 0 | 0 | 0 | 0 | 1 (20.0) | 1 (4.0) |
| Diarrhoea | 1 (25.0) | 1 (33.3) | 0 | 0 | 1 (20.0) | 3 (12.0) |
| Hypotension | 0 | 1 (33.3) | 1 (20.0) | 1 (12.5) | 0 | 3 (12.0) |
| Infusion related reaction | 0 | 0 | 1 (20.0) | 1 (12.5) | 1 (20.0) | 3 (12.0) |
| Rash | 0 | 0 | 0 | 3 (37.5) | 0 | 3 (12.0) |
| Chills | 1 (25.0) | 0 | 1 (20.0) | 0 | 0 | 2 (8.0) |
| Dizziness | 0 | 1 (33.3) | 0 | 0 | 1 (20.0) | 2 (8.0) |
| Headache | 0 | 0 | 1 (20.0) | 0 | 1 (20.0) | 2 (8.0) |
| Myalgia | 0 | 0 | 1 (20.0) | 1 (12.5) | 0 | 2 (8.0) |
| Rash maculo-papular | 0 | 0 | 1 (20.0) | 0 | 1 (20.0) | 2 (8.0) |
| Abdominal pain | 1 (25.0) | 0 | 0 | 0 | 0 | 1 (4.0) |
| Abdominal pain upper | 1 (25.0) | 0 | 0 | 0 | 0 | 1 (4.0) |
| Arthralgia | 0 | 0 | 1 (20.0) | 0 | 0 | 1 (4.0) |
| Atrial fibrillation | 0 | 0 | 0 | 0 | 1 (20.0) | 1 (4.0) |
| Back pain | 0 | 0 | 0 | 1 (12.5) | 0 | 1 (4.0) |
| Bone pain | 1 (25.0) | 0 | 0 | 0 | 0 | 1 (4.0) |
| Candida infection | 0 | 0 | 1 (20.0) | 0 | 0 | 1 (4.0) |
| Capillary leak syndrome | 0 | 0 | 0 | 0 | 1 (20.0) | 1 (4.0) |
| Constipation | 0 | 0 | 0 | 0 | 1 (20.0) | 1 (4.0) |
| Cough | 0 | 0 | 1 (20.0) | 0 | 0 | 1 (4.0) |
| Dry mouth | 0 | 0 | 0 | 1 (12.5) | 0 | 1 (4.0) |
| Flushing | 0 | 1 (33.3) | 0 | 0 | 0 | 1 (4.0) |
| Gastritis | 1 (25.0) | 0 | 0 | 0 | 0 | 1 (4.0) |
| Hepatic enzyme increased | 0 | 1 (33.3) | 0 | 0 | 0 | 1 (4.0) |
| Hypoxia | 0 | 0 | 0 | 0 | 1 (20.0) | 1 (4.0) |
| Grade 3 | 0 | 0 | 0 | 0 | 1 (20.0) | 1 (4.0) |
| Insomnia | 0 | 0 | 0 | 0 | 1 (20.0) | 1 (4.0) |
| Lymphocyte count | 0 | 0 | 1 (20.0) | 0 | 0 | 1 (4.0) |
| decreased | | | | | | |
| Lymphopaenia | 1 (25.0) | 0 | 0 | 0 | 0 | 1 (4.0) |
| Grade 4 | 1 (25.0) | 0 | 0 | 0 | 0 | 1 (4.0) |
| Malaise | 0 | 0 | 0 | 1 (12.5) | 0 | 1 (4.0) |
| Mouth ulceration | 0 | 0 | 1 (20.0) | 0 | 0 | 1 (4.0) |
| Muscle spasms | 0 | 0 | 0 | 1 (12.5) | 0 | 1 (4.0) |
| Muscular weakness | 0 | 0 | 0 | 0 | 1 (20.0) | 1 (4.0) |
| Neuropathy peripheral | 0 | 0 | 0 | 0 | 1 (20.0) | 1 (4.0) |
| Oedema | 0 | 0 | 0 | 1 (12.5) | 0 | 1 (4.0) |
| Pain | 0 | 0 | 0 | 1 (12.5) | 0 | 1 (4.0) |
| Platelet count decreased | 0 | 0 | 1 (20.0) | 0 | 0 | 1 (4.0) |
| Pleural effusion | 0 | 0 | 0 | 1 (12.5) | 0 | 1 (4.0) |
| Post procedural discomfort | 1 (25.0) | 0 | 0 | 0 | 0 | 1 (4.0) |
| Pruritus | 0 | 0 | 0 | 1 (12.5) | 0 | 1 (4.0) |
| Taste disorder | 0 | 0 | 0 | 0 | 1 (20.0) | 1 (4.0) |
| Thrombocytopaenia | 0 | 0 | 0 | 0 | 1 (20.0) | 1 (4.0) |
| Grade 3 | 0 | 0 | 0 | 0 | 1 (20.0) | 1 (4.0) |
| Weight decreased | 1 (25.0) | 0 | 0 | 0 | 0 | 1 (4.0) |
| White blood cell count | 0 | 0 | 1 (20.0) | 0 | 0 | 1 (4.0) |
| decreased | | | | | | |

⁴Columns with “Grade 3” or “Grade 4” correspond to the highest grade detected for the TEAE indicated in the row directly above.

Part 2: Combination Dose Escalation with Pembrolizumab (Phase 1)

Data as of 3 Aug. 2023 are presented for treatment-emergent adverse events (TEALs) related to compound 1 or pembrolizumab (Table 3.2). Three participants experienced Grade 3 TEALs related to compound 1. One participant at 20 μg/kg experienced G3 aspartate aminotransferase (AST) increase, one participant at 40 μg/kg experienced G3 fatigue, G3 vomiting and G3 weight loss and one participant at 120 μg/kg experienced G3 nausea and G3 vomiting. There were no fatal TEALs (Grade 5) related to compound 1. The most common TEALs related to compound 1 (>15% o) were: myalgia, cytokine release syndrome, nausea and rash (Table 4.2).

TABLE 3.2
Summary of Participant Status from Part 2, Combination Dose Escalation

| | Number of participants, n (%) | | | | | |
|---|---|---|---|---|---|---|
| | Compound 1 Dose (μg/kg) | | | | | |
| | 20 | 40 | 80 | 120 | Total | |
| | (N = 4) | (N = 5) | (N = 5) | (N = 7) | (N = 21) | |
| ≥Grade 3 TEAE | 2 (50.0) | 2 (40.0) | 1 (20.0) | 2 (28.6) | 7 | (33.3) |
| ≥Grade 3 TEAE related to | 1 (25.0) | 1 (20.0) | 0 | 1 (14.3) | 3 | (14.2) |
| compound 1 | | | | | | |
| ≥Grade 3 TEAE related to | 0 | 1 (20.0) | 0 | 1 (14.3) | 2 | (9.5) |
| Pembrolizumab | | | | | | |
| Serious TEAE | 2 (50.0) | 2 (40.0) | 0 | 2 (28.6) | 6 | (28.5) |
| Serious TEAE related to | 0 | 1 (20.0) | 0 | 1 (14.3) | 2 | (9.5) |
| compound 1 | | | | | | |
| Serious TEAE related to | 0 | 1 (20.0) | 0 | 1 (14.3) | 2 | (9.5) |
| Pembrolizumab | | | | | | |
| TEAE leading to | 0 | 0 | 0 | 0 | 0 | |
| discontinuation of compound 1 | | | | | | |
| or Pembrolizumab | | | | | | |
| TEAE leading to reduction of | 0 | 0 | 1 (20.0) | 1 (14.3) | 2 | (9.5) |
| compound 1 | | | | | | |
| TEAE related leading to | 0 | 0 | 1 (20.0) | 1 (14.3) | 2 | (9.5) |
| reduction of compound 1 | | | | | | |
| TEAE leading to interruption | 0 | 2 (40.0) | 0 | 0 | 2 | (9.5) |
| of compound 1 | | | | | | |
| TEAE leading to interruption | 0 | 2 (40.0) | 0 | 0 | 2 | (9.5) |

of Pembrolizumab

TABLE 4.2
Combination Dose Escalation Part 2: TEAEs related to compound 1

| | Number of participants, n (%) | | | | | |
|---|---|---|---|---|---|---|
| | Compound 1 Dose (μg/kg) | | | | | |
| Preferred Term | 20 | 40 | 80 | 120 | Total | |
| Highest Grade⁵ | (N = 4) | (N = 5) | (N = 5) | (N = 7) | (N = 21) | |
| Subjects with any TEAEs | 2 (50) | 5 (100) | 4 (80) | 6 (85.7) | 17 | (81) |
| related to compound 1 | | | | | | |
| Fatigue | 0 | 2 (40.0) | 0 | 1 (14.3) | 3 | (14.3) |
| Grade 3 | 0 | 1 (20.0) | 0 | 0 | 1 | (4.8) |
| Pyrexia | 0 | 2 (40.0) | 1 (20.0) | 0 | 3 | (14.3) |
| Asthenia | 0 | 0 | 0 | 2 (28.6) | 2 | (9.5) |
| Chills | 0 | 1 (20.0) | 1 (20.0) | 0 | 2 | (9.5) |
| Influenza like illness | 1 (25.0) | 0 | 1 (20.0) | 0 | 2 | (9.5) |
| Localized oedema | 0 | 0 | 0 | 1 (14.3) | 1 | (4.8) |
| Nausea | 0 | 1 (20.0) | 2 (40.0) | 2 (28.6) | 5 | (23.8) |
| Grade 3 | 0 | 0 | 0 | 1 (14.3) | 1 | (4.8) |
| Vomiting | 0 | 2 (40.0) | 0 | 1 (14.3) | 3 | (14.3) |
| Grade 3 | 0 | 1 (20.0) | 0 | 1 (14.3) | 2 | (9.5) |
| Dry mouth | 0 | 0 | 1 (20.0) | 1 (14.3) | 2 | (9.5) |
| Diarrhea | 0 | 1 (20.0) | 0 | 0 | 1 | (4.8) |
| Gastritis | 0 | 1 (20.0) | 0 | 0 | 1 | (4.8) |
| Glossodynia | 0 | 1 (20.0) | 0 | 0 | 1 | (4.8) |
| Cytokine release syndrome | 0 | 2 (40.0) | 0 | 3 (42.9) | 5 | (23.8) |
| Anaemia | 0 | 0 | 1 (20.0) | 0 | 1 | (4.8) |
| Hyperthyroidism | 0 | 1 (20.0) | 0 | 0 | 1 | (4.8) |
| Hepatitis | 0 | 0 | 1 (20.0) | 0 | 1 | (4.8) |
| Infusion related reaction | 0 | 0 | 1 (20.0) | 0 | 1 | (4.8) |
| Rash | 0 | 2 (40.0) | 1 (20.0) | 1 (14.3) | 4 | (19) |
| Pruritus | 0 | 0 | 2 (40.0) | 1 (14.3) | 3 | (14.3) |
| Rash maculo-papular | 0 | 1 (20.0) | 0 | 2 (28.6) | 3 | (14.3) |
| Dermatitis | 0 | 0 | 1 (20.0) | 0 | 1 | (4.8) |
| Rash macular | 0 | 0 | 1 (20.0) | 0 | 1 | (4.8) |
| Rash papular | 0 | 0 | 1 (20.0) | 0 | 1 | (4.8) |
| Myalgia | 0 | 1 (20.0) | 1 (20.0) | 3 (42.9) | 5 | (23.8) |
| Muscle spams | 0 | 0 | 1 (20.0) | 0 | 1 | (4.8) |
| AST increased | 1 (25.0) | 0 | 0 | 1 (14.3) | 2 | (9.5) |
| Grade 3 | 1 (25.0) | 0 | 0 | 0 | 1 | (4.8) |
| Weight decreased | 0 | 1 (20.0) | 1 (20.0) | 0 | 2 | (9.5) |
| Grade 3 | 0 | 1 (20.0) | 0 | 0 | 1 | (4.8) |
| ALT increased | 0 | 0 | 0 | 1 (14.3) | 1 | (4.8) |
| ALP increased | 0 | 0 | 0 | 1 (14.3) | 1 | (4.8) |
| Platelet decreased | 0 | 0 | 0 | 1 (14.3) | 1 | (4.8) |
| Decreased appetite | 0 | 2 (40.0) | 1 (20.0) | 0 | 3 | (14.3) |
| Gout | 0 | 0 | 0 | 1 (14.3) | 1 | (4.8) |
| Headache | 0 | 0 | 1 (20.0) | 0 | 1 | (4.8) |
| Uncoded Cytokine Release | 0 | 0 | 0 | 1 (14.3) | 1 | (4.8) |

Syndrome

⁵Columns with “Grade 3” or “Grade 4” correspond to the highest grade detected for the TEAE indicated in the row directly above.

### Part 3: Indication Specific Dose Expansion (Phase 2), Cohort 3 and Cohort 4

As of 16 Apr. 2024, 12 participants were treated in Part 3, Cohort 3 PROC. The most common treatment related adverse events (TRAEs) related to compound 1 and standard of care chemotherapy were fatigue (66.7%), nausea (41.7%), and neutropenia (33.3%). TRAEs >Grade 3 were neutropenia (25%), fatigue (16.7%), decreased appetite (8.3%), chronic inflammatory demyelinating polyneuropathy (8.3%) and thrombocytopenia (8.3%). There were no fatal TEAEs (Grade 5) related to compound 1 or standard of care chemotherapy.

As of 16 Apr. 2024, 6 participants were treated in Part 3, Cohort 4 post anti-PD-1 melanoma. The most common treatment related adverse events (TRAEs) related to compound 1 and compound 2 were nausea (83.3%), vomiting (66.7%), CRS (66.7%) and chills (66.7%). 4 participants experienced G3 TRAEs related to compound 1 or compound 2. One participant experienced G3 adrenal insufficiency related to compound 1, one participant experienced G3 acute kidney injury related to compound 1, one participant experienced G3 muscle weakness and G3 Guillain-Barre Syndrome related to compound 1 and compound 2 and G3 injection site reaction related to compound 2, and one participant experienced G3 hyponatremia related to compound 1 and compound 2. There were no fatal TEAEs (Grade 5) related to compound 1 or compound 2.

### Example 5—Pharmacokinetic Assessment

Plasma exposure to compound 1 in terms of both maximum plasma concentration (Cₘₐₓ) and area under the curve (AUC) after the first dose administration of compound 1 was dose proportional in the dose range from 20 to 160 μg/kg. The preliminary half-life (t1/2) of compound 1 was at least 35 hours. Further analysis showed that the half-life of compound 1 was approximately 37 hours. The half-life was independent of the dose level. The mean PK parameters are in Table 5. The plasma concentrations of compound 1 in samples collected pre-dose at the second cycle were all below the lower limit of quantification (LLOQ), indicating that compound 1 does not accumulate when dosed Q3W. Moreover, similar plasma concentration-time profiles of compound 1 were observed after monotherapy and in combination therapy with pembrolizumab.

TABLE 5
PK Parameters Following the First Administration of Compound 1

| Dose | | Cₘₐₓ | Tₘₐₓ | Tₗₐₛₜ | AUC₀₋ₜₗₐₛₜ |
|---|---|---|---|---|---|
| (μg/kg) | Statistic | (ng/mL) | (h) | (h) | (h*ng/mL) |
| 20 | Mean | 479 | 0.88 | 210 | 23,500 |
| (n = 4) | CV % | 37 | 85.7 | 40 | 31 |
| 40 | Mean | 836 | 0.5 | 168 | 39,200 |
| (n = 3) | CV % | 8.6 | 0 | 0 | 16 |
| 80 | Mean | 2,090 | 1.1 | 302 | 103,000 |
| (n = 5) | CV % | 29 | 75 | 25 | 35 |
| 160 | Mean | 3,300 | 2.25 | 252 | 149,000 |
| (n = 4) | CV % | 20 | 115 | 38 | 13 |

Abbreviations:
AUC₀₋ₜₗₐₛₜ = area under the curve from the time of dosing to the last measured concentration;
Cₘₐₓ = maximum plasma concentration of compound 1;
CV = coefficient of variation;
Tₗₐₛₜ = time of last measured concentration;
Tₘₐₓ = time of maximum concentration

### Example 6—Biomarkers as Surrogates for Safety

### Part 1: Monotherapy Dose Escalation (Phase 1)

Increased eosinophil counts and high Interleukin 5 (IL-5) levels are associated with vascular leak syndrome, a common and serious side effect of classical IL-2 treatment, such as aldesleukin. Eosinophil counts in peripheral blood from compound 1 treated patients were determined in the first cycle of treatment on Day 1 (pre-treatment) and Day 8 (7 days after dosing) using standard clinical hematology measurements. No significant changes in eosinophil counts were observed 7 days after administration (Table 6.1). All patients in all cohorts had a blood median eosinophil count within the normal range of 0-500 cells/μL (Klion, et al., Annu Rev Pathol. 2020 Jan. 24; 15:179-209; Valent, et al., J Allergy Clin Immunol. 2012 Sep;130(3):607-612.e9).

Plasma IL-5 levels in blood from compound 1 treated patients were determined in the first cycle of treatment on Day 1 (pre-treatment), Day 2 and Day 5 (1 and 4 days after dosing, respectively) using the clinically validated Meso Scale Discovery Technology platform (V-PLEX Human IL-5 Kit). No patient showed IL-5 levels that significantly exceeded the normal range of 5-15 μg/mL (Joseph, et al., BMC Pulm Med., 4, 2, 2004) 1 or 4 days after treatment with compound 1. Most patients had undetectable IL-5 levels (below 6.3 μg/mL) or levels within the normal range.

TABLE 6.1
Eosinophil Counts in Each Dose Level Pre-Treatment and 7 Days After First Dose

| | Median Eosinophil Counts (cells/μL) | | | | |
|---|---|---|---|---|---|
| | 20 μg/kg | 40 μg/kg | 80 μg/kg | 120 μg/kg | 160 μg/kg |
| | (n = 3-4) | (n = 3) | (n = 5) | (n = 6-8) | (n = 2-5) |
| Pre-treatment | 190 | 70 | 90 | 140 | 110 |
| 7 days post dose | 130 | 90 | 90 | 180 | 20 |

Part 2: Combination Dose Escalation with Pembrolizumab (Phase 1)

Eosinophil counts in peripheral blood from patients treated with compound 1 in combination with pembrolizumab were determined in the first cycle of treatment on Day 1 (pre-treatment) and Day 8 (7 days after dosing) using standard clinical hematology measurements. No significant changes in eosinophil counts were observed 7 days after administration (Table 6.2). All patients had a blood median eosinophil count within the normal range of 0-500 cells/μL, as observed for compound 1 monotherapy (Klion, et al., Annu Rev Pathol. 2020 Jan. 24; 15:179-209; Valent, et al., J Allergy Clin Immunol. 2012 September;130(3):607-612.e9).

TABLE 6.2
Eosinophil Counts in Each Dose Level of combination
therapy Pre-Treatment and 7 Days After First Dose

| | Median Eosinophil Counts (cells/μL) | | | |
|---|---|---|---|---|
| | 20 μg/kg | 40 μg/kg | 80 μg/kg | 120 μg/kg |
| | (n = 3-4) | (n = 4-5) | (n = 5) | (n = 7) |
| Pre-treatment | 340 | 200 | 150 | 150 |
| 7 days post dose | 420 | 150 | 220 | 150 |

Overarching data analysis revealed no difference in surrogate-safety biomarkers between patients treated with monotherapy of compound 1 and those treated in combination with compound 1 and pembrolizumab.

### Example 7—Biomarkers as Surrogates for Clinical Response

### Part 1: Monotherapy Dose Escalation (Phase 1)

Lymphocytes, in particular cytotoxic lymphocytes like CD8+ T cells and NK cells, enable the patient's immune system to eradicate cancer cells. Increasing lymphocyte numbers is a key mode of action in interleukin-2 based therapies.

Lymphocyte counts in peripheral blood were determined in the first cycle of compound 1 treatment on Day 1 (pre-treatment) and Day 8 (7 days after dosing) using standard clinical hematology measurements. A significant and dose-proportional increase in lymphocyte counts was observed 7 days after treatment. At a compound 1 dose of 120 μg/kg, patients showed a median lymphocyte count of 4540 cells/μL, which was a clear increase over the lower dose levels, which only increased the median lymphocyte count up to a max of 3360 cells/μL (Table 7.1). A further increase in compound 1 dose (160 μg/kg) did not lead to an additional increase in the median lymphocyte count (Table 7.1).

Lymphocyte proliferation was measured by flow cytometry, using the intracellular marker Ki67, on PBMC samples obtained in the first cycle of compound 1 treatment on Day 1 (pre-treatment), Day 5 and Day 8 (4 and 7 days after dosing). All patients showed an increased percentage of proliferating lymphocyte subsets (CD8+ T cells and Natural Killer (NK) cells) 4 and 7 days after treatment, indicating a sustained lymphocyte proliferation for at least 3 days. This prolonged proliferation of CD8+ T cells and NK cells over several days is most likely due to the controlled and sustained release of active IL-2 3/γ and has not been reported for other IL-2 molecules, including bempegaldesleukin (NKTR-214), MDNA11, THOR-707 (SAR444245), or ANV419 (Bentebibel, et al., Cancer Discov. 2019; 9(6):711-721; Bucher, et al., Journal for ImmunoTherapy of Cancer 2022;10:doi: 10.1136/jitc-2022-SITC2022.0631; Fu, et al., AACR 2023, abstract 2155; To, et al., MDNAll presentation at the Frontiers in Cancer Immunotherapy 2022, 9-11 May 2022, New York, US. https://ir.medicenna.com/static-files/e61d510c-cdad-451b-9695-beb86cef76e0).

Cytotoxic lymphocyte (CD8+ T cells and NK cells) concentration in peripheral blood was determined using flow cytometry in the first cycle of compound 1 treatment on Day 1 (pre-treatment) and Day 8 (7 days after dosing). NK cell counts increased in a dose-proportional fashion 7 days after administration of compound 1, reaching concentrations above 2000 cells/μL. CD8+ T cell counts increased markedly in all patients 7 days after treatment, reaching concentrations above 1000 cells/μL in individual patients who received 120 μg/kg or 160 μg/kg of compound 1. The concentrations of CD8+ T cells and NK cells reached after administration of compound 1 are high compared to what has been reported for other IL-2 molecules, including THOR-707, nemvaleukin alfa, bempegaldesleukin, MDNA11 and ANV419 (Bentebibel, et al., Cancer Discov. 2019; 9(6):711-721; Janku, et al., Cancer Res (2021) 81 (13_Supplement): LB041; Vaishampayan, et al., Journal of Clinical Oncology 40, no. 16_suppl (Jun. 1, 2022) 2500-2500; To, et al., Journal for ImmunoTherapy of Cancer 2022;10: doi: 10.1 136/jitc-2022-SITC2022.0743; Garralda, et al., AACR meeting 2022, New Orleans, 11 Apr. 2022. Abstract CT140).

Regulatory T cells (Treg) regulate the intensity of cytotoxic and other immune responses, by, among other things, inhibiting CD8+ T cell and NK cell function. Classical IL-2 immunotherapy, such as aldesleukin, induces a strong Treg expansion, leading to an unwanted immune-dampening effect. Treg concentration in peripheral blood of compound 1 treated patients was determined using flow cytometry. Tregs did not show a consistent or dose-dependent increase after treatment with compound 1. The majority of the patients showed levels of Tregs within the normal range of 20-120 cells/μL (Liotta, et al., BJU Int. 2011 May; 107(9):1500-6; Li, et al., Oncol Lett. 2019 August; 18(2):1424-1430) after treatment.

The robust expansion of cytotoxic lymphocytes over Tregs in response to compound 1 can be expected for an IL-2Rβ/γ-biased IL-2 molecule such as compound 1. This is in contrast to what is seen with aldesleukin or bempegaldesleukin that bind to the trimeric IL-2Rα/β/γ and expand the Treg population in peripheral blood (Bentebibel, et al., Cancer Discov. 2019; 9(6):711-721; Ahmadzadeh, et al., Blood. 2006; 107:2409-2414).

TABLE 7.1
Lymphocyte Counts in Each Dose Level Pre-
Treatment and 7 Days After First Dose

| | Median Lymphocyte Counts (cells/μL) | | | | |
|---|---|---|---|---|---|
| | 20 μg/kg | 40 μg/kg | 80 μg/kg | 120 μg/kg | 160 μg/kg |
| | (n = 3-4) | (n = 3) | (n = 5) | (n = 7-8) | (n = 5) |
| Pre-treatment | 870 | 580 | 1220 | 1230 | 710 |
| 7 days post dose | 1500 | 1960 | 3360 | 4540 | 4740 |

Part 2: Combination Dose Escalation with Pembrolizumab (Phase 1)

Lymphocyte counts in peripheral blood were determined in the first cycle of compound 1 plus pembrolizumab combination treatment on Day 1 (pre-treatment) and Day 8 (7 days after dosing) using standard clinical hematology measurements. A significant and dose-proportional increase in lymphocyte counts was observed 7 days after treatment, which was similar to that observed after compound 1 monotherapy. At a compound 1 dose of 120 μg/kg, patients showed a median lymphocyte count of 4740 cells/μL, which was a clear increase over the lower dose levels, which only increased the median lymphocyte count up to a max of 2745 cells/μL (Table 7.2).

TABLE 7.2
Lymphocyte Counts in Each Dose Level Pre-
Treatment and 7 Days After First Dose

| | Median Lymphocyte Counts (cells/μL) | | | |
|---|---|---|---|---|
| | 20 μg/kg | 40 μg/kg | 80 μg/kg | 120 μg/kg |
| | (n = 3-4) | (n = 4-5) | (n = 4-5) | (n = 7) |
| Pre-treatment | 845 | 770 | 1330 | 1400 |
| 7 days post dose | 1250 | 1570 | 2745 | 4740 |

Overarching data analysis revealed no difference in surrogate-response biomarkers between patients treated with monotherapy of compound 1 and those treated in combination with compound 1 and pembrolizumab.

Part 1 and 2: Biomarkers Indicating Sustained Immune Activation and Correlation with Clinical Benefit

Cytokines like Interferon-gamma (IFN-7) as well as the released IL-2 receptor alpha (soluble CD25, sCD25) indicate activation of cytotoxic lymphocytes, in particular activation of CD8+ T cells. Chemokines like C—X—C Motif Chemokine Ligand 10 (CXCL10) and chemokine (C—C motif) ligand 2 (CCL2) indicate a broader immune response, as they are coordinating the tissue infiltration by cytotoxic lymphocytes.

At a compound 1 dose of 120 μg/kg, patients showed a significant median IFN-γ increase from 7 μg/ml to 222 μg/ml 24 h after administration (p=0.0051, paired t-test). Surprisingly, 3 weeks after the first dose of compound 1, patients still showed significantly elevated IFN-γ levels (median=15 pg/ml, p=0.0395, paired t-test), indicating a sustained immune activation that has not been described before for other IL-2 compounds. CXCL10 and CCL2 show similar results of sustained increased levels after first compound 1 dose of 120 μg/kg.

Patients who responded to compound 1 at any dose-level or derived clinical benefit by having stable-disease for more than 6 months, showed significantly higher levels of sCD25 4 days and 3 weeks after first dose of compound 1 (p=0.0106 & p=0.0067 respectively, Mann-Whitney test), indicating that cytotoxic lymphocyte activation is indicative of response to compound 1.

Patients who responded to compound 1 at any dose-level or derived clinical benefit by having stable-disease for more than 6 months, showed significantly higher levels of CD8+ T cells 3 weeks after first dose and 1 week after second dose (p=0.024 & p=0.028 respectively, Mann-Whitney test), indicating that CD8+ cells levels and/or proliferation is indicative of response to compound 1.

### Example 8—Clinical Efficacy

### Part 1: Monotherapy Dose Escalation (Phase 1)

As of 28 Apr. 2023, 18 participants in Dose Escalation Part 1 were evaluable for efficacy assessment by investigator per RECIST v1.1. One participant with microsatellite high metastatic colorectal cancer reported an unconfirmed partial response at a dose of 120 μg/kg. This participant had previously progressed on nivolumab and is still undergoing treatment with compound 1. Four participants had a best overall response of stable disease: one at 80 μg/kg (microsatellite stable pancreatic cancer), one at 120 μg/kg (clear cell renal cell carcinoma (ccRCC) with prior anti-PD(L)1 treatment) and two at 160 μg/kg (triple negative breast cancer (TNBC) with prior anti-PD(L)1 treatment and pancreatic cancer). The participant with stable disease at 80 μg/kg has metastatic pancreatic cancer with stable disease >45 weeks with ongoing treatment.

As of 15 Aug. 2023, 18 participants in Dose Escalation Part 1 were evaluable for efficacy assessment by investigator per RECIST v1.1. The participant with the microsatellite high metastatic colorectal cancer in the 120 μg/kg group had a confirmed partial response after completing nine cycles of treatment and is still undergoing treatment with compound 1.

As of 16 Apr. 2024, 18 participants in Dose Escalation Part 1 were evaluable for efficacy assessment by investigator per RECIST v1.1. All patients had discontinued treatment at the time of data cut-off.

Part 2: Combination Dose Escalation with Pembrolizumab (Phase 1)

As of 3 Aug. 2023, 15 participants in Combination Dose Escalation Part 2 were evaluable for efficacy assessment by investigator per RECIST vi.i. Three participants had confirmed partial responses (two participants in 40 μg/kg with Acinic Cell Carcinoma and Endometrial Carcinoma and one participant in 80 μg/kg with Small Cell Lung Cancer). The participant with metastatic Small Cell Lung Cancer progressed on prior anti PD-L1 therapy, has received 7 cycles of therapy and is ongoing with treatment.

As of 2 Oct. 2023, one participant with Small Cell Lung Cancer receiving 120 μg/kg of compound 1 had an unconfirmed complete response. This participant progressed on prior anti-PD-1 treatment, has received four cycles of therapy and is ongoing with treatment.

As of 16 Apr. 2024, 21 participants in Dose Escalation Part 2 were evaluable for efficacy assessment by investigator per RECIST vi.i. The participant with Small Cell Lung Cancer receiving 120 μg/kg of compound 1 had a confirmed complete response after completing eight cycles of treatment and is off treatment lost to follow up.

### Part 3: Indication Specific Dose Expansion (Phase 2), Cohort 3 and Cohort 4

As of May 6, 2024, 6 participants in Part 3, Cohort 3 PROC were evaluable for efficacy assessment by investigator per RECIST v1.1. 2 participants had a confirmed partial response.

As of 16 Apr. 2024, 5 participants in Part 3, Cohort 4 post anti-PD-1 melanoma were evaluable for efficacy assessment by investigator per RECIST v1.1.2 participants had a confirmed partial response.

### Example 9—RP2D for Part 3 Dose Expansion cohorts

### Part 1: Monotherapy Dose Escalation (Phase 1)

The recommended phase 2 dose (RP2D) of compound 1 monotherapy was declared to be 120 μg/kg by the Safety Review Committee. One dose-limiting toxicity (DLT) (Grade 3, related cytokine release syndrome) occurred at the 160 μg/kg dose.

Part 2: Combination Dose Escalation with Pembrolizumab (Phase 1)

The RP2D of compound 1 in combination with Pembrolizumab was declared to be 120 μg/kg by the Safety Review Committee. There were no DLTs reported in Part 2.

### Abbreviations

μg Microgram
μl Microliter
AE Adverse event
ALP Alkaline phosphatase
ALT Alanine transaminase
AST Aspartate aminotransferase
AUC Area under the curve
bp Base pair
CAR Chimeric Antigen Receptor
CCL2 Chemokine (C—C motif) ligand 2
CD8 Cluster of Differentiation 8
Cₘₐₓ Maximum plasma concentration
CXCL10 C—X—C Motif Chemokine Ligand 10
DLT Dose-limiting toxicity(ies)
DNA Deoxyribonucleic acid
h Hour
IFN-γ Interferon-gammav
IL-2 Interleukin-2
IL-2R Interleukin-2 receptor
IL-5 Interleukin-5
IV Intravenous(ly)
kDa Kilodalton
kg Kilogram
LLOQ Lower limit of quantification
mg Milligram
min Minutes
mol eq. Molar equivalents
mPEG Methoxypolyethylene glycol
MTD Maximum tolerated dose
NK cell Natural killer cell
PBMC Peripheral blood mononuclear cells
PD Pharmacodynamic(s)
pg Picogram
PK Pharmacokinetic(s)
Q3W Every 3 weeks
RECIST Response evaluation criteria in solid tumors
RP2D Recommended Phase 2 Dose
sCD25 Soluble CD25
SOC Standard-of-care
SRC Safety Review Committee
t_{1/2} Terminal half-life
TEAE Treatment-emergent adverse events
Tₗₐₛₜ Time of last measured concentration
Tₘₐₓ Time of maximum concentration
Treg Regulatory T cell

1-18. (canceled)

19. A method of treating a patient having cancer, wherein the method comprises administering to such patient in a dose ranging from 80 μg IL-2/kg to 160 μg IL-2/kg an IL-2 conjugate or a pharmaceutically acceptable salt thereof, wherein the IL-2 conjugate is of formula (I) 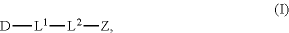
wherein
—D is —D′—M_{mod},
wherein
—D′— is an IL-2 moiety;
—M_{mod} is a first polymeric moiety that is stably conjugated to a side chain of an amino acid residue of —D′—;
—L¹— is a linker moiety covalently and reversibly attached to —D′—;
—L²— is absent or a spacer moiety; and
—Z is a second polymeric moiety.

20. The method of claim 19, wherein the dose is about 120 μg IL-2/kg.

21. The method of claim 19, wherein the dose is 120 μg IL-2/kg.

22. The method of claim 19, wherein the dose is administered multiple times.

23. The method of claim 19, wherein the interval between two consecutive administrations ranges from two to eight weeks.

24. The method of claim 19, wherein the interval between two consecutive administrations is three weeks.

25. The method of claim 19, wherein the interval between two consecutive administrations is six weeks.

26. The method of claim 19, wherein the IL-2 conjugate is administered at a frequency dependent on disease progression.

27. The method of claim 19, wherein the dose is administered once.

28. The method of claim 19, wherein the treatment starts directly on diagnosis of the cancer.

29. The method of claim 19, wherein the treatment is a first line of treatment.

30. The method of claim 19, wherein the treatment is the second, third or further line of treatment.

31. The method of claim 19, wherein the IL-2 conjugate is administered via external application, injection or infusion, including intraarticular, periarticular, intradermal, subcutaneous, intramuscular, intravenous, intraosseous, intraperitoneal, intrathecal, intracapsular, intraorbital, intravitreal, intratympanic, intravesical, intracardiac, transtracheal, subcuticular, subcapsular, subarachnoid, intraspinal, intraventricular, intrasternal injection and infusion; direct delivery to the brain via an implanted device allowing delivery of the IL-2 conjugate to brain tissue or brain fluids (e.g., Ommaya Reservoir), direct intracerebroventricular injection or infusion, injection or infusion into brain or brain associated regions, injection into the subchoroidal space, retro-orbital injection or ocular instillation.

32. The method of claim 19, wherein the IL-2 conjugate is administered intravenously.

33. The method of claim 19, wherein —D′— has the amino acid sequence of SEQ ID NO:1, in which one amino acid residue selected from the group consisting of K35, R38, M39, T41, F42, K43, F44, Y45, E62, P65, E68, V69, and L72 is replaced with a cysteine residue.

34. The method of claim 19, wherein —D′— has the amino acid sequence of SEQ ID NO. 4.

35. The method of claim 19, wherein —M_{mod} is of formula (A-1d) or (A-1e) 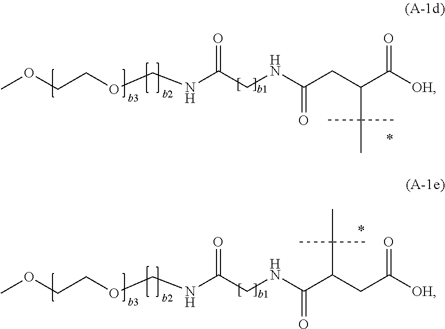
wherein
the dashed line marked with the asterisk indicates attachment to the sulfur of a cysteine residue of —D′—;
b1 is an integer selected from the group consisting of 1, 2, 3, 4, 5, 6, 7, 8, 9, 10, 11, 12, 13, 14, 15, 16, 17, 18, 19 and 20;
b2 is an integer selected from the group consisting of 1, 2, 3, 4, 5, 6, 7, 8, 9, 10, 11, 12, 13, 14, 15, 16, 17, 18, 19 and 20; and
b3 is an integer ranging from about 22 to about 340.

36. The method of claim 19, wherein —L¹—L²—Z is of formula (XI) 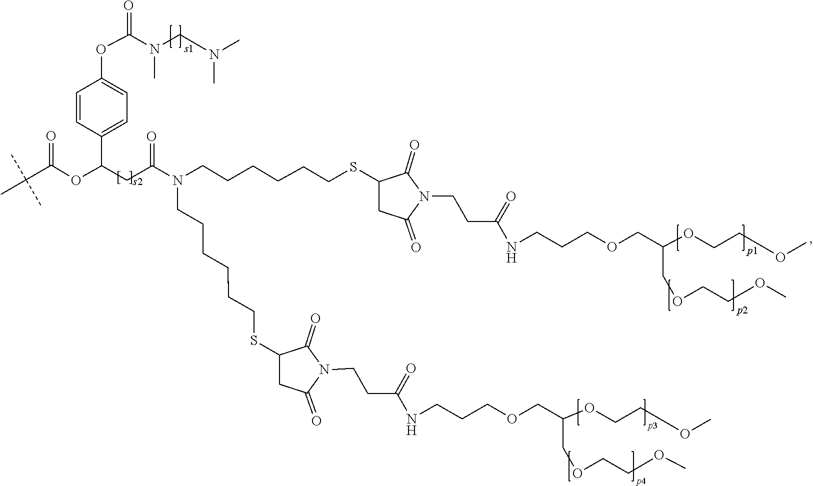
wherein the dashed line indicates attachment to a nitrogen of —D′—;
s1 is an integer selected from the group consisting of 1, 2, 3, 4, 5, 6, 7, 8, 9 and 10;
s2 is an integer selected from the group consisting of 0, 1, 2, 3, 4, 5, 6, 7, 8, 9, 10, 11, 12, 13, 14, 15, 16, 17, 18, 19 and 20; and
p1, p2, p3, p4 are independently of each other an integer ranging from 110 to 345.

37. The method of claim 19, wherein the IL-2 conjugate is of formula (I), wherein —D is —D′—M_{mod} and wherein —D′— has the amino acid sequence of SEQ ID NO. 4, —M_{mod} has the structure of (A-1d) or (A-1e) 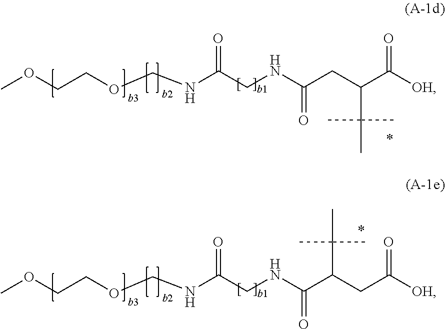
wherein
the dashed line marked with the asterisk indicates attachment to the sulfur atom of the side chain of the cysteine at position 38;
b1 is 2;
b2 is 2;
b3 ranges from 90 to 140, from 95 to 135 or from 100 to 125;
and wherein —L¹—L²—Z is of formula (XI-a) 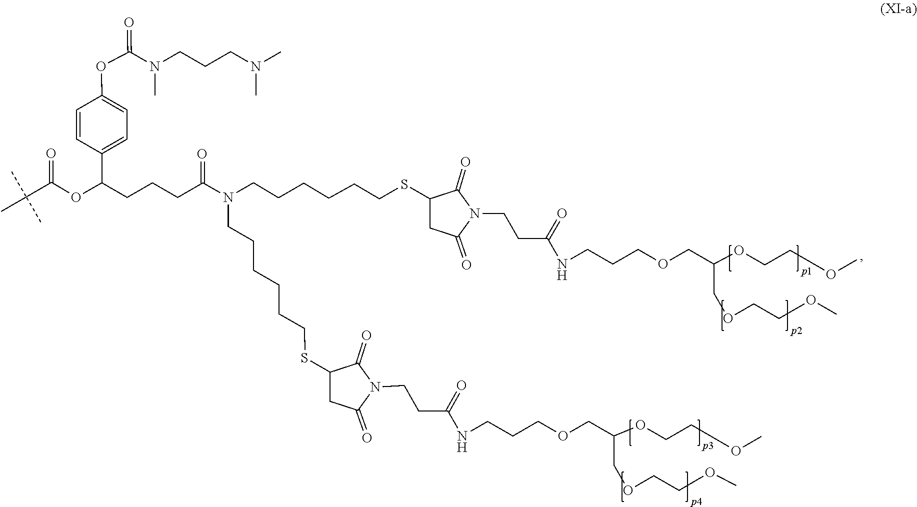
wherein the dashed line indicates attachment to the nitrogen of the ε-amino group of a lysine residue of —D′— or to the nitrogen of the N-terminal amine of —D′—; and
p1, p2, p3, p4 are independently of each other an integer ranging from 110 to 345, from 140 to 315, from 170 to 285, from 195 to 255, from 200 to 250, from 210 to 250 or from 220 to 240.

38. The method of claim 19, wherein the IL-2 conjugate is administered to the patient prior to, simultaneously with or after administration of one or more additional drug, which one or more additional drug is selected from the group consisting of pattern recognition receptor agonists (PRRA), cytotoxic/chemotherapeutic agents, immune checkpoint inhibitors or antagonists, immune checkpoint agonists, immune activating receptor agonists, multi-specific drugs, antibody-drug conjugates (ADC), antibody-adjuvant conjugates (AAC), radionuclides or targeted radionuclide therapeutics, DNA damage repair inhibitors, tumor metabolism inhibitors, pattern recognition receptor agonists, protein kinase inhibitors, chemokine and chemoattractant receptor agonists, chemokine or chemokine receptor antagonists, cytokine receptor agonists, death receptor agonists, CD47 or SIRPα antagonists, oncolytic drugs, signal converter proteins, epigenetic modifiers, tumor peptides or tumor vaccines, heat shock protein (HSP) inhibitors, proteolytic enzymes, ubiquitin and proteasome inhibitors, adhesion molecule antagonists, hormones including hormone peptides and synthetic hormones, and adoptive cellular therapies such as Tumor Infiltrating Lymphocyte (TIL) therapy, Chimeric Antigen Receptor (CAR) therapy, T cell therapy, Natural Killer (NK) cell therapy, CAR-T therapy, CAR-NK therapy, CAR-76 therapy, CAR-Macrophage therapy or any other cellular therapy with a genetically modified or genetically unmodified immune cell type.
